SEQUENCE LISTING (264 sequences)

SEQ ID NO: 1 - protein, 545 aa, Streptococcus pneumoniae
MSIFIGGAWPYANGSLHIGHAAALLPGDILARYYRQKGEEVLYVSGSDCNGTPISIRAKKENKSVKEIADFYHKEFKETFEKLGFTYDLYSRTDSPLHHEIVQELFLQLYEKKFLYTKKIKQLYCTFDNQFLPDRFVEGKCPNCGTHSRGDQCDNCSAILDPIDLVDKRCSICSNEPEVRETEHFYYVFSEFQNLLETYLNDAEETVRWRKNAINLTKRYLREGLPDRAVTRDLPNGIPVPIDGFRDKKIYVWFEAVAGYYTASVDWAQKLQNNITDFWNNRTKSYYVHGKDNIPFHTIIWPAILSGLEIEPLPEYIISSEYLTLENKKISTSNNWAIWLNDIIKKYDADSIRYFLTINAPEMKDANFSWREFIYSHNSELLGSYGNFINRTLKFIEKYFESEIPTKYLEGEILYNLKELYTTVGNLVESGHMKQALEEIFEYIRSANKFYDDMKPWALRESDIEKCKEVLATCVIIILNLGQMLNPFIPFSGKKIEDMFKTKLNTWNYISNLPNKLSDVSMLFDRIDLKKIDEEVLELQQTSSR

SEQ ID NO: 2 - protein, 222 aa, Streptococcus pneumoniae
MEFMLDTLNLDEIKKWSEILPLAGVTSNPTIAKREGSINFFERIKDVRELIGSTPSIHVQVISQDFEGILKDAHEIRRQAGDDIFIKVPVTPAGLRAIKVLKKEGYHITATAIYTVIQGLLAIEAGADYLAPYYNRMENLNIDSNSVIRQLALAIDRQNSPSKILAASFKNVAQVNNALAAGAHAVTAGADVFESAFAMPSIQKAVDDFSDDWFVTQNSRSI

SEQ ID NO: 3 - protein, 222 aa, Streptococcus pneumoniae; identical to SEQ ID NO: 2

SEQ ID NO: 4 - protein, 255 aa, Streptococcus pneumoniae
LNNLTLLKEYNFRDLGNHLTQTGQKIKPKTLFRSSKLFGISKIDVDLLQSYGITKVIDFRSANEIKKAPDPDIKNIKNIVIPIFYNDDSELTEFPIEFFNKSDAGFQHMIKTYDQMINQKQSKLGYKKFFKLLLSHPKDESLLFHCSMGKDRTGIASLFLLYILGVDMNDIFHDYLLSNKYLINVRKENIEYVNNHSGNVILMHNLLSLSSAKEEYINRVLNVLDKEYGGILRYINTELGISSQEIEELKDRYLF

SEQ ID NO: 5 - protein, 104 aa, Streptococcus pneumoniae
MAKAITDATFEQETKDGLVLVDFWATWCGPCRMQGPILDKLSEELSEDVLKIVKMDVDENPNTARAFGIMSIPTLLFKKDGQVVKQVAGVHTAEQIKAIIAELS

SEQ ID NO: 6 - protein, 559 aa, Streptococcus pneumoniae
MKKIKPHGPLPSQTQLAYLGDELAAFIHFGPNTFYDQEWGTGQEDPERFNPSQLDAREWVRVLKETGFKKLILVVKHHDGFVLYPTAHTDYSVKVSPWRRGKGDLLLEVSQAATEFDMDMGVYLSPWDAHSPLYHVDREADYNAYYLAQLKEILSNPNYGNAGKFAEVWMDGARGEGAQKVNYEFEKWFETIRDLQGACLIFSTEGTSIRWIGNERGYAGDPLWQKVNPDKLGTEAELNYLQHGDPSGTIFSIGEADVSIRPGWFYHEDQDPKSLEELVEIYFHSVGRGTPLLLNIPPNQAGLFDAKDIERLYEFATYRNELYKEDLALGAEVSGPALSADFACRHLTDGLETSSWANDAALPIQLELDLGSPKTFDVIELREDLKLGQRIAAFHVQVEVDGVWQEFGSGHTVGYKRLLRGAVVEAQKIRVVITESQVLPLLTKISLYKTPRLSQTVAVQGLAFAEKSLAVAKGETLHFRIERSESSSSLEAKISIQPGTGVHGVAYQDEIYVLAFQAGETEKSLTLPTLYFAGDKTLDFYLNLTVDGQLVDQLQVQVS

SEQ ID NO: 7 - protein, 503 aa, Streptococcus pneumoniae
MSEKAKKGFKMPSSYTVLLIIIAIMAVLTWFIPAGAFIEGIYETQPQNPQGIWDVLMAPIRAMLGTHPEEGSLIKETSAAIDVAFFILMVGGFLGIVNKTGALDVGIASIVKKYKGREKMLILVLMPLFALGGTTYGMGEETMAFYPLLVPVMMAVGFDSLTGVAIILLGSQIGCLASTLNPFATGIASATAGVGTGDGIVLRLIFWVTLTALSTWFVYRYADKIQKDPTKSLVYSTRKEDLKHFNVEESSSVESTLSSKQKSVLFLFVLTFILMVLSFIPWTDLGVTIFDDFNAWLTGLPVIGNIVGSSTSALGTWYFPEGAMLFAFMGILIGVIYGLKEDKIISSFMNGAADLLSVALIVAIARGIQVIMNDGMITDTILNWGKEGLSGLSSQVFIVVTYIFYLPMSFLIPSSSGLASATMGIMAPLGEFVNVRPSLIITAYQSASGVLNLIAPTSGIVMGALALGRINIGTWWKFMGKLVVAIIVVTIALLLLGTFLPFL

SEQ ID NO: 8 - protein, 191 aa, Streptococcus pneumoniae
MYHIKADKRSLASARLISQGLNRLLQAKDFDSITVTDIVNEAGVGRATFYRLFDNVTDVITYSYYIIAHPTLEKIQNKFISNILDFSKSLINDFYSQDSILLISTLLFSVQNGYHFLSMLNLYKNDLLVLLKNEKNLKTKSQDYKYLMEIYLMTLMVTVGSYIQKPEITRELLLEKVSNVWQDLTHLHINN

SEQ ID NO: 9 - protein, 378 aa, Streptococcus pneumoniae
MKHSVHFGAGNIGRGFIGEILFKNGFHIDFVDVNNQIIHALNEKGKYEIEIAQKGQSRIEVTNVAGINSKEHPEQVIEAIQKTDIITTAIGPNILPFIAELLAKGIEARRVAGNTQVLDVMACENMIGGSQFLYQEVKKYLSPEGLTFADNYIGFPNAAVDRIVPAQSHEDSLFVVVEPFNEWVVETKRLKNPDLRLKDVHYEEDLEPFIERKLFSVNSGHATSAYIGAHYGAKTILEALQNPNIKSRIESVLAEIRSLLIAKWNFDKKELENYHKVIIERFENPFIVDEVSRVARTPIRKLGYNERFIRPIRELKELSLSYKNLLKTVGYAFDYRDVNDEESIRLGELLAKQSVKDVVIQVTGLDDQELIEQIVEYI

SEQ ID NO: 10 - protein, 378 aa, Streptococcus pneumoniae; identical to SEQ ID NO: 9

SEQ ID NO: 11 - protein, 489 aa, Streptococcus pneumoniae
MKGLWSNNLTCGYDEKIILENINIKIPEEKISVIIGSNGCGKSTLIKTLSRLIKPLEGEVLLDNKSINSYKEKDLAKHIAILPQSPIIPESITVADLVSRGRFPYRKPFKSLGKDDLEIINRSMVKANVEDLANNLVEELSGGQRQRVWIALALAQDTSILLLDEPTTYLDISYQIELLDLLTDLNQKYKTTICMILHDINLTARYADYLFAIKEGKLVAEGKPEMKGLWSNNLTCGYDEKIILENINIKIPEEKISVIIGSNGCGKSTLIKTLSRLIKPLEGEVLLDNKSINSYKEKDLAKHIAILPQSPIIPESITVADLVSRGRFPYRKPFKSLGKDDLEIINRSMVKANVEDLANNLVEELSGGQRQRVWIALALAQDTSILLLDEPTTYLDISYQIELLDLLTDLNQKYKTTICMILHDINLTARYADYLFAIKEGKLVAEGKPEDILNDKLVKDIFNLEAKIIRDPISNSPLMIPIGKHHVNS

SEQ ID NO: 12 - protein, 528 aa, Streptococcus pneumoniae
MKAFAIYNDYEIVGEYEDAGKSGKSIEGRIQFNRMMEDIKSGKDGVSFVLVFKLSRFARNAADVLSTLQIMQDYGVNLICVEDGIDSSKDAGKLMISVLSAVAEIERENIRIQTMEGCIQKAREGKWNGGFAPYGYKLEDGKLFINEEEAVAIRTIFDQYVNTTIGANGISKYLENHGIRKIPRQNGKNPLFDAGLIRKILKNPVYHGKIAFGRRTLEKVHGTRNEYKQVEQDEYLISEGIHEAIVSDEVWQAAQVKLKSQAKKYEHVNKGKDTRTHLLSGIVKCPICGVGMFGNKCIKKKKDGTKYKDFYYYGCKHRQMIRGHKCTFSKQIREELLDDAVAEVIVKIVSNPKFASMMQEKINMKVDTSEIEKEIDNYQKELRKSHSTKFKLIEEIDNLDVEDKHYKRRKQDLDDRLYRMYDKIDELESSLIDAKAKKQTIEAEKLTGDNIYKVLIYFDKLYKVMNDVERRQLISALISEIQVYEEKQSNGQWLKSITFKLPIIEENLNIDLDNDEQVECVSLLEKRS

SEQ ID NO: 13 - protein, 261 aa, Streptococcus pneumoniae
MKKYQRMHLIFIRQYLKQIMEYKADFLVGVVGVFLTQGLNMLFLNILFQHIPLLDGWSFHQVAFIYGFSLIPKGIDHLFFDNLWALGQHLIRKGEFDKYLTRPISPLFHILVETFQIDALGELLVGVLLLLMTITSLTWTWAKVFLFLISIPFATLIYTSLKIVTASIAFWTKQSGAIIYIFYMFNDFAKYPIAIYHSFLRWLISFIIPFAFTAYYPASYFLKDKDGLFNIGGLILISLIFFTLSLKLWNKGLDAYESAGS

SEQ ID NO: 14 - protein, 272 aa, Streptococcus pneumoniae
MKIVIAPDSFKESLTAQQVAEAIKRGFQQSIADVEYLLCPVGDGGEGTVDAIRHSLDLKEKWIQVTDPFGQKEAMCYFQKGELALFEVADLVGLGKIPLEKRNPLQIQTCGIGELILHLISKGIKDIYIGVGSTASNDGGLGIAAGLGYQFYDRDGNVLPASSQSLLNLASVSTENCYKIPEGVQIHILADVVSPLCGHQGATYTFGNQKGLHPTMFAVVDQAIQDFYEKFSPATLEIKGAGAGGGLAGGLCAFAQASIVSGIDTLLGLNQL

SEQ ID NO: 15 - protein, 1058 aa, Streptococcus pneumoniae
MPKRTDIQKIMVIGSGPIIIGQAAEFDYAGTQACLSLKEEGYEVVLVNSNPATIMTDKEIADKVYIEPITLEFVTRILRKEGPDALLPTLGGQTGLNMAMELSKNGILDELGVELLGTKLSAIDQAEDRDLFKQLMEELEQPIPESEIVNTVEEAVAFAATIGYPVIVRPAFTLGGTGGGMCANEKELREITENGLKLSPVTQCLIERSIAGFKEIEYEVMRDSADNALVVCNMENFDPVGIHTGDSIVFAPAQTMSDYENQMLRDASLSIIRALKIEGGCNVQLALDPNSFKYYVIEVNPRVSRSSALASKATGYPIAKLAAKIAVGLTLDEVINPVTGSTYAMFEPALDYVVAKIPRFPFDKFEKGERRLGTQMKATGEVMAIGRNIEESLLKACRSLEIGVHHNEIPELAAVSDDTLIEKVVKAQDDRLFYVSEAIRRGYTPEEIAELTKIDIFYLDKLLHIFEIEQELGAHPQDLEVLKTAKLNGFSDRKIAELWGTTDDKVRQLRLENKIVPVYKMVDTCAAEFDSETPYFYSTYGWENESIKSDKESVLVLGSGPIRIGQGVEFDYATVHSVKAIQAAGYEAIIMNSNPETVSTDFSVSDKLYFEPLTFEDVMNVIDLEQPKGVIVQFGGQTAINLAEPLAKAGVTILGTQVADLDRAEDRDLFEQALKELDIPQPPGQTATNEEEAALAARKIGFPVLVRPSYVLGGRAMEIVENEEDLRSYMRTAVKASPDHPVLVDSYIVGQECEVDAISDGKNVLIPGIMEHIERAGVHSGDSMAVYPPQTLSQKVQETIADYTKRLAIGLHCLGMMNIQFVIKDEKVYVIEVNPRASRTVPFLSKVTNIPMAQVATKLILGQSLSELGYQNGLYPESTRVHIKAPVFSFTKLAKVDSLLGPEMKSTGEVMGSDATLEKALYKAFEASYLHLPTFGNVVFTIADDAKEEALNLARRFQNIGYGILATEGTAAFFASHGLQAQPVGKIGDDDKDIPSFVRKGRIQAIINTVGTKRTADEDGEQIRRSAIEHGVPLFTALDTANAMLKVLESRSFVTEAI

SEQ ID NO: 16 - protein, 307 aa, Streptococcus pneumoniae
MSENQQALNHVVSMEDLTVDQVMKLIKRGIEFKNGAQLPYEDHPIVSNLFFEDSTRTHKSFEVAEIKLGLERLDFDVKTSSVNKGETLYDTILTLSALGVDVCVIRHPEVDYYRELIASPTITTSIINGGDGSGQHPSQSLLDLMTIYEEFGHFEGLKVAIAGDLDHSRVAKSNMQILKRLGSELFFAGPEEWRSQEFADYGQFVTIDEIIDQVDVMMFLRVQHERHDSGAVFSKEDYHAQHGLTQERYDRLKETAILMHPAPINRDVEIADHLVEAPKSRIVQQMTNGVFVRMAILESVLASRNAN

SEQ ID NO: 17 - protein, 250 aa, Streptococcus pneumoniae
MTIHIIITMLLLLAFLIGSIWFAKKKYQINLAVLGLGAVAFFVSSQILEKLVHILILHPQKDGSIALLQDHPLIYIIYGLAMAAFFEETARLVFFKWLEKKRSLEKADALAYGLGHGGLELIFLGLTSLLNLYIVLSAVQTQNPQVMQLLSENMLKTIQSLSVWQIYLLGFERILALGFQLLLTVWVYQAVRQKKWIYLLAAYGLHAFFDLAPSLFQVGWLTNPVLVEVILALELVLVAYGTKEIFCKKS

SEQ ID NO: 18 - protein, 803 aa, Streptococcus pneumoniae
MIRNKKQDYVLAYKQPASTTYMSWEEEALPIGNGSLGAKVFGLIGAERIQFNEKSLWSGGPLPDSSDYQGGNLQDQYVFLAEIRQALEKRDYNLAKELAEQHLIGPKTSQYGTYLSFGDIHIEFSQQGTTLSQVTDYQRQLNISKALVTTSYVYKGTRFEREAFASFPDDLLVQRFTKEGAETLDFTIELSLTCDLASDEKYEQEKSDYKECKLDITDSHILMKGRVKDNDLRFASYLAWETDGDIRVWSDRVQISGASYANLFLAAKTDFAQNPASNYRKKLDLEQQVIDLVDTAKEKGYTQLKSRHIEDYQALFQRVQLDLEADVDASTTDDLLKNYKPQEGQALEELFFQYGRYLLISSSRDCPDALPANLQGVWNAVDNPPWNSDYHLNVNLQMNYWPAYVTNLLEAVFPVINYVDDLRVYGRLAAVKYAGIVSQKGEENGWLVHTQATPFGWTAPGWDYYWGWSPAANAWMMQTVYEAYSFYRDQDYLREKIYPMLRETVRFWNAFLHKDQQAQRWVSSPSYSPEHGPISIGNTYDQSLIWQLFHDFIQAAQELGLDEDLLTEVKEKSDLLNPLQITQSGRIREWYEEEEQYFQNEKVEAQHRHASHLVGLYSGNLFSYKGQEYIEAARASLNDRGDGGTGWSKANKINLWARLGDGNRAHKLLAEQLKTSTLPNLWCSHPPFQIDGNFGATSGMAEMLLQSHAAYLVPLAALPDAWSTGSVSGLMARGHFEVSMSWEDKKLLQLTILSRSGGDLRVSYPDIEKSVIKMNQEKIKAKCMGKDCISVATAEGDLVQFYF

SEQ ID NO: 19 - protein, 583 aa, Streptococcus pneumoniae
MQNKQEQWAVLKRLMSYLKPYGLLTFLALSFLLATTVIKSVIPLVASHFIDQYLSNLNQLAVTVLLVYYGLYILQTVVQYVGNLLFARVSYSIVRDIRRDAFANMEKLGMSYFDKTPAGSIVSRLTNDTETISDMFSGILSSFISAVFIFLTTLYTMLVLDFRLTALVLLFLPLIFLLVNLYRKKSVKIIEKTRSLLSDINSKLAENIEGIRIIQAFNQEKRLQAEFDEINQEHLVYANRSVALDALFLRPAMSLLKLLGYAVLMAYFGYRGFSIGITVGTMYAFIQYINRLFDPLIEVTQNFSTLQTAMVSAGRVFALIDERTYEPLQENGQAKVQEGNIRFEHVCFSYDGKHPILDDISFSVNKGETIAFVGHTGSGKSSIINVLMRFYEFQSGRVLLDDVDIRDFSQEELRKNIGLVLQEPFLYHGTIKSNIAMYQEISDDQVQAAAAFVDADSFIQELPQGYDSPVSERGSSFSTGQRQLLAFARTVASQPKILILDEATANIDSETESLVQASLAKMRQGRTTIAIAHRLSTIQDANCIYVLDKGRIIESGTHEELLTLGGTYHKMYSLQAGAMAYTL

SEQ ID NO: 20 - protein, 152 aa, Streptococcus pneumoniae
MTPEEMYLTERLDVQIAHFLKKSVQHRRRYKVLKITEIVAGFLIAVFCAIPMPGDRYRLISVALSSLGLLCEGIINLYNAKENWISYQKTAQLLEKEKFLYQCQTEKYAGKTKAFALFVKTCEGLISEEINQWESIQSKEVAASADAPVKKE

SEQ ID NO: 21 - protein, 255 aa, Streptococcus pneumoniae
MSQKLNHLDVGEFVLLLPEHLRSEEEHYKSVFEDDLTSRMSSQDERQQMTATVGYLESGQDRFVYNTTPISYQQFLKDPIIIVITPQSTGPQSILFWIDAVQNYVLFNQLSDAQELIQRQGIENWVSEMQTGYHNYITLLDNIQRERWVMLAGAVLGIATSILLFNTMNRLYFEEFRRAIFIKRIAGLRFLEIHRTYLFAQLGVFLLGFVASVFLQVEVGVAFLVLLLFTGLSLLQLHVQMQKENKMSMLVLKGG

SEQ ID NO: 22 - protein, 316 aa, Streptococcus pneumoniae
MGKKRWARNGSESNDASYAQVVSLYEDTSISVSNNETDKVLAGSLYTDTNEQGLTIPSSLLKNWNEQTGKNLTANDLIGKSVSASIVESAAEASKIAQFQTKIVRVINDEDDMEDSNSFMLSYQMETILKEAGFTKAVSYFILELKDPSQTKVVTEELQKNKKYTVLSQQRVLDIVITFIRVIQGLLIVLSSQAIVVAAVMIGIIIYINIMQRSKEIGVMKAVGYQNRDVKGIFIYEAIWIVGIALLLAFLVAQGVGSLANAIVSHFYPSITKVFELNLLSVLGTLVFALLLGYVSAYFPARKISKMDPVESLRYE

SEQ ID NO: 23 - protein, 282 aa, Streptococcus pneumoniae
MNTNLKPKLQRFASATAFACPICQENLTLLETNFKCCNRHSFDLAKFGYVNLAPQIKQSANYNKENFQNRQQILEAGFYQAILDAVSDLLASSKTTTTILDIGCGEGFYSRKLQESHSEKTFYAFDISKDSVQIAAKSEPNWAVNWFVGDLARLPIKDANMDILLDIFSPANYGEFRRVLSKDGILIKVIPTENHLKEIRQRVQDQLTNKEYSNQDIKEHFQEHFTILSSQTASLTKTITAEQLQALLSMTPLLFHVDQSKIDWSQLTEITIEAEILVGKAF

SEQ ID NO: 24 - protein, 667 aa, Streptococcus pneumoniae
MKPLLETIDTRFGTASKHAFSRGNTLPYTGVPFGMNYFVPQTSDQDGSCFFDPHLPIFQGIRLTHQPSPWIGDYSWLLLTPVTSQLGGDSLFHRQSSYDRDKASFQPHYLKIFSLRYQIETQLTPTCYGASIRLEQKQGKTLSLYLHVADELTVEQVDKRTLALRQEGKTETNKNSLTMFTALQMNTDILAISQEAGDWRIDLASSQTEMQLATSFISPSQALINLPQEDFDSCKASAQADWENLLHRFDIIETGEADRTFFDHCLYRLFLFPQTFYEVDESGQAIHMDLATGTVKPGVLFSNNGFWDTFRTTFPLFALIIPEHYQRFLEGFLNSYRDTGFLPKWLAPDERGMMPGTLLDGIIADSACKDMAPDLEGELFQAMLKTASKADPLGINGRHGLAQYQELGYLSTDHHESVSHTLDYAYSDFCIANCAKKLKNIEIAETYKAASQNYRQLFDAETGYMRARDNQGNFHPDFSPYSWGRDYAECSAIQATLGVLHDIPGLIQLMGGKETFSNYLLKACQDAPLFETTGYGYEIHEMSEMATAPFGQIAISNQPSFHIPYLFRYSDYPDYTALLIKTLRQKAFHPSWEAYPGDEDNGSLSAWYIWSALGFYPTCPGKPSYDLGIPLFDHLRVYLAKENKWLDIHTEQNHSHFNFVKECRLDK

SEQ ID NO: 25 - protein, 409 aa, Streptococcus pneumoniae
MDFFLLVDLILYFLIISHSYRLICKDQINRKELFFFGAYTLLTEIVLDFPFYLLYLDGLGIATFLFPLGLYSYFRWMKQYERDRGLFLSLLLSLLYESTHNFLSVTFSSITGDNFVLQYYGLFFFVVTVLTYFVTLKIIYYFHLELAYFDKDYLYPFLKKVFFALLLLHIVSFVSDMVSTIKHLNSFGSILSSIVFISLLLTFFAMNSHKVQMEKEIALKQKKFEQKHLQNYTDEIVGLYNEIRGFRHDYAGMLVSMQMAIDSGNLQEIDRVYNEVLVKANHKLRSDKYTYFDLNNIEDSALRSLVAQSIVYARNNGVEFTLEVKDTITKLPIELLDLVRIMSVLLNNAVEGSADSYKKQMEVAVIKMETETVIVIQNSCKMTMTPSGDLFALGFSTKGRNRGVGLNNV

SEQ ID NO: 26 - protein, 325 aa, Streptococcus pneumoniae
MKNKRYFFDTILIILLLISTIFCVSPVFIKLDILGTPSHAILTFVLAIPLFYILSQCLHTLLLLVSSIFCKLRPIYFYFIFVIIIGARKYYRILFHQLMGFSPGIAVFYKESQTTKNLFKFYYFLYFTTLISYYFFFTFVYDKPLLLPLISFSIIIALVQKLYRIENQQLFLLKSKVLTILESKKNCEFNLQDYHEIWKLQSKSELPCVALSYISLIKPYLSESVREQIDLLEVKRFKKINHPISLYGMLDVIKLNLYLRHYNEKNKYESMLKKILEVRPDFVLIEQNIDDSLNSSQPLSLSLAISEIQLLLEVYMGIKHVSIRR

SEQ ID NO: 27 - protein, 699 aa, Streptococcus pneumoniae
MNKKKMILTSLASVAILGAGFVTSQPTFVRAEEAPVASQSKAEKDYDAAMKKSEAAKKAYEEAKKKAEDAQKKYDEGQKKTEEKARKAEEASKEIAKATSEVQNAYVKYQRVQRNSRLNEKERKKQLAEIDEEINKAKQILNEKNEDFKKVREEVIPEPTELAKDQRKAEEAKAEEKVAKRKYDYATLKVALAKSKVEAEEAELDNKAENLQNKVADLEKEIANAEKTVADLEKEVAKLEKDVEDFKNSNGEQAEQYLAAAEKDLVAKKAELAEAKIKAATKKAELEKAEAELENLLSTLDPEGKTQDELDKEAAEAELNKKVEALQNQVAELEEELSKLEDNLKDAETNNVEDYIKEGLEEAIATKKAELEKTQKELDAALNELGPDGDEEETPAPAPQPEKPAPAPAPKPEQPAPAPKPEQPAPAPKPEKSADQQAEEDYARRSEEEYNRLTQQQPPKAEKPAPAPAPKPEQPAPAPKTGWKQENGMWYFYNTDGSMATGWLQNNGSWYYLNSNGAMATGWLQYNGSWYYLNANGAMATGWAKVNGSWYYLNANGSMATGWVKDGDTWYYLEASGAMKASQWFKVSDKWYYVNSNGAMATGWLQYNGSWYYLNANGAMATGWAKVNGSWYYLNANGSMATGWVKDGDTWYYLEASGAMKASQWFKVSDKWYYVNGSGSLAVNTTVDGYTVNENGEWV

SEQ ID NO: 28 - protein, 185 aa, Streptococcus pneumoniae
MDKMKPVFQALNKELIQENLTLTIICVGGYVLEYHGLRATQDVDAFYDQNQKINEIIARVGKQFNLNTHEELWLNNHVANMNKQPPLSLCESLYSFENLTVLVVPIEYVLGMKMMSIREQDLKDIGAIIKYKNFHSPFDTFKYLKDMGFDTIDLSVLLEGFSYAYGMDWLEKFFKENQDKLREFY

SEQ ID NO: 29 - protein, 449 aa, Streptococcus pneumoniae
MKIILVGGGKVGFALCRSLVAEKHDVLLIEQDEAVLNHIVSRFDIIGILGNGADFAILEQASVQDCDIFIALTEHDEVNMIAAVLAKKMGAKETIVRVRNPEYSNSYFKEKNILGFSLIVNPELLAARAIANIIDFPNALSVERFAGGRVSLMEFVVKSTSGLCQMPISDFRKKFGNVIVCAIERDHQIIIPSGDMTVQDKDRIFVTGNRVDMILFHNYFKSRAVKSLLIVGAGRIAYYLLGILKDSRIDTKVIEINPEIASFFSEKFPNLYIVQGDGTAKDILLEESAQHYDAVATLTGVDEENLITSMFLDRVGVQKNITKVNRTSLLEIINAPDFSSIITPKSIAVDTIMHFIRGRVNAQYSDLQAMHHLANGQIETLQFHIKEANKMTAKPLSQLKLKKGVLIAAIIRKGKTIFPTGEDMLEVGDKLLVTTLLPNITKIYDLIAR

SEQ ID NO: 30 - protein, 254 aa, Streptococcus pneumoniae
MALLEVKQLTKHFGGLTAVGDVTLELNEGELVGLIGPNGAGKTTLFNLLTGVYEPSEGTVTLDGHLLNGKSPYKIASLGLGRTFQNIRLFKDLTVLDNVLIAFGNHHKQHVFTSFLRLPAFYKSEKELKDKALELLKIFDLDGDAETLAKNLSYGQQRRLEIVRALATEPKILFLDEPAAGMNPQETAELTELIRRIKDEFKITIMLIEHDMNLVMEVTERIYVLEYGRLIAQGTPDEIKTNKRVIEAYLGGEA

SEQ ID NO: 31 - protein, 328 aa, Streptococcus pneumoniae
MMDSPKKLGYHMPAEYEPHHGTLMIWPTRPGSWPFQGKAAKRAFTQIIETIAEGERVYLLVEQAYLSEAQSYLGDKVVYLDIPTNDAWARDTGPTILVNDKGKKLAVDWAFNAWGGTYDGLYQDYEEDDQVASRFAEALERPVYDAKPFVLEGGAIHSDGQGTILVTESCLLSPGRNPNLTKEEIENTLLESLGAEKVIWLPYGIYQDETNEHVDNVAAFVGPAELVLAWTDDENDPQYAMSKADLELLEQETDAKGCHFTIHKLPIPAVRQVVTEEDLPGYIYEEGEEKRYAGERLAASYVNFYIANKAVLVPQFEDVNDQVALDIL

SEQ ID NO: 32 - protein, 746 aa, Streptococcus pneumoniae
MLQWIKNFSIPLIYLSFLLLWLYYAIFSASYLALLGFVFLLVCLFIQFPWKSAGKVLIICGIFGFWFVFQNWQQSQASQNLADSVERVRILPDTIKVNGDSLSFRGKSNGRAFQVYYKLQSEEEKEAFQALTDLHEIGLEGKLSEPEGQRNFGGFNYQAYLKTQGIYQTLNIKTIQSLQKIGSWDIGENLSSLRRKAVVWIKTHFPDPMGNYMTGLLLGHLDTDFEEMNELYSSLGIIHLFALSGMQVGFFMNGFKKLLLRLGLTQEKLKWLTYPFSLIYAGLTGFSASVIRSLLQKLLAQHGVKGLDNFALTVLVLFIVMPNFFLTAGGVLSCAYAFILTMTSKEGEGLKAVTSESLVISLGILPILSFYFAEFQPWSILLTFVFSFLFDLVFLPLLSILFVLSFLYPVIQLNFIFEWLEGIIRLVSQVARRPLVFGQPNAWLLILLLISLALVYDLRKNIKGLTVLSLLITGLFFLTKYPLENEITMLDVGQGESIFLRDVTGKTILIDVGGKAESYKKIKKWQEKMTTSNAQRTLIPYLKSRGVAKIDQLILTNTDKEHVGDLSEMTKAFHVGEILVSKDSLKQKEFVAELQATQTKVRSMIVGENLPIFGSQLEVLSPRKMGDGGHDDTLVLYGKFLDKQFLFTGNLEEKGEKDLLKHYPDLKVNVLKASQHGNKKSSSPAFLEKLKPELTLISVGKSNRMKLPHQETLTRLEGINSKVYRTDQQGAIRFKGLDSWKIESVR

SEQ ID NO: 33 - protein, 166 aa, Streptococcus pneumoniae
MYYKQQISEVMFNLLDSHDTERILWTANEDVQLVKSALAFFFLQKGTPCIYYGTELALTGGPDPDCRRCMPWERVSSDNDMLNFMKRLIKIRKYASVIISHGKYSLQEIKSDLVALEWKYEGRILKVIFNQSTEDYLLEKEAVALASNCQELENQLVISPDGFVIF

SEQ ID NO: 34 - protein, 253 aa, Streptococcus pneumoniae
MEIQDYTDSEFKHALARNLRSLTRGKKSSKQPIAILLGGQSGAGKTTIHRIKQKEFQGNIVIIDGDSFRSQHPHYLELQQEYGKDSVEYTKDFAGKMVESLVTKLSSLRYNLLIEGTLRTVDVPKKTAQLLKNKGYEVQLALIATKPELSYLSTLIRYEELYIINPNQARATPKEHHDFIVNHLVDNTRQLEELAIFERIQIYQRDRSCVYDSKENTTSAADVLQELLFGEWSQVEKEMLQVGEKRLNELLEK

SEQ ID NO: 35 - protein, 186 aa, Streptococcus pneumoniae
MTKLWKRYKPFVSAGIQELITYRVNFFLYRIGDVMGAFVAFYLWKAVFDSSHQSLIQGFTLSDMTLYIIMSFVTNLLTKSDSSFMIGWEVKDGSIIMRLLRPVHFAMSYLFTEIGSRWLVFVSVGLPFVILIAGLKLLSGESFLQIVLITTVYLLSLILAFLINFFSIFALVFQLLCLKTYGDQIF

SEQ ID NO: 36 - protein, 759 aa, Streptococcus pneumoniae
MYNYPMRIHYHRKNGEYDTCSFVKSQDQRIDLLTYKEDYFGALFSFEHPSSHVIESLNFVVHTGQTSKEYSIRFNHYPLLTEVWILEGDDRIYYSENPAIASPFYKNQNPFAFDKAINSASFDHHWGYQGELGCRVEDNQAHFSLWSPTATEVQVVVYESAANDAPVWKTFEMKRGNSYSYNHKENTIGVWSLDVEENLVGKTYQYQVQFPHHQTLTRDPYTIATSPDGKRSAILSHVEKQVENFEVKHGSEATWRLENPCKAVICEMHIRDLTKSPTSGVDEHLRGTFLGAAQAGTVNQYGQSTAFDYIKKLGYNYVQLQPIADRHKEYDEDGNVTYNWGYDPQNYNAPETSFSTNPDDPAQVIRDLKVMVQAYHDAGIGVIMDVVYNHTFSVVDAPFQTTVPDYYYRMNPDGTFQNGTGVGNETASEHEMFRKYMIDSLLYWVQEYNIDGFRFDLMGIHDVKTMQMIRQSLDEIDSNIILYGEGWDMGTGLAPYDKAKKDNAYQMPNIGFFNDNQRDAVKGGEVYGAIKSGFVSGAATEPILAKAILGSRELGSYTHPNQVLNYVEAHDNYNLHDLLATLHPDQSSEQIMRKVETATAMNLLMQGMAFMEIGQEFGRTKLVATGENGELTHDDRERAMNSYNAPDSVNQVNWNLINERQDSIEFIRQVIRLKTKTGAFSYSSYDEIYHHVFVHSAIEHSGCLIYEVHGKEHLLVVVNAKSEPYQFENAGNLAMLVTNSRSKEDNVLNDISLAVLSVL

SEQ ID NO: 37 - protein, 474 aa, Streptococcus pneumoniae
LTRYQNLVNGKWKSSEQEITIYSPINQEELGTVPAMTQTEADEAMQAARAALPAWRALSAVERAAYLHKTAAILERDKEEIGTILAKEVAKGIKAAIGEVVRTADLIRYAAEEGLRITGQAMEGGGFEAASKNKLAVVRREPVGIVLAIAPFNYPVNLSASKIAPALIAGNVVMFKPPTQGSISGLLLAKAFEEAGIPAGVFNTITGRGSEIGDYIIEHKEVNFINFTGSTPIGERIGRLAGMRPIMLELGGKDAALVLEDADLEHAAKQIVAGAFSYSGQRCTAIKRVIVLESVADKLATLLQEEVSKLTVGDPFDNADITPVIDNASADFIWSLIEDAQEKEAQALTPIKREGNLLWPVLFDQVTKDMKVAWEEPFGPVLPIIRVASVEEAIAFANESEFGLQSSVFTNDFKKAFEIAEKLEVGTVHINNKTQRGPDNFPFLGVKGSGAGVQGIKYSIEAMTNVKSIVFDVK

SEQ ID NO: 38 - protein, 591 aa, Streptococcus pneumoniae
LANNRKTETLGVSYLSTFIDKHELLQSYFESNDKTPVWDGEIHVLKSPSEKKDEILGKVPVQIKTTRQKKDVLKSFSLDTRDLELYKPNGGVVLFVVWLNEDNGLRDIYYKSLPPLSIKNLLKKSKLKNKSTNRKKLSIEIFKLDEKKMYPMLVDFINNSQKQYSFINVEGISVEDIPDDKTLKFYFYGQEKEEIFNYQEEHDLFIYYLDPITGIEIPLENTIKIVETEEETDLIIKIGDYVFQDVKRHRFPDGSVQLHFGESFTMSFDIKKKQFKFNYTRPDLLSKAIKCTQVFQELGKIGYFTLNGNKIELDERSIKDISSLDLEADIKGLLKISNFMKKMGIQKDVDLSCFDKQSQRNLNILYSGLVLKKKVALNYNESKLLHLNIANIHIITLYSFLSDKNGTMIDIFTETPWCREGETEDEDYLDISIFEVFEPNDWLKIDNCKIDSVIASYQRLVDNKLKYEGADRTILKIVIAADMAEDKTKRELLLNWAQCLSDWNLKYSKNCEMAIINDLQIKSRVRKLNSKETETLTNILVNSNDNYELCFGSSVLLKSKPQADLFWNKLDNETKERYKDFPIYTLYMKLS

SEQ ID NO: 39 - protein, 406 aa, Streptococcus pneumoniae
MKKILLSTVALLSLVASLLANNPVSAQESSSQATYSKSSGSWIKSGNRWWYKHSDGSYTTNGWEKINGTWYYFDSEGWMKTGWIKEYGKWYYLDDSGAMKTGWCLVSGSWYYLNSSGVMQTGLQTINGKQYYLAAGGAMQTGWHNIGDDTYFFANSGENQNINRRALVLGETSTRAVPIADVNAMEKVFNNQNFSEVVRFPDRTKSEIIAKMQELFESSSEGDVNYLYFTCHGGRDGRIYIGSDGLAFSGWELASVLKQYKGKFVVMLDCCHAGTIISKDNTGEGNEGASTEYFDLDEFVSGFSNMDGNEKSGEMIDSKFLVLCSSRGAEYSSGGSLSLATKYWSLGSGWNPLQNSQAYLAADQNNNRRITLNELYTYSREQVLKQNSNQHIEVYPDNSQFVLFKK

SEQ ID NO: 40 - protein, 228 aa, Streptococcus pneumoniae
METKKIKNLKGQIIVSCQALEGEPLYTPNGGVMPLLAKAAFQAGAKGIRANSVRDISEIKEEVDLPIIGIIKRDYDGFEPFISATMKEIDELVSEGVDILALDCTNRSRPGYDNITDFIHDIKVKYPNQLLMADISTFEEGKVAAESGVDFVGTTLSGYTPYSPKKDNPDFELVERLVKELDVPVIAEGRISTPEQARKMLDLGAYAVVVGGAITRPLEIAKKFIEVV

SEQ ID NO: 41 - protein, 173 aa, Streptococcus pneumoniae
MKFKKIVTSALVVLSLFSIMSPSIASVRVFADDVTTTEVSEISTQEQKQIDDVANVLEQMFRNGVNEKNFTEYVYKNFSQKDIALAENELETNINNPYDRVPWDEMGGCIAGKIRDEFFAMINVSLIVKYAQKKAWSELAKVVLRFVKANGLKTNIYIIAGQLAIWAVQCGLE

SEQ ID NO: 42 - protein, 162 aa, Streptococcus pneumoniae
MKNRFYYYQLLDEREEQLFNKAGSESFYICIALSLLSYIISVLAPSLFNSNMLLIVIIIGTFYFFNRARYLGVTYYSRFHFTILGCFFLTLAITALLMLQNYQFNIEIYQHNPLNFKYLSAWVITYVIYLPWVFIGNLGLKSYGEWAQKKFEQDMDELESGE

SEQ ID NO: 43 - protein, 396 aa, Streptococcus pneumoniae
MTVLKGDNLEILKTIESSSIDLIYMDPPFFTQKTQKLSNNKNIMYSFEDTWTSIEDYKEFLSVRLEECKRVLKNSGSIFVHCDKIANHHIRLILDNIFGADMFQSEIIWNYKRWSNSKKGLLNNHQNIYFYSKSKDFKFNTIFTEYSSTTNIDQILVERKRDGNSKTIYKVDNNGNYILAKEKNGVPLSDVWNIPFLNPKAKERVGYPTQKPILLLEQIIKIATDKNDIVLDPFCGSGTTLVASKILNRNYMGIDLSEEAINITQQRLENVIKTSSNLLNKGIEAYRTKTEEEENILKLLQAKIVQRNKGIDGFLPKHFQKKPIPIKIQKNNECLNESISLLQNAINSKKLDFGVVIKTHSDNLLFDFDTIPENIIVVDHFELTIEKWLSKSQQLL

SEQ ID NO: 44 - protein, 241 aa, Streptococcus pneumoniae
MNVNQIVRIIPTLKANNRKLNETFYIETLGMKALLEESAFLSLGDQTGLEKLVLEEAPSMRTRKVEGRKKLARLIVKVENPLEIEGILSKTDSIHRLYKGQNGYAFEIFSPEDDLILIHAEDDRASLVEVGEKPEFQTDLASISLSKFEISMELHLPTDIESFLESSEIGASLDFIPAQGQDLTVDNTVTWDLSMLKFLVNELDIASLRQKFEFTEYFIPKSEKFFLGKDRNNVELWFEEV

SEQ ID NO: 45 - protein, 408 aa, Streptococcus pneumoniae
MKSNRYIIAFAGVILHLMLGSTYAWSVYRNPIIEKTGWDQASVAFAFSLAIFCLGLSAAFMGRLVEKFGPRVMGSLSAFLYAGGNILTGFAIDRQELWLLYLAYGILGGLGLGAGYITPVSTIIKWFPDKRGLATGLAIMGFGFASLLTSPIAQHLIAGVGLVETFYILGASYFIIMLLASQFIKRPNEQELAILSASGKEKTDSLTQGMTANQALKSNRFYILWIIFFINIACGLGLISAASPMAQEMAGLSTSHAAVMVGVLGIFNGFGRLLWASLSDYIGRPLTFSILLLVNLFFSLSLWLFTDSVLFVVAMSILMTCYGAGFSLIPAYLSDIFGTKELAALHGYILTAWAMAGLAGPILLAETYKMAHSYTQTLFVFLILYSIALALSYYLGRSIKKESQKPLT

SEQ ID NO: 46 - protein, 283 aa, Streptococcus pneumoniae
MDKPDIATIIDSHFEEMTDLEQEIARYFLQAETITDDLSSQQVTQKLHISQAALTRFAKKCGFTGYREFIFQYQHQAENQANQVSKHSPLTKRVLRSYSNMREQTQDLIDEIQLERIAQLIEDAERIYFFGTGSSGLVAREMKLRFMRLGVVCEALTDQDGFAWTTSIMDENCLVLGFSLSGSTPSILDSLLDAKEMGAKTVLFTSVPNKDSQTYTETVLVATHSQPSYIQRISAQLPMLFFIDLIYAYFLEINRESKEKIFNSYWENKKLNGYRRQKRVRKS

SEQ ID NO: 47 - protein, 279 aa, Streptococcus pneumoniae
MQSTEKKPLTAFTVISTIILLLLTVLFIFPFYWILTGAFKSQPDTIVIPPQWFPKMPTMENFQQLMVQNPALQWMWNSVFISLVTMFLVCATSSLAGYVLAKKRFYGQRILFAIFIAAMALPKQVVLVPLVRIVNFMGIHDTLWAVILPLIGWPFGVFLMKQFSENIPTELLESAKIDGCGEIRTFWSVAFPIVKPGFAALAIFTFINTWNDYFMQLVMLTSRNNLTISLGVATMQAEMATNYGLIMAGAALAAVPIVTVFLVFQKSFTQGITMGAVKG

SEQ ID NO: 48 - protein, 367 aa, Streptococcus pneumoniae
MVKYGVVGAGYFGAELARYMQKNDGAEITLLYDPDNAEAIAEELGAKVASSLDELVSSDEVDCVIVATPNNLHKEPVIKAAQHGKNVFCEKPIALSYQDCREMVDACKENNVTFMAGHIMNFFNGVHHAKELINQGVIGDVLYCHTARNGWEEQQPSVSWKKIREKSGGHLYHHIHELDCVQFLMGGMPETVTMTGGNVAHEGEHFGDEDDMIFVNMEFSNKRFALLEWGSAYRWGEHYVLIQGSKGAIRLDLFNCKGTLKLDGQESYFLIHESQEEDDDRTRIYHSTEMDGAIAYGKPGKRTPLWLSSVIDKEMRYLHEIMEGAPVSEEFAKLLTGEAALEAIATADACTQSMFEDRKVKLSEIVK

SEQ ID NO: 49 - protein, 210 aa, Streptococcus pneumoniae
MRLEIINGQKIYGKRPILNQLNLVFQSGKIYGLKGDNGSGKTVLLKILAGYIKLDKGKVLQDGKVYGVKNHYIQDAGILIEKVEFLSHLSLRENLELLRYFSSKVTEKRIAYWIQYYDLQEFEDIEYRHLSLGTKQKMALIQAFISSPSILFLDEPMNALDEKSVRITKQVILSYLKKENGLVILTSHISEDISDLCTDVLVVENGHIQM

SEQ ID NO: 50 - protein, 439 aa, Streptococcus pneumoniae
MGDKKYTVEKANRFIAENKHLVNTQYKPEEHFSAEIGWINDPNGFVYFRGEYHLFYQFYPYDSVWGPMHWGHAKSKDLVTWEHLPVALAPDQDYDRNGCFSGSAIVKDDRLWLMYTGHIEEETGVRQVQNMAFSDDGIHFEKIEQNPVATGSDLPDELIAADFRDPKLFEKDGRYYSVVAAKHKNNVGCIVLLGSDNLVEWQFESIFLKGVEHQGFMWECPDYFELDGKDCLIMSPMRYQREGDSYHNINSSLLFTGKVDWGEKCFIPESVQEIDHGQDFYAPQTLLDDQNRRILIAWMQTWGRTLPTHDQEHKWACAMTLPRILRLEDGKLRQFPVKKGQYQIQIDKDCHYHLGNDIDYLEFGYDSNAQQVYIDRSHLIQKILGEEEQDTSRRYVDIEAKELEVVLDKNSIEIFVNQGEASLTATYYLTVPAELSRID

SEQ ID NO: 51 - protein, 298 aa, Streptococcus pneumoniae
MKRLISANPSEILQMNAEELKQSILASEGRVVLSENVVTRETFVGDITNSEIARAFGADMILLNCVDVFEPKIYALDSSGDDVIHRLHQLVACPIGVNLEPIDPSAKMLEETQEIVAGRVASVETLNRIEELGFDFVCLTGNPGTGVSNQEIIKAVQSAKENFSGLIIAGKMHGAGVNEPVAELSVAEQLLEAGADVILVPAVGTVPAFHDQELREVVDLVHSKGGLVLSAIGTSQETSDTDTIKEIALRNKICGVDIQHIGDAGYGGLATVDNIYALSKAIRGVRHTVSRLARSVNR

SEQ ID NO: 52 - protein, 392 aa, Streptococcus pneumoniae
MTLSKKQLQLRAKILETVYTLGPISRIEIATKTGITPATTSSITNDLIKENILLELGEDEHDTSVGRKKILLDIQAKRFYYIGCELSEKHFTFALGDNLGNILKEEKEIVTKQLIQEKGNQLINQTLKQFLNNCSDYEIEAIGIALPGRYLDNYKITTNNPLWQHIDLEMIQSHFDKPLFFSNNVNCMAIGKRLFSRQQNDPNFAYFHFARGMHCSYIYDGNIYGKGNLMIGEIGHTVVSSEGEECSCGRKGCLQTFAGESWLIKKSKILYHQSPYSLLPSLVKNADDIDIQVILTAYQLGDTGIITLIHQALLYLSQTILNISMMIDSQKIYLHSPLLTNQHIIQKLYSEMNYKPKLLYNRLPEVIIEPYNDFTAAHSAIALCLYHTILHS

SEQ ID NO: 53 - protein, 213 aa, Streptococcus pneumoniae
MLRLYLENEIIELSNNLETIWVSVLDKYEKLFEYLSNEERIELIKEDLIINESVLNVDKKGYELICLQHPVSYDLRRIISVIKISTDIERIGDRIVEILKNLQIIQNNEILKKIISEIKILHEVIGLHMNRAISCYREEQSGCLDMVVIQKQNEIEELSINMEKKIMNYIFEDDGNVSEVISALDIIHHLDKIAHTTQAIYKWIMYRKYGNIN

SEQ ID NO: 54 - protein, 264 aa, Streptococcus pneumoniae
MNVKKIMSIFQSFYVDVSIEELTLTLPISFVKRFEYTQMTFHKESFLLIKEKRRGSLSSFVTQARTMGEKANMDVVLVFSKLSDSEKKQLLQARVPFVDFKGNLFFPPLGLVLNANDTEVPKELTPSEQLTWIAFLLTKGQKVVDVDLLSQVTGLPNSTIYRCLRTFKALYWLNKQNKLYTYTVSKKELFLKSVSCLFNPIKKRILLPDGDIKQIKSVSNLLYGGAYALSHSTFLAETDENISYVIWQRKFNQLSLPLSQHVLK

SEQ ID NO: 55 - protein, 85 aa, Streptococcus pneumoniae
LVAKGSAEIVSIDIGTEEYALYRDLTRNHDNNKIIGKGEAASISLAKKHNGILGSNNLRDVKSYVEEFSLEHMTTGDILIEAFKA

SEQ ID NO: 56 - protein, 299 aa, Streptococcus pneumoniae
MLIGQKIKEIRIEKGISRPDFCGDEQELTVRQLSRIESGASQPSLPKLDYIARRLGVPVYSLMPDFSALPSAYLELKYQILREPIYGKEEEYDKKEACLEEIYKTYFDNLPKEEQLACEVLQACLDTSRTRRPEYAELILEEHMSQIIEKEVYSTNDMLLIRLFFYQMLIRKDLAKFINQIEKLMLFLLEQKKVTQIENYFIIRDTLISGMCCLEKVGVTDCFNDYLSCLQEIMDKTQDYQKKPLVFMFLWKQALRVERDFSLAESFYQSSKTFAKLIGDGFLVKKLTEEWQEDVKKYL

SEQ ID NO: 57 - protein, 363 aa, Streptococcus pneumoniae
MPNVKEITRESWILATFPEWGTWLNEEIEEEVVPEGNFAMWWLGNCGTWIKTPAGANVVMDLWSNRGKSTKKVKDMVRGHQMANMAGVRKLQPNLRVQPMVIDPFAINELDYYLVSHFHSDHIDPYTAAAILNNPKLEHVKFIGPYHCGRIWEGWGVPKERIIVVKPGDTIELKDMKIHAVESFDRTCLVTLPVNGADETGGELAGLAVTDEEMAQKAVNYIFETPGGTIYHGADSHFSNYFAKHGKDFKIDVALNNYGENPVGIQDKMTSIDLLRMAENLRTKVIIPVHYDIWSNFMASTNEILELWKMRKDRLQYDFHPFIWEVGGKYTYPQDQHLVEYHHPRGFDDCFEQDSNIQFKALL

SEQ ID NO: 58 - protein, 225 aa, Streptococcus pneumoniae
MKEIFDRRYPVTSFFLLVTALVFLLMLVTAGGNFDRADTLFRFGAMYGPAIRLFPEQVWRLLSAIFVHIGWEHFIVNMLSLYYLGRQVEEIFGSKQFFFLYLLSGMMGNLFVFVFSPKSLAAGASTSLYGLFAAIIVLRYATRNPYIQQLGQSYLTLFVVNIIGSVLIPGISLAGHIGGAVGGAFLAVIFPVRGEKRMYNTSQRLGAVVLFVGLAILLFYKGMGM

SEQ ID NO: 59 - protein, 328 aa, Streptococcus pneumoniae
MPVTIKDVAKAAGVSPSTVTRVIQNKSTISDETKKRVRKAMKELNYHPNLNARSLVSSYTQVIGLVLPDDSDAFYQNPFFPSVLRGISQVASENHYAIQIATGKDEKERLNAISQMVYGKRVDGLIFLYAQEEDPLVKLVAEEQFPFLILGKSLSPFIPLVDNGNVQAGFDATEYFIKKGCKRIAFIGGSKKLFVTKDRLTGYEQALKHYKLTTDNNRIYFADEFLEEKGYKFSKRLFKHDPQIDAIITTDSLLAEGVCNYIAKHQLDVPVLSFDSVNPKLNLAAYVDINSLELGRVSLETILQIINDNKNNKQICYRQLIAHKIIEK

SEQ ID NO: 60 - protein, 335 aa, Streptococcus pneumoniae
MTEKLINSKPNGVFALILIELTIVLGIFIFIMGVGSENIFGIIIGPLLIVIAGLAHAGLKVVKPQEALVLTLFGNYTGTIKEPGFYFVNPFSVAVNPANHTRLGQSGDVSTKSPFLGAKSSNDNDVNLEIGKKHISLKVMTLSNSRQKINDCLGNPVEIGIAVTWRVVDTAKAVFNVDNYKEYLSLQCDSSLRNIVRIYPYDVSPNVDTTGDGQADEGSLRGSSEIVANRIREEIQSRVEDAGLEILEARITYLAYAPEIAAVMLQRQQASAIIDARKMIVDGAVGMVEMALERLNEGELVELDEERKAAMVSNLLVVLCGNHDAQPIVNTGSLY

SEQ ID NO: 61 - protein, 395 aa, Streptococcus pneumoniae
MIYSKEIVREWLDEVAERAKDHPEWVDVFERCYTDTLDNTVEILEDGSTFVLTGDIPAMWLRDSTAQLRPYLHVAKRDALLRQTIAGLVKRQMTLVLKDPYANSFNIEENWKGHHETDHTDLNGWIWERKYEVDSLCYPLQLSYLLWKETGETSQFDETFVAATKEILHLWTVEQDHKNSPYRFVRDTDRKEDTLVNDGFGPDFAVTGMTWSAFRPSDDCCQYSYLIPSNMFAVVVLGYVQEIFAALNLADSQSVIADAKRLQDEIQEGIKNYAYTTNSKGEKIYAFEVDGLGNASIMDDPNVPSLLAAPYLGYCSVDDEVYQATRRTILSSENPYFYQGEYASGLGSSHTFYRYIWPIALSIQGLTTRDKAEKKFLLDQLVACDGGTGVMHESF

SEQ ID NO: 62 - protein, 409 aa, Streptococcus pneumoniae
MSSHPIQVFSEIGKLKKVMLHRPGKELENLLPDYLERLLFDDIPFLEDAQKEHDAFAQALRDEGIEVLYLEQLAAESLTSPEIRDQFIEEYLDEANIRDRQTKVAIRELLHGIKDNQELVEKTMAGIQKVELPEIPDEAKDLTDLVESDYPFAIDPMPNLYFTRDPFATIGNAVSLNHMFADTRNRETLYGKYIFKYHPIYGGKVDLVYNREEDTRIEGGDELVLSKDVLAVGISQRTDAASIEKLLVNIFKKNVGFKKVLAFEFANNRKFMHLDTVFTMVDYDKFTIHPEIEGDLHVYSVTYENEKLKIVEEKGDLAELLAQNLGVEKVHLIRCGGGNIVAAAREQWNDGSNTLTIAPGVVVVYDRNTVTNKILEEYGLRLIKIRGSELVRGRGGPRCMSMPFEREEV

SEQ ID NO: 63 - protein, 219 aa, Streptococcus pneumoniae
MSKMFKCMCLLVMSVFCMSSISPSLVVFADDSLSANSKVVSGEAQFENGSSVRFGDTQVNILSDEVLEVVNPDGSVDTIERRADGVYINGAFYMAYQKNEIDLNISFRSYDPNVWNYVNTIHGNKQANTFANFMTGAGISYMIGRIGALLGGPWGAIIGGAYFGIQAYQSYLDSQSPYPYYITSTYIHVAQRKWKFITEYYRNSNYTGYVKTVTTYVNF

SEQ ID NO: 64 - protein, 469 aa, Streptococcus pneumoniae
MEKAECGQFSILSFLLQESQTTVKAVMEETGFSKATLTKYVTLLNDKALDSGLELTIHSEDENLRLSIGAATKGRDIRSLFLESAVKYQILVYLFYHQQFLAHQLAQELVISEATLGRHLAGLNQILSEFDLSIQNGRWRGPEHQIRYFYFCLFRKVWSSQEWEGHMQKPERKQEIANLEEICGASLSVGQKLDLVLWAHISQQRLRVNACQFQVIEEKMRGYFDNIFYLRLLRKVPSFFAGQHIPLGVEDGEMMIFFSFLLSHRILPLHTMEYILGFGGQLADLLTQLIQEMKKEELLGDYTEDHVTYELSQLCAQVYLYKGYILQDRYKYQLENRHPYLLMEHDFKETAEEIFHALPAFQQGTDLDKKILWEWLQLIEYMAENGGQHMRIGLDLTSGFLVFSRMAAILKRYLEYNRFITIEAYDPSRHYDLLVTNNPIHKKEQTPVYYLKNDLDMEDLVAIRQLLFT

SEQ ID NO: 65 - protein, 1241 aa, Streptococcus pneumoniae
MDKRIFVEKKADFQVKSESLVRELQHNLGLSSLKSIRIVQVYDVFDLAEDLFAPAEKHIFSEQVTDHVLDEVSVQADLANYAFFAIESLPGQFDQRAASSQEALLLLGSSSDVTVNTAQLYLVNKDIDVTELEAVKNYLLNPVDSRFKDITTGIAKQEFSESDKTIPKLTFFESYTAEDFARYKAEQGMAMEVDDLLFIQDYFKSIGRVPTETELKVLDTYWSDHCRHTTFETELKHIDFSASKFQKQLQSTYDKYIAMREELGRSEKPQTLMDMATIFGRYERANGRLDDMEVSDEINACSVEIEVDVDGVKEPWLLMFKNETHNHPTEIEPFGGAATCIGGAIRDPLSGRSYVYQAMRISGAGDITAPISETRAGKLPQQVISKTAAHGYSSYGNQIGLATTYVREYFHPGFVAKRMELGAVVGAAPKGNVVREKPEAGDVIILLGGKTGRDGVGGATGSSKVQTVESVETAGAEVQKGNAIEERKIQRLFRNGNVTRLIKKSNDFGAGGVCVAIGELADGLEIDLNKVPLKYQGLNGTEIAISESQERMAVVVRPEDVDTFVAECNKENIDAVVVATVTEKPNLVMHWNGETIVDLERRFLDTNGVRVVVDAKVVDKDVKLPEERQTSAETLESDTLTVLSDLNHASQKGLQTIFDCSVGRSTVNHPLGGRYQLTPTEASVQKLPVQHGVTHTASVIAQGFNPYVAEWSPYHGAAYAVIEATARLVAAGANWSKARFSYQEYFERMDKQAERFGQPVAALLGSIEAQIQLGLPSIGGKDSMSGTFEELTVPPTLVAFGVTTADSRKVLSPEFKAVGENIYYIPGQALSAEIDFDLIKKNFAQFEASQADHKVTSASAVKYGGVVESLALATFGNYIGAEVTLPELKTALTAQLGGFVFTSPEEIAGVEKIGQTKADFTLTVNGVKLDGHKLDSAFQGTLEEVYPTEFTQAKELEEVPAVASDVVIKAKEKVEKPVVYIPVFPGTNSEYDSAKAFEKEGAEVNLVPFVTLNEEAIVKSVETMVDNIDKTNILFFAGGFSAADEPDGSAKFIVNILLNEKVRVAIDSFIARGGLIIGICNGFQALVKSGLLPYGNFEDANSTSPTLFYNDANQHVAKMVETRIANTNSPWLAGVQVGDIHAIPVSHGEGKFVVTAEEFAELRDNGQIFSQYVDFNGKPSMDSKYNPNGSVHAIEGITSKNGQIIGKMGHSERYEDGLFQNIPGNKDQHLFASAVKHFTGK

SEQ ID NO: 66 - protein, 480 aa, Streptococcus pneumoniae
MTYEVKSLNEECGVFGIWGHPDAAKLTYFGLHSLQHRGQEGAGILSNDQGQLKRHRDMGLLSEVFRNPANLDKLTGAGAIGHVRYATAGEASVDNIQPFLFRFHDMQFGLAHNGNLTNAASLKKELEQRGAIFSATSDSEILAHLIRRSHNPSLMGKIKEALSLVKGGFAYILLFEDKLIAALDPNGFRPLSIGKMANGAVVVSSETCAFEVIGVEWICDLKPGEIVIIDDEGIQYDSYTDDTQLAICSMEYIYFARPDSNIHGVNVHTARKRMGAQLAREFKHEADIVVGVPNSSLSAAMGFAEESGLPNEMGLIKNQYTQRTFIQPTQELREQGVRMKLSAVSGVVKGKRVVMVDDSIVRGTTSRRIVQLLKEAGATEVHVAIGSPALAYPCFYGIDIQTRQELIAANHTVEETRQIIGADSLTYLSIDGLIESIGIETDAPNGGLCVAYFDGDYPTPLYDYEEDYRRSLEEKTSFYK

SEQ ID NO: 67 - protein, 1009 aa, Streptococcus pneumoniae
MKFNPNQRYTRWSIRRLSVGVASVVVASGFFVLVGQPSSVRADVVNPTPGQVLPEETSGTKEGDLSEKPGDTVLTQAKPEGVTGNTNSLPTPTERTEVSEETNSSSLDTLFEKDEEAQKNPELTDVLKEAVDTDDVDGTQASPAETTPEQVKGGVKENTKDSIDVPAAYLEKAEGKGPFTAGVNQVIPYELFAGDGMLTRLLLKASDNAPWSDNGTAKNPALPPLEGLTKGKYFYEVDLNGNTVGKQGQALIDQLRANGTQTYKATVKVYGNKDGKADLTNLVATKNVDININGLVAKETVEKAVKDNVKDSIDVPAAYLEKAKGEGPFTAGVNHVIPYELFAGDGMLTRLLLKASDKAPWSDNGDAKNPALSPLGENVKTKGQYFYQVALDGNVAGKEKQALIDQFRANGTQTYSATVNVYGNKDGKPDLDNIVATKKVTININGLISKETVQKAVADNVKDSIDVPAAYLEKAKGEGPFTAGVNHVIPYELFAGDGMLTRLLLKASDKAPWSDNGDAKNPALSPLGENVKTKGQYFYQVALDGNVVGKEKQALIDQFRANGTQTYSATVNVYGNKDGKPDLDNIVATKKVTININGLISKETVQKAVADNVKDSIDVPAAYLEKAKGEGPFTAGVNHVIPYELFAGDGMLTRLLLKASDKAPWSDNGDAKNPALSPLGENVKTKGQYFYQVALDGNVVGKEKQALIDQFRANGTQTYSATVNVYGNKDGKPDLDNIVATKKVTININGLISKETVQKAVADNVKDSIDVPAAYLEKAKGEGPFTAGVNHVIPYELFAGDGMLTRLLLKASDKAPWSDNGDAKNPALSPLGENVKTKGQYFYQVALDGNVAGKEKQALIDQFRANGTQTYSATVNVYGNKDGKPDLDNIVATKKVTIKINVKETSDTANGSLSPSNSGSGVTPMNHNHATGTTDSMPADTMTSSTNTMAGENMAASANKMSDTMMSEDKAMLPNTGETQTSMASIGFLGLALAGLLGGLGLKNKKEEN

SEQ ID NO: 68 - protein, 354 aa, Streptococcus pneumoniae
MKQEWFESNDFVKTTSKNKPEEQAQEVADKAEETIADFDTPIEKNTQLEEEVSQAEVELESQQEEKIEAPEDSEARTGIEEKKASNSTEEEPDLSKETEKVTIAEESQEALPQQKATTKEPLLISKSLESPYIPDQAPKSRDKWKEQVLDFWSWLVEAIKSPTSKLETSITHSYTAFLLLILFSASSFFFSIYHIKHAYYGHIASINSRFPEQLAPLTLFSIVSILVATTLFFFSFLLGSFVVRRFIHQEKDWTLDKVLQQYSQLLAIPIFLTAIASFFAFFDSLRFTALLCVISIGIILLASLHIITRPSQASETDSFYQLFLSVLVNGVIILLFFVAEVALIGDYLRILAFL

SEQ ID NO: 69 - protein, 388 aa, Streptococcus pneumoniae
MKKQSLFFVPGIILIGVSLRTPFTVLPIILGNISQGLEVEVSSLGVLTSLPLLMFTLFSPFSTQLAQKIGLEHLFTYSLFFLTIGSLIRLINLPLLYLGTLMVGASVAVINVLLPSLIQANQPKKIGFLTTLYVTSMGIATALASYLAVPITQASSWKGLILLLTLLCLATFLVWLPNHRYNHRLAPQTKQKSQIKVMRNKQVWAIIIFSGFQSLIFYTAMTWLPTMSIHAGLSSHEAGLLTSILSLISIPFSMTIPSLTTSLSTRNRQLMLTLVSLAGVVGISMLFFPINNFIYWLAIHLLIGTATSALFPYLMVNFSLKTSAPEKTAQLSGLSQTGGYILAAFGPTLFGYSFDLFHSWVPSVAALLLIDILMTVALFTVDRADKIL

SEQ ID NO: 70 - protein, 50 aa, Streptococcus pneumoniae
MKIKEQTRKLAAGCSKHCFEVADRTDEVSSKRCFEVVDRTDEVSNIYTAK

SEQ ID NO: 71 - protein, 211 aa, Streptococcus pneumoniae
MFTGIIEEIGKVERIQKDSRNCKLSIKASKILTDIHLGDSIAVNGICLTVTHFNHQSFTVDVMNETWSRTALTLLKHGSEVNLERALSVNGRLGGHVVTGHIDGTGKISSIKKDDNAVWYQINTQKEILDLIVEKGSITIDGISLTVAKVSKVNFSVSVIPHTLEQTILKSKQVGSTVNLENDILGKYVQKLMDNSPKSEISKELLYQNGF

SEQ ID NO: 72 - protein, 326 aa, Streptococcus pneumoniae
MNQDRNKLLSKIAYLYYIENLNQSQIAAKLGIYRTSISRMLTEARNAGIVKIEIENFDTNMFKLENYVKEKYGLESLEIIPNEFDDNPTILSERISQVAAGVLRNLIDDNMKIGFSWGKSLSNLVDLIHSKSVRNVHFYPLAGGPSHIHAKYHVNTLIYEMSRKFHGECTFMNATIVQENKLLADGILQSRYFENLKNSWKDLDIAVVGIGDFSNKGKHQWLDMLTEDDFKELTKVKTVGEICCRFFDSKGKEVYENLQERTIAISLEDLKNIPQSLAVAYGDTKVSSILSVLRANLVNHLITDKNTILKVLEEDGDLTFREILGE

SEQ ID NO: 73 - protein, 387 aa, Streptococcus pneumoniae
MVENVGQLAIEQAKANGGKLEVVSKVTVNNRHDLSIAYSPGVASVSSAIAENADLAYTLTTKKNTVAVVSDGTAVLGLGDIGPAAAMPVMEGKAALFKRFADVDAVPIVLDTKDPEEIIRIVKAMAPTFGGVNLEDISAPRCFEIEQRLIEECDIPIFHDDQHGTAIVVLAGLFNSLKLAQKELDKVKIVVNGGGSAGLSITRKLLAAGATDVTVVDRFGIISEEDHDKLAPHHQDIAKVTNRRLASGSLADALEDADVFLGVSAPGALNPEWIAKMNDKPIIFAMANPTPEIFPDEALAAGAYIVGTGRSDFPNQINNVLAFPGIFRGALDARAKKITVEMQIAAAKGIASLIPDSELSPTNIIPDPFQEGVAKVVAKSVSDAVKD

SEQ ID NO: 74 - protein, 233 aa, Streptococcus pneumoniae
MYPALKKHYGEDQEGFYQALEENCEFYNTNPHFLPFITSLHLVMLENGRPTKETRSIKMALMGPLAGIGDSLSQFCLAPLFSTIAASFAQEGLVVGPILFFLAMNTILTAIKLSTGLYGYKLGTTVIDKLSEQMATISRIANIIGVTVIAGLAATSVKIMVPITFAAGEVKADAKQSIVSIQGMLDKVAPALLPALFTLLVYYLIKEKKWTTYKLVILTVIIGIIGSWLKIIA

SEQ ID NO: 75 - protein, 464 aa, Streptococcus pneumoniae
MSKKRRNRHKKEGQEPQFDFDEAKELTVGQAIRKNEEVEAGVLPEDSILDKYVKQHRDEIEADKFATRQYKKEEFVETQSLDDLIQEMREAVEKSEASSEEVPSSEDILLPLPLDDEEQGLDPLLLDDENPTEMTEEVEEEQNLSRLDQEDSEKKSKKGFILTVLALVSVIICVSAYYVYRQVARSTKEIETSQSTTANQSDVDDFNTLYDAFYTNSNKTALKNSQFDKLSQLKTLLDKLEGSREHTLAKSKYDSLATQIKAIQDVNAQFEKPAIVDGVLDTNAKAKSDAKFTDIKTGNTELDKVLDKAISLGKSQQTSTSSSSSSQTSSSSSSQASSNTTSEPKPSSSNDTRSSRSEVNMGLSSAGVAVQRSASRVAYNQSAIDDSNNSAWDFADGVLEQILATSRSRGYITGDQYILERVNIVNGNGYYNLYKPDGTYLFTLNCKTGYFVGNGAGHADDLDY

SEQ ID NO: 76 - protein, 112 aa, Streptococcus pneumoniae
VTIRTTHTYEYQYSLLFGDAGYLWLLLHLFSISKNQYYLQLANVTAKKLIENYDTLEEIDFALGKSGVLLSLIKYYQFTNDNTLKTFIHNSIWGNLSLFPTKRYSQRKHFRL

SEQ ID NO: 77 - protein, 182 aa, Streptococcus pneumoniae
MEIKFTIKQVVAVGIGAALFVVIGMINIPTPVPNTSIQLQYAVQALLSIIFGPIIGLLVGLIGHAIKDSLAGYGLWWTWIIASGLFGLVVGLFRKYVRVINGVFDWKDILIFNLIQLLANALVWGVLAPLGDVVIYQEAAEKVFAQGIVAGIANGVSVAIAGTLLLLAYAGTQTRAGSLKKD

SEQ ID NO: 78 - protein, 84 aa, Streptococcus pneumoniae
MNTKMMEQFHEMDITMLSSIEGGKNNWQTNVLEGGGAAFGGWGLGTAICAASGVGAPFMGACGYIGAKFGVDLWAGVTGATGGF

SEQ ID NO: 79 - protein, 210 aa, Streptococcus pneumoniae
LLSTGKYIGEGFDLPQLDTLILAAPFSWKNNLIQYSGRIHRNYKDKSLVRIFDYVDIHVPYLEKMFQKRQVAYRKMDYRAIEGEEKQFVYVDSRYEKVLIEDLAGERQECLLILPYVHQTKLMNFLKEFRISQIEICIPETVANKAWLDQLKSQKIKVSFTQSKIVTPILLVNKTIVWYDAMPLLGKVDEMTILRLESASIVSELVAGLR

SEQ ID NO: 80 - protein, 219 aa, Streptococcus pneumoniae
MESILEVLTPDNLVFIFKGFGLTLYISLIAIILSTIIGTVLAVMRNGKNPILRIISSIYIEFVRNVPNLLWIFTIFLVFKMKSTPAGITAFTLFTSAALAEIIRGGLNAVDKGQYEAGMSQGFTSAQILYYIILPQAIRKMLPAIISQFVTVIKDTSLLYSVIALQELFGASQILMGRYFEPEQVFSLYILIALIYFSFNLAISSLSHMLAKRWQQAAE

SEQ ID NO: 81 - protein, 266 aa, Streptococcus pneumoniae
MKKFSLLLAILPFLVACGNQATPKETSAQKTIVLATAGDVPPFDYEDKGNLTGFDIEVLKAVDEKLSDYEIQFQRTAWESIFPGLDSGHYQAAANNLSYTKERAEKYLYSLPISNNPLVLVSNKKNPLTSLDQIAGKTTQEDTGTSNAQFINNWNQKHTDNPATINFSGEDIGKRILDLANGEFDFLVFDKVSVQKIIKDRGLDLSVVDLPSADSPSNYIVFSSDQKEFKEQFDKALKELYQDGTLEKLSNTYLGGSYLPDQSQLQ

SEQ ID NO: 82 - protein, 249 aa, Streptococcus pneumoniae
MNKRLFLKMSLVTLPILALFSQPVLAEENIHFSSCKEAWANGYSDIHEGEPGYSAKLDRDHDGVACELKNAPKGAFKAKQSTVAQTNTSSATTSGWVEQDGAWYYFDENGNPVKNAWQGNYYLKSDGKMAQGEWIYDSSYQAWYYLTSDGSYAYSTWQGNYYLKSDGKMAANEWVDGGRYYVGADGVWKEGQASTASSSNDSNSEYSAALGKAKSYNSLFHMSKKRMYRQLTSDFDKFSNDAAQYAIDH

SEQ ID NO: 83 - protein, 260 aa, Streptococcus pneumoniae
MTSLKLLKEKAPLVICITNDVVKNFTANGLVALGASPAMSEFPADLEDLLKYAGGLLINIGTLTDENWKLYQAALKIAEKYNVPAVLDPVACGAGEYRKKVADDLINNYKLAAIRGNAGEIASLVGIDVASKGVDSAGVDNIDEIALAANEKFNIPIVVTGEVDAIAVNGEVVTIHNGSAMMPKVIGTGCLLGAVVASFIGLEKGQELKSLETAMLVYNIAGEIAEKRPNGHLPGTFKAEFINALYEITDEDVKEFKRVK

SEQ ID NO: 84 - protein, 513 aa, Streptococcus pneumoniae
MSILEVKNLSHGFGDRAIFEDVSFRLLKGEHIGLVGANGEGKSTFMSIVTGKMLPDEGKVEWSKYVTAGYLDQHSVLAERQSVRDVLRTAFDELFKAEARINDLYMKMAEDGADVDALMEEVGELQDRLESRDFYTLDAKIDEVARALGVMDFGMDTDVTSLSGGQRTKVLLAKLLLEKPDILLLDEPTNYLDAEHIDWLKRYLQNYENAFVLISHDIPFLNDVINIVYHVENQQLTRYSGDYYQFQEVYAMKKSQLEAAYERQQKEIADLKDFVARNKARVATRNMAMSRQKKLDKMDIIELQSEKPKPSFDFKPARTPGRFIFQAKNLQIGYDRPLTKPLNLTFERNQKVAIIGANGIGKTTLLKSLLGIISPIAGEVERGDYLELGYFEQEVEGGNRQTPLEAVWNAFPALNQAEVRAALARCGLTTKHIESQIQVLSGGEQAKVRFCLLMNRENNVLVLDEPTNHLDVDAKDELKRALKEYRGSILMVCHEPDFYEGWIDQIWDFNNLT

SEQ ID NO: 85 - protein, 227 aa, Streptococcus pneumoniae
MNFSFLPKYLPYFNYGAVVTILISICVIFLGTILGVVLAFGQRSKFKPLVWLANLYVWIFRGTPMMVQIMIAFALMHINAPTIQIGILGVDFSRLIPGILIISMNSGAYVSETVRAGINAVPKGQLEAAYSLGIRPKNAMRYVILPQAVKNILPALGNEFITIIKDSSLLSAIGVMELWNGATTVSTTTYLPLTPLLFAAFYYLIMTSILTVALKAFEKHMGQGDKK

SEQ ID NO: 86 - protein, 220 aa, Streptococcus pneumoniae
MKLNKYIDHTLLKQDAKKKQIDSLLSEAREYGFASVCVNPTWVEHAKKGLEGTDVKVCTVVGFPLGATTSAVKAFETKEAIQNGADEIDMVINVGALKSGNLALVESDIRAVVEASGDKLVKVIIEACLLTDQEKVVVCQLAQKAGADFVKTSTGFSTGGATIADVTLMRETVGSDMGVKAAGGARSYADALAFVEAGATRIGTSAGVAILKGELADGDY

SEQ ID NO: 87 - protein, 307 aa, Streptococcus pneumoniae
MTELAKQLLELTYIVIGCQFLHTAYCSYKDKTNPVRLGTSAFWTLLSITFIGGSYMPNMSIGIIVILLSLLTLFKQVRIGTLPSLDEMKANIESNRLKNKIFIPVMLMAILALVLAQMIPEFSKISISLAALFATISVLVITNSHPKSLLSENNRMTQQVSTSGIVPQLLGALGAIFTVAGVGDVISHLISGIVPSDSRFIGVLAYVLGMVLFTMIMGNAFAAFTVITAGVGVPFVFALGANPIVAGALAMTAGYCGTLLTPMAANFNALPAALMDMKDQNGVIKAQAGVALVMIVIHIFLMYFLAF

SEQ ID NO: 88 - protein, 519 aa, Streptococcus pneumoniae
MHYEHSRKGNHMIKINHLTITQNKDLRDLVSDLSMTIQDGEKVAIIGEEGNGKSTLLKILMGEALSDFTIKGNIQSDYQSLAYIPQKLPEELKKKTLHDYFFLDSIDLDYSILYRLAEELHFDSNRFASDQKIGNLSGGEALKIQLIHELAKPFEILFLDEPSNDLDLETVDWLKGQIQKTRQTVIFISHDEDFLSETADTIVHLRLVKHRKEAETLVEHLDYDSYSEQRKANFAKQSQQAANNQRAYDKTMEKHRRVKQNVETALRATKDSTAGRLLAKKMKTVLSQEKRYEKTAQSMTQKPLEEEQIQLFFSDIQPLPASKVLVQLEKENLSIDDRVLVQKLQLTVRGQEKIGIIGPNGVGKSTLLAKLKQLLNDKREISLGFMPQDYHKKLQLDLSPIAYISKTGEKEELQKIQSHLASLNFSYPEMQHQIRSLSGGQQGKLLLLDLVLRKPNFLLLDEPTRNFSPTSQPQIRKLFATYPGGLITVSHDRRFLKEICSIIYRMTEHGLKLVNLEDL

SEQ ID NO: 89 - protein, 284 aa, Streptococcus pneumoniae
MENFGAVLKDIRISKNFRLKDLSCNEISESTISRFENGITKLSINHFYILLNRLGISFSEFEELVHCYYSKKECLFEELEHAVNSSDIFLLQELVDKIELKQKQEKSLCNYHIKLIAEQQINRLANLPYNSSKCNELIKYLLSVDTWMEYELKLFYNSVFFMNTRTISLLYRIVIKKTRYFLKTNTGTHRIIPLYLFNLKLLLKNNLLGSAQFFIDDLENLLTRQGYYFEKNYLLFLKGIYLIKTNQIELGKKECSKAMRIFKEYNDSDTINELNQKFKLDLTI

SEQ ID NO: 90 - protein, 338 aa, Streptococcus pneumoniae
MKKKWMYYAACSSNESADDSSSDKGDGGSLVVYSPNSEGLIGATIPAFEEKYGIKIELIQAGTGELFKKLESEKEVPVADVIFGGSYTQYATHGELFENYTSKENDNVIKEYQNTTGYSTPYTLDGSVLIVNPDLTKGMNIEGYNDLLKPELKGKIATADPANSSSAFAQLTNMLQAQGGYKDDKAWSYVKDLFTLIDGKIGSSSSSVYKAVADGEMAVGLSYEDPAVKLLNDGANIKVVYPKEGTVFLPASAAIVKKAKNMENAKKFIDFIISQEVQDTLGTTTTNRPVRKNAKTSENMKPIDKIKTLTEDYDYVIKNKSDIVKKYNEVFTDIQSKQ

SEQ ID NO: 91 - protein, 472 aa, Streptococcus pneumoniae
MDKLFKLKENGTDVRTEVLAGLTTFFAMSYILFVNPQILSQTGMPAQGVFLATIIGAVAGTLMMAFYANLPYAQAPGMGLNAFFTFTVVFGLGYSWQEALAMVFICGIISLVITLTNVRKMIIESIPNALRSAISAGIGVFLAYVGIKNAGLLKFTIDPGNYTVVGEGADKAQATIAANSSAVPGLVSLNNPAVLVALAGLAITIFFVIKGIKGGIILSILTTTVLAIAVGLVDLSSIDFANNHVGAAFEDLKTIFGAALGSEGLGALVSDTARLPETLMAILAFSLTDIFDTIGTLIGTGEKVGIVATNGENHQSAKLDKALYSDLIGTTVGAIAGTSNVTTYVESAAGIGAGGRTGLTALVVAICFAISSFFSPLLAIVPTAATAPILIIVGIMMLGSLKNIHWDDMSEAVPAFFTSIFMGFSYSITQGIAVGFLTYTLTKLVKGQVKDVHAMIWILDALFILNYISMAL

SEQ ID NO: 92 - protein, 563 aa, Streptococcus pneumoniae
MKRSSLLVRMVISIFLVFLILLALVGTFYYQSSSSAIEATIEGNSQTTISQTSHFIQSYIKKLETTSTGLTQQTDVLAYAENPSQDKVEGIRDLFLTILKSDKDLKTVVLVTKSGQVISTDDSVQMKTSSDMMAEDWYQKAIHQGAMPVLTPARKSDSQWVISVTQELVDAKGANLGVLRLDISYETLEAYLNQLQLGQQGFAFIINENHEFVYHPQHTVYSSSSKMEAMKPYIETGQGYTPGHKSYVSQEKIAGTDWTVLGVSSLEKLDQVRSQLLWTLLGASVTSLLVCLCLVWFSLKRWIAPLKDLRETMLEIASGAQNLRAKEVGAYELREVTRQFNAMLDQIDQLMVAIRSQEETTRQYQLQALSSQINPHFLYNTLDTIIWMAEFHDSQRVVQVTKSLATYFRLALNQGKDLICLSDEINHVRQYLFIQKQRYGDKLEYEINENVAFDNLVLPKLVLQPLVENALYHGIKEKEGQGHIKLSVQKQDSGLVIRIEDDGVGFQDAGDSSQSQLKRGGVGLQNVDQRLKLHFGANYQMKIDSRPQKGTKVEIYINRIETS

SEQ ID NO: 93 - protein, 816 aa, Streptococcus pneumoniae
MKINKKYLVGSAAALILSVCSYELGLYQARTVKENNRVSYIDGKQATQKTENLTPDEVSKREGINAEQIVIKITDQGYVTSHGDHYHYYNGKVPYDAIFSEELLMKDPNYKLKDEDIVNEVKGGYVIKVDGKYYVYLKDAAHADNVRTKEEINRQKQEHSQHREGGTPRNDGAVALARSQGRYTTDDGYIFNASDIIEDTGDAYIVPHGDHYHYIPKNELSASELAAAEAFLSGRGNLSNSRTYRRQNSDNTSRTNWVPSVSNPGTTNTNTSNNSNTNSQASQSNDIDSLLKQLYKLPLSQRHVESDGLVFDPAQITSRTARGVAVPHGDHYHFIPYSQMSELEERIARIIPLRYRSNHWVPDSRPEQPSPQPTPEPSPGPQPAPNLKIDSNSSLVSQLVRKVGEGYVFEEKGISRYVFAKDLPSETVKNLESKLSKQESVSHTLTAKKENVAPRDQEFYDKAYNLLTEAHKALFENKGRNSDFQALDKLLERLNDESTNKEKLVDDLLAFLAPITHPERLGKPNSQIEYTEDEVRIAQLADKYTTSDGYIFDEHDIISDEGDAYVTPHMGHSHWIGKDSLSDKEKVAAQAYTKEKGILPPSPDADVKANPTGDSAAAIYNRVKGEKRIPLVRLPYMVEHTVEVKNGNLIIPHKDHYHNIKFAWFDDHTYKAPNGYTLEDLFATIKYYVEHPDERPHSNDGWGNASEHVLGKKDHSEDPNKNFKADEEPVEETPAEPEVPQVETEKVEAQLKEAEVLLAKVTDSSLKANATETLAGLRNNLTLQIMDNNSIMAEAEKLLALLKGSNPSSVSKEKIN

SEQ ID NO: 94 - protein, 285 aa, Streptococcus pneumoniae
MILSKNREDELRKFATNIRLNTLRTLNHLGFGHYGGSLSIVEVLAVLYGEIMPMTPEIFAARDRDYFILSKGHGGPALYSTLYLNGFFDKEFLYSLNTNGTKLPSHPDRNLTPGIDMTTGSLGQGISVATGLAYGQRIRKSPFYTYAIVGDGELNEGQCWEAIQFASHQQLSNLIVFVDDNKKQLDGFTKDICNPGDFVEKFSAFGFESIRVKGSDIREIYEGIVQLKQSNNSSPKCIVLDTIKGQGVQELEEMKSNHHLRPTVEERQMLTSVVERLSQELEETE

SEQ ID NO: 95 - protein, 626 aa, Streptococcus pneumoniae
MVRFIGLSPKQTQAIEVLKGHISLPDVEVAVTQSDQASISIEGEEGHYQLTYRKPHQLYRALSLLVTVLAEADKVEIEEQAAYEDLAYMVDCSRNAVLNVASAKQMIEVLALMGYSTFELYMEDTYQIEGQPYFGYFRGAYSAEELQEIEAYAQQFDMTFVPCIQTLAHLSAFVKWGVKEVQELRDVEDILLIGEEKVYDLIDGMFATLSKLKTRRVNIGMDEAHLVGLGRYLILNGVVDRSLLMCQHLERVLDIADKYGFHCQMWSDMFFKLMSADGQYDRDVEIPEETRVYLDRLKDRVTLVYWDYYQDSEEKYNRNFRNHHKISHDLAFAGGAWKWIGFTPHNHFSRLVAIEANKACRANQIKEVIVTGWGDNGGETAQFSILPSLQIWAELSYRNDLDGLSAHFKTNTGLTVEDFMQIDLANLLPDLPGNLSGINPNRYVFYQDILCPILDQHMTPEQDKPHFAQAAETLANIKEKAGNYAYLFETQAQLNAILSSKVDVGRRIRQAYQADDKESLQQIARQELPELRSQIEDFHALFSHQWLKENKVFGLDTVDIRMGGLLQRIKRAESRIEVYLAGQLDRIDELEVEILPFTDFYADKDFAATTANQWHTIATASTIYTT

SEQ ID NO: 96 - protein, 309 aa, Streptococcus pneumoniae
MKKFSKTLRDNWIFLLMVLPGTLWLILFFYIPVFGNVVAFKDYHMTSNGFIDSIINSKWVGLDNFRFLFSSRDAFIITRNTVLYNLGFIFLGLVVSVGIAIILSELRSKRMVKIFQTSMLFPYFLSWVIISFFTDAFLNIDKGVFNHLLESLGLKEVNFYADLGIWPYLLLFLGIWKGFGYSSVMYYATIMGIDPTYYEAATVDGASKWQRIRNVTIPQLTPLVTVLTILAVGNIFRADFGLFYQIPHNAGQLYNVTNVLDVYVFNGLTQTADIGMAAAAGLYQSVVGLILVILSNLLARRVDPNSALF

SEQ ID NO: 97 - protein, 242 aa, Streptococcus pneumoniae
MKLNKLNFLKENIRNLYSSGVIYLSLLISFIPPILLTFFILKTQGTSLGIKHISNFYAMLGMLMAVIHANRVISRDFSHNTVSLFYNQQKNRMIYVLSNFLYAISVSIIYALNGIVLLVIVSKLGIPGDLGLDFIVAIVVNTILLVLFYFLLSYIFYLYKLKSGLVFGILVALLLFIPNILNTMMMNTSNDLFIKAIELLPFYSLPLFVASNTMSISQYLVLITTIILLYFFTLKKSKKYSF

SEQ ID NO: 98 - protein, 598 aa, Streptococcus pneumoniae
MEQKHRSEFPEKELWDLTALYQDREDFLRAIEKAREDINQFSRDYKGNLHTFEDFEKAFAELEQIYIQMSHIGNYGFMPQTTDYSNDEFANIAQAGMEFETAASVALTFFDDALVVADEEVLDRLGELPHLTAAIRQAKIKKAHYLGADVEKALTNLGEVFYSPQDIYTKMRAGDFEMADFEAHGKTYKNSFVTYENFYQNHEDAEVREKSFRSFSEGLRKHQNTAAAAYLAQVKSEKLLADMKGYDSVFDYLLAEQEVDRVMFDRQIDLIMKDFAPVAQRYLKHVAKVNGLEKMTFADWKLDLDSALNPEVTIDDAYDLVMKSVEPLGQEYCQEVARYQEERWVDFAANSGKDSGGYAADPYRVHPYVLMSWTGRLSDVYTLIHEIGHSGQFIFSDNHQSYFNAHMSTYYVEAPSTLNELLLSDYLENQSNDPRQKRFALAHRLTDTYFHNFITHLLEAAFQRKVYTLIEEGETFGASKLNSIMKEVLTDFWGDAIEIDDDAALTWMRQAHYYMGLYSYTYSAGLVISTAGYLHLKNSGTGAEDWLNLLKSGGSKTPLESAMIIGADISTDKPLRDTIQFLSDTVDQIIAYSAELGE

SEQ ID NO: 99 - protein, 318 aa, Streptococcus pneumoniae
MEINVSKLRTDLPQVGVQPYRQVHAHSTGNPHSTVQNEADYHWRKDPELGFFSHIVGNGCIMQVGPVDNGAWDVGGGWNAETYAAVELIESHSTKEEFMTDYRLYIELLRNLADEAGLPKTLDTGSLAGIKTHEYCTNNQPNNHSDHVDPYPYLAKWGISREQFKHDIENGLTIETGWQKNDTGYWYVHSDGSYPKDKFEKINGTWYYFDSSGYMLADRWRKHTDGNWYWFDNSGEMATGWKKIADKWYYFNEEGAMKTGWVKYKDTWYYLDAKEGAMVSNAFIQSADGTGWYYLKPDGTLADKPEFTVEPDGLITVK

SEQ ID NO: 100 - protein, 313 aa, Streptococcus pneumoniae
MVQEIAQEIIRSARKKGTQDIYFVPKLDAYELHMRVGDERCKIGSYDFEKFAAVISHFKFVAGMNVGEKRRSQLGSCDYAYDQKMASLRLSTVGDYRGHESLVIRLLHDEEQDLHFWFQDIEELGKQYRQRGLYLFAGPVGSGKTTLMHELSKSLFKGQQVMSIEDPVEIKQDDMLQLQLNEAIGLTYENLIKLSLRHRPDLLIIGEIRDSETARAVVRASLTGATVFSTIHAKSIRGVYERLLELGVSEEELAVVLQGVCYQRLIGGGGIVDFASRDYQEHQAAKWNEQIDQLLKDGHITSLQAETEKISYS

SEQ ID NO: 101 - protein, 595 aa, Streptococcus pneumoniae
MTRFEIRDDFYLDGKSFKILSGAIHYFRVPPEDWYHSLYNLKALGFNTVETYVAWNLHEPREGEFHFEGDLDLEKFLQIAQDLGLYAIVRPSPFICAEWEFGGLPAWLLTKNMRIRSSDPAYIEAVGRYYDQLLPRLVPRLLDNGGNILMMQVENEYGSYGEDKAYLRAIRQLMEECGVTCPLFTSDGPWRATLKAGTLIEEDLFVTGNFGSKAPYNFSQMQEFFDEHGKKWPLMCMEFWDGWFNRWKEPIITRDPKELADAVREVLEQGSINLYMFHGGTNFGFMNGCSARGTLDLPQVTSYDYDALLDEEGNPTAKYLAVKKMMATHFSEYPQLEPLYKESMELDAIPLVEKVSLFETLDSLSSPVESLYPQKMEELGQSYGYLLYRTETNWDAEEERLRIIDGRDRAQLYVDGQWVKTQYQTEIGEDIFYQGKKKGLSRLDILIENMGRVNYGHKFLADTQRKGIRTGVCKDLHFLLNWKHYPLPLDNPEKIDFSKGWTQGQPAFYAYDFTVEEPKDTYLDLSEFGKGVAFVNGQNLGRFWNVGPTLSLYIPHSYLKEGANRIIIFETEGQYKEEIHLTRKPTLKHIKGENL

SEQ ID NO: 102 - protein, 141 aa, Streptococcus pneumoniae
MTVVFEFVSEMLKQFVGLDGQGLNQSNIQSTFQEQPLLIAVFACVIGPLVEELFFRQVLLHYLQERLSGLLSIILVGLVFALTHMHSLALSEWIGAVGYLGGGLAFSIIYVKEKENIYYPLLVHMLSNSLSLIILAISIVK

SEQ ID NO: 103 - protein, 163 aa, Streptococcus pneumoniae
MTMPNIIMTRIDERLIHGQGQLWVKYLGCNTVIVANDEVSTDKMQQTLMKTVVPDSVAMRFFPLQKVIDIIHKANPAQTIFIVVKDVKDALTLVEGGVPIKEINIGNIHNATGKEQVTRSIFLGEEDKAALKELSQTHQVTFNTKTTPTGNDGAVQVNIMDYI

SEQ ID NO: 104 - protein, 453 aa, Streptococcus pneumoniae
MNPNLFRSVEFYQRRYHNYATVLIIPLSLLFTFILIFSLVATKEITVTSQGEIAPTSVIASIQSTSDNPILANHLVANQVVEKGDLLIKYSETMEESQKTALETQLQRLEKQKEGLGILKQSLEKATDLFSSEDEFGYHNTFMNFTKQSHDIELGISKTNTEVSNQANLTNSSSSAIEQEITKVQQQIGEYQELRDAIVNKRARLPTGNPHQSILNRYLIASQEQTQGTAEEPFLSQINQSIAGLESSIASLKLQQAGIGSVATYDNSLATKIEVLRTQFLQTASQQQLTVENQLTELKVQLDQATQRLENNTLTAPSKGIVHLNSEFEGKNRIPTGTEIAQIFPIITDTREVLITYYVSSDYLPLLDKGQTVRLKLEKIGNHGTTIIGQLQTIDQTPTRTEQGNLFKLTALAKLSNEDSKLIQYGLQGRVTSVTAKKTYFDYFKDKILTHSD

SEQ ID NO: 105 - protein, 215 aa, Streptococcus pneumoniae
MTLLQLQDVTYRYKNTAEAVLYQIDYNFEPGKFYSIIGESGAGKSTLLSLLAGLDSPVEGSILFQGEDIRKKGYSYHRMHHISLVFQNYNLIDYLSPLENIRLVNKKASKDTLLELGLDESQIKRNVLQLSGGQQQRVAIARSLVSEAPVILADEPTGNLDPKTAGDIVELLKSLAQKTGKCVIVVTHSKEVAQASDITLELKDKKLTETRNTSK

SEQ ID NO: 106 - protein, 267 aa, Streptococcus pneumoniae
MQEFTNPFPIGSSSLIHCMTNEISCEMLANGILALGCKPVMADDPREVLDFTKQSQALFINLGHLSAEKEKAIRMAASYANQSSLPMVVDAVGVTTSSIRKSLVKDLLNYRPTVIKGNMSEIRSLVGLKHHGVGVDASAKDQETEDLLQVLKDWCQTYPGMSFLVTGPKDLIVSKNQVAVLENGCTELDWITGTGDLVGALTAVFLSQGKTGFEASCLAVSYLNIAAEKIVVQGMGLEEFRYQVLNQLSLLRRDENWLDTIKGEAYE

SEQ ID NO: 107 - protein, 462 aa, Streptococcus pneumoniae
MADSFHFSVNIISRGKGKSAVASAAYISGEKIKNEWDGVTHDYTRKEKILVKNIILPDHIPKEFNDRSTLWNKVEMAEKNSNAQLARQFIIGLPKELSLSENKNLVERYIKENLTSQGMIVDYAIHDESQDKNGNIHCHIMTIMRPINEKGEFLAKSKKEYILDEKGEKVLNKNGKPKTRKVELTTWNDTGNVEQWRENFSDLCNKYLERAGAEKRVDHRSFKRQNSDYLPTIHLGSAASAMERKGIETDKGNYNREIRKYNQLVKTIKEEIKTLKGWIGNLLDNLSTAYEKFKDIERDKVIDNPKLFNLTNYLLTYSEIQKEKSKYLKGYAKTNKEKYDFKKLTSAYSYLRKNNIETIGQLQTKIETLKSNSYRLNKKAKTIHKEMEDVEKKILYYEIYKAKKEVYEEYQKKNIFTKEAFYNKHKKDIDQYKVVSGKLKKLLSDKEKLSPKKWNEEKILLM

SEQ ID NO: 108 - protein, 633 aa, Streptococcus pneumoniae
MIILQANKIERSFAGEVLFDNINLQVDERDRIALVGKNGAGKSTLLKILVGEEEPTSGEINKKKDISLSYLAQDSRFESENTIYDEMLHVFDDLRRTDKQLRQMELEMGEKSGEDLDKLMSDYDRLSENFRQAGGFTYEADIRAILNGFKFDESMWQMKIAELSGGQNTRLALAKMLLEKPNLLVLDEPTNHLDIETISWLENYLVNYSGALIIVSHDRYFLDKVATITLDLTKHSLDRYVGNYSRFVELKEQKLATEAKNYEKQQKEIAALEDFVNRNLVRASTTKRAQSRRKQLEKMERLDKPEAGKKAANMTFQSEKTSGNVVLTVENAAVGYDGEVLSQPINLDLRKMNAVAIVGPNGIGKSTFIKSIVDQIPFIKGEKRFGANVEVGYYDQTQSKLTPSNTVLDELWNDFKLTPEVEIRNRLGAFLFSGDDVKKSVGMLSGGEKARLLLAKFSMENNNFLILDEPTNHLDIDSKEVLENALIDFDGTLLFVSHDRYFINRVATHVLELSENGSTLYLGDYDYYVEKKATAEMSQTEEASTSNQAKEASPVNDYQAQKESQKEVRKLMRQIESLEAEIEELESQSQAISEQMLETNDADKLMELQAELDKISHRQEEAMLEWEELSEQV

SEQ ID NO: 109 - protein, 563 aa, Streptococcus pneumoniae
MNKLIAFIEKGKPFFEKLSRNIYLRAIRDGFIAGMPVILFSSIFILIAFVPNSWGFKWSDEVVAFLMKPYSYSMGILALLVAGTTAKSLTDSVNRSMEKTNQINYMSTLLAAIVGLLMLAADPIESGLATGFLGTKGLLSAFLAAFVTVAIYKVCVKNNVTIRMPDEVPPNISQVFKDVIPFTLSVVSLYALDLLARHFVGASVAESIGKFFAPLFSAADGYLGITIIFGAFAFFWFVGIHGPSIVEPAIAAITYANAEVNLNLLQQGMHADKILTSGTQMFIVTMGGTGATLVVPFMFMWLTKSKRNRAIGRASVVPTFFGVNEPILFGAPLVLNPIFFIPFIFVPIANVWIFKFFIETLGMNSFTANLPWTTPAPLGLVLGTNFQVLSFILAALLIVVDVVIYYPFLKVYDEQILEEERSGKSNDELKEKVAANFNTAKADAILEKAGVEAAQNTITKETNVLVLCAGGGTSGLLANALNKAAAEYNVPVKAAAGGYGAHREMLPEFDLVILAPQVASNFEDMKAETDKLGIKLAKTEGAQYIKLTRDGKGALAFVQAQFD

SEQ ID NO: 110 - protein, 479 aa, Streptococcus pneumoniae
LQNLKKYLLLDSGLVVGYTLENAQLKLNKLKKDTENNPLFGEEYFAENPKLWEVRFDNSYPNVIYDKELKLYRCWYQTFVSDEASEETPLAERGEKEYIVKSSRMTALCYAESKDGVKWEKPNLNLVKFKGSKDNNIVLMHAHGAGIMLDEQELDPKKRYKLVSKLEFSPNLHYMVVVFSEDGIHFDEPIPWKKWNPAADSHNFPFRDPKTGKYAVITRTWANNVRLSAITFSDDFINWSEPKEILRGDGFEDQIYSMPVISYADIYLGFASMYHEGDTLNENYDKVDLELKFSANLEQWDSVCKGDYLIERGGGVYPQNADFDASCIFTAAPIIEDDKIWFYYMGGNGNHTGFRESSLARGYIKKDQFAYYTNKREGIPAKISTVSFQTYDDEIYILADIPENGYVKVAVGTKYGNAYSDFGQEDISLVQVSDGKYKLEFNKKSILDLGNSPVAFKFEIENAKLYGIEGSIQQHYLKY

SEQ ID NO: 111 - protein, 467 aa, Streptococcus pneumoniae
MNNDEWVYQYPIGKFVERQGWKIHISSEYNSSHELLQDVAKICHEMRIPFKHLSTEDKFIMRNGKLVSRGFSGKFITCYPNQNELESVLQRLESALKQYNGPYILSDKRWDEAPIYLRYGVFRPSRDDEKKVVIDELIVGDEVVKDERLPVFKIPKGIVPPDFLNKWLDKKDKKQGDFPFIIDNAIRFSNSGGIYNARLKEDGKKIILKEARPYTGLGFDGTYSSERLASECKALKILNEWSEMPKIYWYGKIWEHTFLGIEHMKGVPLNRWVTNNFPLYEVVDKTKDYLLRVSKIVEKLIDLTNKFHSENVYHQDLHLGNILVKDEDEISIIDWEQAVFSNDEKVVHKVAAPGFRAWRETLPSEIDWYGIRQIAHYLYMPLVTTSDLTYNYVSQTRIEGKKLFESLGYTREHIDYVESLLSYLDSKCPQIENISRKKVLKPMHEIRTIESEQDIQDFIIKAFLLVN

SEQ ID NO: 112 - protein, 368 aa, Streptococcus pneumoniae
MNIILIAKLLRENTNTKANALNNGWARSGSEEFKKFSHFVGVDKGIVRTNVLTGKKLSDKIRKEVGSGDSKLGKGGYFSTGDVLLGKDVVSYTVQVFSENNERVGVNTQSHRVQYNLPILADFSVIQDTVEPSRTVVEKIIPKLNIPEEEKGKITEEIKKKKKTSELAELISENVKVRYVDEQGRLLSLKNDTGIGEKESDGTYITNKKQLIGTSYNVTDKKLSSMTTTDGKYYTFKEVDTNSASLTGNIVSEGRTVTLVYRESEAPTTVTVTANYYKEGSQEKLAESVIKADLAIGSEYTTESKTIEGKTTTEDKENRVITRKTTYTLVATPANAYQKTVQQLTITTVRMLRKQWFPKQQPLLRRRL

SEQ ID NO: 113 - protein, 652 aa, Streptococcus pneumoniae
MKKSKSKYLTLAGLVLGTGVLLSACGNSSTASKTYNYVYSSDPSSLNYLAENRAATSDIVANLVDGLLENDQYGNIIPSLAEDWTVSQDGLTYTYKLRKDAKWFTSEGEEYAPVTAQDFVTGLQYAADKKSEALYLVQDSVAGLDDYITGKTSDFSTVGVKALDDQTVQYTLVKPELYWNSKTLATILFPVNADFLKSKGDDFGKADPSSILYNGPFLMKALVSKSAIEYKKNPNYWDAKNVFVDDVKLTYYDGSDQESLERNFTAGAYTTARLFPNSSSYEGIKEKYKNNIIYSMQNSTSYFFNFNLDRKSYNYTSKTSDIEKKSTQEAVLNKNFRQAINFAFDRTSYGAQSEGKEGATKILRNLVVPPNFVSIKGKDFGEVVASKMVNYGKEWQGINFADGQDPYYNPEKAKAKFAEAKKELEAKGVQFPIHLDKTVEVTDKVGIQGVSSIKQSIESVLGSDNVVIDIQQLTSDEFDSSGYFAQTAAQKDYDLYHGGWGPDYQDPSTYLDIFNTNSGGVLQNLGLEPGEANDKAKAVGLDVYTQMLEEANKEQDPAKRYEKYADIQAWLIDSSLVLPSVSRGGTPSLRRTVPFAAAYGLTGTKGVESYKYLKVQDKIVTTDEYAKAREKWLKEKEESNKKAQEELAKHVK

SEQ ID NO: 114 - protein, 330 aa, Streptococcus pneumoniae
MDLLEKECLKCDKNFQQGDIWNYYYLSDKMPAQGWKIHISSQIKDAVNIFKIVYKLSQLNNCSFKVVKNLEELKKINSPREMSPTANKFITLYPKSESKAKSMICNLTNRLSEFKAPKILSDYQCGMHSPVHYRYGAFLKKQAYDEKNKKVIYLLLDEKRKNYVEDKRQNFPSLPSWKMDLFSEEEKRIYFQTTCEVSSKDSAINKYKMEKIIKRSNKGNVYRAIRKSDGQKVIIKQSRPFVNYDAEGEWTALDDIKNEAHMLKKLADKSYTTNLTDEFYIVDDYFLVQEQVDGLNFEEFIRETEHSLNIREKTLDNIVNIVSYIHKLGI

SEQ ID NO: 115 - protein, 423 aa, Streptococcus pneumoniae
MPDNLALRMRPKTIDQVIGQEHLVGPGKIIRRMVEANRLSSMILYGPPGIGKTSIASAIAGTTKYAFRTFNATVDSKKRLQEISEEAKFSGGLVLLLDEIHRLDKTKQDFLLPLLESGLVIMIGATTENPFFSVTPAIRSRVQIFELEPLSNQDVKEALQIALSNPERGFDFPIELDEDALDFIATSTNGDLRSAFNSLDLAVLSTPENDEGIRHITLDIMENSLQRSYITMDKDGDGHYDVLSALQKSIRGSDVDASLHYTARLIEAGDLPSLARRLTVIAYEDIGLANPEAQIHTVTALDAAQKIGFPEARILIANVVIDLALSPKSNSAYVAMDKALADLKTSGHLPIPRHLRDGHYSGSKELGNAQDYLYPHNYPGNWVKQDYLPEKIRNHHYFQAEDTGKYERALAQRKEAIDRLRKI

SEQ ID NO: 116 - protein, 493 aa, Streptococcus pneumoniae
MRDLLSKKSHRQLELLELLFEHKRWFHRSELAELLNCTERAVKDDLSHVKSAFPDLIFHSSTNGIRIINTDDSDIEMVYHHFFKHSTHFSILEFIFFNEGCQAESICKEFYISSSSLYRIISQINKVIKRQFQFEVSLTPVQIIGNERDIRYFFAQYFSEKYYFLEWPFENFSSEPLSQLLELVYKETSFPMNLSTHRMLKLLLVTNLYRIKFGHFMEVDKDSFNDQSLDFLMQAEGIEGVAQSFESEYNISLDEEVVCQLFVSYFQKMFFIDESLFMKCVKKDSYVEKSYHLLSDFIDQISVKYQIEMENKDNLIWHLHNTAHLYRQELFTEFILFDQKGNTIRNFQNIFPKFVSDIKKELSHYLETLEVCSSSMMVNHLSYTFITHTKHLVINLLQNQPKLKVLVMSNFDQYHAKFVAETLSYYCSNNFELEVWTELELSKESLEDSPYDIIISNFIIPPIENKRLIYSNNINTVSLIYLLNAMMFIRLDE

SEQ ID NO: 117 - protein, 321 aa, Streptococcus pneumoniae
MKTSLKLYFTALVASFLLLLGACSTNSSTSQTETSSSAPTEITIKSSLDEVKLSKVPEKIVTFDLGAADTIRALGFEKNIVGMPTKTVPTYLKDLAGTVKNVGSMKEPDLEAIAALEPDLIIASPRTQKFVDKFKEIAPTVLFQASKDDYWTSTKANIESLASAFGETGTQKAKEELAKLDKSIQEVATKNESSDKKALAILLNEGKMAAFGAKSRFSFLYQTLKFKPTDTTFEDSRHGQEVSFESVKEINPDILFVINRTLAIGGDNSSNDGVLENALIAETPAAKNGKIIQLTPDLWYLSGGGLESTKLMIEDIQKALK

SEQ ID NO: 118 - protein, 199 aa, Streptococcus pneumoniae
MKVLVAEDQSMLRDAMCQLLAFQADVESVLQAKNGQEAIQLLEKESVDIAILDVEMPVKTGLEVLEWIRAEKLETKVVVVTTFKRPGYFERAVKAGVDAYVLKERNIADLMQTLHTVLEGGKEYSPELMEVVMMHPNPLTEQEIAVLKGIAQGLSNQEIADQLYLSNGTVRNYVTNILSKLDAGNRTEAANIAKESGWL

SEQ ID NO: 119 - protein, 365 aa, Streptococcus pneumoniae
MLERLKSIHYMFWISLIFMIFPILPVVTGWLSAWHLLIDILFVVAYLGVLITKSQRLSWLYWGLMLSYVVGNTAFVAVNYIWFFFFLSNLLSYHFGVRSFNSLHVRTFLLAQVLVVGQLLIFQEVEVEFLVYLLGILTFIDLMTFGLVRIRIMEDLKEAQAKQNAQINLLLAENERSRIGQDLHDSLGHTFAMLSVKTDLALQLFQIQAYPQVEKELREIQQISKESMREVRTIVENLKSRTLISELETVKKMLEIAGIEVETDNQLDTASLTQKLESTASMILLELVTNIIKHAKASKVYLKLERTEKELILTVRDDGCGFASIKGDDLHTVRDRILPFSGEVKVISWKQPTEVQVRLPYKERK

SEQ ID NO: 120 - protein, 557 aa, Streptococcus pneumoniae
MVLDKASCDLLQYLMDQETSKTIMAISKDLKESRRKIYYHIDKINAALGDEALHIISIPRIGIHLTEEQRDACCKLLSEVDSYDYIMSAHERMMIMLLWIGISKERITIEKLIELTEVSRNTVLNDLNSIRYQLTLEQYQVTLQVSKSQGYHLHAHPLNKIQYLQSLLYHIFMEENATFVSILEDKMKERLDDECLLSVEMNQFFKEQVPLVEQDLGKKINHHEITFMLQVLPYLLLSCHNVEQYQERHQDIEKEFSLIRKRIEYQVSKKLGERLFQKFEISLSGLEVSLVAVLLLSYRKDLDIHAESDDFRQLKLALEEFIWYFESQIRMEIENKDDLLRNLMIHCKALLFRKTYGIFSKNPLTKQIRSKYGELFLATRKSAEILEGAWFIRLTDDDIAYLTIHIGGFLKYTPSSQKNMKKVYLVCDEGVAVSRLLLKQCKLYFPNEQIDTVFTTEQFKSVEDIAQVDVVITTNDDLDSRFPILRVNPILEAEDILKMLDYLKHNIFRNKSKSFSENLSSLISSYIVDSKLASKFQEEVQTLINQEIVVQAFLEDI

SEQ ID NO: 121 - protein, 752 aa, Streptococcus pneumoniae
MLSLQEFVQNRYNKTIAECSNEELYLALLNYSKLASSQKPVNTGKKKVYYISAEFLIGKLLSNNLINLGLYDDVKKELAAAGKDLIEVEEVELEPSLGNGGLGRLAACFIDSIATLGLNGDGVGLNYHFGLFQQVLKNNQQETIPNAWLTEQNWLVRSSRSYQVPFADFTLTSTLYDIDVTGYETATKNRLRLFDLDSVDSSIIKDGINFDKTDIARNLTLFLYPDDSDRQGELLRIFQQYFMVSNGAQLIIDEAIEKGSNLHDLADYAVVQINDTHPSMVIPELIRLLTARGIELDEAISIVRSMTAYTNHTILAEALEKWPLEFLQEVVPHLVPIIEELDRRVKAEYKDPAVQIIDESGRVHMAHMDIHYGYSVNGVAALHTEILKNSELKAFYDLYPEKFNNKTNGITFRRWLMHANPRLSHYLDEILGDGWHHEADELEKLLSYEDKAAVKEKLESIKAHNKRKLARHLKEHQGVEINPNSIFDIQIKRLHEYKRQQMNALYVIHKYLDIKAGNIPARPITIFFGGKAAPAYTIAQDIIHLILCMSEVIANDPAVAPHLQVVMVENYNVTAASFLIPACDISEQISLASKEASGTGNMKFMLNGALTLGTMDGANVEIAELVGEENIYIFGEDSETVIDLYAKAAYKSSEFYAREAIKPLVDFIVSDAVLAAGNKERLERLYNELINKDWFMTLLDLEDYIKVKEQMLADYEDRDAWLDKVIVNISKAGFFSSDRTIAQYNEDIWHLN

SEQ ID NO: 122 - protein, 408 aa, Streptococcus pneumoniae
MKVFLQNRDFRQLTINQWISTLGDTIFYLAFLNYVADASFAPLTILLITISETLPQILQIFLGVLADFQHHRVLKYTVISFAKFLLYSIVSLSLSGQSFSLLLVAFICLLNLLSDTLSYFSGAMLTPIFIRIIGQDHLAEAIGFKQSTVSLVKTISNILGGVLLGILSIQFISLLNALTFLIAFLGILFIKTDLLKVEKTISYQEGLSVKSFCQHLLQSSKLIWNMNKVLLVLFIISTSQAVINVTVPVSTLFLRNQPFLNLQTGQSLALLSTFELSALIVGSLVSGYLQHTISIKTALYASLVIQLLLLVGFATVRFDWILIFSTLDAFFAGVLSPRLQELVFKQIPEESMGAVQSSIGAITVVLPSLFTIALVTIATSFGTLAVSFVLLLFLLVAFVMLLNIRESI

SEQ ID NO: 123 - protein, 401 aa, Streptococcus pneumoniae
MEYRKIQEALEALQKGRLVLVIDDKDRENEGDLICSAQAATTENVNFMATYAKGLICMPMSESLANQLMLSPMVENNTDNHKTAFTVSIDYKETTTGISAEERGLTARMCVAEDITPSDFRRPGHMFPLIAKKGGVLERSGHTEATVDLLKLAGLKECGLCCEIMNHDGKMMRTDDLIQFSKKHNIPLITIKELQEYRKVYDQLVERVSTVNMPTRYGNFKAISYIDKLNGEHHLALIMGNIEDEANVLCRVHSECLTGDVLGSLRCDCGQQFDKAMKMIVENGSGVLLYLRQEGRGIGLINKLKAYHLQDQGMDTLDANLALGFEGDLREYHIGAQMLKDLGLQSLHLLTNNPDKVEQLEKYGITISSRISIEIEANPYDSFYLETKKNRMGHILNMEEK

SEQ ID NO: 124 - protein, 478 aa, Streptococcus pneumoniae
MSKFPKEFLWGGATAANQYEGAYNVGDKGLSVQDVTPRGGVPVSDNDPNPFITELPTEDNLKLEGIDFYHRYKEDVALFAEMGFKVFRTSIAWSRIFPNGDELEPNEEGLQFYDNLFDELAKYGIEPLVTLSHYETPLHLARKYNGWVNRDLIGFYERYVRTVFNRYKNKVKYWLTFNEINSVLHVPFISGGIATPVEKLSKQDLYQAVHHELVASALATKVGHEINPEFKIGCMVLAMPTYPMTPKPEDVLAAREFENQNYLFSDIHARGKYPKYIQRFFKENDINIKFESGDKELLAENTVDFISFSYYMSSVQAHDPENYQSGIGNLLGGIANPYLESSEWGWQVDPIGLRIVLNNLYDRYQLPLFIVENGLGAKDVLIEGADGPTVDDDYRIDYLKKHLQQVGEAIEDGVELLGYTTWGCIDLVSASTAQMSKRYGFIYVDRNDDGSGTLNRYKKKSFYWYKEVIESNGETLYD

SEQ ID NO: 125 - protein, 234 aa, Streptococcus pneumoniae
LEMGKLSSHMWRLNQIIYTKYFWGYVLFWILICLGLWYWLEGNDRLVIEILKGPNLSQNSFLVLSIWLLHWFIIHTFFLAVVYRRRASDFFMEVIRFSSIKLWIRYQIWTCFLYGLILIMVKVLVIQFMLQLPNWDIGVLFIVDSLNACVLSLLCFMLYALGANVQMNFACVSFFLLMIVFGGLFVGNRTNYLFYILNRGNGDIGRDLFLQLLFLVFLFKSIFYFTRQKRRFIE

SEQ ID NO: 126 - protein, 702 aa, Streptococcus pneumoniae
MKKVRFIFLALLFFLASPEGAMASDGTWQGKQYLKEDGSQAANEWVFDTHYQSWFYIKADANYAENEWLKQGDDYFYLKSGGYMAKSEWVEDKGSFYYLDQDGKMKRNAWVGTSYVGATGAKVIEDWVYDSQYDAWFYIKADGQHAEKEWLQIKGKDYYFKSGGYLLTSQWINQAYVNASGAKVQQGWLFDKQYQSWFYIKENGNYADKEWIFENGHYYYLKSGGYMAANEWIWDKESWFYLKFDGKIAEKEWVYDSHSQAWYYFKSGGYMTANEWIWDKESWFYLKSDGKIAEKEWVYDSHSQAWYYFKSGGYMTANEWIWDKESWFYLKSDGKIAEKEWVYDSHSQAWYYFKSGGYMTANEWIWDKESWFYLKSDGKMAEKEWVYDSHSQAWYYFKSGGYMAKNETVDGYQLGSDGKWLGGKTTNENAAYYQVVPVTANVYDSDGEKLSYISQGSVVWLDKDRKSDDKRLAITISGLSGYMKTEDLQALDASKDFIPYYESDGHRFYHYVAQNASIPVASHLSDMEVGKKYYSADGLHFDGFKLENPFLFKDLTEATNYSAEELDKVFSLLNINNSLLENKGATFKEAEEHYHINALYLLAHSALESNWGRSKIAKDKNNFFGITAYDTTPYLSAKTFDDVDKGILGATKWIKENYIDRGRTFLGNKASGMNVEYASDPYWGEKIASVMMKINEKLGGKD

SEQ ID NO: 127 - protein, 252 aa, Streptococcus pneumoniae
MVVYIRQSKLPSEVSINKYHAQVGAYLQGEEAVLYQSFSEIKELTSEDIVVDYIMETRALLKMMGLNVPVHDYPIELKEFYGRKIYAGILGEIVNIPDNWGKFIKPKAGSKVFTGRVVNGTHDLIGIGLPFDYPIWISEVVEFIAEWRCFVLDGRVLDVRPYTGDYHAQFDASVIDEAISCWKDAPIAYGLDIGVTRDGRTLVVEVNDGYALGNYGLSPLKSINFHRARWKEMVKPYFEKNEIFKIQQDVVF

SEQ ID NO: 128 - protein, 290 aa, Streptococcus pneumoniae
MLAEAIIKNGIEIVVVTDHNTTKGIKKLQMAVSIIMKNYPIYDIHPHILHGVEISAADKLHIVCIYDYEQESWVNQWLSENIISEKDGSYQHSLTIMKDFNNQKIVNYIAHFNSYDILKKGSHLSGAYKRKIFSKENTRFLEFNINSKESSQQLDILYKEVGVLSLGQKVVAMLDFLLAYSDYSKDFRPLIIDQPEDNLDNRYIYRHLVQQFRDVKAQRQIILATHNATIVTNSMTDQVVIMESDGVNGWIESQGYVSEKYIKNHIINQLEGGKDSFKHKMSIYETALSE

SEQ ID NO: 129 - protein, 200 aa, Streptococcus pneumoniae
MSEVDFNEAVNYEFTSDTCQLANSIYQSLFKFFDKKNFSGDLIFTWKSPSLVKEGDYIGRRDSQVDNLRVIGNIFPNYLTNRKYSLNMNRNGCMGDFPHDFFDIYLDHVAKYAYEQKVNNIKEYYPLKRAILHQENALYFRFFSNFDDFLEKNYLKTIWQVSKETPFSEMDFNMFKNISEKIIFERGSKMLNDLKSNYKK

SEQ ID NO: 130 - protein, 243 aa, Streptococcus pneumoniae
MVNNVAVKVSNLSKEFLLGQDKTVSILKDISLSVNYGEFVSILGVSGSGKSTLLSCLSSLSEPTSGEVVINGVNPYTLKEGKLAKFRRQDIAIIFQNYNLVPALPVLENVTLPLRLSGKSVDSNKVKKMLDSLNFKAELSSLVATLSGGEQQKVAITRAIIADSKIIFADEPTGALDSVSRKLIFETLRNLASQGKCVLMVTHDIELASKTDRALILKDGKISRQIIKPSADELYQALESSKD

SEQ ID NO: 131 - protein, 1032 aa, Streptococcus pneumoniae
MAKLIPGKIRIEGVALYETGKVDIIKEKNNRLYARVAEEELRYSLEDDLVFCACDFFQKRGYCVHLAALEHFLKNDERGQEILWSLEEGHEEKEAVETKVTLGGKFLDRILSPKSECAYELSAVGQVEAGTNHILWTLRIGQINSQKYYVIRDIPLFLRIVEQRKSYMIGKIYEESLSWEAFDDASQELLTFLHGLTEEGLVSDLFFQNQGRHLFFPLTFFEQGVNLLMALPHFQFDHQVDSYQTLLFQDMHADANLFAFTVTEYSDYFEMEISESPRVNVFYQGAVLFHKGQVYFLTDQQMRLLKEIKALPVGQQGKKYLQFDSSDRDKLASCLTLFIQMGTVSAPERLQIKTFAPSFYFDREEDNRIRLEIQFDYGNRQVSSRQELEELPFSSDADLEERIFQVCLAAGFEADFQSWRQALKAESVYHFFHDIIPVFEKLGHVDLSDKLEELYSLASPQVQIASKGGLLEIQFDFQDIAQEEIDQAMQALVANQDFYIDSSNQVYFFDEETKKIRQNLQELGQFELKDGTLQARKSLAYSLAHLFEGRDRVSFSQEFQNLAQDLTHPEDFPRQATQVQADLRDYQEKGIGWLQMLHHYGFGGILADDMGLGKTLQTIAFLTSQVTKESRVLILAPSGLIYNWADEFQKFAPQLDVAVVHGLKASREEILAESHQIYVTSYATFRQDSELYQGMAFDFLFLDEAQVMKNAQTKIAQTLRQFVVPSVFALSGTPIENHLGELWSIFQIVMPGLLPSKKEFMKLPAERVAQFIKPFVMRRKKEEVLTELPDLIEVVYKNELEDQQKAIYLAQLQQMRDHLAQVSEQEFQRSRVEILSGLMRLRQICDTPALFMEDYQGASGKLDSLRDLLVQVADGGHRVLIFSQFKGMLEKIEQELPDLGLTSFKITGSTPAKERQDMTKAFNQGERDAFLISLKAGGVGLNLTGADTVILVDLWWNPAVEAQAIGRAHRMGQEETVEVYRLVTKGTIEEKIQELQEQKKHLVSQVLDGTESRGSLTLAEIREILGISEAST

SEQ ID NO: 132 - protein, 179 aa, Streptococcus pneumoniae
LTEKIQEHELIKTNQAEKSVQDVLDNCIERVQNNSLKSDRVTSFETPFALLFIFATIAVMLTYGGYRVSAGYISVGTLVSFLIYLFQLLNPISNIANFVTVYSRSKGSSVALDNLLAVPKEKFEGGKSVSGQGLNFNHVYFGYDENRPVLKDITCSIFKGQKLLLLDHLDQENQRLCVC

SEQ ID NO: 133 - protein, 434 aa, Streptococcus pneumoniae
MTPIKDKVRRVKTPMMVNPDTDLTISSVQQDYFSLALIGFSLLTGDFLSFSKGDQKTGLSAFIKICHLIKIARLDNKITKQQEYWLYDLLMMSQGEKIQKIKQLKQVTSDILLNTPDFSSYFEKYNFKEEAENIKSYLLAKSMDKSGRLFPSNEFGEFVSPVSFQHGFGGVLFFMNKYYVEEDENTVKEWLTKLENYEAANFLHGYSLLFGKAGFLFGILDRYEKTKERYLIDISKRLVDHLMRVYDNISNLDFALGKSGILLSLMKYCTIFDDKKLANFIKNNINDAYSLLESEDNGDIYSNNFAHGRSGAAYVLKAYTDIFGDSRYQNHLQKFSDGISELLEEKLSSFSKLDNLGLSWCDGVSGLILYLCLIDKERYSEIIYKSQLEMVQQYEAMGTSFCHGLSSLLQTTIYNKNQKVEQFIKKNLIDSFLS

SEQ ID NO: 134 - protein, 277 aa, Streptococcus pneumoniae
MKKKSGIYLDILSHVLLVGATIVAVFPLVWIIISSVKGKGELTQYPTRFWPEQFTLDYFTHVINDLHFIDNIRNSLIIALATTLIAIIISAMAAYGIVRFFPKLGAIMSRLLVITYIFPPILLAIPYSIAIAKVGLTNSLFGLMMVYLSFSVPYAVWLLVGFFQTVPIGIEEAARIDGANKFVTFYKVVLPIVAPGIVATAIYTFINAWNEFLYALILINNTGKMTVAVALRSLNGSEILDWGDMMAASVIVVLPSIIFFSIIQNKIASGLSEGSVK

SEQ ID NO: 135 - protein, 255 aa, Streptococcus pneumoniae
MFWNLVHYEFKNVNKWYLALYAAVLVLSALIGIQTQGFKNLPYQESQATMLLFLATVFGGLMLTLAISTIFLIIKRFKGSVYDRQGYLTLTLPVSEHHIITAKLIGAFIWSLISTAVLALSAVIILALTAPEWIPLSYVITFVETHLPQIFLTGISFLLNTISGILCIYLAISIGQLFNEYRTALAVAAYIGIQIVIGFIELFFNLSSNFYVNSLVGLNDHFYMGAGIAIVKELIFIAIFYLGTYYILRNKVNLL

SEQ ID NO: 136 - protein, 399 aa, Streptococcus pneumoniae
MRNMWVVIKETYLRHVKSWSFFFMVISPFLFLGISVGIGHIQGSSMAKNNKVAVVTTVPSVAEGLKNVNGVNFDYKDEASAKEAIKEEKLKGYLTIDQEDSVLKAVYHGETSLENGIKFEVTGTLNELQNQLNRSTASLSQEQEKRLAQTIQFTEKIDEAKENKKFIQTIAAGALGFFLYMILITYAGVTAQEVASEKGTKIMEVVFSSIRASHYFYARMMALFLVILTHIGIYVVGGLAAVLLFKDLPFLAQSGILDHLGDAISLNTLLFILISLFMYVVLAAFLGSMVSRPEDSGKALSPLMILIMGGFFGVTALGAAGDNLLLKIGSYIPFISTFFMPFRTINGYAGGAEAWISLAITVIFAVVATGFIGRMYASLVLQTDDLGIWKTFKRALSYK

SEQ ID NO: 137 - protein, 645 aa, Streptococcus pneumoniae
MDYSKVAAEVIEAVGKDNLVAAAHCATRLRLVLKDEAKVNQAALDNNADVKGTFSTNGQYQIIIGPGDVNFVYAEIIKKTGLKEVSTDDLKEIANKDKKFNPLMDLIKLLSDIFVPIIPALVAGGLLMALRNFLTSPDLFGPQSIEDMYPAIKGFSAMIQLMSAAPFMFLPVLVGISAAKRFGANQFLGAAIGMIMTTPDLGGKEAFWDILGFHVTQTNYAYQVIPVLVAVWLLANLEKFFHKKLPSAVDFTFTPLLSVMITGFLTFTVIGPVMLVVSDAITNAIVWLYNTTGAFGMGLFGGTYSLIVMTGLHQSFPAIETQLLSAYNNNGTGFGDYIFVVASMANVAQGAATLAVYFLTKNAKTKGLSSSAAVSAFLGITEPALFGVNLKYKFPFFCALAGSAIGAFVAGLTHVIAVSLGAAGFIGFLSIKAGSIPMYIIAEIMSFVAAFAFTYFYGKTKAASVFADEAATATAETVTEPTVEAPVVEETDTLQNETLVTPIVGDVVALADVNDPVFSSGAMGQGIAVKPSQGVVYAPADAEVSIAFPTGHAFGLKTRNGAEVLIHVGIDTVSMNGDGFEAKVAQGNKVKAGDVLGTFDSNKIAAAGLDDTTMVIVTNTADYASVAPVATGSVAKGDAVIEVKI

SEQ ID NO: 138 - protein, 256 aa, Streptococcus pneumoniae
VVEIIYFLIIIIASGLGSISGMGGGIIIKPLMDSFGYHSVSDIAFYSSFSVFIMAIISTTKRFSQSKEIKWRLIFTVSFSSVLGGFLGHLIFQVLLSQLSVRLVSIVQMILLFVMLLVSFVLTDFKKTYQFDKIGFYMICGLLLGLISSFLGIGGGPLNVSLLMVFFSISIKEATMYSLAIIFFSQLSHLATIVVVTGLNQYHLAPVPVIFLASICGGVLGTVVSKVLPENWVRYCFKGMLFFVMGMTLYNLFHIL

SEQ ID NO: 139 - protein, 451 aa, Streptococcus pneumoniae
MNTMLDKMQEKLSPIAMKVGNQKFLVALRDSFVGTMPVIMTGSIALLLNAFLVDLPQQFHLESITKTFQWLVDINNLVFKGSIPIVSLLFIYCLGVNIAKIYKVDTVSAGLVSLASFVISIGSTVTKSFPLANVGDVKLDQILQGIDNLAFDGKNLMVTIGNVIPGNHINARGYFTAMMIGFLASIIFCKVMKKNWVIKLPDSVPPAIAKPFTSIIPGFMAMYIVAILTYVFHLLSNDLLIDWVYKVLQTPLLGLSQSFFAVILMIFLNKLFWFFGLHGGNVLAPIMEGLFGVAMLANLDAFQKGEPIPYIWTSGSFGAFVWFGGLGLVLAILIFSRNSHYRKVAKLGLAPVLFNIGEPVNYGLPVVLNPLLFIPFVLSPVFMATVAYWATSWGLVSPVTQNVTWVMPPILYGFFSTAFDWRAIILSVVCLIISVLTYFPFVKMAYKTELS

SEQ ID NO: 140 - protein, 236 aa, Streptococcus pneumoniae
MTATSLGLSNIEIVVRIVLSVVIGSIIGLERGSKSQPAGIRTYSIVCLAACLIMMTNEYVSYKFGTGDPTRLGAQVISGVGFLGAGTILITDKKKITGLTTAAGIWASAGIGLAIGVGFYEGALLVAISVWGVISMFQPLKKYLQNRSKMIELYIVVKSTEAYNRVLVYCAENGIRMTDSRTAFGDVNSDRIEYFDVPDKKIASFITLELSGRFEHLRLMEEIANIVGVIYVEEIS

SEQ ID NO: 141 - protein, 219 aa, Streptococcus pneumoniae
MKNLTKIKFKENGEFNHFSGNTVVANLYTKQDLMEVVDIIQSRYRELPFIDKFTLTPRNSIHMTVIELLCHENRETEFWSSNLPLDTPLQEIHDYFAKQLEIFPLLDEEIHMRITEMGKQNILVEPADEASAKRLEEIRTYVSEKAGVCFPNHDRYQFHISIGYLRIPLTEEEEEEFTKVRAELTEILLEKIPTITVNRIDYTVFEDMRQFVPYHEKFK

SEQ ID NO: 142 - protein, 254 aa, Streptococcus pneumoniae
MELHFNLELVETYKSNSQKARILTEDWVYRQSYCPNCGNNPLNHFENNRPVADFYCNHCSEEFELKSKKGNFSSTINDGAYATMMKRVQADNNPNFFFLTYTKNFEVNNFLVLPKQFVTPKSIIQRKPLAPTARRAGWIGCNIDLSQVPSKGRIFLVQDGQVRDPEKVTKEFKQGLFLRKSSLSSRGWTIEILNCIDKIEGSEFTLEDMYRFESDLKNIFVKNNHIKEKIRQQLQILRDKEIIEFKGRGKYRKL

SEQ ID NO: 143 - protein, 309 aa, Streptococcus pneumoniae
MLKILNNSVIYLCLIPLLFLLLIIFPNDSLIYYFRLILISLISITLHELGHFLVGRCLSYKLEMLATPFFFYFRKKIYFKFPVLLAFGYCQMSNRNITNEKNSDRNLVFYFFGGGGANLIVAILALLGFVPYASEFFILNIILFLVTVCLPIDGTDGNAIREIVLYSKDSKTYQRFFANSLYNNPYITIDDFAKLTSKEKSFFSKFEKCLINLYFEVKGEGSAFTIANHEVFDSNIQENIIHEYYKLLHKSSDWIIPQNLSLEEVVFTLSNYIYSKNNKYLEKIKYLKKLVDFRQEEIIDFILNKEEVL

SEQ ID NO: 144 - protein, 380 aa, Streptococcus pneumoniae
MSDSPIKYRLIKKEKHTGARLGEIITPHGTFPTPMFMPVGTQATVKTQSPEELKEMGSGIILSNTYHLWLRPGDELIARAGGLHKFMNWDQPILTDSGGFQVYSLADSRNITEEGVTFKNHLNGSKMFLSPEKAISIQNNLGSDIMMSFDECPQFYQPYDYVKKSIERTSRWAERGLKAHRRPHDQGLFGIVQGAGFEDLRRQSAHDLVSMDFSGYSIGGLAVGETHEEMNAVLDFTTQLLPENKPRYLMGVGAPDSLIDGVIRGVDMFDCVLPTRIARNGTCMTSQGRLVVKNAQFAEDFTPLDPECDCYTCNNYTRAYLRHLLKADETFGIRLTSYHNLYFLLNLMKQVRQAIMDDNLLEFREYFVEKYGYNKSGRNF

SEQ ID NO: 145 - protein, 271 aa, Streptococcus pneumoniae
MSKYLLKLLVYCFSALTFGSLFLIIGFILIKGLPHLSLSLFSWTYTSENISLMPAIISTVILVFGALLLALPIGIFAGFYLVEYTKKDSLCVKIMRLASDTLSGIPSIVFGLFGMLFFVVFLGFQYSLLSGILTSVIMVLPVIIRSTEEALLSVSDSMRQASYGLGAGKLRTVFRIVLPVAMPGILAGVILAIGRIVGETAALMYTLGTSTNTPSSLMSSGRSLALHMYMLSSEGLHVNEAYATGVILIITVLMINTLSSLLSRKLVKGAS

SEQ ID NO: 146 - protein, 423 aa, Streptococcus pneumoniae
MSSKFMKSTAVLGTVTLASLLLVACGSKTADKPADSDSSEVKELTVYVDEGYKSYIEEVAKAYEKEAGVKVTLKTGDALGGLDKLSLDNQSGNVPDVMMAPYDRVGSLGSDGQLSEVKLSDGAKTDDTTKSLVTAANGKVYGAPAVIESLVMYYNKDLVKDAPKTFADLENLAKDSKYAFAGEDGKTTAFLADWTNFYYTYGLLAGNGAYVFGQNGKDAKDIGLANDGSIAGINYAKSWYEKWPKGMQDTEGAGNLIQTQFQEGKTAAIIDGPWKAQAFKDAKVNYGVATIPTLPNGKEYAAFGGGKAWVIPQAVKNLEASQKFVDFLVATEQQKVLYDKTNEIPANTEARSYAEGKNDELTTAVIKQFKNTQPLPNISQMSAVWDPAKNMLFDAVSGQKDAKTAANDAVTLIKETIKQKFGE

SEQ ID NO: 147 - protein, 502 aa, Streptococcus pneumoniae
MSQEKYIMAIDQGTTSSRAIIFNKKGEKVSSSQKEFTQIFPQAGWVEHNANEIWNSVQSVIAGAFIESGVKPNQIEAIGITNQRETTVVWDKKTGLPIYNAIVWQSRQTAPLAEQLKSQGYVEKFHEKTGLIIDAYFSATKVRWILDHVEGAQERAEKGELLFGTIDTWLVWKLTDGAAHVTDYSNAARTMLYNIKELKWDDEILEILNIPKAILPEVRSNSEIYGKTAPFHFYGGEVPISGMAGDQQAALFGQLAFEPGMVKNTYGTGSFIIMNTGEEMQLSENNLLTTIGYGINGKVYYALEGSIFIAGSAIQWLRDGLRMVENSPESEKYARDSHNNDEVYVVPAFTGLGAPYWNQNARGSVFGLTRGTSKEDFIKATLQSIAYQVRDIIDTMQVDTQTAIQVLKVDGGAAMNNFLMQFQADILGIDIARAKNLETTALGAAFLAGLSVGYWKDLDELKLLNETGELFEPSMNESRKEQLYKGWKKAVKATQVFAEVDD

SEQ ID NO: 148 - protein, 804 aa, Streptococcus pneumoniae
MRYSIRKFSVGVASVAVASLFMGSVVHATEKEGSTQAATSFNRGNGSQIERRKAAEQAIVSLSGYMTSLLNDGLDTSDSRFEELLKRAKGIVEEYRKKIDGASDREKIEKLQQEGQTKLDELVDKFKKGLSSSEQNGHATPKDPSRNDGVQGDDSSVGQGEQPGASNQKNPESVPPTASGETDSPTKPKTSNDSVNSLERELKEVKETAKTTLNTYMLTRLKQENPGVFWFADLLRESKESVEKYKRMFDEASSKENVENLVKEAKQEIEALVVKHKGREIDLERTKAKTVIAKYLTGLLDDIKKNLKKEQHINTVELIKKLGDIKRTYLYKLDESTQKAQLQELVTESQSKLDEAFSKFKNGLSSSSSSGSSTKPETPQPEKPEHQKPTTPAPDTKPSPQPEGKKPSVPDINQEKEKAKLAVATYMSKILDDIQKHHLQKEKHRQIVALIKELDELKKQALSEIDNVNTKVEIENTVHKIFADMDAVVTKFKKGLTQDTPKEPGNKKPSAPKPGMQPSPQPEVKPQLEKPKPEVKPQPEKPKPEVKPQLEKPKPEVKPQPEKPKPEVKPQLEKPKPEVKPQLEKPKPEVKPQPEKPKPEVKPQLEKPKPEVKPQPEKPKPEVKPQLEKPKPEVKPQPEKPKPEVKPQLEKPKPEVKPQLEKPKPEVKPQLEKPKPEVKPQPEKPKPEVKPQLEKPKPEVKPQPEKPKPEVKPQPEKPKPEVKPQLEKPKPDNSKPQADDKKPSTTNNLSKDKQPSNQASTNEKATNKPKKSLPSTGSISNLALEIAGLLTLAGATILAKKRMK

SEQ ID NO: 149 - protein, 289 aa, Streptococcus pneumoniae
MESNLKKTTFIIVMIGISLIPALYNIIFLSSMWDPYGQLSDLPVAVVNNDKEASYNGNTMAIGKDMVSNLKENKTLDFHFVDEEEGKKGLEDGDYYMVVTLPSDLSEKATTLSNIQSTAAYQSLTSEQQTEISDSVSQNSTDSIQSAQSIVALVQDLQGSLENLQNQSSNLSTLKNQANQVLPITSTSLIGLSSGLTEIQGAVTSKLVPASQSIASGVNAYTTGVDKVSQGASQLSEKNATLTGSLDQLVSGSNTLTQKSSSLTAGVGQLVEKTPELVSGIEKLSTGSN

SEQ ID NO: 150 - protein, 417 aa, Streptococcus pneumoniae
MKCFVGLDISSTKLDVCIMLSDTSTPVTASLSNDLSGATEIKNHILDLNHTYHFERIVIGMEATSLYSFHPAMFFHEDRQLKELHVEIMVEQPNKIKKYRDAFEENKNDTIDAFYIADYFRIERFSPSFLKEEKYMALQHLTRTRLQIIEQLTRTKQHFIENIYYKCNTLAYQFKKEELSTNLWSSTMMTLMTDEMTLDELANMPLQELDDWLQKLGKGHFKDSEKLAQTIQAAIRGSYRLSKLQQDSVNVILGLLAREIRNLEKMIKEIDKAIEDMVETIPEYQCLTSIPGVGKVYAAGIIAEIGQIERFKDHPQVAKFAGLNWREKQSGNSNSQHTSLVNRGNRYLRYYLVEAANSVRRYDDEYKDFYKKKYHEVPKHQHKRAILLTARKFVRLVDVLLRNHQLYTPPRRIMEDN

SEQ ID NO: 151 - protein, 307 aa, Streptococcus pneumoniae
MAKKKIKKEKIDNVGIHSFSKKADIFFSIISGLIALSCILPFVFVIIISVTDEKSLLQYGYSFFPSQFGLDGFEFLAQFKDKILQALFISVFVTVVGTLTNVFITTTYAYAISRTTFKYRRFFTIFVLLSMLFNAGLVPGYIMVTRVLQLGDTVWALIVPMLLSPFNIILMRSFFKKTIPEAILESARIDGASEARIFFQICLPLSLPGIATITLLTALGFWNDWFNALLYIKSDNLYPLQYLLMQIQQNMDYIAKAVGLTGQLGVALPKETGRMAMVVVATLPIAILYPFFQRYFVKGLTIGGVKE

SEQ ID NO: 152 - protein, 352 aa, Streptococcus pneumoniae
MKDDQKYLLAGLYSLLVAIFYFPLIESKGIFVSILMAVLLLYLIYFIGTVIHIVIIKFIRKKSFKYLVLYPFTYDGSWRFQPINLLYFPEMVRDVIPINLVQEYCQGQPYGLLKKMLKRIRLSREIALLLATVIVYFFTHRILPLSVFTFIFSYILLFAQSYLGGNTVWIGNRRLIIDDEFEKILLSKSYIKEISSARYSEYLTCEYKNFTPIILLAIFENLLDSYLIQNQSKVDLDIFYKVLPLLYKEKYTMGFNYFVSLNYLLYKVGFLGIIYDNEALRDLSKQYLNKNISELQDGSFEGGIQDAVASKQIVVINEFIACLNSRCVPSQYDRFFYKDRPYIFSRKSPIKG

SEQ ID NO: 153 - protein, 428 aa, Streptococcus pneumoniae
MYKVLLVDDEYMVTEGLKRLIPFDKWDMEVVATASHADEALEYVQENPVDVIISDVNMPDKTGLDMIREMKEILPDAAYILLSGYQEFDYVKRAMNLSVVDYLVKPVDKVELGNLLEKIAGQLGERGKKSQTLSQELDEAGFVSYLGDKENWWIGLSKEKQGSFTIPYYVLGQDWQIFISDQPLDGLVVTPFEAPYQEHFERWKLNAEKTLFYGSVNLQQSESLFAYYEPIYRVIIQGNLNQIVEELNLLEKVVLENTPRVPITKQLFIQFVMDVFHLFEHLKADDLTDIVKTIHAIQSFDELVPYIKETLTSFFGQYRMNENVVSVLEVIGRDYQKELSLKDISKALFINPVYLGQLIKRETDSTFAELLNKQRIKAAQQLLLSTSDSIEDICYAVGYSNLGYFYKVFRKLCGKSPKAYRKQVETIL

SEQ ID NO: 154 - protein, 185 aa, Streptococcus pneumoniae
MDKKNLLVLVTGSVGASNVDTYLYYIKKFYNVKVILSENSKKFISKELISYFCDKVYDEIFVEEVVPHVFLPYESDLLLILPATANVIGKIANGIADDLVTATVLNFNKKIIFCPNMNSTMWDNHIVQRNVSILKELGHIFLFESKKTYEVGLRKAIDSTCSMLQPQSLVKELIKLENIVLEEGH

SEQ ID NO: 155 - protein, 343 aa, Streptococcus pneumoniae
MVSTIGIVSLSSGIIGEDFVKHEVDLGIQRLKDLRLNPIFLPHSLKGLDFIKDHPEARAEDLIHAFSDDSIDMILCAIGGDDTYRLLPYLFENDQLQKVIKQKIFLGFSDTTMNHLMLHKLGIKTFYGQSFLADICELDKEMLAYSLHYFKELIETGRISEIRPSDIWYEERTDFSPTALGTPRVSHTNTGFDLLQGRAQFEGKILGGCLESLYDIFDNSRYADSTELCQKYKLFPDLSDWEGKILLLETSEEKPKPEDFKKMLLTLKDTGIFAVINGLLVGKPMDETFHDDYKEALLDIIDSNIPIIYNLNVGHATPRAIVPFGVHAHVDAQEQVIRFDYNK

SEQ ID NO: 156 - protein, 349 aa, Streptococcus pneumoniae
MVELGISTFGEITELEGTGQTYSHAERIRQLVAEIELADKVGLDVYGIGEHHRADFAVSAPEIVLAAGAVNTKKIRLTSAVSILSSMDPIRLFQQYATIDALSNGRSEIMAGRGSFTESFPLFGYDLKDYDSLFDEKLDLLQLVNEKTKLDWQGRLTQTIAGKEVYPRPVQDKLPLWIATGGHVESTVKIAQAGLPIVYAIIGGNPRYFKKLIQAYREIGSEAGHADKDLKVGAHSWGWIAEDGEQAVKDYFHPTKQVVDAISKDRPHWQELRYEQYLEQVGPNGAMFVGNPDQVAEKLIRMIEDLGLDRFMLHLPLGSMPHDQVLRAIELFGTQVAPKVRAYFAMKEA

SEQ ID NO: 157 - protein, 339 aa, Streptococcus pneumoniae
MKAVVVNPESTGVAIEEKVLRPLETGEALVEVEYCGVCHTDLHVAHGDFGQVPGRVLGHEGIGIVKEIAPDVKSLKVGDRVSVAWFFEGCGTCEYCTTGRETLCRTVKNAGYSVDGGMAEQCIVTADYAVKVPDGLDPAQASSITCAGVTTYKAIKEAKVEPGQWVVLYGAGGLGNLAVQYAKKVFNAHVIAVDINNDKLALAKEVGADIVINGLEVEDVAGLIKEKTDGGAHSAVVTAVSKVAFNQAVDSVRAGGRVVAVGLPSETMELSIVKTVLDGIQVIGSLVGTRKDLEEAFQFGAEGLVVPVVQKRPVEDAVAIFDEMEKGQIQGRMVLDFTH

SEQ ID NO: 158 - protein, 254 aa, Streptococcus pneumoniae
MSYIQLDFHSHSLNRNVNLNIFLPDGELLPAPEQPYPTIYFLPGFSASGKELSTFLHFRTQCLLNGIAVVIPDGDNSFYLDKPERLALYSQYVSQELIDYTCSILPLSHKREETYIGGISMGGYGALVHGFRYPEKFGKIVALSPAIDVAAILNGNASQFGTTFADLFKTEEDYYSSTDHPVNALKELLDKGEDIPELFIACGFQDELVYASNRSFAEELEKLGVNLTYIEDDGGHDVFFWDKYLPAVFEFLRK

SEQ ID NO: 159 - protein, 158 aa, Streptococcus pneumoniae
MTSLYDFSVLNQNNQATPLDSYRGKVLLIVNTATGCGLTPQYQGLQELYERYQDQGFEILDFPCNQFMGQAPGSAEEINAFCSLHFQTTFPRFAKIKVNGKEADPLYVWLKDQKSGPLGKRVEWNFAKFLIGRDGQVFERFSSKTDPKQIEEAIQTLL

SEQ ID NO: 160 - protein, 701 aa, Streptococcus pneumoniae
MNNNFNNFNNMDDLFNQLMGGMRGYSSENRRYLINGREVTPEEFAHYRATGQLPGNAETDVQMPQQASGMKQDGVLAKLGRNLTAEAREGKLDPVIGRNKEIQETSEILSRRTKNNPVLVGDAGVGKTAVVEGLAQAIVNGDVPAAIKNKEIISIDISGLEAGTQYRGSFEENVQNLVNEVKEAGNIILFFDEIHQILGAGSTGGDSGSKGLADILKPALSRGELTVIGATTQDEYRNTILKNAALARRFNEVKVNAPSAENTFKILQGIRDLYQQHHNVILPDEVLKAAVDYSVQYIPQRSLPDKAIDLVDVTAAHLAAQHPVTDVHAVEREIETEKDKQEKAVEAEDFEAALNYKTRIAELEKKIENHTEDMKVTASVNDVAESVERMTGIPVSQMGASDIERLKDMAHRLQEKVIGQDKAVEVVARAIRRNRAGFDEGNRPIGSFLFVGSTGVGKTELAKQLALDMFGTQDAIIRLDMSEYSDRTAVSKLIGTTAGYVGYDDNSNTLTERVRRNPYSIILLDEIEKADPQVITLLLQVLDDGRLTDGQGNTVNFKNTVIIATSNAGFGYEANLTEDADKPELMDRLKPFFRPEFLNRFNAVIEFSHLTKEDLSKIVDLMLAEVNQTLAKKDIDLVVSQAAKDYITEEGYDEVMGVRPLRRVVEQEIRDKVTDFHLDHLDAKHLEADMEDGGLVIREKS

SEQ ID NO: 161 - protein, 141 aa, Streptococcus pneumoniae
MKLLKKMMQVALATFFFGLLGTSTVFADDSEGWQFVQENGRTYYKKGDLKETYWRVIDGKYYYFDPLSGEMVVGWQYIPAPHKGVTIGPSPRIEIALRPDWFYFGQDGVLQEFVGKQVLEAKTATNTNKHHGEEYDSPAEK

SEQ ID NO: 162 - protein, 208 aa, Streptococcus pneumoniae
MKGSFALLLFVILGYMVKFYPEMLVNFDQSIQTAIRGDLPDYLTILFRALTRLIDIPVIITWVVITAFVFYRKRWKIESFFMLGNLALAGLLIVTFKNIYQRPRPAILHLVEEKGFSFPSGHSLAVTLMVGTLIVILSQRIKNPVWRKIVQIVLGLYLVSVLVSRVYLGVHYPSDVLASLCVGLGVLFIEFPFYDKLRFQWRFKGKQK

SEQ ID NO: 163 - protein, 333 aa, Streptococcus pneumoniae
MIKTFLSALSVILFVIPIITYSFFPSSNLNIWLSTRPILAQIYAFPLATATMAAILSFVFFSLSFYKKNKQIRFYSGILLLLSLILLLFGTDKTLSSASNKTKTLKLVTWNVANQIEAQHIERIFSHFDADMAIFPELATNIRGEQENQRIKLLFHQVGLSMANYDIFTSPPTDSGIAPVTVIVKKSYDFYTKAETFHTTRFGTIVLHSRKQDIPDIIALHTAPPLPGLMEIWKQDLNIIHNQLASKYPKAIIAGDFNATMRHGALAKISSHRDALNALPPFERGTWNSQSPKLFNATIDHILLPKNHYYVKDLDIVSFQNSDHRCIFTEITF

SEQ ID NO: 164 - protein, 673 aa, Streptococcus pneumoniae
MKKLFILLSTFFLSFFLAWIIVLRAPQYLYASYDSVSLLRVKKDTQEPTREVFEQELENFANSEQSLIARRIVEPSKDGTTHFTYATYGQGTLPKEFQEASQESRERSDPLNSYLLLSGSLTKEKLADKLGDLGYKASADRKIPPYFLAFRILLNPLILISLAIFGLSFFALVIITRIKEMRAAGIKLFSGQTLLSIMGHSLSTDIKWLLLSALLSFLGGGVVLFSQGLFYPILLATYGFGISFYLLFLLAISILLMLLYLMSLNYKALVPVIKGRLPLKRLMILTLLCQLVAVFTVGYAVKTGLTSYQRLKELEISKQAWQDRADYYQISFGLGDRGKDTENQSKWYAFAKEAIEEEQALYVKDNLLHFANPQGKNEQGETLDTYSPDANTLYVSPSYLDKEKVVVDAETKQKLAHLQKGEFILLLPEHLRSREVELKKVFEERLSYYGKSGEEASAPLDYEMKAHVSYLSMGEKRFVYNNGENPVSTQYLTDPILVVFTPTSTGDSFISLSSWSINAGKQLFIKGYESGLELLKKVGIYEQVSYLKEGRSVYLTRYNEVQTETATLILGAIVGIASSLLLFYSVNLLYFEQFRRDILIKRISGLRFFETHAQYMVSQFASFVFGASLFILSSRDLVIGLLTLLVFLASAVLTLYRQAQKESRVSMTIMKGK

SEQ ID NO: 165 - protein, 237 aa, Streptococcus pneumoniae
MLKRFLALVWLRCQIILSNKSILLQVLVPFAFTYFYKYLMETQGKVNDQQALVLLMMCLPFSLALAVGSPITIILSEEKEKYNLQTLLLSGVKGSEYILSTMFLPFLLTFVIMGTTPLILGVTIVHTFNYITIVLLTSLSIILFYLLIGLTAKSQVVAQVISLPAMILVAFLPMLSGLDKTVAKITDYSFMGLFTKFFTKWEGFSWNETLIPNLTLLIWIVLLLTLITITIRKKKIS

SEQ ID NO: 166 - protein, 461 aa, Streptococcus pneumoniae
MGLELRAIQSPIFSEPFDFTFHAQAFTLLVGSSGSGKSSLFQVIAQVSSLPYSGQVLIDGSEVSQLSIIARVQKVGILFQNPNHQFTMENLFEELIFTLENIGYHLQEIDSKIAEVVQQCRCEAILHRPIHHLSGGEKQKAALAVLFAMNPRVYLLDEPFASIDRKSRIEILEILKELALDGKTVILCDHDLSDYKAYIDHMVELRDGKLREVFQIPSYEMTQVASKEVASSPELFHMNRVTGELGNRPLFSIADFTFYQGISCILGDNGVGKSTLFRSILQFQKYKGRIAWKGTVLKKKKSLYRDLTGVVQEAEKQFIRVSLREELQLDGPDSERNQRIFQALRYFDLEQAVDKSPYQLSGGQQKILQLLTILTSKASVILLDEPFAGLDDRACHYFCKWIVEERNQGRSFLLISHRLDPLISVVDYWIEMTSQGLRHVKEVTITKPLTSQSSNTQGEVR

SEQ ID NO: 167 - protein, 747 aa, Streptococcus pneumoniae
MTEIVKASLENGIQKIRIRSEKGYHPAHIQLQKGIPAEITFHRATPSNCYKEILFEEEGILEPIGVDEEKVIRFTPQELGRHEFSCGMKMQKGSYTVVEKTRKSLSLLQRFWITSIFTVPLVILMIGMLTGSISHQVMHWGTFLATTPIMLVAGKPYIQSAWASFKKHNANMDTLVALGTLVAYFYSLVALFAGLPVYFESAGFILFFVLLGAVFEEKMRKNTSQAVEKLLDLQAKTAEVLSDDSYVQVPLEQVKVGDLIRVRPGEKIAVDGVVVEGVSSIDESMVTGESLPVDKTVGDTVIGSTINHSGTLVFRAEKVGSETVLAQIVDFVKKAQTSRAPIQDLTDKISGIFVPVVVILGIMTFWVWFVLLRDSVVVLGASFVSSLLYGVAVLIIACPCALGLATPTALMVGTGRSAKMGVLLKNGTVLQEIQKVQTLVFDKTGTLTEGKPVVTDVIGDEVEVFGLAASLEDASQHPLAEAIVKRASEAGLEFQTVENFQTLHGKGVSGRINGKQVLLGNAKMLDGMDISNTYQDKLEELEKEAKTVVFLAVDNEIKGLLALQDIPKENAKLAISQLKKRGLRTVMLTGDNAGVARAIADQIGIEEVIAGVLPEEKAHEIHKLQQSGKVAFVGDGINDAPALSVADVGIAMGAGTDIAIESADLVLTTNNLLGVVRAFDMSKKTFHRILLNLFWAFIYNVVGIPIAAGVFSGVGLALNPELAGLAMAFSSVSVLTSSLLLNFSKID

SEQ ID NO: 168 - protein, 726 aa, Streptococcus pneumoniae
MMKDTFKNVLSFEFWQKFGKALMVVIAVMPAAGLMISIGKSIVMINPTFTPLVITGGILEQIGWGVIGNLHILFALAIGGSWAKERAGGAFAAGLAFILINRITGTIFGVSGDMLKNPDAMVTTFFGGSIKVADYFISVLEAPALNMGVFVGIISGFVGATAYNKYYNFRKLPDALSFFNGKRFVPFVVILRSAIAAILLAAFWPVVQTGINNFGIWIANSQETAPILAPFLYGTLERLLLPFGLHHMLTIPMNYTALGGTYDILTGAAKGTQVFGQDPLWLAWVTDLVNLKGTDASQYQHLLDTVHPARFKVGQMIGSFGILMGVIVAIYRNVDADKKHKYKGMMIATALATFLTGVTEPIEYMFMFIATPMYLVYSLVQGAAFAMADVVNLRMHSFGSIEFLTRTPIAISAGIGMDIVNFVWVTVLFAVIMYFIANFMIQKFNYATPGRNGNYETAEGSEETSSEVKVAAGSQAVNIINLLGGRVNIVDVDACMTRLRVTVKDADKVGNAEQWKAEGAMGLVMKGQGVQAIYGPKADILKSDIQDILDSGEIIPETLPSQMTEAQQNTVHFKDLTEEVYSVADGQVVGLEQVKDPVFAQKMMGDGFAVEPANGNIVSPVSGTVSSIFPTKHAFGIVTEAGLEVLVHIGLDTVSLEGKPFTVHVAEGQKVAAGDLLVTADLDAIRAAGRETSTVVVFTNGDAIKSVKLEKTGSLAAKTAVAKVEL

SEQ ID NO: 169 - protein, 271 aa, Streptococcus pneumoniae
MKKMKYYEETSALLHEFSEENQKYFEELWESFNLAGFLYDEDYLREQIYLMMLDFSEAERDGMSAEDYLGKNPKKIMKEILKGAPRSSIKESLLMPILVLAVLRYYQLLSDFSKGPLLTVNLLTFLGQLLIFLIGFGLVATILRRSLVQDSPKMKIGTYIVVGTIVLLVVLGYVGMASFIQEGAFYIPAPWDSLSVFTISLVIGIWNWKEAVFRPFVSMIIAHLVVGSLLRYYEWMGISNVFLTKVIPSAVLFIGIFVLFRGFKKIKWSEV

SEQ ID NO: 170 - protein, 752 aa, Streptococcus pneumoniae
MLCQNCKINDSTIHLYTNLNGKQKQIDLCQNCYKIIKTDPNNSLFKGMTDLNNRDFDPFGDFFNDLNNFRPSSNTPPIPPTQSGGGYGGNGGYGSQNRGSAQTPPPSQEKGLLEEFGINVTEIARRGDIDPVIGRDDEIIRVIEILNRRTKNNPVLIGEPGVGKTAVVEGLAQKIVDGDVPHKLQGKQVIRLDVVSLVQGTGIRGQFEERMQKLMEEIRKREDIILFIDEIHEIVGAGSASDGNMDAGNILKPALARGELQLVGATTLNEYRIIEKDAALERRMQPVKVDEPTVDETITILKGIQKKYEDYHHVQYTDAAIEAAATLSNRYIQDRFLPDKAIDLLDEAGSKMNLTLNFVDPKVIDQRLIEAENLKSQATREEDFEKAAYFRDQIAKYKEMQKKKITDQDTPIISEKTIEHIIEQKTNIPVGDLKEKEQSQLIHLAEDLKSHVIGQDDAVDKIAKAIRRNRVGLGTPNRPIGSFLFVGPTGVGKTELSKQLAIELFGSADSMIRFDMSEYMEKHSVAKLVGAPPGYVGYDEAGQLTEKVRHNPYSLILLDEVEKAHPDVMHMFLQVLDDGRLTDGQGRTVSFKDAIIIMTSNAGTGKTEASVGFGAAREGRTNSVLGELGNFFSPEFMNRFDGIIEFKALSKDNLLQIVELMLADVNKRLSSNNIRLDVTDKVKEKLVDLGYDPKMGARPLRRTIQDYIEDTITDYYLENPSEKDLKAVMTSKGNIQIKSAKKAEVKSSEKEK

SEQ ID NO: 171 - protein, 375 aa, Streptococcus pneumoniae
MKLEQVPTPAYVIDLAKLEANCRILQYVQEEAGCKVLLAQKAYSLYKTYPLISQYLSGTTASGLYEAKLAREEFPGEVHVFAPAFKDADLEELLEIMDHIVFNSERQLRKHGPRCREAGVSVGLRLNPQCSTQGDHALYDPCAPGSRFGVTIDKIPSDLLDLVDGLHFHTLCEQGADDLQTTLKAVEEQFGPYLHEVKWLNMGGGHHITREGYDVDLLISEIKRIRKTYNLEIYIEPGEAIALNAGYLATEVLDIVENGMEILVLDASATCHMPDVLEMPYRPPLRNGFESQEKAHTYRLSSNTCLTGDVIGDYSFENPVQIGDRLYFQDMAIYSFVKNNTFNGIGLPSLYLMDEQGDCSLLKAFGYQDFKGRLS

SEQ ID NO: 172 - protein, 627 aa, Streptococcus pneumoniae
MKKKLTSLALVGAFLGLSWYGNVQAQESSGNKIHFINVQEGGSDAIILESNGHFAMVDTGEDYDFPDGSDSRYPWREGIETSYKHVLTDRVFRRLKELGVQKLDFILVTHTHSDHIGNVDELLSTYPVDRVYLKKYSDSRITNSERLWDNLYGYDKVLQTAAEKGVSVIQNITQGDAHFQFGDMDIQLYNYENETDSSGELKKIWDDNSNSLISVVKVNGKKIYLGGDLDNVHGAEDKYGPLIGKVDLMKFNHHHDTNKSNTKDFIKNLSPSLIVQTSDSLPWKNGVDSEYVNWLKERGIERINAASKDYDATVFDIRKDGFVNISTSYKPIPSFQAGWHKSAYGNWWYQAPDSTGEYAVGWNEIEGEWYYFNQTGILLQNQWKKWNNHWFYLTDSGASAKNWKKIAGIWYYFNKENQMEIGWIQDKEQWYYLDVDGSMKTGWLQYMGQWYYFAPSGEMKMGWVKDKETWYYMDSTGVMKTGEIEVAGQHYYLEDSGAMKQGWHKKANDWYFYKTDGSRAVGWIKDKDKWYFLKENGQLLVNGKTPEGYTVDSSGAWLVDVSIEKSATIKTTSHSEIKESKEVVKKDLENKETSQHESVTNFSTSQDLTSSTSQSSETSVNKSESEQ

SEQ ID NO: 173 - protein, 642 aa, Streptococcus pneumoniae
MDNREALKTFMTGENFYLQHYLGAHREELNGEYGYTFRVWAPNAQAVHLVGDFTNWIENQIPMVRNDFGVWEVFTNMAQEGHIYKYHVTRQNGHQLMKIDPFAVRYEARPGTGAIVTELPDKKWRDGLWLARRKRWGFAERPVNIYEVHAGSWKRNPDGSPYSFAQLKDELIPYLVEMNYTHIEFMPLMSHPLGLSWGYQLMGYFALEHAYGRPEEFQDFVEECHTHNIGVIVDWVPGHFTINDDALAYYDGTPTFEYQDHNKAHNHGWGALNFDLGKNEVQSFLISCIKHWIDVYHLDGIRVDAVSNMLYLDYDDAPWTPNKDGGNLNYEGYYFLQRLNEVIKLEYPDVMMIAEESSSATNITGMKEIGGLGFDYKWNMGWMNDILRFYEEDPIYRKYDFNLVTFSFMYVFKENYLLPFSHDEVVHGKKSMMHKMWGDRYNQFAGLRNLYTYQICHPGKKLLFMGSEYGQFLEWKSEEQLEWSNLEDPMNAKMKYFTSQLNQFYKDHRCLWEIDTSYDGIEIIDADNRDQSVLSFIRKGKKGEMLVCIFNMVPVERKDFTIGLPVAGIYEEVWNTELEEWGGVWKEHNQTVQTQEGLWKDYEQTLTFTLPAMGASVWKIKRRLKSTKTVTNKNQKGVENEK

SEQ ID NO: 174 - protein, 380 aa, Streptococcus pneumoniae
MKNEMLALILAGGQGTRLAKLTQSIAKPAVQFGGRYRIIDFALSNCANSGIHNVGVVTQYQPLALNNHIGNGSSWGLDGIDSGVSILQPYSASEGNRWFEGTSHAIYQNIDYIDSVNPEYVLILSGDHIYKMDYDDMLQSHKDNNASLTVAVLDVPLKEASRFGIMNTDANNRIVEFEEKPAQPKSTKASMGIYIFDWQRLRNMLVAAEKSKVGMSDFGKNVIPNYLELGESVYAYEFSGYWKDVGTIESLWEANMEYISPENALDSRNRQWKIYSRNLISPPNFLGANAHVEDSLVVDGCFVDGTVKHSILSTGAQVREGAEVLDSVIMSGAIIGQGAKIKRAIIGEGAIISDGVEIDGTDEVQVVGYNEVVGVATDED

SEQ ID NO: 175 - protein, 149 aa, Streptococcus pneumoniae
MELVEFKNGHVNTVHEHIAITKVKEFLNNHDIPKDKGGIHKISTDFFYNIIDMETTTEENRPWESHKEYYDVHYLLNGEEIILYNFLSQMELSEYKVDDDWQQMNGTALFSIKLKKDMLLLLEPNDAHKTGLLVEEPINIKKVVFKVKI

SEQ ID NO: 176 - protein, 194 aa, Streptococcus pneumoniae
MKDVVYSITPENKLSIDFSFKSPFRVLLTGTSGSGKTTILNLINGSLKPQKGYVNLLSHGKKSSDSIPTVDQTPYIFDTTIRENVTLFQNEYFSDDQIIEVLKKVNLYEELEKIDILNYQCGENGSNLSGGQKQKIALARALIRNNKVYLFDEISANLDNDNSNSIHDILFNLGISFIEVSHHYDLNDKRYTDI

SEQ ID NO: 177 - protein, 195 aa, Streptococcus pneumoniae
MKRIIPVYIFQQVNVLLVSLYLLKFLCIGELTILQILYGSSLISFLWMYGQRKQAHKVNMKSRMKWLGVEFVSLLIISLCFSLIHAQGSTNQANLIGLQHQIPWFSFLLFLINASMVEEFLYREILWNLVRNLDIRVALTSVLFALAHHTGTILAWCLYVSLGMFLGMVRYKSDLWGSMGLHLVWNLLVYSLLLF

SEQ ID NO: 178 - protein, 211 aa, Streptococcus pneumoniae
VAVQLLENWLLKEQEKIQTKYRHLNHISVVEPNILFIGDSIVEYYPLQELFGTSKTIVNRGIRGYQTGLLLENLDAHLYGGAVDKIFLLIGTNDIGKDVPVNEALNNLEAIIQSVARDYPLTEIKLLSILPVNEGEKYQQAVYIRSNEKIQNWNQAYQELASAYMQVEFVPVFNCLTDQAGQLKKEYTTDGLHLSIAGYQALSKSLKDYLY

SEQ ID NO: 179 - protein, 531 aa, Streptococcus pneumoniae
MKRIVFELIFIATTWYIFLPPLNLTSWEFLFFLCGHLLVVAILFGFGKGINLVKTVHVRHGKAEAALNLEGFKINRLGKILLASIGGILLLAALVSLVTSSMFQAKNYANVVTVTEKDFTEFPRSDTSKVPILDRSTAEKIGDRYLGSLTDKVSQYVAADTYTQLTIDGKPYRVTPLEYADPIKWFNNQAKGIGEYIKVDMVTGNADLVDLKTPIKYSDSEYFNRDVKRHLRLKYPTKIFKTPSFEVDDEGNPFYVATVYQKQFGLAVPRPASVIILDATNGETKEYSLSDVPEWVDRIYPAEETIEQINYNGKYKDGFLNAMISKKNVTQTTKGYNYLSIGNDIYLYTGVTSANADESNLGFILENMRTGEITKYSLASATEESARESAEGAVQEKSYKATFPILINLNDKPLYIMGLKDNAGLVKEYALVDAVEYQNVIVATTVEEMLSKYANKNDLEIDNATTESINGVVADLKSAVIKGDTVYFFKVDGKIYKVKASVSDDLPYLENGKTFEGQVGKDNYLKTFKLR

SEQ ID NO: 180 - protein, 490 aa, Streptococcus pneumoniae
LKTKIGLASICLLGLATSHVAANETEVAKTSQDTTTASSSSEQNQSSNKTQTSAEVQTNAAAHWDGDYYVKDDGSKAQSEWIFDNYYKAWFYINSDGRYSQNEWHGNYYLKSGGYMAQNEWIYDSNYKSWFYLKSDGAYAHQEWQLIGNKWYYFKKWGYMAKSQWQGSYFLNGQGAMMQNEWLYDPAYSAYFYLKSDGTYANQEWQKVGGKWYYFKKWGYMARNEWQGNYYLTGSGAMATDEVIMDGTRYIFAASGELKEKKDLNVGWVHRDGKRYFFNNREEQVGTEHAKKVIDISEHNGRINDWKKVIDENEVDGVIVRLGYSGKEDKELAHNIKELNRLGIPYGVYLYTYAENETDAESDAKQTIELIKKYNMNLSYPIYYDVENWEYVNKSKRAPSDTGTWVKIINKYMDTMKQAGYQNVYVYSYRSLLQTRLKHPDILKHVNWVAAYTNALEWENPHYSGKKGWQYTSSEYMKGIQGRVDVSVWY

SEQ ID NO: 181 - protein, 412 aa, Streptococcus pneumoniae
MHIFLKNRAFRQLTVNEWISSFGDTIFYLAFINYVSSYAFAPLAIFLISLSETIPQVLQLFTGVIADFQKNRISKYISILFIKVLLYSGVTLLLTSTDFSLFSVFFICSMNLISDTIGFLAGYMLTPIYIRLINDDMTEAMGFRQSTSSIVRLIGNLSGGVFLGLFSISTLAFVNVLTFLFAFLGSLLIRNRLKKEEEKIEVPPYVGMSSFFQHLKESMKLLMTMEDVMVLLWILSISQAVLMMVEPVSAILLIHHPFMGLSTGQSLAILIMISLLHVILGGLLSGFLSKKISIRLNIYWSLLMESLIVIDFLRGSFLLILLGSAGDAFSAGVLSPRLQAMIFGIIPEELMGSVQSSINVINLLIPAVLSLALVFLATSAGLEVVAFALIILLLIAAYLVHQMKNLPNQEEV

SEQ ID NO: 182 - protein, 246 aa, Streptococcus pneumoniae
MATYETFAAVYDAVMDDSLYDKWTNFSLRHLPKTKERKKLLELACGTGIQSVRFSQAGFDVTGLDLSADMLKIAEKRATSAKQKIAFIEGNMLNLSKAGKYDFVTCYSDSICYMQDEVEVGDVFKDVYNALNEEGVFIFDVHSTYQTDEVFPGYSYHENAEDFAMLWDTYEDEAPHSIVHELTFFIKEADGSFSRHDEVHEERTYEILTYDILLEQAGFKSFKLYADFEDKEPTETSTRWFFVAQK

SEQ ID NO: 183 - protein, 333 aa, Streptococcus pneumoniae
MKNINGKKVTIYDIARLSGFSPKTVSRVINGGVNVKEETYQAIQKVIEELSYIPNAYAKNLTKKEAINILISVKKIDSFPLIWFHTLLDKVLRTCKEFGVNAIVEYFGEEDTISNSIISSTGSLVDGVIVFYESVDDIRIQYLKKNHMPFLVFGESQTSGVVYVSNNNFQATYDMMKAVTEEKFKNMWLLMGGESHVNKDRERGVRSFLNDKNYFMDLKVIYGLSTIDSVYSYAMQHLTTQNYPDIIFVSGDEKVQGLIRACYEKGILIPDDISIIGFDNIPISQYYTPALSTIAPNYVKLAKEMIEGVLAIIKGESVTSVEVSPKFVRRQSF

SEQ ID NO: 184 - protein, 242 aa, Streptococcus pneumoniae
MIEYKNVALRYTEKDVLRDVNLQIEDGEFMVLVGPSGSGKTTMLKMINRLLEPTDGNIYMDGKRIKDYDERELRLSTGYVLQAIALFPNLTVAENIALIPEMKGWSKEEITKKTEELLAKVGLPVAEYGHRLPSELSGGEQQRVGIVRAMIGQPKIFLMDEPFSALDAISRKQLQVLTKELHKEFGMTTIFVTHDTDEALKLADRIAVLQDGEIRQVANPETILKAPATDFVADLFGGSVHD

SEQ ID NO: 185 - protein, 319 aa, Streptococcus pneumoniae
MKLSHYLIGLLLLLVFLSISIGTSDFSWGKLFDFDQQTWLLFQESRLPRTISILLTASSMSMAGLLMQTITQNQFAAPSTVGTTEAAKLGMVLSLFVFPSASLTQKMLFAFVSSIVFTLFFLAFMTIFTVKERWMLPLIGIIYSGIIGSVTEVIAYRFNLVQSMTAWTQGSFSMIQTHQYEWLFLGLIILITVWKLSQTFTIMNLGKETSESLGISYSLLEKLALFLVALTTSVTMITVGGLPFLGVIVPNLVRKRYGDNLSQTKLMVALVGANLVLACDILSRVLIRPYELSVSLLLGIIGSLVFILLLWRGGRKDAD

SEQ ID NO: 186 - protein, 110 aa, Streptococcus pneumoniae
MYICIDNSMTKNKELVKNQTFKPALLTRRLVKNFMIEEPDLARNPFTRPTLIDLDKVFMLDNTVIPTSYLARRRRNVSEELYEEILDYLVQPRLISLNKSEFMQLNPGTY

SEQ ID NO: 187 - protein, 345 aa, Streptococcus pneumoniae
MKKKIRWPLYVIAALIVTFLAFVVPLPYYIEVPGGSEDIRQVLKVNDTEDKEAGAYQFVTVGVQHATLAHMIYAWLTPFTDIRSAQETTGGSSDVEFMRINQFYMQTSQNMAKYQGLKTAGKDIELKYFGVYVLNVTDNSTFKGILNISDTVTAVNDQTFDSSKDLIDYVSSQKLGDSVKVTYEEDGQTKSAEGKIITLENGKNGIGIGLIDRTEVISNVPISFSTAGIGGPSAGLMFSLAIYTQIAHPDLRNGRIVAGTGTIDRDGNVGDIGGIDKKVVASARAGAAIFFAPDNPVSEEEQKAHPDAKNNYQTALEAAKTIKTDMKIVPVKTLQDAIDYLKNNP

SEQ ID NO: 188 - protein, 227 aa, Streptococcus pneumoniae
MNHTIIHDRAGLNQFYAKVYAFVGLGIGLSALVSGLMLTVFQSQLVYFLMQGRLWLTIATFAELALVFVASSMASRNSPSALPVFLLYSVLNGFTLSFVVAFYTPGTVLSAFVSSALLFFVMAAVGIFTKKDLSGIGRAMMAALIGLLIAMVVNIFLASGFFDYMISVAMVLVFSGLIAWDNQKIRLAYEQSQGRVATGWVVSMALSIYLDFINLFLSILRIFGRND

SEQ ID NO: 189 - protein, 430 aa, Streptococcus pneumoniae
MEKQQPSKAALLSIIPGLGQIYNKQKAKGFIFLGVTIVFVLYFLALATPELSNLITLGDKPGRDNSLFMLIRGAFHLIFVIVYVLFYFSNIKDAHTIAKRINNGIPVPRTLKDMIKGIYENGFPYLLIIPSYVAMTFAIIFPVIVTLMIAFTNYDFQHLPPNKLLDWVGLTNFTNIWSLSTFRSAFGSVLSWTIIWALAASTLQIVIGIFTAIIANQPFIKGKRIFGVIFLLPWAVPAFITILTFSNMFNDSVGAINTQVLPILAKFLPFLDGALIPWKTDPTWTKIALIMMQGWLGFPYIYVLTLGILQSIPNDLYEAAYIDGANAWQKFRNITFPMILAVAAPTLISQYTFNFNNFSIMYLFNGGGPGSVGGGAGSTDILISWIYRLTTGTSPQYSMAAAVTLIISIIVISISMIAFKKLHAFDMEDV

SEQ ID NO: 190 - protein, 287 aa, Streptococcus pneumoniae
MRYDFGKVYKEIRESKGLTQEEVCGGVLSRTSLSKIESGKTTPKYENMEFLLRQINMSFEEFEYICQLYQPSQRTEIMQTYLNMRSIIGTSDLVNLFQKCQDYLKTHHDLPIEEIRDMLEVVIYIRQHGAGELSDHAEQVVKKLWRKIEKQDTWYESDLKILNTILFSFPIEYLHLITGKILQRLEVYKNYQHLYDLRIAILLNLSTLYLYNQDKNMCKQICYTLLEDAKNKKSYDRLAICYVRIGICTDDSKLIQKGFSLLELTEETSMLSHLKKEVEIYYQAKER

SEQ ID NO: 191 - protein, 117 aa, Streptococcus pneumoniae
MELVLPNNYVVIDEEEMMYLDGGAYLSKSACQGICVALAMSPGTFIALTGAAVLTKKLINYIKVGGLGGWLIGAAAGVLATAAGKIAYCIGYGALNRGCDISGNPYPWDGFISATVR

SEQ ID NO: 192 - protein, 230 aa, Streptococcus pneumoniae
MESLIQTYLPNVYKMGWAGQAGWGTAIYLTLYMTVLSFIIGGFLGLVAGLFLVLTAPGGVLENKVVFWILDKITSIFRAVPFIILLAILSPLSHLIVKTSIGPNAALVPLSFAVFAFFARQVQVVLAELDGGVIEAAQASGATFWDIVGVYLSEGLPDLIRVTTVTLISLVGETAMAGAVGAGGIGNVAIAYGFNRYNHDVTILATIIIILIIFAIQFLGDFLTKKLSHK

SEQ ID NO: 193 - protein, 189 aa, Streptococcus pneumoniae
MKKSILTTLLFAVLYFLCMGIGVLLGNLFDQTGNMFYAPAFTALVGGSVYMILVAKVPRFGAITTIGLVIAFFFLGTKHGAGSFLPGIICGLLADEVAHLGKYKDKTKNFLSFIIFAFSTTGPILLMWIAPKAYMATLLARGKSQEYIDRIMVAPNPGTVLLFIASIVIGALVGALIGQALSKKFAQKI

SEQ ID NO: 194 - protein, 392 aa, Streptococcus pneumoniae
MKNLIKLLIIRLIVNLADSVFYIVALWHVSNNYSSSMFLGIFIAVNYLPDLLLIFFGPVIDRVNPQKILIISILVQLAVAVIFLLLLNQISFWVIMSLVFISVMASSISYVIEDVLIPQVVEYDKIVFANSLFSISYKVLDSIFNSFASFLQVAVGFILLVKIDIGIFLLALFILLLLKFRTSNANIENFSFKYYKREVLQGTKFILNNKLLFKTSISLTLINFFYSFQTVVVPIFSIRYFDGPIFYGIFLTIAGLGGILGNMLAPIVIKYLKSNQIVGVFLFLNGSSWLVAIVIKDYTLSLILFFVCFMSKGVFNIIFNSLYQQIPPHQLLGRVNTTIDSIISFGMPIGSLVAGTLIDLNIELVLIAISIPYFLFSYIFYTDNGLKEFSIY

SEQ ID NO: 195 - protein, 512 aa, Streptococcus pneumoniae
MSVYGRVEEVHKENREPLEYQIEQESHHRESSRLPLVKILLWSTLVTGITLGVPLLLDLMSAQEVQDFYAGWALHQTGKIYSDYYGSQGLLYYLLTYVSQGGFFFAIFEWLALVAGGFFLFRSADTLTEQGDQAGHLVTIFYMLVTGLAFGGGYATLLALPFLFAAFSLVAAYLSNPSHDKGFVRIGLALAGGFFFAPLSSLLFIAVVSLGLLVFNLGHRRFAHGFYQFLAVALGFSLVFYPTAYYSAATGSFGDAISGIRYPIDSIRFDFTSKILENMFFYGLLSLGLGFVVCIFLGLFQSKPFKLYVISVPASLVVILGLILLFFSQEPLHASYLMVVFPVFLLLLVTNIKSQQRGRSARRSLRETPVSLWSRFFKGNLYLLVFGFVYLLSVPFLMKFVLYPVPYQERNRLADLVKEETNTEDAIYAWDDTATLYRKSERLSPSAILSPLHYTATEENRNKLLNDLKEKQPKVIVVNDKVVVWSEVETLLKENYQQVKTDYSEFKVYKIK

SEQ ID NO: 196 - protein, 449 aa, Streptococcus pneumoniae
MSKYNVLAKDIVTYVGGAENVKSLRHCATRLRFELKDESQADKEALMNLDILQVVQASGQYQVVVGPHVASVYEAVMAELPVIGGASSINEDTTAEEKKWYDVIFDTISGSFTPLIPVFCGSGLVKALGSVLLMTGLLTAESSTYAVLNAAGNAVFYFLPILLAISVANKLGTNPYIAGAIGAALLEPSFTALAGNESNTFLGLPIIINNYASSVFPAFMAVVGLYFLDKFLKKVVPATIHLVVLPFIEIVAMVPLTIFIFGPFGTYLSTGLSMFVTNLLDLNAMLAGAVLSMIWIYVVMLGLHWALIPIMINNIATNGSDPLMGLMIGTVWVAGGVSLGVALKAKDKKLKGIAWTGMIPCFLSGVSEPIMYGILFRYKKSLMWATVMNAVTGAMAGLIGISGTQIAGGVFTIPTFSPILGYLALIAVSVFGTAALIVIFGFGEEGSKA

SEQ ID NO: 197 - protein, 58 aa, Streptococcus pneumoniae
MSLDIDKEKMTIMGIAFENRSVFKSVWYALSTNMIEGWRPTVSDVEKLRDEALSLGMT

SEQ ID NO: 198 - protein, 396 aa, Streptococcus pneumoniae
MIKKVTIEKIKSPERFLEVPLLTKEEVGQAIDKVIRQLELNLDYFKEDFPTPATFDNVYPIMDNTEWTNGFWTGELWLAYEYSQQDAFKNIAHKNVLSFLDRVNKRVELDHHDLGFLYTPSCMAEYKINGDGEAREATLKAADKLIERYQEKGGFIQAWGDLGKKEHYRLIIDCLLNIQLLFFAYQETGDQKYYDIAESHFYASANNVIRDDASSFHTFYFDPETGQPFKGVTRQGYSDDSCWARGQSWGVYGIPLTYRHLKDESCFDLFKGVTNYFLNRLPKDHVSYWDLIFNDGSAQSRDSSATAIAVCGIHEMLKHLPEVDADKDIYKHAMHAMLRSLIEHYANDQFTPGGTSLLHGVYSWHSGKGVDEGNIWGDYYYLEALIRFYKDWNLYW

SEQ ID NO: 199 - protein, 182 aa, Streptococcus pneumoniae
MKSLARLLIIHVFISIFLFFALISGAVSHTVLLLLLLFLPALNKGLEKIQSKRIPVLNAALFFLLISFPQLLTNPVQWKFSIFLVVTIISSLAYFYNFYQVVKEVDQKQLIMRIKEFEDDDLVSPRTNQLMFIGLTGFMSIICLYRGITAGESYQQLIAYIGAILCLIIMLLLIWGLKYYKK

SEQ ID NO: 200 - protein, 71 aa, Streptococcus pneumoniae
MRIKEFEDDDLVSPRTNQLMFIGLTGFMSIICLYRGITAGESYQQLIAYIGAILCLIIMLLLIWGLKYYKK

SEQ ID NO: 201 - protein, 229 aa, Streptococcus pneumoniae
MKKYQLLFKISAVFSYLFFVFGLSQLTLIVQNYWQFSSQIGNLFWIQNILSLLFIGVMIVVLVKTGHGYLFRIPRKKWLWYSILTVLVVVFQISFNVQTAKHVQSTAEGWAVLIGYSGTNFAELGIYIALFFLVPLMEELIYRGLLQHAFFKHSRFGLDLLLPSILFALPHFLSLPSLLDIFVFATVGIIFAGLTRYTKSIYPSYAVHVINNIVATFPFLLTFLHRVLG

SEQ ID NO: 202 - protein, 425 aa, Streptococcus pneumoniae
MNPIQRSWAYVSRKRLRSFILFLILLVLLAGISACLTLMKSNKTVESNLYKSLNTSFSIKKIENGQTFKLSDLASVSKIKGLENVSPELETVAKLKDKEAVTGEQSVERDDLSAADNNLVSLTALEDSSKDVTFTSSAFNLKEGRHLQKGDSKKILIHEELAKKNGLSLHDKIGLDAGQSESGKGQTVEFEIIGIFSGKKQEKFTGLSSDFSENQVFTDYESSQTLLGNSEAQVSAARFYVENPKEMDGLMKQVENLALENQGYQVEKENKAFEQIKDSVATFQTFLTIFLYGMLIAGAGALILVLSLWLRERVYEVGILLALGKGKSSIFLQFCLEVVLVSLGALLPAFVAGNGITTYLLQTLLASGDQASLQDTLAKASSLSTSILSFAESYVFLVLLSCLSVALCFLFLFRKSPKEILSSIS

SEQ ID NO: 203 - protein, 213 aa, Streptococcus pneumoniae
MIELKNISKKFGSRQLFSDTNLHFEGGKIYALIGTSGCGKTTLLNMIGRLEPYDKGQIIYDGTSLKDIKPSVFFRDYLGYLFQDFGLIESQTVKENLNLGLVGKKLKEKEKISLMKQALNRVNLSYLDLKQPIFELSGGEAQRVALAKIILKDPPLILADEPTASLDPKNSEELLSILESLKNPNRTIIIATHNPLIWEQVDQVIRVTDLSHR

SEQ ID NO: 204 - protein, 222 aa, Streptococcus pneumoniae
METQDYAFQPGLTVGELLKSSQKDWQAAINHRFVKELFAGTIENKVLKDYLIQDYHFFDAFLSMLGACVAHADKLESKLRFAKQLGFLEADEDGYFQKAFKELKVAENDYLEVTLHPVTKAFQDLMYSAVASSDYAHLLVMLVIAEGLYLDWGSKDLALPEVYIHSEWINLHRGPFFAEWVQFLVDELNRVGKNREDLTELQQRWNQAVALELAFFDIGYNV

SEQ ID NO: 205 - protein, 134 aa, Streptococcus pneumoniae
MKILKRYILELCFILSFALPFIKGANADNGRCFVETYYGFTFLMEHAIVTAVFIYSFLIAFLLKKRWAKWIAAGSYCFLVLWIATEGYFFRMSLEDLIRLWTSLEILTQTYQLGFYLNILLGILLIIKYFKVKQ

SEQ ID NO: 206 - protein, 5 aa, synthetic construct
LPXTG

SEQ ID NO: 207 - DNA, 89 bp, synthetic construct
  source 1..89: mol_type = other DNA
cactagtcattctacagtttattcttgacattgcactgtccccctggtataataactatacatgcaagatctaaataggaggaaaatta

SEQ ID NO: 208 - DNA, 90 bp, synthetic construct
  source 1..90: mol_type = other DNA
cactagtcattctacagtttattcttgacattgcactgtccccctggtataataactatacatgcaaggatctaaataggaggaaaatta

SEQ ID NO: 209 - DNA, 99 bp, synthetic construct
  source 1..99: mol_type = other DNA
gaaaaaatatttttcaaaacccacttgacactttagctatatatgtttataataactatgaggatatctgctgctaggatctaaataggaggaaaatta

SEQ ID NO: 210 - protein, 19 aa, Haemophilus influenzae
MKLKTLALSLLAAGVLAGC

SEQ ID NO: 211 - protein, 38 aa, synthetic construct
MKHEKQQRFSIRKYAVGAASVLIGFAFQAQTVAADGVT

SEQ ID NO: 212 - protein, 7 aa, synthetic construct
  VARIANT 1: note = X is Serine or Threonine
XRRXFLK

SEQ ID NO: 213 - protein, 364 aa, Haemophilus influenzae
MKLKTLALSLLAAGVLAGCSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPEFFTDVNQMYDALLNKSGATGVFTDFPDTGVEFLKGIK

SEQ ID NO: 214 - DNA, 1095 bp, Haemophilus influenzae
  source 1..1095: mol_type = other DNA
atgaaattgaagacattggctttatcattgttagctgctggagttttggctggttgttctagtcattcatctaatatggctaatacacaaatgaagtctgataagatcattattgctcatcgtggagctagtggttatttgcctgaacatactttagaatcaaaagctcttgcttttgctcaacaagctgattatttggaacaagatttagctatgacaaaagatggacgtttggttgttatccatgatcattttcttgatggtttgactgatgttgctaaaaaatttccacatcgtcatcgtaaagatggacgttattatgttatcgattttactttgaaggaaattcaatctttggaaatgacagaaaattttgaaactaaagatggtaaacaagctcaagtttatccaaatcgttttcctttgtggaagtctcattttcgtattcatacatttgaagatgaaatcgaatttattcaaggtttggaaaaatctacaggtaagaaagttggtatctatccagaaatcaaggctccttggtttcatcatcaaaatggaaaggatattgctgctgaaacattgaaagttttgaaaaaatatggttatgataagaaaacagatatggtttatttacaaacttttgattttaatgaattgaagcgtatcaagactgaattgttgcctcaaatgggtatggatttgaaattggttcaattgatcgcttatacagattggaaggaaactcaagaaaaagatccaaagggatattgggttaattataattatgattggatgtttaagcctggtgctatggctgaagttgttaaatatgctgatggtgttggaccaggttggtatatgttggttaataaagaagaatctaagccagataatatcgtttatacacctttggttaaggaattagctcaatataatgttgaagttcatccatatacagttcgtaaagatgctttgcctgaatttttcactgatgttaatcaaatgtatgatgctttgttgaataaatcaggagctacaggtgtttttactgattttccagatactggagttgaatttttgaaaggtattaaataa

SEQ ID NO: 215 - protein, 21 aa, synthetic construct
  VARIANT 4: note = X is a hydrophobic amino acid residue
  VARIANT 5: note = X is Lysine or Glutamine
  VARIANT 18: note = X is an amino acid residue that has a small side chain
TGWXXDNGSWYYLNXSGXMXX

SEQ ID NO: 216 - protein, 60 aa, synthetic construct
LWYYLNESGSMATGWVKDKGLWYYLNESGSMATGWVKDKGLWYYLNESGSMATGWVKDKG

SEQ ID NO: 217 - DNA, 180 bp, synthetic construct
  source 1..180: mol_type = other DNA
ttgtggtattatttgaatgaaagtggatcaatggctacaggttgggttaaggataagggtttatggtattatttaaatgaatctggaagtatggctactggatgggtaaaggataagggtctttggtactacttaaatgaatcaggatctatggctactggttgggtaaaagataaagga

SEQ ID NO: 218 - protein, 20 aa, synthetic construct
TGWVKDKGLWYYLNESGSMA

SEQ ID NO: 219 - protein, 447 aa, synthetic construct
MKHEKQQRFSIRKYAVGAASVLIGFAFQAQTVAADGVTSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPEFFTDVNQMYDALLNKSGATGVFTDFPDTGVEFLKGIKLWYYLNESGSMATGWVKDKGLWYYLNESGSMATGWVKDKGLWYYLNESGSMATGWVKDKGGSLQ

SEQ ID NO: 220 - DNA, 1344 bp, synthetic construct
  source 1..1344: mol_type = other DNA
atgaagcatgaaaagcaacaacgttttagtatccgtaagtatgctgttggagctgcttcagttttgatcggttttgcttttcaagctcaaacagttgctgctgatggtgttacttctagtcattcatctaatatggctaatactcaaatgaagtctgataagatcattattgctcatcgtggagcttctggttatttgcctgaacatacattagaaagtaaagctcttgcttttgctcaacaagctgattatttggaacaagatttagctatgacaaaagatggacgtttggttgttatccatgatcattttcttgatggtttgactgatgttgctaaaaaatttccacatcgtcatcgtaaagatggtcgttattatgttatcgattttactttgaaggaaattcaaagtttggaaatgacagaaaattttgaaactaaagatggaaaacaagctcaagtttatccaaatcgttttcctttgtggaagtcacattttcgtattcatacatttgaagatgaaatcgaatttattcaaggtttggaaaaatctactggtaagaaagttggtatctatccagaaatcaaggctccttggtttcatcatcaaaatggaaaggatattgctgctgaaactttgaaagttttgaaaaaatatggttatgataagaaaacagatatggtttatttacaaacttttgattttaatgaattgaagcgtatcaagacagaattgttgcctcaaatgggaatggatttgaaattggttcaattgatcgcttatacagattggaaggaaactcaagaaaaagatccaaagggatattgggttaattataattatgattggatgtttaagcctggtgctatggctgaagttgttaaatatgctgatggtgttggaccaggttggtatatgttggttaataaagaagaatctaagccagataatatcgtttatactcctttggttaaggaattagctcaatataatgttgaagttcatccatatacagttcgtaaagatgctttgcctgaatttttcactgatgttaatcaaatgtatgatgctttgttgaataaatcaggagctacaggtgtttttactgattttccagatacaggagttgaatttttgaagggtatcaaattgtggtattatttgaatgaaagtggatcaatggctacaggttgggttaaggataagggtttatggtattatttaaatgaatctggaagtatggctactggatgggtaaaggataagggtctttggtactacttaaatgaatcaggatctatggctactggttgggtaaaagataaaggaggatccctccagtaa

SEQ ID NO: 221 - protein, 32 aa, synthetic construct
LPETGTHDSAELVVAGLMSTLAAYGLTKRKED

SEQ ID NO: 222 - DNA, 96 bp, synthetic construct
  source 1..96: mol_type = other DNA
ttgcctgaaacaggtactcatgattcagctgaattagttgttgctggattgatgtctacattagctgcttatggtttgactaaacgtaaagaagat

SEQ ID NO: 223 - protein, 419 aa, synthetic construct
MKHEKQQRFSIRKYAVGAASVLIGFAFQAQTVAADGVTSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPEFFTDVNQMYDALLNKSGATGVFTDFPDTGVEFLKGIKLPETGTHDSAELVVAGLMSTLAAYGLTKRKEDGSLQ

SEQ ID NO: 224 - DNA, 1260 bp, synthetic construct
  source 1..1260: mol_type = other DNA
atgaagcatgaaaagcaacaacgttttagtatccgtaagtatgctgttggagctgcttcagttttgatcggttttgcttttcaagctcaaacagttgctgctgatggagttacttctagtcattcatctaatatggctaatacacaaatgaagtcagataagatcattattgctcatcgtggagcttctggttatttgcctgaacatactttagaaagtaaagctcttgcttttgctcaacaagctgattatttggaacaagatttagctatgacaaaagatggacgtttggttgttatccatgatcattttcttgatggtttgactgatgttgctaaaaaatttccacatcgtcatcgtaaagatggacgttattatgttatcgattttacattgaaggaaattcaaagtttggaaatgacagaaaattttgaaactaaagatggtaaacaagctcaagtttatccaaatcgttttcctttgtggaagagtcattttcgtattcatacatttgaagatgaaatcgaatttattcaaggattggaaaaatcaactggtaagaaagttggtatctatccagaaatcaaggctccttggtttcatcatcaaaatggaaaggatattgctgctgaaacattgaaagttttgaaaaaatatggttatgataagaaaacagatatggtttatttacaaacttttgattttaatgaattgaagcgtatcaagactgaattgttgcctcaaatgggtatggatttgaaattggttcaattgatcgcttatacagattggaaggaaactcaagaaaaagatccaaagggatattgggttaattataattatgattggatgtttaagcctggtgctatggctgaagttgttaaatatgctgatggtgttggaccaggttggtatatgttggttaataaagaagaatctaagccagataatatcgtttatacacctttggttaaggaattagctcaatataatgttgaagttcatccatatacagttcgtaaagatgctttgcctgaatttttcactgatgttaatcaaatgtatgatgctttgttgaataaaagtggagctacaggtgtttttactgattttccagatactggagttgaatttttgaagggtatcaaattgcctgaaacaggtactcatgattcagctgaattagttgttgctggattgatgtctacattagctgcttatggtttgactaaacgtaaagaagatggatccctccagtaa

SEQ ID NO: 225 - skipped

SEQ ID NO: 226 - protein, 368 aa, synthetic construct
MKLKTLALSLLAAGVLAGCSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPEFFTDVNQMYDALLNKSGATGVFTDFPDTGVEFLKGIKGSLQ

SEQ ID NO: 227 - DNA, 1107 bp, synthetic construct
  source 1..1107: mol_type = other DNA
atgaaattgaagacattggctttatcattgttagctgctggagttttggctggttgttctagtcattcatctaatatggctaatacacaaatgaagtctgataagatcattattgctcatcgtggagctagtggttatttgcctgaacatactttagaatcaaaagctcttgcttttgctcaacaagctgattatttggaacaagatttagctatgacaaaagatggacgtttggttgttatccatgatcattttcttgatggtttgactgatgttgctaaaaaatttccacatcgtcatcgtaaagatggacgttattatgttatcgattttactttgaaggaaattcaatctttggaaatgacagaaaattttgaaactaaagatggtaaacaagctcaagtttatccaaatcgttttcctttgtggaagtctcattttcgtattcatacatttgaagatgaaatcgaatttattcaaggtttggaaaaatctacaggtaagaaagttggtatctatccagaaatcaaggctccttggtttcatcatcaaaatggaaaggatattgctgctgaaacattgaaagttttgaaaaaatatggttatgataagaaaacagatatggtttatttacaaacttttgattttaatgaattgaagcgtatcaagactgaattgttgcctcaaatgggtatggatttgaaattggttcaattgatcgcttatacagattggaaggaaactcaagaaaaagatccaaagggatattgggttaattataattatgattggatgtttaagcctggtgctatggctgaagttgttaaatatgctgatggtgttggaccaggttggtatatgttggttaataaagaagaatctaagccagataatatcgtttatacacctttggttaaggaattagctcaatataatgttgaagttcatccatatacagttcgtaaagatgctttgcctgaatttttcactgatgttaatcaaatgtatgatgctttgttgaataaatcaggagctacaggtgtttttactgattttccagatactggagttgaatttttgaaaggtattaaaggatccctccagtaa

SEQ ID NO: 228 - DNA, 57 bp, synthetic construct
  source 1..57: mol_type = other DNA
atgaaattgaagacattggctttatcattgttagctgctggagttttggctggttgt

SEQ ID NO: 229 - DNA, 1798 bp, synthetic construct
  source 1..1798: mol_type = other DNA
aaagcactcaaaagtttaccttatgggtgcttttttcgtgcttttttgaaaagacaaaaaaaagaaccttgccaagcaagattcttttgatagggtggtctccgatcgtgtactgcagaaaactagtcgcaaaaaaccccgcttcggcggggttttttcgctctagacggtgatcaacacgctagccaggcatcaaataaaacgaaaggctcagtcgaaagactgggcctttcgttttatctgttgtttgtcggtgaacgctctctactagagtcacactggctcaccttcgggtgggcctttctgcgtttatagaattcactagtcattctacagtttattcttgacattgcactgtccccctggtataataactatacatgcaagatctaaataggaggaaaattaatgaagcatgaaaagcaacaacgttttagtatccgtaagtatgctgttggagctgcttcagttttgatcggttttgcttttcaagctcaaacagttgctgctgatggtgttacttctagtcattcatctaatatggctaatactcaaatgaagtctgataagatcattattgctcatcgtggagcttctggttatttgcctgaacatacattagaaagtaaagctcttgcttttgctcaacaagctgattatttggaacaagatttagctatgacaaaagatggacgtttggttgttatccatgatcattttcttgatggtttgactgatgttgctaaaaaatttccacatcgtcatcgtaaagatggtcgttattatgttatcgattttactttgaaggaaattcaaagtttggaaatgacagaaaattttgaaactaaagatggaaaacaagctcaagtttatccaaatcgttttcctttgtggaagtcacattttcgtattcatacatttgaagatgaaatcgaatttattcaaggtttggaaaaatctactggtaagaaagttggtatctatccagaaatcaaggctccttggtttcatcatcaaaatggaaaggatattgctgctgaaactttgaaagttttgaaaaaatatggttatgataagaaaacagatatggtttatttacaaacttttgattttaatgaattgaagcgtatcaagacagaattgttgcctcaaatgggaatggatttgaaattggttcaattgatcgcttatacagattggaaggaaactcaagaaaaagatccaaagggatattgggttaattataattatgattggatgtttaagcctggtgctatggctgaagttgttaaatatgctgatggtgttggaccaggttggtatatgttggttaataaagaagaatctaagccagataatatcgtttatactcctttggttaaggaattagctcaatataatgttgaagttcatccatatacagttcgtaaagatgctttgcctgaatttttcactgatgttaatcaaatgtatgatgctttgttgaataaatcaggagctacaggtgtttttactgattttccagatacaggagttgaatttttgaagggtatcaaattgtggtattatttgaatgaaagtggatcaatggctacaggttgggttaaggataagggtttatggtattatttaaatgaatctggaagtatggctactggatgggtaaaggataagggtctttggtactacttaaatgaatcaggatctatggctactggttgggtaaaagataaaggaggatccctccagtaactcgagaaaaaaaaaccgcgcccctgacagggcgcggttttttttt

SEQ ID NO: 230 - DNA, 1714 bp, synthetic construct
  source 1..1714: mol_type = other DNA
aaagcactcaaaagtttaccttatgggtgcttttttcgtgcttttttgaaaagacaaaaaaaagaaccttgccaagcaagattcttttgatagggtggtctccgatcgtgtactgcagaaaactagtcgcaaaaaaccccgcttcggcggggttttttcgctctagacggtgatcaacacgctagccaggcatcaaataaaacgaaaggctcagtcgaaagactgggcctttcgttttatctgttgtttgtcggtgaacgctctctactagagtcacactggctcaccttcgggtgggcctttctgcgtttatagaattcactagtcattctacagtttattcttgacattgcactgtccccctggtataataactatacatgcaagatctaaataggaggaaaattaatgaagcatgaaaagcaacaacgttttagtatccgtaagtatgctgttggagctgcttcagttttgatcggttttgcttttcaagctcaaacagttgctgctgatggagttacttctagtcattcatctaatatggctaatacacaaatgaagtcagataagatcattattgctcatcgtggagcttctggttatttgcctgaacatactttagaaagtaaagctcttgcttttgctcaacaagctgattatttggaacaagatttagctatgacaaaagatggacgtttggttgttatccatgatcattttcttgatggtttgactgatgttgctaaaaaatttccacatcgtcatcgtaaagatggacgttattatgttatcgattttacattgaaggaaattcaaagtttggaaatgacagaaaattttgaaactaaagatggtaaacaagctcaagtttatccaaatcgttttcctttgtggaagagtcattttcgtattcatacatttgaagatgaaatcgaatttattcaaggattggaaaaatcaactggtaagaaagttggtatctatccagaaatcaaggctccttggtttcatcatcaaaatggaaaggatattgctgctgaaacattgaaagttttgaaaaaatatggttatgataagaaaacagatatggtttatttacaaacttttgattttaatgaattgaagcgtatcaagactgaattgttgcctcaaatgggtatggatttgaaattggttcaattgatcgcttatacagattggaaggaaactcaagaaaaagatccaaagggatattgggttaattataattatgattggatgtttaagcctggtgctatggctgaagttgttaaatatgctgatggtgttggaccaggttggtatatgttggttaataaagaagaatctaagccagataatatcgtttatacacctttggttaaggaattagctcaatataatgttgaagttcatccatatacagttcgtaaagatgctttgcctgaatttttcactgatgttaatcaaatgtatgatgctttgttgaataaaagtggagctacaggtgtttttactgattttccagatactggagttgaatttttgaagggtatcaaattgcctgaaacaggtactcatgattcagctgaattagttgttgctggattgatgtctacattagctgcttatggtttgactaaacgtaaagaagatggatccctccagtaactcgagaaaaaaaaaccgcgcccctgacagggcgcggttttttttt

SEQ ID NO: 231 - DNA, 1561 bp, synthetic construct
  source 1..1561: mol_type = other DNA
aaagcactcaaaagtttaccttatgggtgcttttttcgtgcttttttgaaaagacaaaaaaaagaaccttgccaagcaagattcttttgatagggtggtctccgatcgtgtactgcagaaaactagtcgcaaaaaaccccgcttcggcggggttttttcgctctagacggtgatcaacacgctagccaggcatcaaataaaacgaaaggctcagtcgaaagactgggcctttcgttttatctgttgtttgtcggtgaacgctctctactagagtcacactggctcaccttcgggtgggcctttctgcgtttatagaattcactagtcattctacagtttattcttgacattgcactgtccccctggtataataactatacatgcaagatctaaataggaggaaaattaatgaaattgaagacattggctttatcattgttagctgctggagttttggctggttgttctagtcattcatctaatatggctaatacacaaatgaagtctgataagatcattattgctcatcgtggagctagtggttatttgcctgaacatactttagaatcaaaagctcttgcttttgctcaacaagctgattatttggaacaagatttagctatgacaaaagatggacgtttggttgttatccatgatcattttcttgatggtttgactgatgttgctaaaaaatttccacatcgtcatcgtaaagatggacgttattatgttatcgattttactttgaaggaaattcaatctttggaaatgacagaaaattttgaaactaaagatggtaaacaagctcaagtttatccaaatcgttttcctttgtggaagtctcattttcgtattcatacatttgaagatgaaatcgaatttattcaaggtttggaaaaatctacaggtaagaaagttggtatctatccagaaatcaaggctccttggtttcatcatcaaaatggaaaggatattgctgctgaaacattgaaagttttgaaaaaatatggttatgataagaaaacagatatggtttatttacaaacttttgattttaatgaattgaagcgtatcaagactgaattgttgcctcaaatgggtatggatttgaaattggttcaattgatcgcttatacagattggaaggaaactcaagaaaaagatccaaagggatattgggttaattataattatgattggatgtttaagcctggtgctatggctgaagttgttaaatatgctgatggtgttggaccaggttggtatatgttggttaataaagaagaatctaagccagataatatcgtttatacacctttggttaaggaattagctcaatataatgttgaagttcatccatatacagttcgtaaagatgctttgcctgaatttttcactgatgttaatcaaatgtatgatgctttgttgaataaatcaggagctacaggtgtttttactgattttccagatactggagttgaatttttgaaaggtattaaaggatccctccagtaactcgagaaaaaaaaaccgcgcccctgacagggcgcggttttttttt

SEQ ID NO: 232 - DNA, 5574 bp, synthetic construct
  source 1..5574: mol_type = other DNA
tgacaaaaaaggtacaaaaatcatggaaatgacttctgggatatgtttgcgccaaaaaatttcaagataagacagactcaaacctgcaagcaaggctgggataacttgggcttggtaaccaatacgattaacagtaaaataaccaaaattccaaacccagtttgctgcgatatcagctgctggcgttgaagcaaccgcataggcattgagcaactgaggcgataccaaacagattccgagaacaattcccaaaatttggctggttcccatcttacgagaaacagaccaagtaatccctactggtaagaactggaagatagcttcaccaggcaaccagaggaagtgattaacccctgcccaaaactgagaggattctgtgatggtcttgccatccaacatcgaccaatggacaccttccaagacattacggaaaccgaggatcaatccaccaacaatcaaggctggaataatcggagtaaagatttccgccagagtggtcataacaccttggaccacgttttgattactcttagctgcagacttggctgcttctttggaaacaccctcaatacctgaaacggctgtaaaatcattataaaagatgggcacgtcatttccaatgattacctgaaattgacctgcatttgtaaaggttcctttaacagctggaattgactcgatagctttaacattagccttcttatcatctcctaaaacaaaccgcatccgtgtcgcacagtgggtgacggcagtcacattctctttacctccaattgcctgaagcaaatctttggcttcttgttcaaattttcccatttgattctccttatacttgtcaaagcaagtttgatttgataagtatattgtaaacgataccaaacttgtctgcaacttgtttggtataaaaagggtgtttttttgtaaattttctagcacttgtatggtaaaatagatacaggtgttcattaaactagactaaaaacctatttaagaaagcaaaatgaagaaataccaacaattatttaagcaaatccaagaaaccattcaaaacgagacttacgctgtcggagattttcttcctagcgagcacgaccttatggagcaatatcaagtgagtcgtgataccgtccgaaaggccctgtctctcctccaagaggaaggattgatcaaaaagataagagggcaaggttctcaagtcgtcaaagaagaaaccgtcaatttccctgtatccaacctaaccagctaccaagaactagttaaagaacttggactgcgctctaaaaccaacgtggtcagtctggacaagattattattgataaaaaatcctcactgataaccggtttcccagagtttcggatggtttggaaggtggtccgccagcgtgtggtggatgatctggtatccgttctggatacggactatctggatatggaactaatcccaaatctcactcgccaaatcgctgagcagtctatctattcttatatagaaaatggcctcaaactccttattgattatgctcagaaggaaatcaccattgaccactcaagcgaccgagacaagattctcatggacattggcaaagacccttatgtcgtttcgattaaatcaaaagtctatctccaagacggacgccaatttcagtttaccgaaagtcgccataagttagaaaaatttagatttgtagattttgcaaaacgcaagaaataaaagactgagacaccagatcaaagcactcaaaagtttaccttatgggtgcttttttcgtgcttttttgaaaagacaaaaaaaagaaccttgccaagcaagattcttttgatagggtggtctccgatcgtgtactgcagaaaactagtcgcaaaaaaccccgcttcggcggggttttttcgctctagacggtgatcaacacgctagccaggcatcaaataaaacgaaaggctcagtcgaaagactgggcctttcgttttatctgttgtttgtcggtgaacgctctctactagagtcacactggctcaccttcgggtgggcctttctgcgtttatagaattcactagtcattctacagtttattcttgacattgcactgtccccctggtataataactatacatgcaagatctaaataggaggaaaattaatgaagcatgaaaagcaacaacgttttagtatccgtaagtatgctgttggagctgcttcagttttgatcggttttgcttttcaagctcaaacagttgctgctgatggtgttacttctagtcattcatctaatatggctaatactcaaatgaagtctgataagatcattattgctcatcgtggagcttctggttatttgcctgaacatacattagaaagtaaagctcttgcttttgctcaacaagctgattatttggaacaagatttagctatgacaaaagatggacgtttggttgttatccatgatcattttcttgatggtttgactgatgttgctaaaaaatttccacatcgtcatcgtaaagatggtcgttattatgttatcgattttactttgaaggaaattcaaagtttggaaatgacagaaaattttgaaactaaagatggaaaacaagctcaagtttatccaaatcgttttcctttgtggaagtcacattttcgtattcatacatttgaagatgaaatcgaatttattcaaggtttggaaaaatctactggtaagaaagttggtatctatccagaaatcaaggctccttggtttcatcatcaaaatggaaaggatattgctgctgaaactttgaaagttttgaaaaaatatggttatgataagaaaacagatatggtttatttacaaacttttgattttaatgaattgaagcgtatcaagacagaattgttgcctcaaatgggaatggatttgaaattggttcaattgatcgcttatacagattggaaggaaactcaagaaaaagatccaaagggatattgggttaattataattatgattggatgtttaagcctggtgctatggctgaagttgttaaatatgctgatggtgttggaccaggttggtatatgttggttaataaagaagaatctaagccagataatatcgtttatactcctttggttaaggaattagctcaatataatgttgaagttcatccatatacagttcgtaaagatgctttgcctgaatttttcactgatgttaatcaaatgtatgatgctttgttgaataaatcaggagctacaggtgtttttactgattttccagatacaggagttgaatttttgaagggtatcaaattgtggtattatttgaatgaaagtggatcaatggctacaggttgggttaaggataagggtttatggtattatttaaatgaatctggaagtatggctactggatgggtaaaggataagggtctttggtactacttaaatgaatcaggatctatggctactggttgggtaaaagataaaggaggatccctccagtaactcgagaaaaaaaaaccgcgcccctgacagggcgcggttttttttttaattcccataaaaattgacatggaaattataaaaccattattagtttagtcctttttgataacgtgctaattcggcttggttcgcccaaacatagtgacctggacggatttctaccatagatggcttatcagtctcatagtcgtgttgacttgggtcgtaaaccttcaagaccttcttacgttccaagattggatctgggattggtaccgctgaaagcaaggcttgagtatatgggtgaattggattgttaaacaattcttctgtttctgcaacctctacaataacacccttgtaaataactgcgatacgatctgaaataaagcgaacaaccgacaagtcatgggcgatgaagagataggtcaagccgagctctttttggaattttttgagcaagttcaagacttgggcacgtacagaaacgtccaaggctgaaattggctcatctgcaataacaaagtctggttgcatgaccaaggcacgggcaataccgatacgttgacgttgaccgcctgagaattcatgagggtaacgagtcaagtgctcagcaagaagacctacttcacggataatactttgaactttctctttacgttcttcttcatccttaaataaacggtgattgtaaagaccttcagaaataatataatcaacagtcgcacgttcattcaaacttgcggcagggtcttggaaaatcatctggattcgacgaatcaattccgcagcttgttcacgcgatttcttaccattaatcttttgaccatcaaaaatgatatctccattacttgtatcatttagaccgatgatagcacgaccaatagttgttttcccactaccggactcacctacaagcgagaaagtttctcccttgttgataaagaagttagcatttttaaccgcgacaaacttcttacttccttcaccgaaggaaatttctaaatctttgatttctactaatttttcagacatttccttcctcctagtcagccagatgggcaaatcccattttttcacggatcttatcatggagatttgcaatcacagctggtttttctacttttggagcatcctcatgaagaagccaagttttagcccaatgtgtctctgatactgagaattgaggagctttttgttcgaagtcaatctgcattgcgtagtcagaacgcaaggcaaaagcatcccctttcaggtcagtataaagtgacggaggtgttcctgggattgagtaaagatcccctttatcatcagcaagctgaggcaagctagacaagagactccatgtatatggatggcgagggtcatagaagacttcctcaaccgttccatactcaacgatttctcctgcatacataaccgctaccttatccgcaatacttgccaccacaccaaggtcgtgggtaataaagattgttgtgaaatgatactcgttttgtaaagattttagcaaatcaataatctgagcttgaatagttacatccaaggcagttgttggctcatcacagatcaagacatcaggtcggcaggcaagggcaatcgcaataacgatacgttgacgcattcctccagaatattggaatgggtattcattaaaacgtctatctgcgtctggaatgccaaccttattcatgtagtcaatggccaattctttcgcttctttagctgtttttccttggtgttttacaataacttctgtaatctgactaccaattgttttaatggggtccaaactagtcattgggtcctggaagatagtcgcaatcttagcaccacgaatttgttcccaatccttgtgagaagataaagctgtcaagtcctgaccacggtagtcaatactaccttgggcaatacgaccattttcttcgagcatacctgtgaaggtctttgtcaaaacagatttacctgatcctgactcacctaccaaggctaatacttctccttcgactagttcaagggaaacgccgcgaatggctgtcaatactttgtcacgaacgtcaaattccacgacaatatcgcgagcagtcaaaattacatttttttcttttgtcat

SEQ ID NO: 233 - DNA, 5490 bp, synthetic construct
  source 1..5490: mol_type = other DNA
tgacaaaaaaggtacaaaaatcatggaaatgacttctgggatatgtttgcgccaaaaaatttcaagataagacagactcaaacctgcaagcaaggctgggataacttgggcttggtaaccaatacgattaacagtaaaataaccaaaattccaaacccagtttgctgcgatatcagctgctggcgttgaagcaaccgcataggcattgagcaactgaggcgataccaaacagattccgagaacaattcccaaaatttggctggttcccatcttacgagaaacagaccaagtaatccctactggtaagaactggaagatagcttcaccaggcaaccagaggaagtgattaacccctgcccaaaactgagaggattctgtgatggtcttgccatccaacatcgaccaatggacaccttccaagacattacggaaaccgaggatcaatccaccaacaatcaaggctggaataatcggagtaaagatttccgccagagtggtcataacaccttggaccacgttttgattactcttagctgcagacttggctgcttctttggaaacaccctcaatacctgaaacggctgtaaaatcattataaaagatgggcacgtcatttccaatgattacctgaaattgacctgcatttgtaaaggttcctttaacagctggaattgactcgatagctttaacattagccttcttatcatctcctaaaacaaaccgcatccgtgtcgcacagtgggtgacggcagtcacattctctttacctccaattgcctgaagcaaatctttggcttcttgttcaaattttcccatttgattctccttatacttgtcaaagcaagtttgatttgataagtatattgtaaacgataccaaacttgtctgcaacttgtttggtataaaaagggtgtttttttgtaaattttctagcacttgtatggtaaaatagatacaggtgttcattaaactagactaaaaacctatttaagaaagcaaaatgaagaaataccaacaattatttaagcaaatccaagaaaccattcaaaacgagacttacgctgtcggagattttcttcctagcgagcacgaccttatggagcaatatcaagtgagtcgtgataccgtccgaaaggccctgtctctcctccaagaggaaggattgatcaaaaagataagagggcaaggttctcaagtcgtcaaagaagaaaccgtcaatttccctgtatccaacctaaccagctaccaagaactagttaaagaacttggactgcgctctaaaaccaacgtggtcagtctggacaagattattattgataaaaaatcctcactgataaccggtttcccagagtttcggatggtttggaaggtggtccgccagcgtgtggtggatgatctggtatccgttctggatacggactatctggatatggaactaatcccaaatctcactcgccaaatcgctgagcagtctatctattcttatatagaaaatggcctcaaactccttattgattatgctcagaaggaaatcaccattgaccactcaagcgaccgagacaagattctcatggacattggcaaagacccttatgtcgtttcgattaaatcaaaagtctatctccaagacggacgccaatttcagtttaccgaaagtcgccataagttagaaaaatttagatttgtagattttgcaaaacgcaagaaataaaagactgagacaccagatcaaagcactcaaaagtttaccttatgggtgcttttttcgtgcttttttgaaaagacaaaaaaaagaaccttgccaagcaagattcttttgatagggtggtctccgatcgtgtactgcagaaaactagtcgcaaaaaaccccgcttcggcggggttttttcgctctagacggtgatcaacacgctagccaggcatcaaataaaacgaaaggctcagtcgaaagactgggcctttcgttttatctgttgtttgtcggtgaacgctctctactagagtcacactggctcaccttcgggtgggcctttctgcgtttatagaattcactagtcattctacagtttattcttgacattgcactgtccccctggtataataactatacatgcaagatctaaataggaggaaaattaatgaagcatgaaaagcaacaacgttttagtatccgtaagtatgctgttggagctgcttcagttttgatcggttttgcttttcaagctcaaacagttgctgctgatggagttacttctagtcattcatctaatatggctaatacacaaatgaagtcagataagatcattattgctcatcgtggagcttctggttatttgcctgaacatactttagaaagtaaagctcttgcttttgctcaacaagctgattatttggaacaagatttagctatgacaaaagatggacgtttggttgttatccatgatcattttcttgatggtttgactgatgttgctaaaaaatttccacatcgtcatcgtaaagatggacgttattatgttatcgattttacattgaaggaaattcaaagtttggaaatgacagaaaattttgaaactaaagatggtaaacaagctcaagtttatccaaatcgttttcctttgtggaagagtcattttcgtattcatacatttgaagatgaaatcgaatttattcaaggattggaaaaatcaactggtaagaaagttggtatctatccagaaatcaaggctccttggtttcatcatcaaaatggaaaggatattgctgctgaaacattgaaagttttgaaaaaatatggttatgataagaaaacagatatggtttatttacaaacttttgattttaatgaattgaagcgtatcaagactgaattgttgcctcaaatgggtatggatttgaaattggttcaattgatcgcttatacagattggaaggaaactcaagaaaaagatccaaagggatattgggttaattataattatgattggatgtttaagcctggtgctatggctgaagttgttaaatatgctgatggtgttggaccaggttggtatatgttggttaataaagaagaatctaagccagataatatcgtttatacacctttggttaaggaattagctcaatataatgttgaagttcatccatatacagttcgtaaagatgctttgcctgaatttttcactgatgttaatcaaatgtatgatgctttgttgaataaaagtggagctacaggtgtttttactgattttccagatactggagttgaatttttgaagggtatcaaattgcctgaaacaggtactcatgattcagctgaattagttgttgctggattgatgtctacattagctgcttatggtttgactaaacgtaaagaagatggatccctccagtaactcgagaaaaaaaaaccgcgcccctgacagggcgcggttttttttttaattcccataaaaattgacatggaaattataaaaccattattagtttagtcctttttgataacgtgctaattcggcttggttcgcccaaacatagtgacctggacggatttctaccatagatggcttatcagtctcatagtcgtgttgacttgggtcgtaaaccttcaagaccttcttacgttccaagattggatctgggattggtaccgctgaaagcaaggcttgagtatatgggtgaattggattgttaaacaattcttctgtttctgcaacctctacaataacacccttgtaaataactgcgatacgatctgaaataaagcgaacaaccgacaagtcatgggcgatgaagagataggtcaagccgagctctttttggaattttttgagcaagttcaagacttgggcacgtacagaaacgtccaaggctgaaattggctcatctgcaataacaaagtctggttgcatgaccaaggcacgggcaataccgatacgttgacgttgaccgcctgagaattcatgagggtaacgagtcaagtgctcagcaagaagacctacttcacggataatactttgaactttctctttacgttcttcttcatccttaaataaacggtgattgtaaagaccttcagaaataatataatcaacagtcgcacgttcattcaaacttgcggcagggtcttggaaaatcatctggattcgacgaatcaattccgcagcttgttcacgcgatttcttaccattaatcttttgaccatcaaaaatgatatctccattacttgtatcatttagaccgatgatagcacgaccaatagttgttttcccactaccggactcacctacaagcgagaaagtttctcccttgttgataaagaagttagcatttttaaccgcgacaaacttcttacttccttcaccgaaggaaatttctaaatctttgatttctactaatttttcagacatttccttcctcctagtcagccagatgggcaaatcccattttttcacggatcttatcatggagatttgcaatcacagctggtttttctacttttggagcatcctcatgaagaagccaagttttagcccaatgtgtctctgatactgagaattgaggagctttttgttcgaagtcaatctgcattgcgtagtcagaacgcaaggcaaaagcatcccctttcaggtcagtataaagtgacggaggtgttcctgggattgagtaaagatcccctttatcatcagcaagctgaggcaagctagacaagagactccatgtatatggatggcgagggtcatagaagacttcctcaaccgttccatactcaacgatttctcctgcatacataaccgctaccttatccgcaatacttgccaccacaccaaggtcgtgggtaataaagattgttgtgaaatgatactcgttttgtaaagattttagcaaatcaataatctgagcttgaatagttacatccaaggcagttgttggctcatcacagatcaagacatcaggtcggcaggcaagggcaatcgcaataacgatacgttgacgcattcctccagaatattggaatgggtattcattaaaacgtctatctgcgtctggaatgccaaccttattcatgtagtcaatggccaattctttcgcttctttagctgtttttccttggtgttttacaataacttctgtaatctgactaccaattgttttaatggggtccaaactagtcattgggtcctggaagatagtcgcaatcttagcaccacgaatttgttcccaatccttgtgagaagataaagctgtcaagtcctgaccacggtagtcaatactaccttgggcaatacgaccattttcttcgagcatacctgtgaaggtctttgtcaaaacagatttacctgatcctgactcacctaccaaggctaatacttctccttcgactagttcaagggaaacgccgcgaatggctgtcaatactttgtcacgaacgtcaaattccacgacaatatcgcgagcagtcaaaattacatttttttcttttgtcat

SEQ ID NO: 234 - DNA, 5337 bp, synthetic construct
  source 1..5337: mol_type = other DNA
tgacaaaaaaggtacaaaaatcatggaaatgacttctgggatatgtttgcgccaaaaaatttcaagataagacagactcaaacctgcaagcaaggctgggataacttgggcttggtaaccaatacgattaacagtaaaataaccaaaattccaaacccagtttgctgcgatatcagctgctggcgttgaagcaaccgcataggcattgagcaactgaggcgataccaaacagattccgagaacaattcccaaaatttggctggttcccatcttacgagaaacagaccaagtaatccctactggtaagaactggaagatagcttcaccaggcaaccagaggaagtgattaacccctgcccaaaactgagaggattctgtgatggtcttgccatccaacatcgaccaatggacaccttccaagacattacggaaaccgaggatcaatccaccaacaatcaaggctggaataatcggagtaaagatttccgccagagtggtcataacaccttggaccacgttttgattactcttagctgcagacttggctgcttctttggaaacaccctcaatacctgaaacggctgtaaaatcattataaaagatgggcacgtcatttccaatgattacctgaaattgacctgcatttgtaaaggttcctttaacagctggaattgactcgatagctttaacattagccttcttatcatctcctaaaacaaaccgcatccgtgtcgcacagtgggtgacggcagtcacattctctttacctccaattgcctgaagcaaatctttggcttcttgttcaaattttcccatttgattctccttatacttgtcaaagcaagtttgatttgataagtatattgtaaacgataccaaacttgtctgcaacttgtttggtataaaaagggtgtttttttgtaaattttctagcacttgtatggtaaaatagatacaggtgttcattaaactagactaaaaacctatttaagaaagcaaaatgaagaaataccaacaattatttaagcaaatccaagaaaccattcaaaacgagacttacgctgtcggagattttcttcctagcgagcacgaccttatggagcaatatcaagtgagtcgtgataccgtccgaaaggccctgtctctcctccaagaggaaggattgatcaaaaagataagagggcaaggttctcaagtcgtcaaagaagaaaccgtcaatttccctgtatccaacctaaccagctaccaagaactagttaaagaacttggactgcgctctaaaaccaacgtggtcagtctggacaagattattattgataaaaaatcctcactgataaccggtttcccagagtttcggatggtttggaaggtggtccgccagcgtgtggtggatgatctggtatccgttctggatacggactatctggatatggaactaatcccaaatctcactcgccaaatcgctgagcagtctatctattcttatatagaaaatggcctcaaactccttattgattatgctcagaaggaaatcaccattgaccactcaagcgaccgagacaagattctcatggacattggcaaagacccttatgtcgtttcgattaaatcaaaagtctatctccaagacggacgccaatttcagtttaccgaaagtcgccataagttagaaaaatttagatttgtagattttgcaaaacgcaagaaataaaagactgagacaccagatcaaagcactcaaaagtttaccttatgggtgcttttttcgtgcttttttgaaaagacaaaaaaaagaaccttgccaagcaagattcttttgatagggtggtctccgatcgtgtactgcagaaaactagtcgcaaaaaaccccgcttcggcggggttttttcgctctagacggtgatcaacacgctagccaggcatcaaataaaacgaaaggctcagtcgaaagactgggcctttcgttttatctgttgtttgtcggtgaacgctctctactagagtcacactggctcaccttcgggtgggcctttctgcgtttatagaattcactagtcattctacagtttattcttgacattgcactgtccccctggtataataactatacatgcaagatctaaataggaggaaaattaatgaaattgaagacattggctttatcattgttagctgctggagttttggctggttgttctagtcattcatctaatatggctaatacacaaatgaagtctgataagatcattattgctcatcgtggagctagtggttatttgcctgaacatactttagaatcaaaagctcttgcttttgctcaacaagctgattatttggaacaagatttagctatgacaaaagatggacgtttggttgttatccatgatcattttcttgatggtttgactgatgttgctaaaaaatttccacatcgtcatcgtaaagatggacgttattatgttatcgattttactttgaaggaaattcaatctttggaaatgacagaaaattttgaaactaaagatggtaaacaagctcaagtttatccaaatcgttttcctttgtggaagtctcattttcgtattcatacatttgaagatgaaatcgaatttattcaaggtttggaaaaatctacaggtaagaaagttggtatctatccagaaatcaaggctccttggtttcatcatcaaaatggaaaggatattgctgctgaaacattgaaagttttgaaaaaatatggttatgataagaaaacagatatggtttatttacaaacttttgattttaatgaattgaagcgtatcaagactgaattgttgcctcaaatgggtatggatttgaaattggttcaattgatcgcttatacagattggaaggaaactcaagaaaaagatccaaagggatattgggttaattataattatgattggatgtttaagcctggtgctatggctgaagttgttaaatatgctgatggtgttggaccaggttggtatatgttggttaataaagaagaatctaagccagataatatcgtttatacacctttggttaaggaattagctcaatataatgttgaagttcatccatatacagttcgtaaagatgctttgcctgaatttttcactgatgttaatcaaatgtatgatgctttgttgaataaatcaggagctacaggtgtttttactgattttccagatactggagttgaatttttgaaaggtattaaaggatccctccagtaactcgagaaaaaaaaaccgcgcccctgacagggcgcggttttttttttaattcccataaaaattgacatggaaattataaaaccattattagtttagtcctttttgataacgtgctaattcggcttggttcgcccaaacatagtgacctggacggatttctaccatagatggcttatcagtctcatagtcgtgttgacttgggtcgtaaaccttcaagaccttcttacgttccaagattggatctgggattggtaccgctgaaagcaaggcttgagtatatgggtgaattggattgttaaacaattcttctgtttctgcaacctctacaataacacccttgtaaataactgcgatacgatctgaaataaagcgaacaaccgacaagtcatgggcgatgaagagataggtcaagccgagctctttttggaattttttgagcaagttcaagacttgggcacgtacagaaacgtccaaggctgaaattggctcatctgcaataacaaagtctggttgcatgaccaaggcacgggcaataccgatacgttgacgttgaccgcctgagaattcatgagggtaacgagtcaagtgctcagcaagaagacctacttcacggataatactttgaactttctctttacgttcttcttcatccttaaataaacggtgattgtaaagaccttcagaaataatataatcaacagtcgcacgttcattcaaacttgcggcagggtcttggaaaatcatctggattcgacgaatcaattccgcagcttgttcacgcgatttcttaccattaatcttttgaccatcaaaaatgatatctccattacttgtatcatttagaccgatgatagcacgaccaatagttgttttcccactaccggactcacctacaagcgagaaagtttctcccttgttgataaagaagttagcatttttaaccgcgacaaacttcttacttccttcaccgaaggaaatttctaaatctttgatttctactaatttttcagacatttccttcctcctagtcagccagatgggcaaatcccattttttcacggatcttatcatggagatttgcaatcacagctggtttttctacttttggagcatcctcatgaagaagccaagttttagcccaatgtgtctctgatactgagaattgaggagctttttgttcgaagtcaatctgcattgcgtagtcagaacgcaaggcaaaagcatcccctttcaggtcagtataaagtgacggaggtgttcctgggattgagtaaagatcccctttatcatcagcaagctgaggcaagctagacaagagactccatgtatatggatggcgagggtcatagaagacttcctcaaccgttccatactcaacgatttctcctgcatacataaccgctaccttatccgcaatacttgccaccacaccaaggtcgtgggtaataaagattgttgtgaaatgatactcgttttgtaaagattttagcaaatcaataatctgagcttgaatagttacatccaaggcagttgttggctcatcacagatcaagacatcaggtcggcaggcaagggcaatcgcaataacgatacgttgacgcattcctccagaatattggaatgggtattcattaaaacgtctatctgcgtctggaatgccaaccttattcatgtagtcaatggccaattctttcgcttctttagctgtttttccttggtgttttacaataacttctgtaatctgactaccaattgttttaatggggtccaaactagtcattgggtcctggaagatagtcgcaatcttagcaccacgaatttgttcccaatccttgtgagaagataaagctgtcaagtcctgaccacggtagtcaatactaccttgggcaatacgaccattttcttcgagcatacctgtgaaggtctttgtcaaaacagatttacctgatcctgactcacctaccaaggctaatacttctccttcgactagttcaagggaaacgccgcgaatggctgtcaatactttgtcacgaacgtcaaattccacgacaatatcgcgagcagtcaaaattacatttttttcttttgtcat

SEQ ID NO: 235 - DNA, 46 bp, synthetic construct
  source 1..46: mol_type = other DNA
ctcgagaaaaaaaaaccgcgcccctgacagggcgcggttttttttt

SEQ ID NO: 236 - DNA, 408 bp, synthetic construct
  source 1..408: mol_type = other DNA
aaagcactcaaaagtttaccttatgggtgcttttttcgtgcttttttgaaaagacaaaaaaaagaaccttgccaagcaagattcttttgatagggtggtctccgatcgtgtactgcagaaaactagtcgcaaaaaaccccgcttcggcggggttttttcgctctagacggtgatcaacacgctagccaggcatcaaataaaacgaaaggctcagtcgaaagactgggcctttcgttttatctgttgtttgtcggtgaacgctctctactagagtcacactggctcaccttcgggtgggcctttctgcgtttatagaattcactagtcattctacagtttattcttgacattgcactgtccccctggtataataactatacatgcaagatctaaataggaggaaaatta

SEQ ID NO: 237 - DNA, 319 bp, synthetic construct
  source 1..319: mol_type = other DNA
aaagcactcaaaagtttaccttatgggtgcttttttcgtgcttttttgaaaagacaaaaaaaagaaccttgccaagcaagattcttttgatagggtggtctccgatcgtgtactgcagaaaactagtcgcaaaaaaccccgcttcggcggggttttttcgctctagacggtgatcaacacgctagccaggcatcaaataaaacgaaaggctcagtcgaaagactgggcctttcgttttatctgttgtttgtcggtgaacgctctctactagagtcacactggctcaccttcgggtgggcctttctgcgtttatagaatt

SEQ ID NO: 238 - DNA, 56 bp, synthetic construct
  source 1..56: mol_type = other DNA
cataaggtaaacttttgagtgctttgatctggtgtctcagtcttttatttcttgcg

SEQ ID NO: 239 - DNA, 59 bp, synthetic construct
  source 1..59: mol_type = other DNA
ccctgacagggcgcggttttttttttaattcccataaaaattgacatggaaattataaa

SEQ ID NO: 240 - DNA, 30 bp, synthetic construct
  source 1..30: mol_type = other DNA
aaagcactcaaaagtttaccttatgggtgc

SEQ ID NO: 241 - DNA, 25 bp, synthetic construct
  source 1..25: mol_type = other DNA
aaaaaaaaaccgcgccctgtcaggg

SEQ ID NO: 242 - DNA, 56 bp, synthetic construct
  source 1..56: mol_type = other DNA
gaaataaaagactgagacaccagatcaaagcactcaaaagtttaccttatgggtgc

SEQ ID NO: 243 - DNA, 30 bp, synthetic construct
  source 1..30: mol_type = other DNA
caaagattgaatttccttcaaagtaaaatc

SEQ ID NO: 244 - DNA, 60 bp, synthetic construct
  source 1..60: mol_type = other DNA
ttttactttgaaggaaattcaatctttggaaatgacagaaaattttgaaactaaagatgg

SEQ ID NO: 245 - DNA, 55 bp, synthetic construct
  source 1..55: mol_type = other DNA
taatttccatgtcaatttttatgggaattaaaaaaaaaaccgcgccctgtcaggg

SEQ ID NO: 246 - DNA, 24 bp, synthetic construct
  source 1..24: mol_type = other DNA
ctttggaaacaccctcaatacctg

SEQ ID NO: 247 - DNA, 24 bp, synthetic construct
  source 1..24: mol_type = other DNA
gcttttgccttgcgttctgactac

SEQ ID NO: 248 - DNA, 60 bp, synthetic construct; identical to SEQ ID NO: 244
  source 1..60: mol_type = other DNA

SEQ ID NO: 249 - DNA, 25 bp, synthetic construct
  source 1..25: mol_type = other DNA
gtgatcaacacgctagccaggcatc

SEQ ID NO: 250 - DNA, 26 bp, synthetic construct
  source 1..26: mol_type = other DNA
ggacgccaatttcagtttaccgaaag

SEQ ID NO: 251 - DNA, 34 bp, synthetic construct
  source 1..34: mol_type = other DNA
taattttcctcctatttagatcttgcatgtatag

SEQ ID NO: 252 - DNA, 25 bp, synthetic construct
  source 1..25: mol_type = other DNA
ctttcagcggtaccaatcccagatc

SEQ ID NO: 253 - DNA, 27 bp, synthetic construct
  source 1..27: mol_type = other DNA
cttggtatcacaatggatcaggtcatg

SEQ ID NO: 254 - DNA, 27 bp, synthetic construct
  source 1..27: mol_type = other DNA
cttggtaaggaaatgatccaaagcatc

SEQ ID NO: 255 - DNA, 25 bp, synthetic construct
  source 1..25: mol_type = other DNA
gtagcgagtttggaagatatcacag

SEQ ID NO: 256 - DNA, 28 bp, synthetic construct
  source 1..28: mol_type = other DNA
cacaataagctcttcttcttgtctatgg

SEQ ID NO: 257 - DNA, 30 bp, synthetic construct
  source 1..30: mol_type = other DNA
ggaagaactgctgattatgagtgatgttgg

SEQ ID NO: 258 - DNA, 27 bp, synthetic construct
  source 1..27: mol_type = other DNA
cctcgagcagttccatcaatcttagtc

SEQ ID NO: 259 - DNA, 26 bp, synthetic construct
  source 1..26: mol_type = other DNA
cagatgtaaatgtggctgaacctgac

SEQ ID NO: 260 - DNA, 28 bp, synthetic construct
  source 1..28: mol_type = other DNA
gacggaaaaatccgtttattctacactg

SEQ ID NO: 261 - protein, 832 aa, Moraxella catarrhalis
MNKIYKVKKNAAGHLVACSEFAKGHTKKAVLGSLLIVGALGMATTASAQATNSKGTGAHIGVNNNNEAPGDYSFIGSGGYNKAEGRYSAIGGGLFNKATNEYSTIVGGGYNKAEGRYSTIGGGSNNEATNEYSTIVGGDDNKATGRYSTIGGGDNNTAEGEYSTVAGGKNNQATGTGSFAAGVENQANAENAVAVGKKNIIEGENSVAIGSENTVKTEHKNVFILGSGTTGVTSNSVLLGNETAGKQATTVKNAEVGGLSLTGFAGESKAENGVVSVGSEGGERQIVNVGAGQISDTSTDAVNGSQLHALATVVDDNQYDIVNNRADILNNQDDIKDLQKEVKGLDNEVGELSRDINSLHDVTDNQQDDIKELKRGVKELDNEVGVLSRDINSLHDDVADNQDDIAKNKADIKGLNKEVKELDKEVGVLSRDIGSLHDDVATNQADIAKNQADIKTLENNVEEELLNLSGRLLDQKADIDNNINNIYELAQQQDQHSSDIKTLKNNVEEGLLDLSGRLIDQKADIAKNQADIAQNQTDIQDLAAYNELQDQYAQKQTEAIDALNKASSENTQNIAKNQADIANNINNIYELAQQQDQHSSDIKTLAKVSAANTDRIAKNKAEADASFETLTKNQNTLIEQGEALVEQNKAINQELEGFAAHADIQDKQILQNQADITTNKTAIEQNINRTVANGFEIEKNKAGIATNKQELILQNDRLNRINETNNRQDQKIDQLGYALKEQGQHFNNRISAVERQTAGGIANAIAIATLPSPSRAGEHHVLFGSGYHNGQAAVSLGAAGLSDTGKSTYKIGLSWSDAGGLSGGVGGSYRWK

SEQ ID NO: 262 - DNA, 2499 bp, Moraxella catarrhalis
  source 1..2499: mol_type = other DNA
atgaataaaatttataaagtgaagaaaaatgccgcaggtcacttggtggcatgttctgaatttgccaaaggtcataccaaaaaggcagttttgggcagtttattgattgttggggcgttgggcatggcaacgacggcgtctgcacaagcaaccaacagcaaaggcacaggcgcgcacatcggtgttaacaataacaacgaagccccaggcgattactctttcatcggtagtggcggttataacaaagccgaaggcagatactctgccatcggtggtggcctttttaacaaagccacaaacgagtactctaccatcgttggtggcggttataacaaagccgaaggcagatactctaccatcggtggtggcagtaacaacgaagccacaaacgagtactctaccatcgttggtggcgatgacaacaaagccacaggcagatactctaccatcggtggtggcgataacaacacagccgaaggcgaatactcaaccgtcgcagggggcaagaataaccaagccacaggtacaggttcatttgccgcaggtgtagagaaccaagccaatgccgaaaacgccgtcgccgtgggtaaaaagaacattatcgaaggtgaaaactcagtagccatcggctctgagaataccgttaaaacagaacacaaaaatgtctttattcttggctctggcacaacaggtgtaacgagtaactcagtgctactgggtaatgagaccgctggcaaacaggcgaccactgttaagaatgccgaagtgggtggtctaagcctaacaggatttgcaggggagtcaaaagctgaaaacggcgtagtttctgtgggtagtgaaggcggtgagcgtcaaatcgttaatgttggtgcaggtcagatcagtgacacctcaacagatgctgttaatggctcacagctacatgctttggccacagttgttgatgacaaccaatatgacattgttaacaaccgagctgacattcttaacaaccaagatgatatcaaagatcttcagaaggaggtgaaaggtcttgataatgaggtgggtgaattaagccgagacattaattcacttcatgatgttactgacaaccaacaagatgacatcaaagagcttaagaggggggtaaaagagcttgataatgaggtgggtgtattaagccgagacattaattcacttcatgatgatgttgctgacaaccaagatgacattgctaaaaacaaagctgacatcaaaggtcttaataaggaggtgaaagagcttgataaggaggtgggtgtattaagccgagacattggttcacttcatgatgatgttgccaccaaccaagctgacattgctaaaaaccaagcggatatcaaaacacttgaaaacaatgtcgaagaagaattattaaatctaagcggtcgcctgcttgatcagaaagcggatattgataataacatcaacaatatctatgagctggcacaacagcaagatcagcatagctctgatatcaaaacacttaaaaacaatgtcgaagaaggtttattggatctaagcggtcgcctcattgatcaaaaagcagatattgctaaaaaccaagctgacattgctcaaaaccaaacagacatccaagatctggccgcttacaatgagctacaagaccagtatgctcaaaagcaaaccgaagcgattgacgctctaaataaagcaagctctgagaatacacaaaacattgctaaaaaccaagcggatattgctaataacatcaacaatatctatgagctggcacaacagcaagatcagcatagctctgatatcaaaaccttggcaaaagtaagtgctgccaatactgatcgtattgctaaaaacaaagctgaagctgatgcaagttttgaaacgctcaccaaaaatcaaaatactttgattgagcaaggtgaagcattggttgagcaaaataaagccatcaatcaagagcttgaagggtttgcggctcatgcagatattcaagataagcaaattttacaaaaccaagctgatatcactaccaataagaccgctattgaacaaaatatcaatagaactgttgccaatgggtttgagattgagaaaaataaagctggtattgctaccaataagcaagagcttattcttcaaaatgatcgattaaatcgaattaatgagacaaataatcgtcaggatcagaagattgatcaattaggttatgcactaaaagagcagggtcagcattttaataatcgtattagtgctgttgagcgtcaaacagctggaggtattgcaaatgctatcgcaattgcaactttaccatcgcccagtagagcaggtgagcatcatgtcttatttggttcaggttatcacaatggtcaagctgcggtatcattgggcgcggctgggttaagtgatacaggaaaatcaacttataagattggtctaagctggtcagatgcaggtggattatctggtggtgttggtggcagttaccgctggaaataa

SEQ ID NO: 263 - protein, 576 aa, Moraxella catarrhalis
MKTMKLLPLKIAVTSAMIVGLGATSTVNAQVVEQFFPNIFFNENHDELDDAYHNMILGDTAIVSNSQDNSTQLKFYSNDEDSVPDSLLFSKLLHEQQLNGFKAGDTIIPLDKDGKPVYTKDTRTKDGKVETVYSVTTKIATQDDVEQSAYSRGIQGDIDDLYDINREVNEYLKATHDYNERQTEAIDALNKASSANTDRIDTAEERIDKNEYDIKALESNVEEGLLELSGHLIDQKADLTKDIKALESNVEEGLLELSGHLIDQKADLTKDIKALESNVEEGLLDLSGRLLDQKADIAKNQADIAQNQTDIQDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 264 - DNA, 1731 bp, Moraxella catarrhalis
  source 1..1731: mol_type = other DNA
atgaaaaccatgaaacttctccccctaaaaatcgctgtaaccagtgccatgattgttggcttgggtgcgacatctactgtgaatgcacaagtagtggaacagttttttccgaatatcttttttaatgaaaaccatgatgaattagatgatgcataccataatatgatcttaggggatactgcgattgtatctaattcacaagataatagtactcaattgaaattttattctaatgatgaagattcagttcctgacagcctactctttagtaaactacttcatgagcagcaacttaatggttttaaagcaggtgacacaatcattcctttggataaggatggcaaacctgtttatacaaaggacacgagaacaaaggatggtaaagtagaaacagtttattcggtcaccaccaaaatcgctacccaagatgatgttgaacaaagtgcatattcacgaggcattcaaggtgatatcgatgatctgtatgacattaaccgtgaagtcaatgaatacttaaaagcaacacatgattataatgaaagacaaactgaagcaattgacgctctaaacaaagcaagctctgcgaatactgatcgtattgatactgctgaagagcgtatcgataaaaacgaatatgacattaaagcacttgaaagcaatgtcgaagaaggtttgttggagctaagcggtcacctcattgatcaaaaagcagatcttacaaaagacatcaaagcacttgaaagcaatgtcgaagaaggtttgttggagctaagcggtcacctcattgatcaaaaagcagatcttacaaaagacatcaaagcacttgaaagcaatgtcgaagaaggtttgttggatctaagcggtcgtctgcttgatcaaaaagcagatatcgctaaaaaccaagctgacattgctcaaaaccaaacagacatccaagatctagccgcttacaacgagctacaagatgcctatgccaaacagcaaaccgaagcgattgacgctctaaacaaagcaagctctgagaatacacaaaacattgctaaaaaccaagcggatattgctaataacatcaacaatatctatgagctggcacaacagcaagatcagcatagctctgatatcaaaaccttggcaaaagcaagtgctgccaatactgatcgtattgctaaaaacaaagccgatgctgatgcaagttttgaaacgctcaccaaaaatcaaaatactttgattgaaaaagataaagagcatgacaaattaattactgcaaacaaaactgcgattgatgccaataaagcatctgcggataccaagtttgcagcgacagcagacgccattaccaaaaatggaaatgctatcactaaaaacgcaaaatctatcactgatttgggtactaaagtggatggttttgacggtcgtgtaactgcattagacaccaaagtcaatgccttagacaccaaagtcaatgcctttgatggtcgtatcacagctttagacagtaaagttgaaaacggtatggctgcccaagctgccctaagtggtctattccagccttatagcgttggtaagtttaatgcgaccgctgcacttggtggctatggctcaaaatctgcggttgctatcggtgctggctatcgtgtgaatccaaatctggcgtttaaagctggtgcggcgattaataccagtggtaataaaaaaggctcttataacatcggtgtgaattacgagttttaa

(19) United States
(12) Patent Application Publication
(10) Pub. No.: US 2025/0302937 A1
(43) Pub. Date: Oct. 2, 2025

(54) IMMUNOGENIC COMPOSITIONS AND METHODS FOR REDUCING TRANSMISSION OF PATHOGENS

(71) Applicant: St. Jude Children's Research Hospital, Memphis, TN (US)

(72) Inventors: Jason W. Rosch, Memphis, TN (US); Amy R. Iverson, Memphis, TN (US); Haley Echlin, Memphis, TN (US)

(73) Assignee: St. Jude Children's Research Hospital, Memphis, TN (US)

(21) Appl. No.: 18/854,820

(22) PCT Filed: Apr. 7, 2023
(86) PCT No.: PCT/IB2023/053589

§ 371 Date: Oct. 7, 2024

Related U.S. Application Data
(60) Provisional application No. 63/329,083, filed on Apr. 8, 2022.

(51) Int. Cl. A61K 39/102; A61K 39/00; A61K 39/09; A61K 39/104; A61P 31/04; C07K 14/21; C07K 14/285; C12N 15/74

(52) U.S. Cl. CPC A61K 39/102; A61K 39/092; A61K 39/1045; A61P 31/04; C07K 14/212; C07K 14/285; C12N 15/746; A61K 2039/522; A61K 2039/70

(57) ABSTRACT

Compositions and methods are provided for reducing the transmission of at least one pathogen including S. pneumoniae through administration to subjects of immunogenic compositions comprising a recombinant, live attenuated S. pneumoniae expressing on its cell surface at least one heterologous immunogenic protein, or an immunogenic fragment or variant thereof. The immunogenic compositions also serve to reduce the incidence rate of at least one disease, such as acute otitis media, caused by at least one pathogen. Examples of heterologous immunogenic proteins, or immunogenic fragments or variants thereof, include H. influenzae protein D and M. catarrhalis UspA polypeptide. The expressed heterologous immunogenic protein, or an immunogenic fragment or variant thereof, is modified with a surface anchor moiety for anchoring at the cell surface of S. pneumoniae.

### CROSS-REFERENCE TO RELATED APPLICATION

This application claims priority to U.S. Provisional Patent Application No. 63/329,083, filed Apr. 8, 2022, which is fully incorporated by reference herein.

### REFERENCE TO A SEQUENCE LISTING SUBMITTED ELECTRONICALLY AS AN XML FILE

The instant application contains a Sequence Listing which has been submitted in XML format via EFS-Web and is hereby incorporated by reference in its entirety. Said XML copy, created on Apr. 7, 2023, is named S88435_1290WO_0086.3_Sequence Listing.xml, and is 332 KB in size.

### FIELD OF THE INVENTION

The invention relates to the field of immunology and bacteriology. In particular, the invention relates to immunogenic compositions for reducing the transmission of bacterial pathogens including Streptococcus pneumoniae. The methods and compositions allow a platform for antigen presentation and can be used to treat diseases associated with bacterial pathogen infection.

### BACKGROUND OF THE INVENTION

Streptococcus pneumoniae (S. pneumoniae) is a member of the human nasal microbiome, especially of children (van den Bergh (2012) PLoS One. 7(10), e47711). S. pneumoniae resides as part of the commensal microbiota in the upper respiratory tract, specifically the nasopharynx, without causing symptoms. Critical to the success of S. pneumoniae is the capacity of the organism to initially colonize the human nasopharynx and subsequently transmit and colonize a new host. In subjects whose immune systems are not able to clear colonization of S. pneumoniae from the upper respiratory tract (such as young children, the elderly, and the immunocompromised), S. pneumoniae can migrate into sterile tissues and organs of the lower respiratory tract and cause pneumococcal diseases including acute otitis media (middle ear infection), sinusitis, pneumonia, bacteremia, and meningitis. Acute otitis media (AOM) is prevalent in young children and it is estimated that at least 70% of children in early childhood experience at least one episode of AOM (Heikkinen and Chonmaitree Clin Microbiol Rev 2003, 16, 230-241; Grijalva et al. Pediatrics 2006, 118, 865-873). Pneumococcal pneumonia is the main type of pneumonia globally and is the leading cause of death in children under the age of five (Brooks and Mias Front. Immunol. 2018, 9, 1366).

However, other bacteria in addition to S. pneumoniae may play a role in infectious diseases. For example, Haemophilus influenzae (H. influenzae) is another commensal bacteria of the human nasopharynx and are detected together with S. pneumoniae in infected tissue. Additionally, Moraxella catarrhalis is a gram-negative diplococcus that causes ear and upper and lower respiratory infections.

Separate vaccines for S. pneumoniae and H. influenzae have been developed. For example, development of the pneumococcal conjugate vaccine (PCV) has greatly reduced the burden of invasive disease by S. pneumoniae. PCV contains purified capsular polysaccharide of 7 or 13 strains (serotypes) of S. pneumoniae conjugated to a carrier protein for greater vaccine efficacy. A vaccine against the encapsulated type b of H. influenzae has been developed to reduce invasive diseases such as meningitis and bacteremia. However, given that there are over 90 serotypes of S. pneumoniae and a multitude of nontypeable strains of H. influenzae, not all serotypes or strains of these bacteria are covered by these separate vaccines. Moreover, a single vaccine that can address multiple pathogens would be desirable for cost and logistical considerations. Thus, there is still a need in the art for immunogenic compositions to address diseases caused by multiple pathogens.

### SUMMARY OF THE INVENTION

Compositions and methods are provided for reducing the incidence rate of at least one disease caused by at least one pathogen and/or reducing transmission of at least one pathogen. This is achieved by administration to subjects of an immunogenic composition including a recombinant, live attenuated S. pneumoniae. In embodiments, the recombinant, live attenuated S. pneumoniae includes a deletion or disruption of a native ftsY gene in the genome of the S. pneumoniae to attenuate virulence. The at least one pathogen includes S. pneumoniae, H. influenzae, and/or M. catarrhalis.

The recombinant, live attenuated S. pneumoniae expresses at least one heterologous immunogenic protein, or an immunogenic fragment or variant thereof, on its surface. The heterologous immunogenic protein, or an immunogenic fragment or variant thereof, is directed and anchored to the surface of the recombinant S. pneumoniae by a surface anchor moiety fused to the heterologous protein. The attachment of the surface anchored heterologous immunogenic protein, or an immunogenic fragment or variant thereof, to the recombinant S. pneumoniae cell surface, may be covalent or non-covalent. In embodiments, the surface anchor moiety includes a lipoprotein anchor for covalent attachment of the heterologous immunogenic protein, or an immunogenic fragment or variant thereof, to the plasma membrane of the recombinant S. pneumoniae. In embodiments, the surface anchor moiety includes a sortase signal for covalent attachment of the heterologous immunogenic protein, or an immunogenic fragment or variant thereof, to peptidoglycan in the cell wall of the recombinant S. pneumoniae through recognition and cleavage of the sortase signal by an endogenous sortase enzyme. In embodiments, the surface anchor moiety includes a choline binding domain (CBD) for non-covalent attachment of the heterologous immunogenic protein, or an immunogenic fragment or variant thereof, to the cell wall of the recombinant S. pneumoniae by binding of the CBD to choline in the cell wall. In embodiments, the at least one heterologous immunogenic protein, or an immunogenic fragment or variant thereof, is from S. pneumoniae. In embodiments, the at least one heterologous immunogenic protein, or an immunogenic fragment or variant thereof, is from H. influenzae. In embodiments, the at least one heterologous immunogenic protein, or an immunogenic fragment or variant thereof, is from M. catarrhalis. In embodiments, the at least one heterologous immunogenic protein, or an immunogenic fragment or variant thereof, includes H. influenzae protein D. In embodiments, the at least one heterologous immunogenic protein, or an immunogenic fragment or variant thereof, includes M. catarrhalis UspA polypeptide. In embodiments, a disease against which compositions of the present disclosure are used include acute otitis media, pneumonia, sinusitis, bacteremia, septicemia, and meningitis.

Genetic constructs to express a heterologous immunogenic protein, or an immunogenic fragment or variant thereof, on the cell surface of a recombinant, live attenuated S. pneumoniae and methods of producing such recombinant, live attenuated S. pneumoniae are also provided.

### BRIEF DESCRIPTION OF THE FIGURES

FIGS. 1A-1G show PCR amplification steps to generate CEP-P3ΩProteinD-CBD, CEP-P3ΩProteinD-sort, and CEP-P3ΩProteinD-lipo. (FIG. 1A) PCR1 and PCR2; (FIG. 1B) PCR3; (FIG. 1C) PCR4; (FIG. 1D) PCR5; (FIG. 1E) PCR6; (FIG. 1F) PCR7; (FIG. 1G) PCR8/9/10. CEP-P3ΩProteinD-CBD, CEP-P3ΩProteinD-sort, and CEP-P3ΩProteinD-lipo amplicons shown in FIG. 1G. have nucleotide sequences set forth as SEQ ID NOs: 232, 233, and 234, respectively.

FIG. 2 shows PCR to generate ΔftsYin::PhunSweetErm amplicon from BHN97ΔftsYin::PhunSweetErm genomic DNA.

FIGS. 3A, 3B show BHN97 CEP-P3ΩProteinD-lipoΔftsYin::PhunSweetErm mutation was confirmed using multiple primer pairs. Primers have corresponding sequences in ΔftsYin::PhunSweetErm mutants (FIG. 3A), in BHN97 (FIG. 3B), or in both.

FIGS. 4A, 4B show production of antibody against Protein D. (FIG. 4A) SDS-Gel of purified Protein D expressed in E. coli (expected size is 39 KDa). (FIG. 4B) Western blot analysis of purified Protein D (lane 1) and whole cell lysis of Haemophilus influenzae (lane 2) using primary polyclonal rabbit antibody against Protein D.

FIGS. 5A, 5B show Western Blot analysis of Protein D-expressing S. pneumoniae strains. Whole cell lysates were run on an SDS-PAGE gel and transferred to a nitrocellulose membrane, which was probed with a polyclonal rabbit antibody against Protein D. (FIG. 5A) Lysates included two clones from each method of anchoring Protein D (choline binding domain (CBD), sortase signal, lipoprotein anchor domain), a positive control (Haemophilus influenzae), and a negative control (native BHN97 S. pneumoniae strain). Expected size: Protein D-CBD: 49 kDa; Protein D-sort: 46 kDa; Protein D-lipo: 41 kDa. Lanes: 1, purified protein D; 2, positive H. influenzae control; 3, Protein D-CBD #1-1; 4, Protein D-CBD #1-8; 5, Protein D-sort #1-3; 6, Protein D-sort #1-37; 7, Protein D-lipo #1-8; 8, Protein D-lipo #1-15; 9, negative BHN97 control. (FIG. 5B) Lysates included a positive control (H. influenzae) (lane 1), a negative control (native BHN97 S. pneumoniae strain) (lane 2), the Protein D-lipo expressing strain (lane 3, Protein D-lipo #1-15), and two clones of the live attenuated Protein D expressing strains (lane 4, Protein D-lipo Δftsy #1-15-1; lane 5, Protein D-lipo Δftsy #1-15-2).

FIG. 6 shows fluorescence microscopy images of Protein D-expressing S. pneumoniae strains. Whole cells were fixed in paraformaldehyde and then stained with a polyclonal rabbit antibody against Protein D and an Alexa-488 secondary. Fixed cells included a positive control (H. influenzae), a negative control (native BHN97 S. pneumoniae strain), two clones from each method of anchoring Protein D (CBD domain, sortase signal, lipoprotein anchor domain), and two clones of the live attenuated Protein D-expressing strains (Protein D-lipo Δftsy).

FIGS. 7A-7F show protection against S. pneumoniae BHN97x for mice vaccinated with immunogenic compositions of the disclosure as measured by bacterial colonization in colony forming units/mL in: (FIG. 7A) lung; (FIG. 7B) nasal passage; (FIG. 7C) right ear; (FIG. 7D) left ear; (FIG. 7E) combined ears; and (FIG. 7F) blood. The immunogenic compositions tested were: Prevnar-13, FDA-approved pneumococcal 13-valent conjugate vaccine (Wyeth Pharmaceutical, Inc.); BHN97 ΔftsY phun:sweet (live attenuated S. pneumoniae strain not expressing any surface-anchored heterologous antigen); production stock (live attenuated S. pneumoniae strain not expressing any surface-anchored heterologous antigen produced in large scale by a manufacturer); BHN97 ΔftsY phun:sweet Protein D (BHN97 CEP-P3ΩProteinD-lipo ΔftsYin::PhunSweetErm described in Examples 1-5); BHN97ΔftsY phun:sweet Protein-D-MoraC17 (live attenuated S. pneumoniae strain expressing H. influenzae protein D and M. catarrhalis UspA polypeptide); and BHN97 ΔltyA ΔftsY phun:sweet (live attenuated S. pneumoniae strain with a further deletion of the ltyA gene to disrupt autolysis for potential ease of strain production). The vehicle was phosphate-buffered saline (PBS).

FIGS. 8A-8F show protection against H. influenzae for mice vaccinated with immunogenic compositions of the disclosure as measured by bacterial colonization in colony forming units/mL in: (FIG. 8A) lung; (FIG. 8B) nasal passage; (FIG. 8C) right ear; (FIG. 8D) left ear; (FIG. 8E) combined ears; and (FIG. 8F) blood. The vaccine compositions tested were: protein D (isolated recombinant H. influenzae protein D); BHN97 ΔftsY phun:sweet Protein D (BHN97 CEP-P3ΩProteinD-lipo ΔftsYin::PhunSweetErm described in Examples 1-5); and BHN97ΔftsY phun:sweet Protein-D-MoraC17 (live attenuated S. pneumoniae strain expressing H. influenzae protein D and M. catarrhalis UspA polypeptide). The vehicle was phosphate-buffered saline (PBS).

FIGS. 9A-9E show protection against M. catarrhalis for mice vaccinated with live attenuated S. pneumoniae strain expressing H. influenzae protein D and M. catarrhalis UspA polypeptide (BHN97ΔftsY phun:sweet Protein-D-MoraC17) as measured by bacterial colonization in colony forming units/mL in: (FIG. 9A) lung; (FIG. 9B) nasal passage; (FIG. 9C) right ear; (FIG. 9D) left ear; and (FIG. 9E) combined ears. The vehicle was phosphate-buffered saline (PBS).

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Overview

Infectious disease may be caused by more than one pathogen. For example, patients afflicted by pneumococcal diseases have infected tissues that contain biofilms of both S. pneumoniae and H. influenzae. S. pneumoniae, H. influenzae, and Moraxella catarrhalis can reside in the nasopharynx of subjects asymptomatically. However, in subjects whose immune systems are reduced in their ability to control pathogens, such as young children, the elderly, or immunocompromised individuals, the pathogens can migrate to sterile tissues and organs and cause disease. For example, migration of S. Pneumoniae and H. influenzae: to the bronchi can cause bronchitis; to the lungs can cause pneumonia; to the blood can cause bacteremia or septicemia; to the sinuses can cause sinusitis; to the middle ear can cause acute otitis media; and to the blood-brain barrier can cause meningitis.

Separate vaccines have been developed for S. pneumoniae and H. influenzae. A vaccine containing polysaccharides from 23 serotypes of S. pneumoniae has been effective at reducing bacteremia and pneumonia in adults. More immunogenic pneumococcal vaccines were subsequently developed that conjugate polysaccharides of 7 or 13 serotypes to diphtheria toxin. These pneumococcal conjugate vaccines have helped reduce incidence of pneumococcal diseases in children. A vaccine (Hib) developed against H. influenzae encapsulated serotype b has been successful in decreasing the incidence rate of invasive diseases such as bacteremia and meningitis. However, these vaccines do not address non-vaccine serotypes. Moreover, a vaccine against multiple pathogens would allow for fewer vaccinations to protect against those pathogens. Additionally, the rise of antibiotic-resistant bacteria provides more impetus for developing vaccines that are effective against multiple bacteria to avoid or reduce antibiotic use.

Therefore, vaccines that can address more than one pathogen would be useful for cost, logistical, and therapeutic reasons. The present disclosure describes an immunogenic composition useful for reducing the incidence rate of at least one disease caused by at least one pathogen including Streptococcus pneumoniae (S. pneumoniae) and/or reducing transmission of at least one pathogen including S. pneumoniae. The immunogenic composition includes a recombinant, live attenuated S. pneumoniae. The live attenuated S. pneumoniae serves as a vaccine against diseases caused by S. pneumoniae. The live attenuated Streptococcus pneumoniae is additionally genetically engineered to express at least one heterologous immunogenic protein, or an immunogenic fragment or variant thereof, on its surface. This allows presentation of at least one additional immunogen to serve as a vaccine against S. pneumoniae or against S. pneumoniae and at least one other different pathogen.

Moreover, the genetically engineered live attenuated S. pneumoniae can serve as a platform for presentation of any heterologous immunogenic protein, or an immunogenic fragment or variant thereof to induce an immune response in a subject. The induction of the immune response can prevent or reduce onset, duration, severity, or a combination thereof, of symptoms of a disease, such as an infection or an invasive disease, when a composition comprising the genetically engineered S. pneumoniae expressing a heterologous immunogenic protein, or an immunogenic fragment or variant thereof, on its cell surface is administered to a subject in need thereof.

Methods are also provided for reducing the incidence rate of at least one disease caused by at least one pathogen including Streptococcus pneumoniae (S. pneumoniae) and/or reducing transmission of at least one pathogen including S. pneumoniae. In some embodiments, the reduction in the incidence rate is greater using an immunogenic composition comprising a recombinant, live attenuated S. pneumoniae modified to express at least one heterologous immunogenic protein, or an immunogenic fragment or variant thereof, on its surface, as compared to using an immunogenic composition comprising a recombinant, live attenuated S. pneumoniae that has not been modified to express at least one heterologous immunogenic protein, or an immunogenic fragment or variant thereof.

Genetic constructs to express a heterologous immunogenic protein, or an immunogenic fragment or variant thereof on the cell surface of a recombinant, live attenuated S. pneumoniae and methods of producing such recombinant, live attenuated S. pneumoniae are also provided.

### II. Definitions

By “variant” is intended substantially similar sequences. Thus, immunogenic variants of the disclosure include sequences that are functionally equivalent to the protein sequence of interest and retain immunogenic activity. Generally, amino acid sequence variants of the invention will have at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to a respective amino acid sequence. Methods of determining sequence identity are also discussed elsewhere herein.

With respect to the amino acid sequences for the various full length polypeptides, variants include those polypeptides that are derived from the native polypeptides by deletion (so-called truncation) or addition of one or more amino acids to the N-terminal and/or C-terminal end of the native polypeptide; deletion or addition of one or more amino acids at one or more sites in the native polypeptide; or substitution of one or more amino acids at one or more sites in the native polypeptide. Such variants may result from, for example, genetic polymorphism or from human manipulation. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488-492; Kunkel et al. (1987) Methods Enzymol. 154:367-382; Sambrook et al. (1989) Molecular Cloning: A Laboratory Manual (2d ed., Cold Spring Harbor Laboratory Press, Plainview, N.Y.); U.S. Pat. No. 4,873,192; and the references cited therein; herein incorporated by reference. Guidance as to appropriate amino acid substitutions that may not affect biological activity of the various proteins may be found in the model of Dayhoff et al. (1978) Atlas of Polypeptide Sequence and Structure (Natl. Biomed. Res. Found., Washington, D.C.), herein incorporated by reference. Conservative substitutions, such as exchanging one amino acid with another having similar properties, may be preferred. The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays.

By “sequence identity” is intended the same nucleotides or amino acid residues are found within the variant sequence and a reference sequence when a specified, contiguous segment of the nucleotide sequence or amino acid sequence of the variant is aligned and compared to the nucleotide sequence or amino acid sequence of the reference sequence. Methods for sequence alignment and for determining identity between sequences are well known in the art. With respect to optimal alignment of two nucleotide sequences, the contiguous segment of the variant nucleotide sequence may have additional nucleotides or deleted nucleotides with respect to the reference nucleotide sequence. Likewise, for purposes of optimal alignment of two amino acid sequences, the contiguous segment of the variant amino acid sequence may have additional amino acid residues or deleted amino acid residues with respect to the reference amino acid sequence. The contiguous segment used for comparison to the reference nucleotide sequence or reference amino acid sequence will comprise at least 20 contiguous nucleotides, or amino acid residues, and may be 30, 40, 50, 100, or more nucleotides or amino acid residues. Corrections for increased sequence identity associated with inclusion of gaps in the variant's nucleotide sequence or amino acid sequence can be made by assigning gap penalties. Methods of sequence alignment are well known in the art.

The determination of percent identity between two sequences can be accomplished using a mathematical algorithm. For example, percent identity of an amino acid sequence can be determined using the Smith-Waterman homology search algorithm using an affine 6 gap search with a gap open penalty of 12 and a gap extension penalty of 2, BLOSUM matrix 62. Alternatively, percent identity of a nucleotide sequence is determined using the Smith-Waterman homology search algorithm using a gap open penalty of 25 and a gap extension penalty of 5. Such a determination of sequence identity can be performed using, for example, the DeCypher Hardware Accelerator from TimeLogic Version G. The Smith-Waterman homology search algorithm is taught in Smith and Waterman (1981) Adv. Appl. Math 2:482-489, herein incorporated by reference. Alternatively, the alignment program GCG Gap (Wisconsin Genetic Computing Group, Suite Version 10.1) using the default parameters may be used. The GCG Gap program applies the Needleman and Wunch algorithm and for the alignment of nucleotide sequences with an open gap penalty of 3 and an extend gap penalty of 1 may be used. Another preferred, non-limiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 2/5:403. BLAST nucleotide searches can be performed with the NBLAST program, score=100, wordlength 12, to obtain nucleotide sequences having sufficient sequence identity. BLAST protein searches can be performed with the XBLAST program, score=50, wordlength=3, to obtain amino acid sequences having sufficient sequence identity. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. See www.ncbi.nlm.nih.gov. Another non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

The article “a” and “an” are used herein to refer to one or more than one (i.e., to at least one) of the grammatical object of the article. By way of example, “a polypeptide” means one or more polypeptides.

Unless specifically indicated otherwise, the word “or” is used in the inclusive sense of “and/or” and not the exclusive sense of “either/or.”

The term “about” or “approximately” usually means within 5%, or more preferably within 1%, of a given value or range.

The terms “comprises”, “comprising”, “includes”, “including”, “having” and their conjugates mean “including but not limited to”.

Various embodiments of this disclosure may be presented in a range format. It should be noted that whenever a value or range of values of a parameter are recited, it is intended that values and ranges intermediate to the recited values are also part of this disclosure. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1-10 should be considered to have specifically disclosed subranges such as from 1 to 3, from 1 to 4, from 1 to 5, from 1 to 6, from 1 to 7, from 1 to 8, from 1 to 9, from 2 to 4, from 2 to 6, from 2 to 8, from 2 to 10, from 3 to 6, etc., as well as individual numbers within that range, for example, 1, 2, 3, 4, 5, 6, 7, 8, 9 and 10. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. The recitation of a numerical range for a variable is intended to convey that the present disclosure may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≥0 and ≤2 if the variable is inherently continuous.

All publications and patent applications mentioned in the specification are indicative of the level of those skilled in the art to which this disclosure pertains. All publications and patent applications are herein incorporated by reference to the same extent as if each individual publication or patent application was specifically and individually indicated to be incorporated by reference.

### Iii. Live, Attenuated S. pneumoniae

The present disclosure includes a live, attenuated S. pneumoniae bacteria that is genetically modified to express on its surface at least one heterologous protein or an immunogenic fragment or variant thereof. The term “attenuated” as used herein describes a cell, culture, or strain of S. pneumoniae exhibiting a detectable reduction in infectivity or virulence in vivo as compared to that of the parent strain of S. pneumoniae from which the attenuated cell, culture, or strain is derived. Reduction in virulence encompasses any detectable decrease in any attribute of virulence, including infectivity in vivo, amount and/or duration of colonization of the nasopharynx, or any decrease in the severity or rate of progression of any clinical symptom or condition associated with infection (e.g., symptoms associated with acute otitis media, lung inflammation, sinus inflammation). The present disclosure further encompasses preparation and use in an immunogenic composition of cells of a strain of S. pneumoniae derived from a pathogenic parent strain of S. pneumoniae and which exhibit attenuated pathogenicity compared to cells of the parent strain and which are capable of triggering an immune response that protects a subject against infection or invasive disease. In embodiments, an attenuated S. pneumoniae of the disclosure colonizes a host but does not cause disease. In embodiments, an attenuated S. pneumoniae of the disclosure maintains a full set of virulence determinants but is compromised in its adaptation to the host environment. In embodiments, an attenuated S. pneumoniae of the disclosure maintains expression of antigenic virulence proteins pneumolysin, CbpA, and/or PspA.

In embodiments, live attenuated microorganisms are highly effective vaccines; immune responses elicited by such vaccines are often of greater magnitude and of longer duration than those produced by non-replicating immunogens. One explanation for this may be that live attenuated strains establish limited infections in the host and mimic the early stages of natural infection. In addition, unlike killed preparations, live vaccines are often more potent in inducing mucosal immune responses and cell-mediated responses, which may be connected with their ability to replicate in epithelial cells and antigen-presenting cells, such as macrophages.

In embodiments, live, attenuated S. pneumoniae bacteria is achieved by deleting or disrupting the ftsY gene in S. pneumoniae bacteria. The ftsY gene encodes a component of the signal recognition particle (SRP) pathway that is responsible for delivering membrane and secretory proteins to the proper cellular destination. In Streptococci, deletion of the ftsY gene is tolerated. A live, attenuated ΔftsY S. pneumoniae strain has been described that protects against otitis media, sinusitis, pneumonia, and invasive pneumococcal disease in mice (Rosch et al. EMBO Molecular Medicine 2014, 6(1), 141-154; U.S. Pat. No. 9,265,819). Deletion or disruption of the ftsY gene can be achieved through homologous recombination as described herein. In embodiments, all or a portion of the ftsY gene is replaced by an expression cassette including selectable and/or counter-selectable markers for selection of S. pneumoniae that have integrated the marker expression cassette to disrupt or delete the ftsY gene. In embodiments, the expression cassette includes an antibiotic resistance marker, such as an erythromycin resistance marker.

### Iv. Genetic Modification of S. pneumoniae

The present disclosure provides live S. pneumoniae genetically engineered to be attenuated in virulence and to express at least one heterologous immunogenic protein, or an immunogenic fragment or variant thereof. In embodiments, the heterologous immunogenic protein, or an immunogenic fragment or variant thereof, is from H. influenzae. In embodiments, the heterologous immunogenic protein, or an immunogenic fragment or variant thereof includes protein D. In embodiments, the heterologous immunogenic protein, or an immunogenic fragment or variant thereof includes a UspA polypeptide.

As used herein, the term “genetically modified” or “genetically engineered” refers to the addition of extra genetic material in the form of DNA or RNA into the total genetic material in a cell or modification of the genome of a cell. In embodiments, the extra genetic material remains separate from the genome of the cell (e.g., the extra genetic material resides on a plasmid or vector that exists in the cell as an entity separate from the cell's genome). In embodiments, the genetic modification results in the genome containing insertions, deletions, mutations, and/or rearrangements of the genomic DNA after introduction of extra genetic material as compared to a cell that is not genetically modified. For clarity the term “genetically modified” or “genetically engineered” also includes the removal of DNA from a genome without the insertion of extra genetic material. The term “genetically modified” or “genetically engineered” includes artificial manipulation of a cell to alter the genotype of that cell to modulate physiology or function of that cell, such as expressing a heterologous gene product, deleting endogenous genes, and/or altering regulation or expression of endogenous genes. The extra genetic material can be derived from the same organism as the genome it is inserted into or it can be derived from a different genome or be synthetic. The terms “genetically modified S. pneumoniae”, “genetically engineered S. pneumoniae”, “modified S. pneumoniae”, and “engineered S. pneumoniae” are used interchangeably. The term “genetically modified” or “genetically engineered” also refers to multiple genetic modifications, e.g. 2, 3, 4, 5, 6, 7, 8, 9, 10 or more genetic modifications, for example, a S. pneumoniae bacterium which has a heterologous gene introduced for expression of a H. influenzae protein D modified with a surface anchor moiety, and another heterologous gene introduced for expression of another H. influenzae protein. The genetically modified cell can be referred to as a “recombinant” cell.

The term “bacterium” or “bacteria” refers to a single-cell microorganism or a class of single-cell microorganisms found almost everywhere on Earth and inside and outside of a body. “Bacterium” and “bacteria” are used interchangeably herein. In embodiments, bacteria include four common forms: Coccus form, which are spherical bacteria (e.g., Streptococcus pneumoniae); Bacillus form, which are rod-shaped bacteria (e.g., Lactobacillus acidophilus); Spirilla form, which are spiral-shaped bacteria (e.g., Spirillum volutans); and Vibrio form, which are comma-shaped bacteria (e.g., Vibrio cholerae). In embodiments, a bacterium can be beneficial or pathogenic to a host organism that the bacterium colonizes. In embodiments, genetically modified S. pneumoniae described herein are pathogenic to a host organism. In embodiments, a bacteria or bacterium can refer to a single bacterial cell (e.g., a S. pneumoniae bacteria includes a single S. pneumoniae cell). In embodiments, bacteria can refer to a population of bacterial cells. In embodiments, bacteria can refer to bacteria of a taxa, a class, a genus, a species, etc. (e.g. Streptococcus bacteria includes a microorganism belonging to the Streptococcus genus). “Pneumococcal” or “pneumococcus” refers to a gram-positive bacterium in the family Streptococcaceae surrounded by a polysaccharide capsule external to its cell wall. In embodiments, pneumococcus or pneumococcal bacteria cause disease including acute otitis media, sinusitis, pneumonia, bacteremia, septicemia, and meningitis.

The term “heterologous” refers to a molecule (e.g., nucleic acid, gene, RNA, protein) that originates outside bacteria and is introduced into bacteria by genetic engineering. In embodiments, a heterologous molecule can include sequences that are not native to bacteria to which the heterologous molecule is introduced. For example, genetically modified S. pneumoniae bacteria of the disclosure include a gene encoding H. influenzae protein D. In embodiments, a heterologous molecule can include sequences that are native to bacteria to which the heterologous molecule is introduced, but the heterologous molecule is synthesized outside the bacteria and introduced into the bacteria or the molecule is synthesized from a gene that is not present in the native location. For example, genetically modified S. pneumoniae bacteria of the disclosure can include extra copies of genes native to S. pneumoniae.

The term “endogenous” refers to a molecule (e.g., nucleic acid, gene, RNA, protein) that is naturally occurring or naturally produced in a given bacterium. For example, genes or proteins found naturally in bacteria are genes or proteins that are endogenous to the bacteria. The term “native” can be used interchangeably with “endogenous”. In embodiments, the term “endogenous” can refer to a wild-type version of a molecule in a given bacterium.

The term “gene” refers to a nucleic acid sequence (used interchangeably with polynucleotide or nucleotide sequence) that encodes, e.g., a protein associated with glycerophosphodiesterase activity (protein D) as described herein. This definition includes various sequence polymorphisms, mutations, and/or sequence variants wherein such alterations do not substantially affect the function of the encoded protein. The nucleic acid sequences can include both the full-length nucleic acid sequences as well as non-full-length sequences derived from a full-length protein coding sequence. The sequences can also include degenerate codons of the native sequence or sequences that can be introduced to provide codon preference in specific bacteria. In embodiments, the term “gene” can include not only coding sequences but also regulatory regions such as promoters, enhancers, 5′ UTR, 3′UTR, termination regions, and non-coding regions. Gene sequences encoding a molecule can be DNA or RNA that directs the expression of the molecule. These nucleic acid sequences can be a DNA strand sequence that is transcribed into RNA or an RNA sequence that is translated into protein. An essential gene is an endogenous (e.g., endogenous to a bacterium) or heterologous gene (e.g., a selectable marker or gene of interest) that produces a polypeptide (e.g., an essential protein) that is necessary for the growth and/or viability of a bacterium.

“Encoding” refers to the property of specific sequences of nucleotides in a gene, such as a complementary DNA (cDNA), or a messenger RNA (mRNA), to serve as templates for synthesis of other macromolecules such as a defined sequence of amino acids or a functional polynucleotide (e.g., siRNA). In embodiments, a gene encodes or codes for a protein if the gene is transcribed into mRNA and translation of the mRNA produces the protein in a cell or other biological system. A “gene sequence encoding a protein” includes all nucleotide sequences that are degenerate versions of each other and that code for the same amino acid sequence or amino acid sequences of substantially similar form and function.

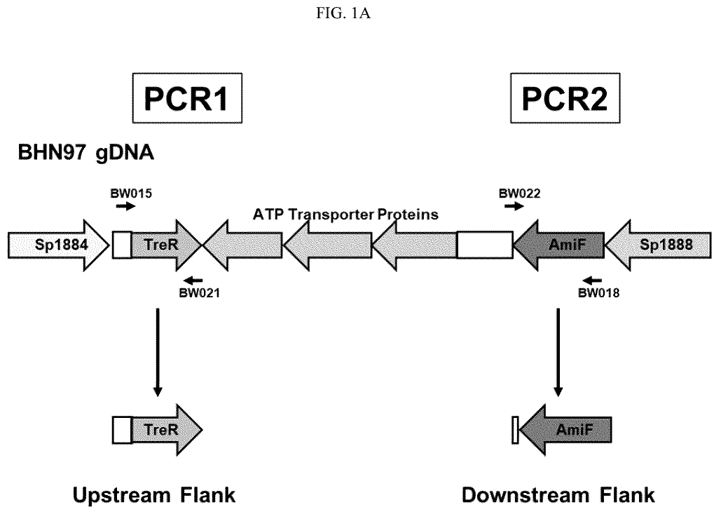

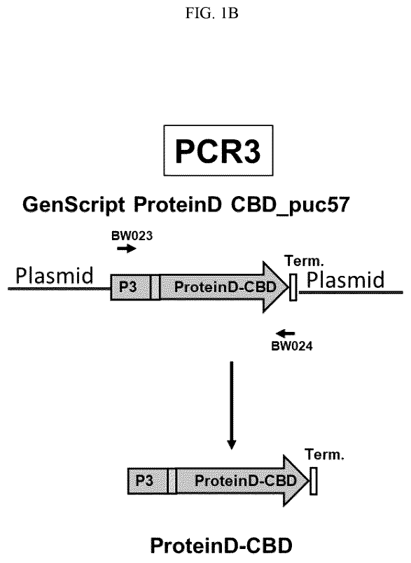

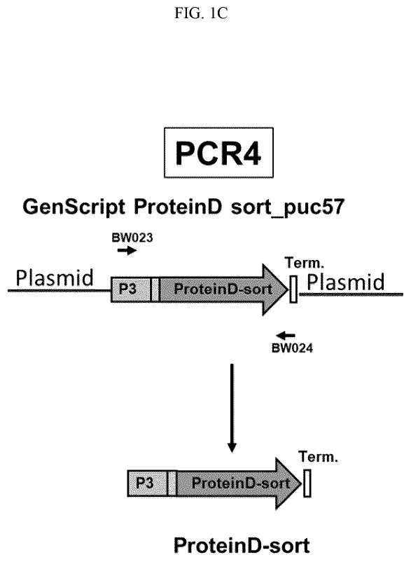

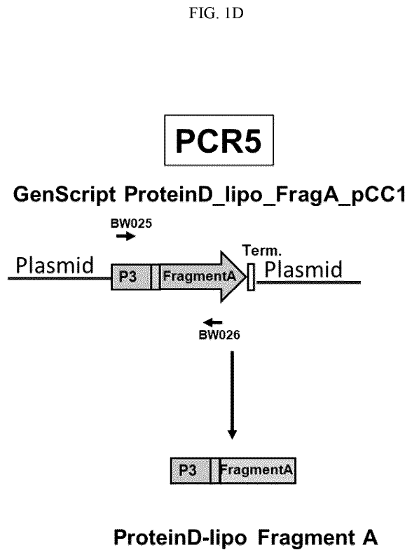

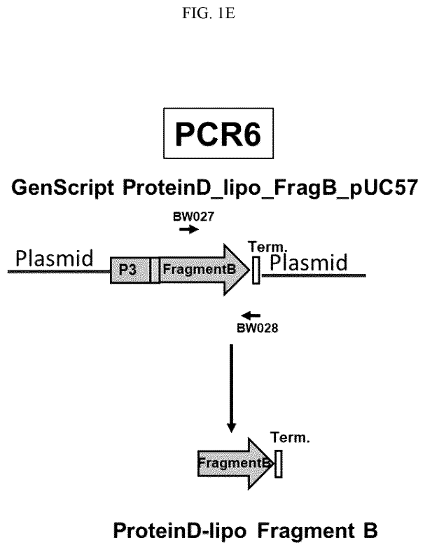

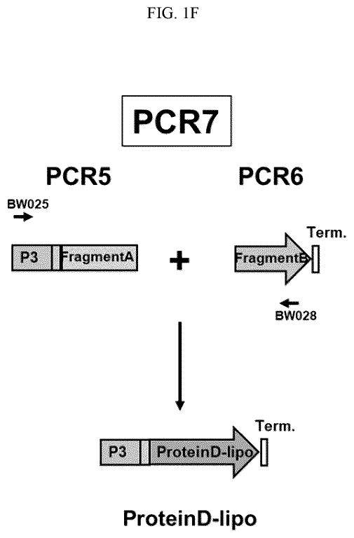

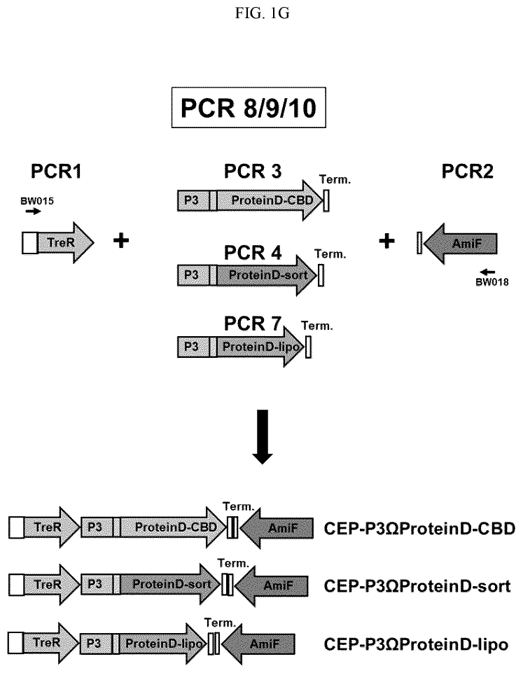

In embodiments, deleting an endogenous gene or locus and replacing it with another gene or a genetic construct in a bacterium can occur by homologous recombination. Homologous recombination includes introducing a genetic construct into a bacterium, where the genetic construct includes homology arms having homology to target sequences of the endogenous gene or locus to be deleted. In embodiments, the genetic construct includes a non-homologous polynucleotide flanked by two polynucleotide regions of homology (i.e., the upstream (5′) and downstream (3′) homology arms), such that homologous recombination between target sequences of the endogenous gene or locus to be deleted and the two flanking homology arms results in insertion of the non-homologous polynucleotide at the target region (see, e.g., FIGS. 1A, 1G).

In embodiments, the target sequence homologous to the upstream homology arm includes sequence 5′ of the coding sequence and/or coding sequence of the endogenous gene or locus to be deleted. In embodiments, the target sequence homologous to the downstream homology arm includes sequence 3′ of the coding sequence and/or coding sequence of the endogenous gene to be deleted or includes sequence 3′ of the locus to be deleted. One of skill in the art will recognize that the upstream and downstream homology arms can have homology to target sequences such that less than the full-length coding sequence of a gene is deleted, a combination of a portion of the full-length coding sequence and sequences upstream (5′) and/or downstream (3′) of the coding sequence is deleted, a combination of the full-length coding sequence and sequences upstream (5′) and/or downstream (3′) of the coding sequence is deleted, or any other variation on deletion of a gene or locus. In embodiments, deletion of a locus by homologous recombination leads to reduction or elimination of expression of one or more genes in the locus. A locus refers to a specific, fixed physical location of a gene or other nucleic acid sequence (e.g., genetic marker) on a chromosome. In embodiments, a locus includes one or more genes.

In embodiments, a homology arm includes sequence having at least 50% sequence identity to a target sequence with which homologous recombination is desired. In embodiments, a homology arm includes sequence having at least 60%, at least 70%, at least 80%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to a target sequence. In embodiments, each homology arm can include 100 to 1000 nucleotides (nt), 200 to 800 nt, or 200 to 500 nt. In embodiments, each homology arm can include 100 nt, 150 nt, 200 nt, 250 nt, 300 nt, 350 nt, 400 nt, 450 nt, 500 nt, 750 nt, 1000 nt, 1250 nt, 1500 nt, 2000 nt, 2500 nt, 3000 nt, or more. In embodiments, each homology arm can include 500 nt. In embodiments, the non-homologous polynucleotide flanked by the upstream and downstream homology arms includes a promoter, a gene, a terminator, a selectable marker, a counter-selectable marker, or a combination thereof. In embodiments, disruption of an endogenous gene or locus in a bacterium by homologous recombination includes deletion of the endogenous gene without any heterologous sequences inserted at the target sequences. In embodiments, disruption of an endogenous gene in a bacterium by homologous recombination includes deletion of the endogenous gene or locus and concurrent insertion of heterologous sequences, such as heterologous expression cassettes including selectable or counter-selectable markers, at the target sequences. In embodiments, disruption of an endogenous gene or locus in a bacterium by homologous recombination reduces or eliminates expression of one or more endogenous genes but portions of the one or more endogenous genes remain in the genome while other portions of the one or more endogenous genes are deleted. As an example, a recombinant S. pneumoniae of the disclosure has its native ftsY gene disrupted by integration of a genetic construct containing an erythromycin selectable marker and PheS and SacB counter-selectable markers by homologous recombination. The disrupted ftsY gene and/or its encoded protein has reduced expression or lacks expression.

S. pneumoniae has a transcriptionally silent site within a truncated IS 1167 element downstream of the ami operon in its genome where a genetic construct to express a surface anchored heterologous immunogenic protein, or an immunogenic fragment or variant thereof (i.e. antigen) can be integrated. In some embodiments, the antigen is H. influenzae protein D. In some embodiments, the antigen is M. catarrhalis uspA. In particular embodiments, the transcriptionally silent site is between the native TreR and AmiF genes of S. pneumoniae (see FIGS. 1A-1G). This transcriptionally silent chromosomal location serves as a site for integration of a heterologous genetic construct, a chromosomal expression platform (CEP), for expression of genes (Guiral et al. Microbiology 2006, 152, 343-349; Sorg et al. ACS Synth. Biol. 2014, 4, 228-239). In embodiments, integration of a heterologous genetic construct at the transcriptionally silent site between the native TreR and AmiF genes of S. pneumoniae does not perturb any known cellular function of the S. pneumoniae. In embodiments, a recombinant live attenuated S. pneumoniae of the disclosure has a genetic construct including a nucleotide sequence encoding a fusion protein of protein D and a surface anchor moiety operably linked to a promoter and a terminator integrated between the native TreR and AmiF genes of S. pneumoniae. In embodiments, a recombinant live attenuated S. pneumoniae expressing a heterologous immunogenic protein at its surface has a nucleotide sequence set forth as SEQ ID NO: 232 (CEP-P3ΩProteinD-CBD) in its genome. In embodiments, a recombinant live attenuated S. pneumoniae expressing a heterologous immunogenic protein at its surface has a nucleotide sequence set forth as SEQ ID NO: 233 (CEP-P3ΩProteinD-sort) in its genome. In embodiments, a recombinant live attenuated S. pneumoniae expressing a heterologous immunogenic protein at its surface has a nucleotide sequence set forth as SEQ ID NO: 234 (CEP-P3ΩProteinD-lipo) in its genome.

As used herein, the terms “peptide,” “polypeptide,” or “protein” are used interchangeably herein and are intended to encompass a singular “polypeptide” as well as plural “polypeptides,” and refers to a molecule composed of monomers (amino acids) linearly linked by amide bonds (also known as peptide bonds). The terms “peptide” and “polypeptide” refer to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “peptide” and “polypeptide”. The terms apply to amino acid polymers in which one or more amino acid residues is an artificial chemical analogue of a corresponding naturally occurring amino acid, as well as to naturally occurring amino acid polymers. Non-limiting examples of artificial amino acid residues include norleucine and selenomethionine. An amino acid residue is a molecule having a carboxyl group, an amino group, and a side chain and having the generic formula H₂NCHRCOOH, where R is an organic substituent, forming the side chain. An amino acid residue, whether it is artificial or naturally occurring, is capable of forming a peptide bond with a naturally occurring amino acid residue.

The term “recombinant” refers to a particular DNA or RNA sequence that is the product of various combinations of cloning, restriction, and/or ligation steps resulting in a construct having a structural coding or non-coding sequence distinguishable from homologous sequences found in natural systems. Generally, DNA sequences encoding the structural coding sequence can be assembled from cDNA fragments and short oligonucleotide linkers, or from a series of oligonucleotides, to provide a synthetic gene which is capable of being expressed in a recombinant transcriptional unit. Such sequences can be provided in the form of an open reading frame uninterrupted by internal non-translated sequences, or introns. Genomic DNA including the relevant sequences could also be used. Sequences of non-translated DNA can be present 5′ or 3′ from the open reading frame, where such sequences do not interfere with manipulation or expression of the coding regions. In embodiments, the term “recombinant” polynucleotide or nucleic acid refers to one which is not naturally occurring or is made by the artificial combination of two otherwise separated segments of sequence. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions.

Similarly, a “recombinant polypeptide” refers to a polypeptide or polyprotein which is not naturally occurring or is made by the artificial combination of two otherwise separated segments of amino acid sequences. This artificial combination can be accomplished by standard techniques of recombinant DNA technology, i.e., a recombinant polypeptide can be encoded by a recombinant polynucleotide. Thus, a recombinant polypeptide is an amino acid sequence encoded by all or a portion of a recombinant polynucleotide.

A “recombinant S. pneumoniae” thus refers to S. pneumoniae bacteria that have been subjected to genetic engineering. For example, a recombinant S. pneumoniae expressing a surface anchored H. influenzae protein D of the present disclosure is produced by transforming S. pneumoniae bacteria with an expression cassette including a nucleic acid encoding an H. influenzae protein D modified with a surface anchor moiety. As another example, a recombinant live S. pneumoniae of the present disclosure attenuated in virulence is produced by transforming S. pneumoniae bacteria with a cassette to delete or disrupt the endogenous ftsY gene. In embodiments, the cassette is an expression cassette that includes selectable and/or counter-selectable markers to select for homologous recombination events that replace or disrupt the ftsY gene with the expression cassette. In embodiments, the expression cassette can include erythromycin as a selectable marker, sacB as a counter-selectable marker, and/or pheS as a counter-selectable marker.

A “genetic construct” or “expression construct” includes a recombinant nucleic acid, generally recombinant DNA, which has been generated for the purpose of the expression of a specific nucleotide sequence(s) or is to be used in the construction of other recombinant nucleotide sequences. The term “expression” or “expressing” refers to the transcription and/or translation of a particular nucleic acid sequence driven by a promoter. An expression construct or expression vector can permit transcription of a particular nucleic acid sequence in a cell (e.g., an S. pneumoniae cell). In embodiments, the term genetic construct includes plasmids and vectors. In embodiments, a genetic construct can be circular or linear. Genetic constructs can include, for example, an origin of replication, a multi-cloning site, a promoter, a gene, a selectable marker, a counter-selectable marker, and/or a terminator. In embodiments, a genetic construct includes nucleic acid (e.g., homology arms) to enable deletion of an endogenous gene or locus in a bacterium. In embodiments, a genetic construct includes an expression cassette. In embodiments, an expression cassette of the disclosure includes: (a) a heterologous promoter; and (b) a heterologous gene encoding an antigen modified with a surface anchor moiety. In some embodiments, the antigen is H. influenzae protein D. In some embodiments, the antigen is M. catarrhalis UspA. In embodiments, the genes in an expression cassette are in an operon. An operon refers to a functioning unit of nucleic acid including a cluster of genes that are operably linked to a single promoter and thus are transcribed together. In embodiments, a genetic construct of the disclosure does not include a selectable marker or a counter-selectable marker. In embodiments, a genetic construct of the disclosure does include a selectable marker and/or a counter-selectable marker.

A genetic construct of the disclosure can include a gene encoding a selectable marker and/or counter-selectable marker. In embodiments, cells expressing a selectable marker can grow in the presence of a selective agent or under a selective growth condition. Examples of selectable markers include antibiotic resistance markers (e.g., erythromycin resistance, chloramphenicol resistance, ampicillin resistance, carbenicillin resistance, kanamycin resistance, spectinomycin resistance, streptomycin resistance, tetracycline resistance, bleomycin resistance, and polymyxin B resistance), markers that complement an essential gene (e.g., alanine auxotrophy (alr), diaminopimelic acid auxotrophy (dapD), thymidine auxotrophy (thyA), proline auxotrophy (proBA), glycine auxotrophy (glyA), carbon source auxotrophy (TpiA)), chemical resistance (e.g., tellurite resistance, Fabl for triclosan resistance, bialaphos herbicide resistance, mercury resistance, arsenic resistance), and visual markers (e.g., green fluorescent protein (GFP), luciferase, β-galactosidase (lacZ)). In embodiments, a genetic construct of the disclosure includes an erythromycin resistance erm gene encoding a methylase. In embodiments, cells are confirmed to have a genetic construct by sensitivity to a selection reagent due to expression of a counter-selectable marker. Examples of genes encoding counter-selectable markers include: sacB (gene encoding levansucrase that converts sucrose to levans, which is harmful to bacteria; thus bacteria expressing sacB are sensitive to sucrose); rpsL (strA) (encodes the ribosomal subunit protein (S12) target of streptomycin); tetAR (confers sensitivity to lipophilic compounds such as fusaric and quinalic acids); pheS (encodes the a subunits of Phe-tRNA synthetase, which renders bacteria sensitive to p-chlorophenylalanine, a phenylalanine analog); thyA (encodes thymidilate synthetase, which confers sensitivity to trimethoprim and related compounds); lacY (encodes lactose permease, which renders bacteria sensitive to t-o-nitrophenyl-β-D-galactopyranoside); gata-1 (encodes a zinc finger DNA-binding protein which inhibits the initiation of bacterial replication); and ccdB (encodes a cell-killing protein which is a potent poison of bacterial gyrase).

The term “expression cassette” includes a polynucleotide construct that is generated recombinantly or synthetically and includes regulatory sequences operably linked to a selected polynucleotide to facilitate expression of the selected polynucleotide in bacteria. For example, the regulatory sequences can facilitate transcription of the selected polynucleotide in bacteria, or transcription and translation of the selected polynucleotide in bacteria. In embodiments, the expression cassette includes an operon, a cluster of genes under the control of a common promoter. Therefore, genes within an operon are expressed together. In embodiments, the expression cassette is introduced as part of a genetic construct into S. pneumoniae bacteria. In embodiments, the expression cassette is subsequently integrated into the genome of S. pneumoniae. A heterologous expression cassette can be integrated into the genome of S. pneumoniae by any method known to one of skill in the art, including by homologous recombination. For example, an expression cassette of the disclosure includes a nucleotide sequence encoding a protein D modified with a surface anchor moiety operably linked to a promoter and a terminator.

In embodiments, an expression cassette including a polynucleotide encoding a fusion protein comprising a protein D and a choline binding domain operably linked to a P3 promoter and a terminator has a nucleotide sequence set forth as SEQ ID NO: 229. In embodiments, an expression cassette including a polynucleotide encoding a fusion protein comprising a protein D and a sortase signal operably linked to a P3 promoter and a terminator has a nucleotide sequence set forth as SEQ ID NO: 230. In embodiments, an expression cassette including a polynucleotide encoding a fusion protein comprising a protein D and a lipoprotein anchor operably linked to a P3 promoter and a terminator has a nucleotide sequence set forth as SEQ ID NO: 231.

The term “overexpression” refers to a greater expression level of a gene encoding a given polypeptide in genetically modified bacteria as compared to expression in the corresponding non-genetically modified bacteria at any developmental or temporal stage for the gene. In embodiments, overexpression can occur when the gene is under the control of a strong promoter (e.g., a P3 promoter). Overexpression can also occur under the control of an inducible promoter. In particular embodiments, overexpression can occur in genetically modified bacteria where endogenous expression of a given polypeptide normally occurs, but such normal expression is at a lower level. In embodiments, overexpression of a given polypeptide can also occur in genetically modified bacteria lacking endogenous expression of a given polypeptide. Overexpression thus results in a greater than normal production or “overproduction” of a given polypeptide in genetically modified bacteria. Increased activity of a protein can result from overexpression or the modification of a peptide or a polypeptide such that it causes the peptide or polypeptide to have a higher activity. For example, in the case where a polypeptide is an enzyme, the enzyme can have an increased catalytic turnover rate.

In embodiments, genetically modified bacteria include a gene where expression of the gene is regulated by a promoter and/or regulatory elements. A promoter and/or regulatory elements are often introduced at a suitable location relative to a gene of interest. For example, a promoter (e.g., a constitutive promoter) is often placed 5′ of a transcription start site of a gene of interest. In embodiments, a nucleic acid includes a promoter and/or regulatory elements necessary to drive the expression of a gene (e.g., a heterologous gene or an endogenous gene). A promoter can be an endogenous promoter, a heterologous promoter, or a combination thereof. In embodiments, a promoter includes a constitutive promoter.

In embodiments, a constitutive promoter includes constitutive synthetic promoters (Jensen and Hammer. Biotechnol. Bioeng. 1998, 58, 191-195; Jensen and Hammer. Appl. Environ. Microbiol. 1998, 64, 82-87; Lindholm and Palva. Biotechnol Lett 2009, 32, 131). In embodiments, a constitutive promoter includes a P3 promoter. In embodiments, a constitutive promoter includes a P3 promoter. In embodiments, a P3 promoter of the disclosure includes a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a nucleotide sequence set forth as SEQ ID NO: 207 or 236, wherein the nucleotide sequence retains P3 promoter function; and/or includes a nucleotide sequence set forth as SEQ ID NO: 207 or 236. In embodiments, a nucleotide sequence retains P3 promoter function when a gene operably linked to the nucleotide sequence is constitutively expressed at a comparable level to a P3 promoter having a nucleotide sequence set forth as SEQ ID NO: 207 or 236. In embodiments, a constitutive promoter includes a P2 promoter. In embodiments, a P2 promoter of the disclosure has a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a nucleotide sequence set forth as SEQ ID NO: 208, wherein the nucleotide sequence retains P2 promoter function; and/or has a nucleotide sequence set forth as SEQ ID NO: 208. In embodiments, a nucleotide sequence retains P2 promoter function when a gene operably linked to the nucleotide sequence is constitutively expressed at a comparable level to a P2 promoter having a nucleotide sequence set forth as SEQ ID NO: 208. In embodiments, a constitutive promoter includes a P1 promoter (Sorg et al. ACS Synth. Biol. 2015, 4, 228-239). In embodiments, a P1 promoter of the disclosure has a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a nucleotide sequence set forth as SEQ ID NO: 209, wherein the nucleotide sequence retains P1 promoter function; and/or has a nucleotide sequence set forth as SEQ ID NO: 209. In embodiments, a nucleotide sequence retains P1 promoter function when a gene operably linked to the nucleotide sequence is constitutively expressed at a comparable level to a P1 promoter having a nucleotide sequence set forth as SEQ ID NO: 209. In embodiments, the heterologous constitutive promoter is derived from Streptococcus genus. In embodiments, the heterologous constitutive promoter is derived from Lactococcus genus. In embodiments, the heterologous constitutive promoter is derived from a gram positive bacteria. In embodiments, a promoter included in genetic constructs of the disclosure includes terminator sequences upstream of the promoter to insulate a genetic construct from transcription from genes upstream of the promoter. For example, a P3 promoter of the disclosure including upstream terminator sequences has a nucleotide sequence set forth as SEQ ID NO: 236.

In embodiments, bacteria are genetically engineered to include a gene under the control of an inducible promoter. An inducible promoter is often a nucleic acid sequence that directs the conditional expression of a gene. An inducible promoter can be an endogenous promoter, a heterologous promoter, or a combination thereof. An inducible promoter can include an operon system. In embodiments, an inducible promoter requires the presence of a certain compound, nutrient, amino acid, sugar, peptide, protein or condition (e.g., light, oxygen, heat, cold) to induce gene activity (e.g., transcription). In embodiments, an inducible promoter includes one or more repressor elements. In embodiments, an inducible promoter including a repressor element requires the absence of a certain compound, nutrient, amino acid, sugar, peptide, protein or condition to induce gene activity (e.g., transcription). Any suitable inducible promoter, system, or operon can be used to regulate the expression of a gene. Non-limiting examples of inducible promoters include temperature inducible promoters (e.g., heat inducible PgroES promoter, heat inducible phage lambda pL promoter, heat inducible phage lambda pR promoter, cold inducible cspA promoter), lactose regulated systems (e.g., lactose operon systems), sugar regulated systems, metal regulated systems, steroid regulated systems, alcohol regulated systems, IPTG inducible systems (e.g., pLac promoter), arabinose regulated systems (e.g., arabinose operon systems, pBad promoter), synthetic amino acid regulated systems (e.g., see Rovner et al. Nature, 2015, 518(7537), 89-93), fructose repressors, a tac promoter/operator (pTac), tryptophan promoters (e.g., Ptrp, induced by tryptophan depletion or by addition of β-indoleacrylic acid), alkaline phosphatase promoters (e.g., PhoA promoter induced by phosphate limitation), recA promoters (e.g., recA promoter induced by UV light), proU promoters (e.g., osmotically inducible proU promoter), cst promoters (e.g., cst promoter inducible by carbon starvation), tetA promoters (e.g., tetracycline inducible tetA promoter), cadA and cadR promoters (e.g., PcadA and PcadR induced by cadmium), nar promoters (e.g., nar promoter induced by oxygen), or combinations thereof.

In embodiments, a Zn2+-inducible promoter can be used (Eberhardt et al. Mol. Microbiol. 2009, 74, 395-408). In embodiments, a fucose-inducible promoter can be used (Chan et al. J. Bacteriol. 2003, 185, 2051-2058). In embodiments, a maltose-inducible promoter can be used (Guiral et al. Microbiology 2006, 152, 343-349). In embodiments, a trehalose-inducible promoter can be used (Sorg et al. ACS Synth. Biol. 2015, 4, 228-239).

In embodiments, expression of a gene can be controlled in additional ways known to one of skill in the art including modifying: gene copy number, number of copies of transcription factors binding the promoter operably linked to the gene; transcription factor binding to the gene promoter; RNA polymerase binding affinity for the gene promoter; ribosome binding affinity for the RBS; mRNA decay rate; and protein decay rate (Brewster et al. (2012) PLoS Comput Biol 8(12): e1002811). In embodiments, a promoter such as T7 can be regulated using a system with a temperature sensitive intein inserted in the protein sequence of T7 RNA polymerase (Korvin and Yadav (2018) Molecular Systems Design & Engineering 3(3):550-559). The polymerase is only active and able to drive gene expression when the intein is spliced out at the appropriate temperature.

The term “operably linked” refers to polynucleotide sequences or amino acid sequences placed into a functional relationship with one another. For instance, a promoter or enhancer is operably linked to a coding sequence if it regulates, or contributes to the modulation of, the transcription of the coding or non-coding sequence. In embodiments, regulatory sequences operably linked to a coding sequence are typically contiguous to the coding sequence. However, enhancers can function when separated from a promoter by up to several kilobases or more. Accordingly, some polynucleotide elements can be operably linked but not contiguous. In embodiments, a heterologous promoter or heterologous regulatory elements include promoters and regulatory elements that are not normally associated with a particular nucleic acid in nature.

A genetic construct or expression cassette of the disclosure can include a “secretion signal,” “signal sequence,” “secretion signal polypeptide,” “signal peptide,” or “leader sequence”. These terms refer to a peptide sequence (or the polynucleotide encoding the peptide sequence) that is useful for targeting an operably linked protein or polypeptide of interest expressed inside a cell to the periplasmic space of the cell or to the extracellular space outside of the cell. In embodiments, a heterologous immunogenic protein, or an immunogenic fragment or variant thereof, of the disclosure is expressed as a pro-protein including the signal sequence and heterologous immunogenic protein, or an immunogenic fragment or variant thereof, where the signal sequence is subsequently cleaved as the heterologous immunogenic protein, or an immunogenic fragment or variant thereof, is secreted from the cell to render a mature heterologous immunogenic protein, or an immunogenic fragment or variant thereof, without the signal sequence. In embodiments, a signal peptide has an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an amino acid sequence set forth as any of SEQ ID NOs: 210-212, wherein said signal peptide retains signal peptide function; and/or has an amino acid sequence set forth as any of SEQ ID NOs: 210-212. In embodiments, a signal peptide retains signal peptide function when the signal peptide is capable of targeting an operably linked protein or polypeptide of interest expressed inside a cell to the periplasmic space of the cell or to the extracellular space outside of the cell. In embodiments, a signal peptide of the disclosure includes a lipoprotein anchor that directs the secretion of a protein having the signal peptide outside of a cell and the anchoring of the protein at the plasma membrane as described herein. In embodiments, a signal peptide from a gram positive bacteria may be used, including a signal peptide from: listeriolysin O (LLO), alkaline phosphatase (phoZ), CITase gene, and the twin arginine translocation system.

A termination region, or terminator, can be provided by the naturally occurring or endogenous transcriptional termination region of the polynucleotide sequence encoding a protein of the disclosure or it can be a termination region of a polynucleotide sequence heterologous to the polynucleotide sequence encoding a protein of the disclosure. In embodiments, a genetic construct of the present disclosure includes a terminator having a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a nucleotide sequence set forth as SEQ ID NO: 235, wherein the nucleotide sequence retains transcription termination function; and/or having a nucleotide sequence set forth as SEQ ID NO: 235. In embodiments, a terminator is 3′ of a gene encoding a heterologous protein or fusion protein. In embodiments, a terminator is included 5′ of a promoter to insulate a genetic construct from transcription from upstream genes (i.e. genes 5′ of the promoter). In embodiments, a genetic construct of the present disclosure includes a termination region available from the Registry for Standard Biological Parts at World Wide Web at parts.igem.org, including BBa_B1002, BBa_B0015, BBa_B1006, and a modified version of BBa_B1006 (Sorg et al. ACS Synth. Biol. 2015, 4, 228-239). In embodiments, a genetic construct of the present disclosure includes a termination region from a ribosomal protein RpsI (Sorg et al. ACS Synth. Biol. 2015, 4, 228-239). In embodiments, a genetic construct of the present disclosure includes a termination region from an elongation factor Tuf (Sorg et al. ACS Synth. Biol. 2015, 4, 228-239). In embodiments, terminator sequences from a ribosomal protein RpsI and an elongation factor Tuf are included 5′ of a promoter of the disclosure. In embodiments, a terminator included 5′ of a promoter of the disclosure has a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a nucleotide sequence set forth as SEQ ID NO: 237, wherein the nucleotide sequence retains transcription termination function; and/or has a nucleotide sequence set forth as SEQ ID NO: 237. In embodiments, a nucleotide sequence retains transcription termination function when it directs cessation of transcription of an RNA from a gene operably linked to the nucleotide sequence and/or directs dissociation of the transcribed RNA, polymerase, and gene. For the most part, the source of the termination region is generally not considered to be critical to the expression of a recombinant protein and a wide variety of termination regions can be employed without adversely affecting expression.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, a process sometimes called “codon optimization” or “controlling for species codon bias.”

Optimized coding sequences containing codons preferred by a particular prokaryotic or eukaryotic host (see also, Murray et al. (1989) Nucl. Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al. (1996) Nucl. Acids Res. 24: 216-218).

In embodiments, a genetic construct of the disclosure can be propagated in vitro in a host cell suitable for replication of the genetic construct. Host cells can include bacterial cells, mammalian cells, yeast cells, insect cells, or plant cells. In embodiments, the host cell is a bacterium, e.g., E. coli or S. pneumoniae bacteria. The selection of an appropriate host is deemed to be within the scope of those skilled in the art. A recombinant host cell includes a host cell into which has been introduced a genetic construct.

In embodiments, a genetic construct is introduced into bacteria using a suitable technique. In embodiments, bacteria are transformed with a genetic construct by a suitable technique. Non-limiting examples of suitable techniques for introducing a nucleic acid into bacteria include conjugation, electroporation, transduction (e.g., injection of a nucleic acid by a bacteriophage), microinjection, by inducing competence (e.g., by addition of alkali cations, cesium, lithium, polyethylene glycol or by osmotic shock), or combinations thereof. Thus, the present disclosure includes a method of producing an immunogenic composition, where the method comprises introducing at least one fusion protein into a recombinant live attenuated S. pneumoniae. Each fusion protein comprises: (i) an immunogenic protein, or an immunogenic fragment or variant thereof; and (ii) a surface anchor moiety. The modified recombinant live attenuated S. pneumoniae that is produced expresses each fusion protein at the cell surface. In embodiments, introducing each fusion protein into the recombinant live attenuated S. pneumoniae comprises introducing at least one nucleic acid molecule comprising a nucleotide sequence encoding the at least one fusion protein. In embodiments, the recombinant live attenuated S. pneumoniae comprises a disruption of an ftsY gene in its genome. In embodiments, the modified recombinant live attenuated S. pneumoniae comprises in its genome a nucleotide sequence set forth as any of SEQ ID NOs: 229, 230, 231, 232, 233, and 234.

As would be appreciated by one of ordinary skill in the art, the genetic engineering strategies described herein to express a surface anchored heterologous protein or immunogenic fragment or variant thereof can also be applied to any strain or serotype of S. pneumoniae, including BHN97 (serotype 19F) strain. BHN97 is a non-invasive serotype and normally causes sinusitis/purulent rhinitis and acute otitis media. There are more than 90 serotypes known, with the most commonly used vaccines targeting 13 (1, 3, 4, 5, 6A, 6B, 7F, 9V, 14, 19A, 19F, 18C, and 23F) or 23 of these (1, 2, 3, 4, 5, 6B, 7F, 8, 9N, 9V, 10A, 11A, 12F, 14, 15B, 17F, 18C, 19F, 19A, 20, 22F, 23F, and 33F). Thus, in embodiments, the recombinant S. pneumoniae is any one of serotypes 1, 2, 3, 4, 5, 6A, 6B, 7F, 8, 9N, 9V, 10A, 11A, 12F, 14, 15B, 17F, 18C, 19A, 19F, 20, 22F, 23F, and 33F. The BHN97 (serotype 19F) genome is disclosed in NCBI Accession No. PRJNA420094. The term “strain”, as used herein, describes variants of a bacterial species (e.g., S. pneumoniae) that can be distinguished by one or more characteristics, such as ribosomal RNA sequence variation, DNA polymorphisms, serological typing, or toxin production, from other strains within that species.

The present disclosure includes heterologous immunogenic proteins or immunogenic fragments or variants thereof expressed by recombinant S. pneumoniae bacteria as described herein. In embodiments, immunogenic fragments include at least 5, at least 10, at least 15, at least 20, at least 30, at least 40, at least 50, at least 60, at least 70, at least 80, at least 90, at least 100, at least 150, at least 200, at least 300, at least 400, at least 500, at least 600, at least 700, at least 800, at least 900, at least 1,000 contiguous amino acid residues, or up to the entire contiguous amino acid residues of the protein. Methods for obtaining such fragments are known in the art and are described in further detail elsewhere herein.

### V. Heterologous Proteins Expressed on the Surface of Live, Attenuated S. pneumoniae

The presently disclosed immunogenic compositions comprise live attenuated S. pneumoniae genetically modified to express on its surface at least one heterologous immunogenic protein, or immunogenic fragment or variant thereof. In embodiments, the at least one heterologous immunogenic protein, or an immunogenic fragment or variant thereof, is required for transmission of at least one pathogen. The at least one pathogen can include S. pneumoniae, H. influenzae, and/or M. catarrhalis. In embodiments, the at least one heterologous immunogenic protein, or an immunogenic fragment or variant thereof, is modified to include a surface anchor moiety as described herein.

The term “immunogenic”, “immunogenicity”, or “immunogenic activity” refers to the ability of a polypeptide to elicit an immunological (i.e. immune) response in a subject (e.g., a mammal). As used herein, to “elicit” an immune response is to induce and/or potentiate an immune response. As used herein, to “enhance” an immune response is to elevate, improve or strengthen the immune response to the benefit of the host relative to the prior immune response status, for example, before the administration of an immunogenic composition of the disclosure. An “immunogenic composition” refers to a composition that can elicit an immune response. In some embodiments, an immunogenic composition can include a vaccine composition. An immunological response to a polypeptide is the development in a subject of a cell-mediated and/or antibody-mediated immune response to the polypeptide. Usually, an immunological response includes but is not limited to one or more of the following effects: the production of antibodies, B cells, helper T cells, suppressor T cells and/or cytotoxic T cells, directed to an epitope or epitopes of the polypeptide. The term “epitope” refers to the site on an antigen to which specific B cells and/or T cells respond so that antibody is produced. The term “antigen” refers to any substance, including a protein, a carbohydrate, a lipid, a nucleic acid, a mixture of these, or a plurality of these, which upon administration stimulates the formation of specific antibodies or memory T cells. The term “antigen” is used interchangeably herein with “immunogen” and “an immunogenic protein or immunogenic fragment or variant thereof”. An antigen can stimulate the proliferation of T-lymphocytes with receptors for the antigen, and can react with the lymphocytes to initiate the series of responses designated cell-mediated immunity. In embodiments, an antigen includes a protein, glycoprotein, lipoprotein, saccharide, polysaccharide, lipopolysaccharide, or a combination thereof. In embodiments, an antigen includes a heterologous immunogenic protein, or an immunogenic fragment or variant thereof, expressed by a recombinant S. pneumoniae as described in the disclosure.

As used herein, the term “antibody response”, “antibody-mediated response”, or “antibody-mediated immune response” refers to an increase in the amount of antigen-specific antibodies in the body of a subject in response to introduction of an antigen into the body of the subject.

An “antibody” is a protein comprising one or more polypeptides substantially or partially encoded by immunoglobulin genes or fragments of immunoglobulin genes. The recognized immunoglobulin genes include the κ, Δ, α, γ, δ, ε and μ constant region genes, as well as myriad immunoglobulin variable region genes. Light chains are classified as either κ or λ. Heavy chains are classified as γ, μ, α, δ, or ε, which in turn define the immunoglobulin classes, IgG, IgM, IgA, IgD and IgE, respectively. A typical immunoglobulin (antibody) structural unit comprises a protein containing four polypeptides. Each antibody structural unit is composed of two identical pairs of polypeptide chains, each having one “light” and one “heavy” chain. The N-terminus of each chain defines a variable region primarily responsible for antigen recognition. Antibody structural units (e.g. of the IgA and IgM classes) may also assemble into oligomeric forms with each other and additional polypeptide chains, for example as IgM pentamers in association with the J-chain polypeptide.

The immunogenicity of a polypeptide can be assayed by measuring the level of antibodies or T cells produced against the polypeptide. One method of evaluating an antibody response is to measure the titers of antibodies reactive with a particular antigen. This may be performed using a variety of methods known in the art such as enzyme-linked immunosorbent assay (ELISA). For example, the titers of serum antibodies which bind to a particular antigen may be determined in a subject both before and after exposure to the antigen. A statistically significant increase in the titer of antigen-specific antibodies following exposure to the antigen would indicate the subject had mounted an antibody response to the antigen. Other assays that may be used to detect the presence of an antigen-specific antibody include, without limitation, other immunological assays (e.g., radioimmunoassays, immunoprecipitation assays, Western blot) and neutralization assays (e.g., neutralization of viral infectivity in an in vitro or in vivo assay). Assays to measure for the level of antibodies are described, for example, in Harlow and Lane (1988) Antibodies, A Laboratory Manual (Cold Spring Harbor Publications, New York), for a standard description of antibody generation, immunoassay formats and conditions that can be used to determine specific immunoreactivity.

A “cell-mediated immune response” refers to an increase in the amount of and/or activation of antigen-specific cytotoxic T-lymphocytes, macrophages, and/or natural killer cells; and/or release of cytokines in the body of a subject in response to introduction of an antigen into the body of the subject. Cellular immunity is an important component of the adaptive immune response and following recognition of antigen by cells through their interaction with antigen-presenting cells such as dendritic cells, B lymphocytes, and/or macrophages, protects the body by various mechanisms including: activation of antigen-specific cytotoxic T-lymphocytes that induce apoptosis in cells displaying epitopes of the antigen on their surface, such as virus-infected cells, cells with intracellular bacteria, and cancer cells displaying tumor antigens; activation of macrophages and natural killer cells to destroy intracellular pathogens; and stimulation of cells to secrete a variety of cytokines that influence the function of other cells involved in adaptive immune responses and innate immune responses. Assays that can be performed to measure cell-mediated immune responses include: flow cytometry to identify antigen presenting cells and/or cytotoxic T-lymphocytes; in vitro cytolytic assays to measure cytotoxic T-lymphocyte activity; immunoassays to detect cytotoxins released from cytotoxic T-lymphocytes; and/or immunoassays or flow cytometry to measure cytokine release. Assays for T cells specific to a polypeptide are described, for example, by Rudraraju et al. (2011) Virology 410:429-36, herein incorporated by reference.

A heterologous immunogenic protein, or an immunogenic fragment or variant thereof, that is expressed on the surface of a recombinant live attenuated S. pneumoniae, includes protein D. In embodiments, the protein D is from an H. influenzae strain listed in Table 1. Table 1 shows representative protein D from different H. influenzae strains and their associated GenBank accession numbers. In embodiments, the protein D is from H. influenzae (SEQ ID NO: 213; GenBank M37487.1). In embodiments, a protein D of the disclosure has an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an amino acid sequence set forth as SEQ ID NO: 213, wherein the protein D retains immunogenicity; and/or has an amino acid sequence set forth as SEQ ID NO: 213. In embodiments, a protein D of the disclosure is encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a nucleotide sequence set forth as SEQ ID NO: 214, wherein the nucleotide sequence encodes a protein D that retains immunogenicity; and/or is encoded by a nucleotide sequence set forth as SEQ ID NO: 214. In embodiments, a protein D retains immunogenicity when the protein D retains the ability to elicit an immunological response in a subject (e.g., a mammal) as described herein. Protein D is a 42 kDa surface-exposed outer membrane lipoprotein found in all H. influenzae, including nontypeable (NT) strains. Protein D (encoded by the hpd or glpQ gene) exhibits limited diversity, with >97% sequence identity to a consensus protein D sequence at the nucleotide and amino acid level for a number of H. influenzae strains (Table 1; Forsgren et al. Clinical Infectious Diseases 2008, 46, 726-731). Protein D is a virulence factor that functions in pathogenesis of respiratory tract infections. Protein D has glycerophosphodiester phosphodiesterase activity, allowing the release of phosphorylcholine from host epithelial cells. Studies in a human nasopharyngeal tissue culture model demonstrated that protein D impaired ciliary function. In rats, protein D enhances the capacity to cause otitis media. Rats and chinchillas vaccinated with protein D show beneficial effects against H. influenzae infection. A clinical trial involving children used protein D as an antigenically active carrier for a pneumococcal conjugate investigational vaccine (Prymula et al. Lancet 2006, 367, 740-748).

TABLE 1
Representative protein D from different H. influenzae strains

| | | GenBank |
|---|---|---|
| Strain | Serotype | Accession No. |
| 772 | Nontypeable | M37487 |
| 86-028NP | Nontypeable | CP000057.2 |
| 3639 | Nontypeable | Z35656 |
| 3640 | Nontypeable | Z35657 |
| 6-7626 | Nontypeable | Z35658 |
| A850079 | Nontypeable | X90495 |
| A850053 | Nontypeable | X90493 |
| A850047 | Nontypeable | X90489 |
| A850048 | Nontypeable | X90491 |
| MinnA | b | L15200 |
| HK695 | b | Z35660 |
| Eagan | b | Z35659 |
| NCTC8468 | b | Z35661 |
| Rd KW20 | d | L42023 |

Adapted from Forsgren et al. Clinical Infectious Diseases 2008, 46, 726-731.

A heterologous immunogenic protein, or an immunogenic fragment or variant thereof, that is expressed on the surface of a recombinant live attenuated S. pneumoniae, includes a UspA polypeptide. In embodiments, the UspA polypeptide is from M. catarrhalis. In embodiments, the UspA polypeptide is an M. catarrhalis high molecular weight outer membrane UspA1 polypeptide having an amino acid sequence set forth as SEQ ID NO: 261 (GenBank AAB96359.2). In embodiments, a UspA polypeptide of the disclosure has an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an amino acid sequence set forth as SEQ ID NO: 261, wherein the UspA polypeptide retains immunogenicity. In embodiments, the UspA polypeptide is an M. catarrhalis high molecular weight outer membrane UspA1 polypeptide encoded by a nucleotide sequence set forth as SEQ ID NO: 262 (GenBank U57551.2). In embodiments, a UspA polypeptide of the disclosure is encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a nucleotide sequence set forth as SEQ ID NO: 262, wherein the nucleotide sequence encodes a UspA polypeptide that retains immunogenicity. In embodiments, the UspA polypeptide is an M catarrhalis high molecular weight surface protein UspA2 polypeptide having an amino acid sequence set forth as SEQ ID NO: 263 (GenBank AAB96391.1). In embodiments, a UspA polypeptide of the disclosure has an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an amino acid sequence set forth as SEQ ID NO: 263, wherein the UspA polypeptide retains immunogenicity. In embodiments, the UspA polypeptide is an M. catarrhalis high molecular weight surface protein UspA2 polypeptide encoded by a nucleotide sequence set forth as SEQ ID NO: 264 (GenBank U86135.2). In embodiments, a UspA polypeptide of the disclosure is encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a nucleotide sequence set forth as SEQ ID NO: 264, wherein the nucleotide sequence encodes a UspA polypeptide that retains immunogenicity. In embodiments, a UspA polypeptide retains immunogenicity when the UspA polypeptide retains the ability to elicit an immune response in a subject (e.g., a mammal) as described herein. The high molecular weight UspA protein of M. catarrhalis is expressed on its surface. This protein can be targeted by the monoclonal antibody Mab17C7, and in a mouse model system, Mab17C7 can enhance pulmonary clearance of M. catarrhalis (M. E. Helminen et al. J. Infect. Dis. 170:867-872, 1994). The uspA1 and uspA2 genes are described in Aebi et al. Infection and Immunity, November 1997, p. 4367-4377.

A heterologous immunogenic protein, or an immunogenic fragment or variant thereof, that is expressed on the surface of a recombinant live attenuated S. pneumoniae, includes factors required for or involved in mammalian transmission of S. pneumoniae, as described in PCT/IB2020/052250. In embodiments, a heterologous immunogenic protein, or an immunogenic fragment or variant thereof, that is expressed on the surface of a recombinant S. pneumoniae, includes proteins required for normal or optimal levels of transmission of S. pneumoniae (SEQ ID NOs: 1-205; Table 2). Thus, where the heterologous immunogenic protein, or an immunogenic fragment or variant thereof, includes a native S. pneumoniae protein, a live attenuated S. pneumoniae that is genetically modified to express on its surface a native S. pneumoniae protein includes at least one additional copy of the gene encoding the native S. pneumoniae protein in its genome. In embodiments, the expressed native S. pneumoniae protein is modified to include a surface anchor moiety. In embodiments, the heterologous immunogenic protein has an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an amino acid sequence set forth as any one of SEQ ID NOs: 1-205.

TABLE 2
Exemplary heterologous proteins for expression in recombinant
live attenuated S. pneumoniae of the disclosure

| SEQ | BHN97 | TIGR4 | | |
|---|---|---|---|---|
| ID | Locus | Locus | Functional | |
| NO: | ID | ID | Classification | KEGG prediction**/Name |
| 1 | peg.0181 | n/a | translation | Is required not only for elongation of protein synthesis |
| | | | | but also for the initiation of all mRNA translation |
| | | | | through initiator tRNA(fMet) aminoacylation (By |
| | | | | similarity) |
| 2 | peg.1585 | SP_0252 | CHO | Transaldolase is important for the balance of |
| | | | metabolism | metabolites in the pentose-phosphate pathway (By |
| | | | | similarity) |
| 3 | peg.0031 | SP_0913 | bacteriocin | ABC transporter, permease |
| 4 | peg.0182 | n/a | signaling | protein tyrosine serine phosphatase |
| 5 | peg.0843 | n/a | phage | NA |
| 6 | peg.1235 | SP_2146 | CHO | Alpha-L-fucosidase |
| | | | metabolism | |
| 7 | peg.1239 | SP_2152 | transport | c4-dicarboxylate anaerobic carrier |
| 8 | peg.1635 | n/a | regulation | |
| 9 | peg.1750 | SP_0397 | CHO | mannitol-1-phosphate 5-dehydrogenase |
| | | | metabolism | |
| 10 | peg.0148 | SP_1032 | Metal | Periplasmic binding protein/PiaA |
| | | | transport | |
| 11 | peg.0152 | SP_1035 | Metal | ABC transporter/PiaD |
| | | | transport | |
| 12 | peg.0159 | SP_1040 | recombination | Resolvase |
| 13 | peg.0186 | SP_0638 | transport | ABC transporter, permease |
| 14 | peg.0256 | SP_1125 | CHO | Glycerate kinase |
| | | | metabolism | |
| 15 | peg.0384 | SP_1275 | CHO | carbamoyl-phosphate synthetase ammonia chain |
| | | | metabolism | |
| 16 | peg.0386 | SP_1277 | Amino acid | aspartate transcarbamylase |
| | | | metabolism | |
| 17 | peg.0483 | SP_1391 | Protein | Putative membrane peptidase family (DUF2324) |
| | | | processing | |
| 18 | peg.0730 | SP_1654 | CHO | Alpha-L-fucosidase |
| | | | metabolism | |
| 19 | peg.0904 | SP_1840 | transport | ABC transporter |
| 20 | peg.1016 | SP_1962 | unknown | NA |
| 21 | peg.1044 | SP_1956 | bacteriocin | bacteriocin-associated integral membrane |
| 22 | peg.1101 | SP_2013 | transport | abc transporter permease protein |
| 23 | peg.1190 | SP_2103 | translation | Methyltransferase |
| 24 | peg.1234 | SP_2145 | CHO | Alpha-1,2-mannosidase |
| | | | metabolism | |
| 25 | peg.1334 | SP_2236 | competence | Histidine kinase/ComD |
| 26 | peg.1455 | n/a | Unknown | |
| 27 | peg.1463 | SP_0117 | unknown | Choline binding protein A |
| 28 | peg.1817 | n/a | unknown | NA |
| 29 | peg.1823 | SP_0480 | transport | potassium transporter peripheral membrane |
| 30 | peg.2077 | SP_0752 | Amino acid | ABC transporter |
| | | | transport | |
| 31 | peg.0039 | SP_0921 | Polyamine | Agmatine deiminase |
| | | | metabolism | |
| 32 | peg.0074 | SP_0954 | competence | Competence protein/ComEC |
| 33 | peg.0166 | SP_1046 | CHO | alpha amylase, catalytic region |
| | | | metabolism | |
| 34 | peg.0169 | SP_1051 | plasmid | zeta toxin |
| 35 | peg.0185 | SP_0637 | transport | transporter, permease |
| 36 | peg.0248 | SP_1118 | CHO | pullulanase |
| | | | metabolism | |
| 37 | peg.0249 | SP_1119 | CHO | Aldehyde dehydrogenase |
| | | | metabolism | |
| 38 | peg.0401 | n/a | unknown | NA |
| 39 | peg.0403 | n/a | unknown | cell wall binding |
| 40 | peg.0430 | n/a | CHO | Converts N-acetylmannosamine-6-phosphate |
| | | | metabolism | (ManNAc-6-P) to N-acetylglucosamine-6-phosphate |
| | | | | (GlcNAc-6-P) (By similarity) |
| 41 | peg.0485 | n/a | Bacteriocin | NA |
| 42 | peg.0514 | SP_1423 | unknown | |
| 43 | peg.0524 | SP_1430 | DNA | DNA methylase n-4 n-6 domain protein |
| | | | methylation | |
| 44 | peg.0529 | SP_1449 | unknown | CppA protein/CppA |
| 45 | peg.0669 | SP_1587 | transport | Major Facilitator |
| 46 | peg.0749 | SP_1674 | regulation | transcriptional regulator |
| 47 | peg.0755 | SP_1681 | CHO transport | ABC transporter (Permease) |
| 48 | peg.0760 | SP_1686 | metabolism | oxidoreductase |
| 49 | peg.0777 | SP_1704 | transport | ABC transporter |
| 50 | peg.0854 | SP_1795 | CHO | sucrose-6-phosphate hydrolase |
| | | | metabolism | |
| 51 | peg.0873 | n/a | metabolism | PEP phosphonomutase family protein |
| 52 | peg.0875 | n/a | regulation | ROK family |
| 53 | peg.0896 | SP_1830 | regulation | Plays a role in the regulation of phosphate uptake |
| 54 | peg.1022 | SP_1933 | regulation | NA |
| 55 | peg.1023 | SP_1934 | unknown | NA |
| 56 | peg.1076 | SP_1989 | regulation | Transcriptional regulator |
| 57 | peg.1118 | SP_2031 | CHO | L-ascorbate 6-phosphate lactonase |
| | | | metabolism | |
| 58 | peg.1181 | SP_2095 | signaling | Rhomboid family |
| 59 | peg.1198 | SP_2112 | Regulation | transcriptional regulator |
| 60 | peg.1221 | SP_2132 | Unknown | Band 7 protein |
| 61 | peg.1233 | SP_2144 | CHO | Conserved Protein |
| | | | metabolism | |
| 62 | peg.1236 | SP_2148 | Amino acid | Arginine dihydrolase |
| | | | metabolism | |
| 63 | peg.1272 | SP_2182 | unknown | |
| 64 | peg.1277 | SP_2187 | Regulation | M protein trans-acting positive regulator |
| 65 | peg.1386 | SP_0045 | CHO | phosphoribosylformylglycinamidine synthase |
| | | | metabolism | |
| 66 | peg.1387 | SP_0046 | CHO | glutamine phosphoribosylpyrophosphate |
| | | | metabolism | amidotransferase |
| 67 | peg.1419 | SP_0082 | adhesin | cell wall surface anchor family protein |
| 68 | peg.1433 | SP_0097 | unknown | |
| 69 | peg.1437 | SP_0101 | transport | Major Facilitator Superfamily |
| 70 | peg.1486 | n/a | unknown | |
| 71 | peg.1515 | SP_0177 | Metabolism | riboflavin synthase, subunit alpha |
| 72 | peg.1580 | SP_0247 | Regulation | regulator |
| 73 | peg.1643 | n/a | CHO | malate dehydrogenase (Oxaloacetate-decarboxylating) |
| | | | metabolism | |
| 74 | peg.1673 | SP_0324 | CHO transport | PTS System |
| 75 | peg.1719 | SP_0374 | unknown | NA |
| 76 | peg.1726 | SP_0380 | bacteriocin | |
| 77 | peg.1825 | SP_0482 | Transport | UPF0397 protein |
| 78 | peg.1869 | SP_0539 | Bacteriocin | |
| 79 | peg.1908 | SP_0575 | regulation | type iii restriction protein res subunit |
| 80 | peg.1937 | SP_0607 | Amino acid | amino acid AbC transporter |
| | | | transport | |
| 81 | peg.1949 | SP_0620 | Amino acid | ABC transporter substrate-binding protein |
| | | | transport | |
| 82 | peg.1997 | SP_0667 | adhesin | surface protein/CbpL |
| 83 | peg.2043 | SP_0717 | Nucleic acid | 4-methyl-5-beta-hydroxyethylthiazole kinase |
| | | | metabolism | |
| 84 | peg.2093 | SP_0770 | Metal | ABC transporter |
| | | | transport | |
| 85 | peg.2140 | SP_0824 | Amino acid | ABC transporter, permease protein |
| | | | transport | |
| 86 | peg.2160 | SP_0843 | Nucleic acid | Catalyzes a reversible aldol reaction between |
| | | | metabolism | acetaldehyde and D-glyceraldehyde 3-phosphate to |
| | | | | generate 2-deoxy- D-ribose 5-phosphate (By similarity) |
| 87 | peg.2177 | SP_0858 | unknown | Membrane |
| 88 | peg.0391 | SP_1282 | transport | abc transporter atp-binding protein |
| 89 | peg.0404 | n/a | unknown | Transcriptional regulator |
| 90 | peg.1576 | SP_0234 | Metal | ABC transporter |
| | | | transport | |
| 91 | peg.1618 | SP_0287 | transport | Xanthine uracil vitamin C permease |
| 92 | peg.1992 | SP_0662 | regulation | Histidine kinase |
| 93 | peg.0287 | SP_1175 | adhesin | (Histidine triad) protein/Histidine triad protein |
| 94 | peg.1217 | SP_2128 | CHO | Transketolase |
| | | | metabolism | |
| 95 | peg.1230 | SP_2142 | CHO | hydrolase, family 20 |
| | | | metabolism | |
| 96 | peg.1426 | SP_0090 | transport | Binding-protein-dependent transport systems, inner |
| | | | | membrane component |
| 97 | peg.0516 | n/a | unknown | NA |
| 98 | peg.0840 | SP_1780 | Protein | Oligoendopeptidase f |
| | | | processing | |
| 99 | peg.1026 | SP_1937 | Cell wall | n-acetylmuramoyl-1-alanine amidase/LytA |
| | | | processing | |
| 100 | peg.1140 | SP_2052 | secretion | Competence protein |
| 101 | peg.1399 | SP_0060 | CHO | beta-galactosidase |
| | | | metabolism | |
| 102 | peg.1520 | SP_0180 | Bacteriocin? | CAAX protease self-immunity |
| 103 | peg.1671 | SP_0323 | CHO transport | PTS System |
| 104 | peg.1866 | SP_0529 | bacteriocin | Transport protein ComB |
| 105 | peg.1929 | SP_0600 | transport | abc transporter atp-binding protein |
| 106 | peg.2050 | SP_0724 | metabolism | 4-methyl-5-beta-hydroxyethylthiazole kinase |
| 107 | peg.0154 | n/a | plasmid | mobA MobL family protein |
| 108 | peg.0244 | SP_1114 | transport | ABC transporter, ATP-binding protein |
| 109 | peg.0298 | SP_1185 | transport | Pts system |
| 110 | peg.0427 | n/a | unknown | NA |
| 111 | peg.0439 | SP_1344 | bacteriocin | serine threonine protein kinase |
| 112 | peg.0575 | SP_1493 | adhesin | surface protein |
| 113 | peg.0613 | SP_1527 | Peptide | Oligopeptide-binding protein/AliB |
| | | | transport | |
| 114 | peg.0695 | SP_1612 | regulation | serine threonine protein kinase |
| 115 | peg.0853 | SP_1790 | recombination | recombination factor protein RarA |
| 116 | peg.0859 | SP_1800 | regulation | M protein trans-acting positive regulator (MGA) PRD |
| | | | | domain |
| 117 | peg.0937 | SP_1872 | transport | Periplasmic binding protein |
| 118 | peg.1088 | SP_2000 | regulation | response regulator |
| 119 | peg.1089 | SP_2001 | regulation | Histidine kinase |
| 120 | peg.1119 | SP_2032 | regulation | PRD domain protein |
| 121 | peg.1192 | SP_2106 | CHO | Phosphorylase is an important allosteric enzyme in |
| | | | metabolism | carbohydrate metabolism. Enzymes from different |
| | | | | sources differ in their regulatory mechanisms and in |
| | | | | their natural substrates. However, all known |
| | | | | phosphorylases share catalytic and structural properties |
| | | | | (By similarity) |
| 122 | peg.1485 | SP_0145 | transport | Major Facilitator |
| 123 | peg.1514 | SP_0176 | CHO | Catalyzes the conversion of D-ribulose 5-phosphate to |
| | | | metabolism | formate and 3,4-dihydroxy-2-butanone 4-phosphate (By |
| | | | | similarity) |
| 124 | peg.1632 | n/a | CHO | glycoside hydrolase family 1 |
| | | | metabolism | |
| 125 | peg.2032 | SP_0706 | unknown | NA |
| 126 | peg.0083 | SP_0965 | Cell wall | endo-beta-N-acetylglucosaminidase |
| | | | processing | |
| 127 | peg.0162 | SP_1043 | unknown | NA |
| 128 | peg.0171 | SP_1052 | unknown | atpase involved in DNA repair |
| | | and | | |
| | | SP_1053 | | |
| 129 | peg.0283 | SP_1170 | unknown | NA |
| 130 | peg.0548 | n/a | transport | abc transporter, ATP-binding protein |
| 131 | peg.0607 | SP_1523 | regulation | SNF2 family |
| 132 | peg.0611 | SP_1526 | transport | ABC transporter transmembrane region |
| 133 | peg.0696 | n/a | bacteriocin | serine threonine protein kinase |
| 134 | peg.0762 | SP_1688 | CHO transport | ABC transporter, permease |
| 135 | peg.7089 | SP_1715 | transport | NA |
| 136 | peg.0790 | SP_1717 | transport | ABC transporter, permease |
| 137 | peg.0796 | n/a | CHO transport | PTS system |
| 138 | peg.0867 | n/a | transport | Sulfite exporter TauE/SafE |
| 139 | peg.0870 | n/a | CHO transport | PTS system |
| 140 | peg.0889 | SP_1823 | Metal | MgtC SapB family protein |
| | | | transport | |
| 141 | peg.0893 | SP_1827 | regulation | Domain of unknown function (DUF1868) |
| 142 | peg.0916 | SP_1850 | restriction | Type II restriction |
| 143 | peg.1041 | SP_1952 | Protein | NA |
| | | | processing | |
| 144 | peg.1145 | SP_2058 | translation | Exchanges the guanine residue with 7-aminomethyl-7- |
| | | | | deazaguanine in tRNAs with GU(N) anticodons (tRNA- |
| | | | | Asp, -Asn, -His and -Tyr). After this exchange, a |
| | | | | cyclopentendiol moiety is attached to the 7- |
| | | | | aminomethyl group of 7-deazaguanine, resulting in the |
| | | | | hypermodified nucleoside queuosine (Q) (7-(((4,5-cis- |
| | | | | dihydroxy-2-cyclopenten-1-yl)amino)methyl)-7- |
| | | | | deazaguanosine) (By similarity) |
| 145 | peg.1174 | SP_2085 | transport | phosphate abc transporter |
| 146 | peg.1194 | SP_2108 | transport | extracellular solute-binding protein family 1 |
| 147 | peg.1276 | SP_2186 | CHO | Key enzyme in the regulation of glycerol uptake and |
| | | | metabolism | metabolism (By similarity) |
| 148 | peg.1280 | n/a | adhesin | NA/PspC |
| 149 | peg.1327 | SP_2233 | phage | domain protein |
| 150 | peg.1329 | n/a | phage | transposase IS116 IS110 IS902 family protein |
| 151 | peg.1427 | SP_0091 | CHO transport | Binding-protein-dependent transport systems, inner |
| | | | | membrane component |
| 152 | peg.1454 | n/a | unknown | NA |
| 153 | peg.1497 | SP_0156 | regulation | regulatoR |
| 154 | peg.1506 | SP_0165 | bacteriocin | Flavoprotein |
| 155 | peg.1521 | SP_0182 | bacteriocin | peptidase U61 LD-carboxypeptidase A |
| 156 | peg.1598 | SP_0267 | regulation | Luciferase-like |
| 157 | peg.1615 | SP_0285 | CHO | alcohol dehydrogenase |
| | | | metabolism | |
| 158 | peg.1638 | n/a | CHO | esterase |
| | | | metabolism | |
| 159 | peg.1662 | SP_0313 | metabolism | Glutathione peroxidase |
| 160 | peg.1686 | SP_0338 | Protein | ATP-dependent Clp protease ATP-binding subunit |
| | | | processing | |
| 161 | peg.1741 | SP_0391 | adhesin | choline binding protein/CbpF |
| 162 | peg.1831 | SP_0488 | regulation | PAP2 Family |
| 163 | peg.1915 | SP_0582 | nuclease | NA |
| 164 | peg.2014 | SP_0686 | bacteriocin | bacteriocin-associated integral membrane protein |
| 165 | peg.2025 | SP_0698 | transport | NA |
| 166 | peg.2046 | SP_0720 | transport | ABC transporter, ATP-binding protein |
| 167 | peg.2055 | SP_0729 | Metal | p-type ATPase |
| | | | transport | |
| 168 | peg.2083 | SP_0758 | CHO transport | PTS System |
| 169 | peg.2115 | SP_0790 | transport | NA |
| 170 | peg.2138 | SP_0820 | Protein | ATP-dependent clp protease, ATP-binding subunit |
| | | | processing | |
| 171 | peg.0038 | SP_0920 | metabolism | Catalyzes the decarboxylation of carboxynorspermidine |
| | | | | and carboxyspermidine (By similarity) |
| 172 | peg.0047 | SP_0930 | Cell wall | choline binding protein E |
| | | | processing | |
| 173 | peg.0250 | SP_1121 | CHO | Catalyzes the formation of the alpha-1,6-glucosidic |
| | | | metabolism | linkages in glycogen by scission of a 1,4-alpha-linked |
| | | | | oligosaccharide from growing alpha-1,4-glucan chains |
| | | | | and the subsequent attachment of the oligosaccharide to |
| | | | | the alpha-1,6 position (By similarity) |
| 174 | peg.0251 | SP_1122 | CHO | Catalyzes the synthesis of ADP-glucose, a sugar donor |
| | | | metabolism | used in elongation reactions on alpha-glucans (By |
| | | | | similarity) |
| 175 | peg.0422 | n/a | CHO | Conserved Protein |
| | | | metabolism | |
| 176 | peg.0438 | SP_1343 | transport | ABC transporter |
| 177 | peg.0440 | SP_1346 | bacteriocin | NA |
| 178 | peg.0530 | SP_1450 | adhesin | lipolytic protein G-D-S-L family |
| 179 | peg.0635 | SP_1548 | unknown | NA |
| 180 | peg.0658 | SP_1573 | Cell wall | Putative cell wall binding repeat/LytC |
| | | | processing | |
| 181 | peg.0697 | n/a | transport | Major Facilitator |
| 182 | peg.0820 | SP_1743 | regulation | Methyltransferase Type |
| 183 | peg.0888 | SP_1821 | regulation | Transcriptional regulator |
| 184 | peg.0927 | SP_1861 | Amino acid | ABC transporter |
| | | | transport | |
| 185 | peg.0934 | SP_1869 | Metal | permease protein |
| | | | transport | |
| 186 | peg.1014 | SP_1925 | unknown | NA |
| 187 | peg.1055 | SP_1967 | signaling | domain protein |
| 188 | peg.1060 | SP_1972 | unknown | Membrane |
| 189 | peg.1195 | SP_2109 | transport | binding-protein-dependent transport systems inner |
| | | | | membrane Component |
| 190 | peg.1450 | n/a | CHO | Inherit from NOG: Transcriptional regulator |
| | | | metabolism | |
| 191 | peg.1459 | n/a | unknown | NA |
| 192 | peg.1493 | SP_0152 | transport | ABC transporter, permease |
| 193 | peg.1494 | SP_0153 | unknown | integral membrane protein TIGR02185 |
| 194 | peg.1509 | SP_0167 | transport | Major Facilitator |
| 195 | peg.1535 | SP_0200 | unknown | NA |
| 196 | peg.1636 | n/a | transport | PTS system |
| 197 | peg.1660 | SP_0311 | unknown | NA |
| 198 | peg.1670 | SP_0322 | bacteriocin | Glycosyl Hydrolase Family 88 |
| 199 | peg.1677 | SP_2241 | unknown | NA |
| 200 | peg.1875 | SP_0536 | unknown | NA |
| | | (weak) | | |
| 201 | peg.1877 | SP_0545 | bacteriocin | caax amino terminal protease family protein |
| 202 | peg.1928 | SP_0599 | transport | ABC transporter, permease |
| 203 | peg.2015 | SP_0687 | Peptide | abc transporter atp-binding protein |
| | | | transport | |
| 204 | peg.2041 | SP_0719 | Metal | TENA THI-4 family protein |
| | | | transport | |
| 205 | peg.2157 | SP_0840 | unknown | NA |
| | | (weak) | | |

**Protein functions were predicted with the eggNOG pipeline, using HMMR on the bacteria database at World Wide Web at academic.oup.com/mbe/article/34/8/2115/3782716.

In embodiments, a heterologous immunogenic protein, or an immunogenic fragment or variant thereof, that is expressed on the surface of a recombinant live attenuated S. pneumoniae, includes an S. pneumoniae protein that is usually naturally expressed on the surface of a non-genetically modified S. pneumoniae. Alternatively, the S. pneumoniae protein is naturally expressed on the surface of a non-genetically modified S. pneumoniae when the bacterium is undergoing autolysis. In embodiments, the S. pneumoniae protein does not include a transmembrane domain.

In embodiments, a heterologous immunogenic protein, or an immunogenic fragment or variant thereof, that is expressed on the surface of a recombinant live attenuated S. pneumoniae, includes an S. pneumoniae choline-binding protein (CBP), or an immunogenic fragment or variant thereof. In embodiments, the CBP has an amino acid sequence selected from the group consisting of SEQ ID NO: 27, 39, and 82; or an immunogenic fragment or variant of any thereof. In embodiments, a CBP of the disclosure has an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an amino acid sequence set forth as any of SEQ ID NOs: 27, 39, and 82, wherein the CBP retains immunogenicity; and/or has an amino acid sequence set forth as any of SEQ ID NOs: 27, 39, and 82. In embodiments, a CBP retains immunogenicity when the CBP retains the ability to elicit an immunological response in a subject as described herein.

In embodiments, a heterologous immunogenic protein, or an immunogenic fragment or variant thereof, that is expressed on the surface of a recombinant live attenuated S. pneumoniae, includes an S. pneumoniae sensor kinase of the competence cascade (ComD), a C3-degrading protease (CppA), or an iron transporter (PiaA). In embodiments, a ComD protein has the amino acid sequence set forth as SEQ ID NO: 92, or an immunogenic fragment or variant thereof. In embodiments, a CppA protein has the amino acid sequence set forth as SEQ ID NO: 44, or an immunogenic fragment or variant thereof. In embodiments, a PiaA protein has the amino acid sequence set forth as SEQ ID NO: 10, or an immunogenic fragment or variant thereof. In embodiments, a heterologous immunogenic protein of the disclosure has an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an amino acid sequence set forth as any of SEQ ID NOs: 10, 44, and 92, wherein the heterologous immunogenic protein retains immunogenicity; and/or has an amino acid sequence set forth as any of SEQ ID NOs: 10, 44, and 92. In embodiments, a heterologous immunogenic protein retains immunogenicity when the heterologous immunogenic protein retains the ability to elicit an immunological response in a subject as described herein.

In embodiments, at least two heterologous immunogenic proteins, or immunogenic fragments or variants thereof are expressed on the surface of a recombinant live attenuated S. pneumoniae. The at least two heterologous immunogenic proteins, or immunogenic fragments or variants thereof, can be from a single pathogen. In embodiments, the single pathogen is H. influenzae. In embodiments, the single pathogen is S. pneumoniae. In embodiments, the single pathogen is H. influenzae. In embodiments, the single pathogen is M. catarrhalis. The at least two heterologous immunogenic proteins, or immunogenic fragments or variants thereof, can be from different pathogens. In embodiments, the different pathogens comprise a combination of pathogens selected from S. pneumoniae, H. influenzae, and M. catarrhalis.

A heterologous immunogenic protein, or an immunogenic fragment or variant thereof, that is expressed on the surface of a recombinant live attenuated S. pneumoniae, can include an additional moiety that provides an advantageous or additional characteristic. For example, the moiety may increase the half-life, solubility, bioavailability, or immunogenicity of the heterologous protein, or immunogenic fragment or variant thereof. As another example, the moiety may direct the heterologous immunogenic protein, or an immunogenic fragment or variant thereof, to be anchored at the plasma membrane or cell wall of the S. pneumoniae. In embodiments, the moiety includes: a surface anchor moiety (as further described herein); an additional immunogen (such as one that inhibits or reduces colonization); a peptide adjuvant; a tag; or a combination thereof. In embodiments, the moiety includes a protein, a peptide, a glycoprotein, a carbohydrate, a lipid, a lipoprotein, poly(ethylene glycol) (PEG), a nucleic acid, a polysaccharide, a polymer, and a particle. There are many different types of PEG, ranging from molecular weights of below 300 g/mol to over 10,000,000 g/mol. PEG chains can be linear, branched, or with comb or star geometries. In embodiments, the polysaccharide includes polysialic acid and N-propanoylated polysialic acid. In embodiments, a lipid moiety is recognized by a Toll-like receptor (TLR) such as TLR2, and activates the innate immune system.

In embodiments, the additional moiety is an adjuvant. Adjuvants generally are substances that can enhance the immunogenicity of polypeptides as further described herein. Adjuvants may play a role in both acquired and innate immunity (e.g., TLRs) and may function in a variety of ways, not all of which are understood. For example, a peptide adjuvant can include at least one of a tetanus toxoid, pneumolysis keyhole limpet hemocyanin, or the like.

In embodiments, the additional moiety is a tag. A tag may facilitate purification, detection, solubility, or confer other desirable characteristics on the heterologous protein. For instance, a tag may include a peptide, oligopeptide, or polypeptide that may be used in affinity purification. Examples of tags include polyhistidine, glutathione S-transferase (GST), tandem affinity purification (TAP), FLAG, myc, hemagglutinin (HA), maltose binding protein (MBP), vesicular stomatitis virus glycoprotein (VSV-G), thioredoxin, V5, biotin, avidin, streptavidin, biotin carboxyl carrier protein (BCCP), calmodulin, Nus, S tag, and β-galactosidase. In embodiments, a tag may be N-terminal, C-terminal, or internal to the heterologous immunogenic protein, or an immunogenic fragment or variant thereof.

The heterologous immunogenic protein, or an immunogenic fragment or variant thereof, may be joined to the additional moiety through chemical conjugation or by recombinant means. The terms “linked,” “fused”, or “fusion”, are used interchangeably. These terms refer to the joining together of two more elements or components, by whatever means including chemical conjugation or recombinant means. Methods of chemical conjugation (e.g., using heterobifunctional crosslinking agents) are known in the art. Recombinant means include the terms “genetically fused,” “genetically linked” or “genetic fusion” and refers to a co-linear, covalent linkage or attachment of two or more proteins, polypeptides, or fragments thereof via their individual peptide backbones, through genetic expression of a single polynucleotide molecule encoding those proteins, polypeptides, or fragments. Such genetic fusion results in the expression of a single contiguous genetic sequence. Preferred genetic fusions are in frame, i.e., two or more open reading frames (ORFs) are fused to form a continuous longer ORF, in a manner that maintains the correct reading frame of the original ORFs. Thus, the resulting recombinant fusion protein is a single polypeptide containing two or more protein segments that correspond to polypeptides encoded by the original ORFs (which segments are not normally so joined in nature). For example, an H. influenzae protein D of the disclosure is expressed as a fusion protein of H. influenzae protein D and a surface anchor moiety that directs the attachment of protein D either non-covalently or covalently to the plasma membrane or to the cell wall of S. pneumoniae. As another example, a fusion protein of the disclosure comprises a surface anchor moiety, an H. influenzae protein D, and an M. catarrhalis UspA polypeptide.

In embodiments, the different region, polypeptides, or fragments of a fusion protein are joined by a peptide linker. Suitable peptide linkers include glycine-serine based linkers, glycine-proline based linkers, proline-alanine based linkers, or any other type of linker that still allows the fusion protein to function (e.g., a linker that connects a protein D to a surface anchor moiety still allows localization of the protein D fusion to the cell surface and anchoring at the plasma membrane or at the cell wall).

### VI. Surface Anchoring of Expressed Heterologous Immunogenic Proteins, or Immunogenic Fragments or Variants Thereof

A heterologous immunogenic protein, or an immunogenic fragment or variant thereof, expressed by a recombinant, live attenuated S. pneumoniae described herein include fusions of at least one heterologous immunogenic protein, or an immunogenic fragment or variant thereof, with a surface anchor moiety. A fusion of at least one heterologous immunogenic protein, or an immunogenic fragment or variant thereof, and a surface anchor moiety may be referred to as a surface anchored heterologous immunogenic protein, or an immunogenic fragment or variant thereof. A surface anchor moiety or a moiety that has surface anchoring activity as used herein includes an amino acid sequence of a peptide or polypeptide that participates in anchoring and/or serves as a signal to anchor a heterologous immunogenic protein, or an immunogenic fragment or variant thereof, to the cell plasma membrane or to the cell wall of a recombinant S. pneumoniae expressing the heterologous immunogenic protein, or an immunogenic fragment or variant thereof modified with the surface anchor moiety or the moiety that has surface anchoring activity. In embodiments, the surface anchor moiety or the moiety that has surface anchoring activity includes one or more amino acid regions that directly participate in attachment of the heterologous immunogenic protein, or an immunogenic fragment or variant thereof, to the cell membrane or cell wall and/or that is recognized by another protein or molecule as a signal to attach the heterologous immunogenic protein, or immunogenic fragment or variant thereof, to the cell membrane or cell wall. In embodiments, a nucleotide sequence encodes a moiety that has surface anchoring activity when the moiety functions to direct and/or anchor a polypeptide to the cell plasma membrane or to the cell wall of a cell (e.g., an S. pneumoniae cell).

In embodiments, a fusion protein of the disclosure has an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an amino acid sequence set forth as any of SEQ ID NOs: 219, 223, and 226, wherein the fusion protein retains immunogenicity and anchoring at the cell surface; and/or has an amino acid sequence set forth as any of SEQ ID NOs: 219, 223, and 226. In embodiments, a fusion protein of the disclosure is encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a nucleotide sequence set forth as any of SEQ ID NOs: 220, 224, and 227, wherein the nucleotide sequence encodes a fusion protein that retains immunogenicity and anchoring at the cell surface; and/or is encoded by a nucleotide sequence set forth as any of SEQ ID NOs: 220, 224, and 227. In embodiments, a fusion protein retains immunogenicity and anchoring at the cell surface when the fusion protein retains the ability to elicit an immunological response in a subject and retains its localization to the cell surface and attachment (either covalently or non-covalently) to the cell plasma membrane and/or to the cell wall of a cell as described herein.

The cell envelope of a gram positive bacteria (e.g., S. pneumoniae) includes: 1) a cell wall made of many layers of peptidoglycan (also known as murein); and 2) a cell plasma membrane. The peptidoglycans of the cell wall are repeating units of the disaccharide N-acetyl glucosamine-N-acetyl muramic acid cross-linked by peptides of 3-5 amino acid residues into a mesh-like framework. The peptidoglycan layers are 30-100 nm thick and form a very large, rigid polymer that confers shape to a bacterial cell. Teichoic acids, long anionic polymers composed of glycerol phosphate, glucosyl phosphate, and/or ribitol phosphate repeats, are found in the peptidoglycan mesh of the cell wall. In embodiments, the basic repeating unit for teichoic acid includes 2-acetamido-4-amino-2,4,6-trideoxy-D-galactose, D-glucose, ribitol-phosphate, and two residues of N-acetylgalactosamine (GalNAc). Wall teichoic acids (WTA, also known as C-polysaccharide) are covalently attached to peptidoglycan, while lipoteichoic acids (LTA, also known as F-antigen) are anchored to the head groups of plasma membrane lipids. The teichoic acids can account for over 60% of the mass of a gram-positive cell wall. Phosphorylcholine (ChoP), composed of a negatively charged phosphate bonded to a small, positively charged choline group, are found on the GalNAc residues of teichoic acids. High expression of ChoP has been associated with S. pneumoniae and NT H. influenzae virulence. The surface of the plasma membrane of gram-positive bacteria are decorated with a variety of proteins, which can be retained on or near the plasma membrane in a number of ways. For example, proteins at or near the plasma membrane can contain membrane-spanning helices, attach to lipid anchors inserted in the plasma membrane, covalently attach to or associate tightly with peptidoglycan, or bind to teichoic acids. Reference to “the surface” of a cell includes the surface of the plasma membrane of the cell and/or the surface of the cell wall of the cell. In embodiments, a surface anchored heterologous immunogenic protein, or an immunogenic fragment or variant thereof, is located in or on the plasma membrane and/or in or on the cell wall. The terms “plasma membrane”, “cytoplasmic membrane”, “cell plasma membrane”, “cell membrane”, and “lipid membrane” are used interchangeably and refer to the membrane that encloses the contents of a cell, including the cytoplasm.

In embodiments, attachment of a surface anchored heterologous immunogenic protein, or an immunogenic fragment or variant thereof, to the plasma membrane or to the cell wall of S. pneumoniae includes covalent bonds or non-covalent interactions. Covalent bonds include the sharing of electrons in a chemical bond. Non-covalent interactions include dispersed electromagnetic interactions such as hydrogen bonds, ionic bonds, van der Waals interactions, and hydrophobic bonds.

### Choline Binding Domain (CBD)

In embodiments, a surface anchor moiety includes a CBD. A CBD may be genetically fused to at least one heterologous immunogenic protein, or an immunogenic fragment or variant thereof, such that the CBD of the expressed fusion protein allows for attachment to the cell wall. Choline binding proteins (CBP) in pneumococcus are surface proteins associated with the cell wall and that function in metabolic processes. CBPs include murein hydrolases that remodel the cell wall, host-cell adhesins, bacteriophage-encoded lytic enzymes, and other virulence factors. A CBD is a domain found in a CBP that allows non-covalent interaction of a CBP with choline (e.g., phosphorylcholine) in the cell wall. A CBD includes a tandem concatenation of 2 to 10 repeats of a 20-amino-acid aromatic-rich sequence, known as choline binding repeats. In embodiments, a consensus sequence for a choline binding repeat includes TGW-b-y-DNGSWYYLN-x-SG-z-M-x₁₋₂ (SEQ ID NO: 215), where b is a hydrophobic amino acid residue, y is K or Q, z is an amino acid residue that has a small side chain, and x is any amino acid residue (Maestro and Sanz Antibiotics 2016, 5, 21). In embodiments, a CBD of the disclosure includes a consensus amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a consensus amino acid sequence set forth as SEQ ID NO: 215, wherein the CBD retains surface anchoring activity; and/or includes a consensus amino acid sequence set forth as SEQ ID NO: 215. Amino acid residues of each choline binding repeat form a (3-hairpin with a strand-turn-strand configuration. In embodiments, a CBD of the present disclosure has an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an amino acid sequence set forth as SEQ ID NO: 216, wherein the CBD retains surface anchoring activity; and/or has an amino acid sequence set forth as SEQ ID NO: 216. In embodiments, a CBD of the present disclosure is encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a nucleotide sequence set forth as SEQ ID NO: 217, wherein the nucleotide sequence encodes a CBD that retains surface anchoring activity; and/or is encoded by a nucleotide sequence set forth as SEQ ID NO: 217. In embodiments, a CBD of the present disclosure includes a choline binding repeat having an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an amino acid sequence set forth as SEQ ID NO: 218, wherein the CBD retains surface anchoring activity; and/or includes an amino acid sequence set forth as SEQ ID NO: 218. In embodiments, a CBD retains surface anchoring activity when the CBD retains the ability to direct and/or anchor a polypeptide to the cell plasma membrane or to the cell wall of a cell as described herein. In embodiments, a CBD of the present disclosure includes 2 to 10 choline binding repeats. In embodiments, a CBD of the present disclosure includes 2 choline binding repeats, 3 choline binding repeats, 4 choline binding repeats, 5 choline binding repeats, 6 choline binding repeats, 7 choline binding repeats, 8 choline binding repeats, 9 choline binding repeats, 10 choline binding repeats, or more. In embodiments, a CBD of the present disclosure includes 2 choline binding repeats. In embodiments, a CBD of the present disclosure includes 3 choline binding repeats. In embodiments, a CBD is fused at the C-terminal portion of a heterologous immunogenic protein, or an immunogenic fragment or variant thereof. In embodiments, a CBD includes all or a portion of a CBD found in the following CBPs of pneumococcal strains: LytA (N-acetylmuramoyl L-alanine amidase; WP_000405235), LytB (endo-β-N-acetylglucosaminidase; COF07485.1), LytC (lysozyme; WP_010976587.1), CbpA (adhesin; also known as PspC, SpsA, PbcA; WP_000458116.1), CbpD (murine hydrolase, AAF87768.1), CbpE (phosphorylcholine esterase; also known as Pee and LytD; AAF87769.1), CbpF (regulator of lytC lysozyme; also known as CbpK; AAF87771.1), CbpG (proteolytic and adhesin functions; WP_168961039.1), CbpI (putative adhesin; AAF87772.1), CbpJ (putative adhesin; AAF87773.1), CbpL (endo-β-N-acetylglucosaminidase; AVN85730.1), CpbM (interacts with C-reactive protein; AVN86347.1), PcpA (putative adhesin; CAG5665959.1), PspA (surface protein A that binds lactoferrin; WP_001035315.1), CPL1 (phage lysozyme; NP_044837.1), and Pal (phage amidase; 003979.1). In embodiments, anchoring of a CBD-bearing protein at the surface of a cell can involve the following. A CBD-bearing protein is secreted to outside of a cell due to the presence of an N-terminal signal sequence. Signal sequences are described elsewhere herein. After secretion of the CBD-bearing protein to the outside of a cell, the CBD-bearing protein non-covalently associates with choline in the cell wall through the CBD. In embodiments, the present disclosure includes a protein D-CBD fusion protein having an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an amino acid sequence set forth as SEQ ID NO: 219, wherein the protein D-CBD fusion protein retains immunogenicity and anchoring at the cell surface; and/or having an amino acid sequence set forth as SEQ ID NO: 219. In embodiments, the present disclosure includes a protein D-CBD fusion protein encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a nucleotide sequence set forth as SEQ ID NO: 220, wherein the nucleotide sequence encodes a protein D-CBD fusion protein that retains immunogenicity and anchoring at the cell surface; and/or is encoded by a nucleotide sequence set forth as SEQ ID NO: 220. In embodiments, the present disclosure includes a fusion protein comprising CBD, a protein D, and a UspA polypeptide. In embodiments, a protein D-CBD fusion protein or a fusion protein comprising CBD, a protein D, and a UspA polypeptide retains immunogenicity and anchoring at the cell surface when the protein D-CBD fusion protein or a fusion protein comprising CBD, a protein D, and a UspA polypeptide retains the ability to elicit an immunological response in a subject and retains its localization to the cell surface and attachment to the cell plasma membrane and/or to the cell wall of a cell as described herein.

### Sortase Signal (LPXTG Type Motif)

In embodiments, a surface anchor moiety includes a sortase signal (i.e. LPXTG type motif) that is recognized and cleaved by a sortase enzyme. A sortase signal, including an LPXTG (Leucine-Proline-X-Threonine-Glycine) amino acid sequence (SEQ ID NO: 206) where “X” is any amino acid, is genetically fused to at least one heterologous immunogenic protein, or an immunogenic fragment or variant thereof, such that the sortase signal of the expressed fusion protein allows for attachment to the cell wall. In proteins that are naturally displayed on the cell surface using a sortase signal, the sortase signal is usually followed by a hydrophobic region and a cluster of positively charged amino acids. Thus, in embodiments, the terms “LPXTG type motif”, “LPXTG type sortase signal”, or “sortase signal”, used interchangeably herein, include not only the 5 amino acid residues of the amino acid sequence LPXTG (SEQ ID NO: 206) but also includes a hydrophobic region and a cluster of positively charged amino acids C-terminal to the 5 amino acid residues. In embodiments, the cluster of positively charged amino acids in the sortase signal is referred to as the positively charged tail. In embodiments, a sortase signal is located at the C-terminal portion of at least one heterologous immunogenic protein, or an immunogenic fragment or variant thereof.

In embodiments, sortase signals include ones from Staphylococcus aureus protein A, S. aureus FnBPB, Streptococcus pyogenes M6, and L. lactis PrtP (Liljeqvist et al. Appl. Environ. Microbiol. 1997, 63, 2481-2488; Strauss et al. Mol. Microbiol. 1996, 21, 491-500; Oggioni & Pozzi Gene 1996, 169, 85-90; Pozzi et al. Res. Microbiol. 1992, 143, 449-457; Norton et al. FEMS Immunol. Med. Microbiol. 1996, 14, 167-177; Norton et al. Folia Microbiol. 1995, 40, 225-230). In embodiments, a sortase signal that allows anchoring of a protein at the surface of a cell has an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an amino acid sequence set forth as SEQ ID NO: 221, wherein the sortase signal retains surface anchoring activity; and/or has an amino acid sequence set forth as SEQ ID NO: 221. In embodiments, a sortase signal that allows anchoring of a protein at the surface of a cell is encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a nucleotide sequence set forth as SEQ ID NO: 222, wherein the nucleotide sequence encodes a sortase signal that retains surface anchoring activity; and/or is encoded by a nucleotide sequence set forth as SEQ ID NO: 222. In embodiments, a sortase signal of the disclosure includes an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an amino acid sequence set forth as SEQ ID NO: 206, wherein the sortase signal retains surface anchoring activity; and/or includes an amino acid sequence set forth as SEQ ID NO: 206.

In embodiments, anchoring of a sortase signal-bearing protein at the surface of a cell can involve the following. A sortase signal-bearing protein is secreted to the outside of a cell due to the presence of an N-terminal signal sequence. Signal sequences are described elsewhere herein. Once at the surface of the cell, the sortase signal-bearing protein is held at the plasma membrane by the hydrophobic region and positively charged tail of the sortase signal. A class A sortase enzyme, located at the plasma membrane, cleaves between the threonine and glycine in the LPXTG sequence (SEQ ID NO: 206) of the sortase signal via a transpeptidation reaction, forming a thioester acyl bond between the sortase and the cleaved protein. The cleaved protein having the “LPXT” portion then is released from sortase and forms an intermediate with lipid II via nucleophilic attack. Lipid II includes the peptidoglycan precursors, N-acetylglucosamine and N-acetylmuramic acid, along with a pentapeptide peptidoglycan cross bridge. The cleaved protein having the “LPXT” portion is believed to attach to the pentapeptide cross bridge of lipid II. The protein having the “LPXT” portion is then covalently attached to the peptidoglycan of the cell wall as part of normal cell wall construction.

Sortase A (SrtA) is a membrane-anchored transpeptidase expressed by gram-positive bacteria including lactic acid bacteria, S. aureus, and S. pneumoniae (Call and Klaenhammer Frontiers in Microbiology 2013, 4, 73; Paterson and Mitchell Microbes Infect. 2006, 8(1), 145-153). In embodiments, an S. pneumoniae sortase A includes an amino acid described in GenBank accession no. CMY05290.1.

In embodiments, the present disclosure includes a protein D-sort fusion protein (i.e. protein D-sortase signal fusion protein) having an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an amino acid sequence set forth as SEQ ID NO: 223, wherein the protein D-sort fusion protein retains immunogenicity and anchoring at the cell surface; and/or having an amino acid sequence set forth as SEQ ID NO: 223. In embodiments, the present disclosure includes a protein D-sort fusion protein encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a nucleotide sequence set forth as SEQ ID NO: 224, wherein the nucleotide sequence encodes a protein D-sort fusion protein that retains immunogenicity and anchoring at the cell surface; and/or is encoded by a nucleotide sequence set forth as SEQ ID NO: 224. In embodiments, the present disclosure includes a fusion protein comprising a sortase signal, a protein D, and a UspA polypeptide. In embodiments, a protein D-sort fusion protein or a fusion protein comprising a sortase signal, a protein D, and a UspA polypeptide retains immunogenicity and anchoring at the cell surface when the protein D-sort fusion protein or a fusion protein comprising a sortase signal, a protein D, and a UspA polypeptide retains the ability to elicit an immunological response in a subject and retains its localization to the cell surface and attachment to the cell plasma membrane and/or to the cell wall of a cell as described herein.

### Lipoprotein Anchor

In embodiments, a surface anchor moiety includes a lipoprotein anchor. The lipoprotein anchor may be genetically fused to a heterologous immunogenic protein, or an immunogenic fragment or variant thereof, such that the lipoprotein anchor of the expressed fusion protein allows for attachment to the plasma membrane. A lipoprotein includes a soluble hydrophilic protein that is associated with a lipid bilayer through covalent attachment by a lipid anchor.

A lipoprotein anchor includes an N-terminal signal sequence with a signal peptidase (SPase II) cleavage site. The N-terminal signal sequence allows secretion of a target protein (e.g., H. influenzae protein D) to the outside of a cell. Upon SPase II-mediated cleavage, a lipid is coupled through an N-acyl diglyceride modification of the N-terminal cysteine residue of the SPase II-cleaved protein, allowing association of the protein to the cell membrane through the lipid moiety (Tjalsma & van Dijl. Proteomics 2005, 5, 4472-4482). In embodiments, a lipoprotein anchor includes a lipobox motif. In embodiments, a lipomotif box of the disclosure includes a consensus amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a consensus amino acid sequence set forth as X₁X₂X₃C, wherein the lipoprotein anchor retains surface anchoring activity; and/or includes a consensus amino acid sequence of X₁X₂X₃C, where X₁ is L (leucine), V (valine), or I (isoleucine); X₂ is A (alanine), S (serine), T (threonine), V (valine), or I (isoleucine); and X₃ is G (glycine), A (alanine), or S (serine). In embodiments, a lipoprotein anchor includes a consensus amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a consensus amino acid sequence set forth as SEQ ID NO: 212, wherein the lipoprotein anchor retains surface anchoring activity; and/or includes a consensus signal sequence of X₁RRX₂FLK (SEQ ID NO: 212), where X₁ is S (serine) or T (threonine); and where X₂ is any amino acid residue. In embodiments, the N-terminus of a heterologous immunogenic protein, or an immunogenic fragment or variant thereof, is fused to an N-terminal portion of a natural lipoprotein, with the N-terminus of the heterologous immunogenic protein, or an immunogenic fragment or variant thereof, C-terminal of a conserved cysteine of the N-terminal portion of the natural lipoprotein. In embodiments, lipoprotein anchors include ones from L. lactis oligopeptide binding protein OppA and L. lactis Nlp1-4 (Leenhouts et al. Antonie van Leeuwenhoek 1999,76, 367-376; Poquet & Gruss J. Bacteriol. 1998, 180, 1904-1912). In embodiments, a lipoprotein anchor that allows anchoring of a protein at the surface of a cell has an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an amino acid sequence set forth as SEQ ID NO: 210, wherein the lipoprotein anchor retains surface anchoring activity; and/or has an amino acid sequence set forth as SEQ ID NO: 210. In embodiments, a lipoprotein anchor that allows anchoring of a protein at the surface of a cell is encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a nucleotide sequence set forth as SEQ ID NO: 228, wherein the nucleotide sequence encodes a lipoprotein anchor that retains surface anchoring activity; and/or is encoded by a nucleotide sequence set forth as SEQ ID NO: 228. In embodiments, the present disclosure includes a protein D-lipoprotein anchor fusion protein having an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an amino acid sequence set forth as SEQ ID NO: 226, wherein the protein D-lipoprotein anchor fusion protein retains immunogenicity and anchoring at the cell surface; and/or having an amino acid sequence set forth as SEQ ID NO: 226. In embodiments, the present disclosure includes a protein D-lipoprotein anchor fusion protein encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a nucleotide sequence set forth as SEQ ID NO: 227, wherein the nucleotide sequence encodes a protein D-lipoprotein anchor fusion protein that retains immunogenicity and anchoring at the cell surface; and/or is encoded by a nucleotide sequence set forth as SEQ ID NO: 227. In embodiments, the present disclosure includes a fusion protein comprising a lipoprotein anchor, a protein D, and a UspA polypeptide. In embodiments, a protein D-lipoprotein anchor fusion protein or a fusion protein comprising a lipoprotein anchor, a protein D, and a UspA polypeptide retains immunogenicity and anchoring at the cell surface when the protein D-lipoprotein anchor fusion protein or a fusion protein comprising a lipoprotein anchor, a protein D, and a UspA polypeptide retains the ability to elicit an immunological response in a subject and retains its localization to the cell surface and attachment to the cell plasma membrane and/or to the cell wall of a cell as described herein.

One of skill in the art recognizes that other strategies to anchor a protein at a cell surface may be used. In embodiments, a fusion of at least one heterologous immunogenic protein, or an immunogenic fragment or variant thereof, with one or more transmembrane domains may be generated such that the fusion protein is anchored to the cell surface by interaction of the transmembrane domain with lipids in the plasma membrane. A transmembrane domain includes any polypeptide structure that is thermodynamically stable in a membrane. This is typically an alpha helix that includes several hydrophobic amino acid residues. The transmembrane domain of any transmembrane protein can be used. The presence and span of a transmembrane domain of a protein can be determined by those skilled in the art using known algorithms such as the TMHMM algorithm (World Wide Web at cbs.dtu.dk/services/TMHMM-2.0/). In embodiments, an artificially designed transmembrane domain may also be used (U.S. Pat. No. 7,052,906 describes synthetic transmembrane components).

In embodiments, a fusion of a heterologous immunogenic protein, or an immunogenic fragment or variant thereof, with a cell wall binding domain (including peptidoglycan-binding domains, teichoic acid-binding domains, domains of S-layer proteins, and poly-sugar-binding domains) may be generated such that the fusion protein is anchored to the cell wall. In embodiments, peptidoglycan-binding domains include LysM, SH3b, WxL domain, peptidoglycan-binding domain type 1, and cell wall binding domains of Listeria phage endolysins. Surface anchor moieties are described in, e.g., Leenhouts et al. Antonie van Leeuwenhoek 1999,76, 367-376; Visweswaran et al. Appl Microbiol Biotechnol 2014, 98, 4331-4345; Gründling and Schneewind J Bacteriol 2006, 188, 2463-2472; Lu et al. J Biol Chem 2006, 281, 549-558; Schmelcher et al. Future Microbiol 2012, 7, 1147-1171; Brinster et al. J Bacteriol 2007, 189, 1244-1253; Ghuysen et al. FEBS Lett 1994, 342, 23-28; Li et al. J Bacteriol 2011, 193, 197-204; Tolba et al. Analyst 2012, 137, 5749-5756; and Regulski et al. J Biol Chem 2013, 288, 20416-20426; Mathiesen et a. Scientific Reports 2020, 10, 9640; Call and Klaenhammer Frontiers in Microbiology 2013, 4, 73; and Desvaux et al. FEMS Microbiol Lett 2006, 256, 1-15.

### VII. Immunogenic Compositions

Immunogenic compositions are provided that include a recombinant, live attenuated S. pneumoniae expressing on its surface at least one heterologous immunogenic protein, or an immunogenic fragment or variant thereof. An “immunogenic composition” refers to a composition that can elicit or enhance an immune response. In some embodiments, an immunogenic composition includes a vaccine composition. An immunogenic composition may additionally include further components including, for example, an adjuvant or an immunomodulator.

The terms “vaccine” or “vaccine composition”, used interchangeably herein, refer to pharmaceutical compositions including at least one immunogenic composition that induces an immune response in a mammalian subject. A vaccine or vaccine composition may protect the mammalian subject from disease or possible death due to an infection, and may or may not include one or more additional components that enhance the immunological activity of the active component. A vaccine or vaccine composition may additionally include further components typical to pharmaceutical compositions, for example, an adjuvant or an immunomodulator. In embodiments, a “vaccine composition” includes a formulation including a recombinant, live attenuated S. pneumoniae genetically engineered to express on its surface at least one heterologous immunogenic protein involved in mammalian transmission of at least one pathogen including S. pneumoniae. The vaccine composition is in a form suitable for administration to a subject that results in a reduction in the transmissibility of the at least one pathogen including S. pneumoniae upon infection.

The presently disclosed immunogenic compositions include a recombinant, live attenuated S. pneumoniae genetically engineered to express on its surface at least one heterologous immunogenic protein, or an immunogenic fragment or variant thereof. Immunogenic and vaccine compositions of the disclosure can include a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. A pharmaceutically acceptable carrier can include a diluent, adjuvant, excipient, stabilizer, preservative, and/or vehicle with which a vaccine composition is administered. A pharmaceutically acceptable carrier can include: a sterile liquid, such as water and oils, including those of petroleum, animal, vegetable or synthetic origin (e.g., peanut oil, soybean oil, mineral oil, sesame oil and the like); saline solutions (e.g., phosphate-buffered saline, Ringer's solutions); aqueous dextrose solutions; and glycerol solutions.

Immunogenic and vaccine compositions of the disclosure may include auxiliary substances such as: wetting or emulsifying agents; surfactants; pH buffering agents (e.g., citrate buffer containing sucrose, bicarbonate buffer alone, bicarbonate buffer containing ascorbic acid, lactose); gelling or viscosity enhancing additives; preservatives (e.g., thimerosal and/or EDTA); and flavoring agents. The pharmaceutically acceptable carrier is non-toxic and does affect the biological activity of the vaccine composition. Suitable pharmaceutical carriers are described in “Remington's Pharmaceutical Sciences” by E. W. Martin, 18^{th} Edition. A wide variety of carriers are well known in the art, and the selection of specific carriers is well within the level of those skilled in the art. Supplementary active compounds can also be incorporated into a vaccine composition of the disclosure. In embodiments, the pharmaceutically acceptable carrier is not normally found in nature or not normally found in nature in combination with the recombinant, live attenuated S. pneumoniae.

In embodiments, the immunogenic or vaccine composition is in bulk or in unit dosage form. The unit dosage form is any of a variety of forms, including, for example, a capsule, an IV bag, a tablet, a single pump on an aerosol inhaler, or a vial. The quantity of active ingredient in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like.

Dosage forms for the topical or transdermal administration of an immunogenic or a vaccine composition of the disclosure include powders, sprays, ointments, pastes, creams, lotions, gels, solutions, patches, and inhalants. In embodiments, the active agent (i.e., a recombinant, live attenuated S. pneumoniae genetically engineered to express on its surface at least one heterologous immunogenic protein, or an immunogenic fragment or variant thereof) is mixed under sterile conditions with a pharmaceutically acceptable carrier, and with any preservatives, buffers or propellants that are required. In embodiments, the immunogenic or vaccine composition includes a solution, dispersion, suspension, powder, capsule, tablet, pill, time release capsule, time release tablet, and/or time release pill.

Immunogenic or vaccine compositions suitable for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active agent in the required amount in an appropriate solvent with one or a combination of ingredients enumerated herein, as required, followed by filter sterilization. Generally, dispersions are prepared by incorporating the active agent into a sterile vehicle that contains a basic dispersion medium and the required other ingredients from those enumerated herein. In the case of sterile powders for the preparation of sterile injectable solutions, methods of preparation are vacuum drying and freeze-drying that yields a powder of the active agent plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active agent can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In embodiments, an immunogenic or a vaccine composition of the disclosure is formulated for intranasal administration (i.e., inhalation) or pulmonary delivery. For administration by inhalation, the compounds are delivered in the form of an aerosol spray from a pressured container or dispenser, which contains a suitable propellant, e.g., a gas such as carbon dioxide, or a nebulizer.

In embodiments, an immunogenic or a vaccine composition of the disclosure can also be formulated for systemic administration, such as by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

An immunogenic or vaccine composition of the present disclosure may include an adjuvant. Alternatively, the immunogenic or vaccine composition is separate from the adjuvant. Many substances, both natural and synthetic, have been shown to function as adjuvants. For example, adjuvants may include, but are not limited to, mineral salts, squalene mixtures, muramyl peptide, saponin derivatives, mycobacterium cell wall preparations, certain emulsions, mineral gels such as aluminum hydroxide, surface active substances such as lysolecithin, pluronic polyols, polyanions, peptides, oil or hydrocarbon emulsions, dinitrophenol, monophosphoryl lipid A, mycolic acid derivatives, nonionic block copolymer surfactants, Quil A, complete Freund's adjuvant, incomplete Freund's adjuvant, cholera toxin B subunit, polyphosphazene and derivatives, immunostimulating complexes (ISCOMs), cytokine adjuvants, MF59 adjuvant, lipid adjuvants, mucosal adjuvants, certain bacterial exotoxins and other components, certain oligonucleotides, poly-α-L-glutamine (PLG), and other adjuvants.

### VIII. Methods of Use

Methods are provided for inducing an immune response in a subject by administering an immunogenic composition comprising a recombinant, live attenuated S. pneumoniae expressing on its surface at least one heterologous immunogenic protein, or an immunogenic fragment or variant thereof. The at least one heterologous immunogenic protein, or an immunogenic fragment or variant thereof can be expressed on the surface of the recombinant, live attenuated S. pneumoniae as a fusion protein comprising a surface anchor moiety. The induced immune response includes an antibody immune response and/or a cell-mediated immune response as described elsewhere herein. The induction of an immune response can prevent or reduce onset, duration, severity, or a combination thereof, of symptoms of at least one disease caused by at least one pathogen. The at least one pathogen can include S. pneumoniae, H. influenzae, and/or M. catarrhalis.

In embodiments, methods are provided for reducing the mammalian transmission of at least one pathogen including S. pneumoniae by administering to a mammalian subject infected with at least one pathogen including S. pneumoniae or at risk of infection by at least one pathogen including S. pneumoniae an immunogenic composition comprising a recombinant, live attenuated S. pneumoniae expressing on its surface at least one heterologous immunogenic protein, or an immunogenic fragment or variant thereof, and a non-naturally occurring pharmaceutically acceptable carrier. In embodiments, the at least one heterologous immunogenic protein, or an immunogenic fragment or variant thereof, is required for or involved in mammalian transmission of at least one pathogen including S. pneumoniae.

In embodiments, the at least one heterologous immunogenic protein includes protein D, or an immunogenic fragment or variant thereof. In embodiments, the protein D is from H. influenzae. In embodiments, the at least one heterologous immunogenic protein includes a UspA polypeptide, or an immunogenic fragment or variant thereof. In embodiments, the UspA polypeptide is from M. catarrhalis.

In embodiments, a fusion protein comprising the at least one heterologous immunogenic protein, or an immunogenic fragment or variant thereof, useful for the methods has an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an amino acid sequence set forth as any one of SEQ ID NOs: 219, 223, and 226, wherein the at least one heterologous immunogenic protein, or an immunogenic fragment or variant thereof, retains immunogenicity; and/or the fusion protein comprising the at least one heterologous immunogenic protein, or an immunogenic fragment or variant thereof, has an amino acid sequence set forth as any one of SEQ ID NOs: 219, 223, and 226.

In embodiments, the at least one heterologous immunogenic protein, or an immunogenic fragment or variant thereof, useful for the methods has an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an amino acid sequence set forth as any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 213, 261, and 263, wherein the at least one heterologous immunogenic protein retains immunogenicity; and/or has an amino acid sequence set forth as any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 213, 261, and 263.

As used herein, a “subject” can be any mammal, e.g., a human, primate, bird, mouse, rat, fowl, dog, cat, cow, horse, goat, rabbit, camel, sheep or a pig. In embodiments, the mammal is a human. In embodiments, the human being administered the immunogenic composition can be a newborn, infant, toddler, preadolescent, adolescent, or adult. In embodiments, the subject is a child.

In embodiments, the subject is infected with at least one pathogen or is at risk of infection by at least one pathogen. The at least one pathogen includes S. pneumoniae, H. influenzae, M. catarrhalis, or a combination thereof. Although any individual has a certain risk of becoming infected with at least one pathogen, certain sub-populations have an increased risk of infection. Those with a higher risk of infection include, but are not limited to, subjects whose immune system is compromised and/or have chronic illnesses, newborns, infants, toddlers, seniors, children or adults with asplenia, splenic dysfunction, sickle-cell disease, cochlear implants or cerebrospinal fluid leaks, childcare workers, and healthcare workers.

As used herein, the term “transmission” refers to the mammal-to-mammal spread of at least one pathogen (e.g., S. pneumoniae, H. influenzae, M. catarrhalis, or a combination thereof) by direct contact with respiratory secretions, such as saliva or mucus. In embodiments, the presently disclosed compositions and methods reduce transmission of at least one pathogen (e.g., S. pneumoniae, H. influenzae, M. catarrhalis, or a combination thereof) from a mother to an offspring—prenatally, postnatally, or both.

Administration of an immunogenic composition described herein can induce an immune response in a subject. In embodiments, the induced immune response is an antibody immune response and/or a cell-mediated immune response. A subject administered an immunogenic composition of the disclosure may induce an antibody immune response that is at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.5-fold, at least 3-fold, at least 3.5-fold, at least 4-fold, at least 4.5-fold, at least 5-fold, at least 10-fold, or greater as compared to the subject not administered the immunogenic composition. In embodiments, the antibody titer (expressed in terms of log 10 value) from the serum of a subject treated with an immunogenic composition of the disclosure is at least 0.05, at least 0.10, at least 0.15, at least 0.20, at least 0.25, or at least 0.30 higher than that of the subject not administered the immunogenic composition. A subject administered an immunogenic composition of the disclosure may induce a cell-mediated immune response that is at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.5-fold, at least 3-fold, at least 3.5-fold, at least 4-fold, at least 4.5-fold, at least 5-fold, at least 10-fold, or greater as compared to the subject not administered the immunogenic composition. A subject administered an immunogenic composition of the disclosure may induce a memory T cell population that is at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.5-fold, at least 3-fold, at least 3.5-fold, at least 4-fold, at least 4.5-fold, at least 5-fold, at least 10-fold, or greater as compared to the subject not administered the immunogenic composition. Assays to measure antibody immune responses and cell-mediated immune responses are described elsewhere herein.

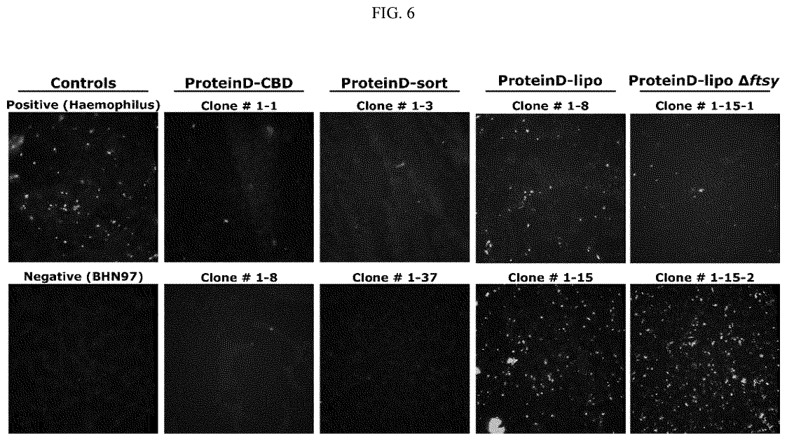

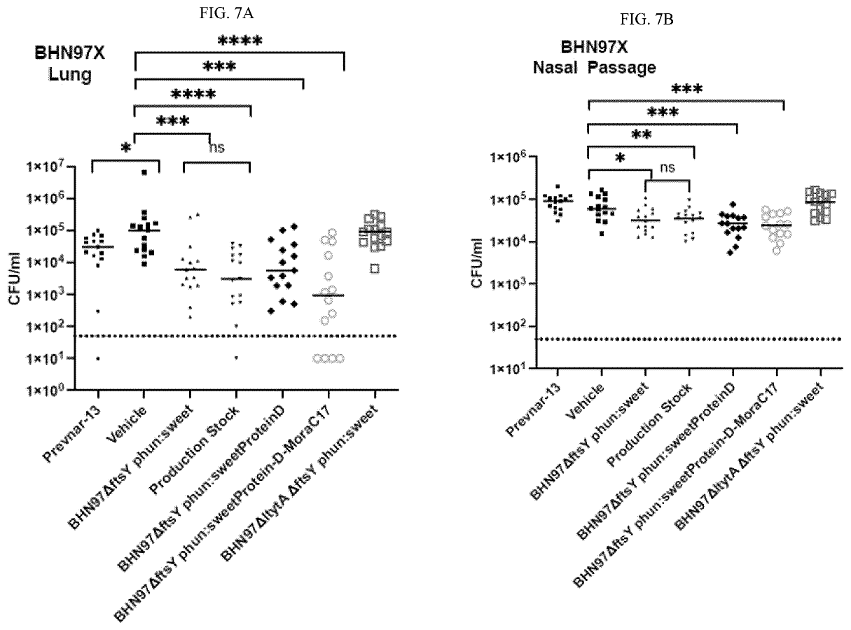

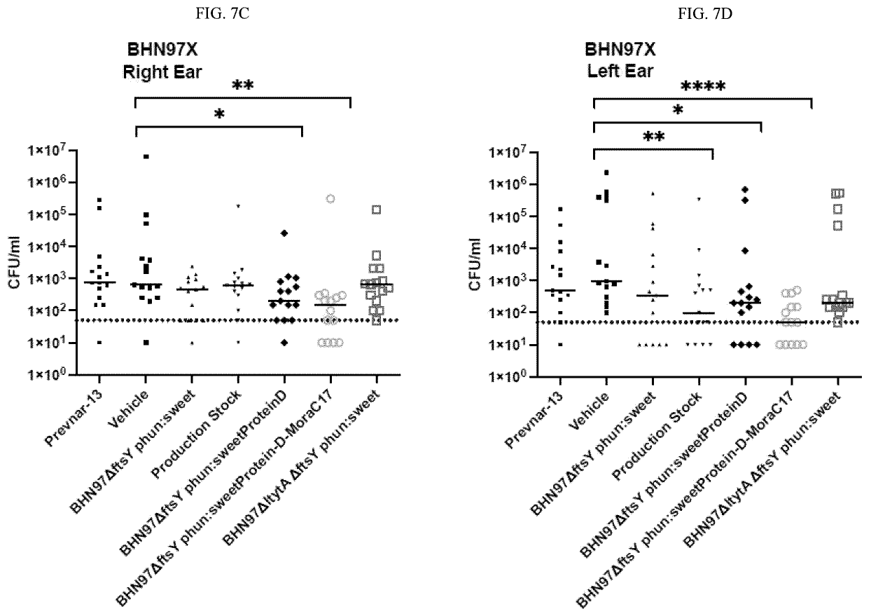

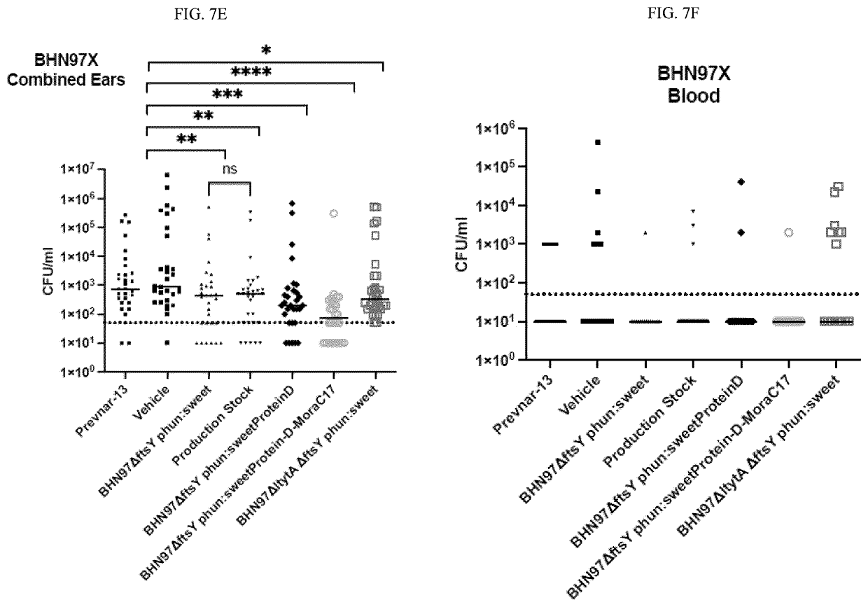

Administration of an immunogenic composition described herein can enhance an immune response in a subject against S. pneumoniae as compared to the subject administered a control immunogenic composition (see FIGS. 7A-7F). In embodiments, the enhanced immune response is an antibody immune response and/or a cell-mediated immune response. A subject administered an immunogenic composition of the disclosure may have an enhanced antibody immune response that is at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.5-fold, at least 3-fold, at least 3.5-fold, at least 4-fold, at least 4.5-fold, at least 5-fold, at least 10-fold, or greater as compared to the subject administered a control immunogenic composition. In embodiments, the antibody titer (expressed in terms of log 10 value) from the serum of a subject treated with an immunogenic composition of the disclosure is at least 0.05, at least 0.10, at least 0.15, at least 0.20, at least 0.25, or at least 0.30 higher than that of the subject administered a control immunogenic composition. A subject administered an immunogenic composition of the disclosure may have an enhanced cell-mediated immune response that is at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.5-fold, at least 3-fold, at least 3.5-fold, at least 4-fold, at least 4.5-fold, at least 5-fold, at least 10-fold, or greater as compared to the subject administered a control immunogenic composition. A subject administered an immunogenic composition of the disclosure may have a memory T cell population that is at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.5-fold, at least 3-fold, at least 3.5-fold, at least 4-fold, at least 4.5-fold, at least 5-fold, at least 10-fold, or greater as compared to the subject administered a control immunogenic composition. In embodiments, a control immunogenic composition does not contain at least one component of the immunogenic composition of the disclosure. In embodiments, a control immunogenic composition includes the same recombinant live attenuated S. pneumoniae as the administered immunogenic composition but does not express on its cell surface the at least one heterologous immunogenic protein, or an immunogenic fragment or variant thereof. In embodiments, a control immunogenic composition includes a known immunogenic composition available as standard of care for prevention or reduction of at least one disease, or its symptoms, caused by S. pneumoniae (e.g., Prevnar-13, Wyeth Pharmaceuticals, Inc.).

The compositions and methods disclosed herein result in the reduced mammalian transmission of at least one pathogen including S. pneumoniae. A pathogen refers to an organism that can cause disease, including bacteria, viruses, fungi, protozoa, and worms. In embodiments, compositions and methods of the disclosure are useful against pathogens including S. pneumoniae, H. influenzae, and/or M. catarrhalis. In embodiments, the at least one pathogen includes S. pneumoniae and H. influenzae. In embodiments, the at least one pathogen includes S. pneumoniae and M. catarrhalis. In embodiments, a mammal that has been administered an immunogenic composition disclosed herein is less likely to transmit the least one pathogen including S. pneumoniae if or when the mammal is infected with the least one pathogen including S. pneumoniae as compared to a control. In embodiments, a control includes a mammal that has not received the immunogenic composition. In embodiments, a control includes a mammal that has received an immunogenic composition that does not contain at least one component of the immunogenic composition of the disclosure. In embodiments, a control includes a mammal that has been administered an immunogenic composition comprising the same recombinant live attenuated S. pneumoniae as the administered immunogenic composition but does not express on its cell surface the at least one heterologous immunogenic protein, or an immunogenic fragment or variant thereof. In embodiments, the transmission rate from a mammal or population thereof administered an immunogenic composition of the disclosure) to another mammal or population thereof that has or has not been administered an immunogenic composition is reduced by at least about 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100% as compared to the transmission rate of a mammal or population thereof who were not administered the immunogenic composition. A “population” refers to a group of more than one subject.

The mammalian transmission rate can be measured using any method known in the art. For example, mammalian transmission rates can be determined by measuring the colonization of at least one pathogen including S. pneumoniae within members of a population comprising at least one individual subject that has been infected with the at least one pathogen including S. pneumoniae and wherein all other members of the population have been brought into physical contact with the infected individual(s), followed by determining the infection burden of the contact mammals by quantification of viable bacteria and/or pathogen present in the anterior nares by nasal lavage of the nares and culturing lavage fluid, or by direct contact of the anterior nares with bacteriological growth media.

As used herein, “mammalian transmissibility” refers to the ability of a pathogen (e.g., S. pneumoniae, H. influenzae, and/or M. catarrhalis bacteria) to be transmitted from one infected mammal to another mammal by direct contact with respiratory secretions, such as saliva or mucus.

Methods are also provided for reducing the incidence rate of at least one disease caused by at least one pathogen in a mammalian population by administering to at least one mammalian subject within the mammalian population an immunogenic composition comprising a recombinant, live attenuated S. pneumoniae expressing on its surface at least one heterologous immunogenic protein, or an immunogenic fragment or variant thereof. The at least one pathogen can include S. pneumoniae, H. influenzae, and/or M. catarrhalis. In embodiments, the at least one heterologous immunogenic protein, or an immunogenic fragment or variant thereof, is required for or involved in mammalian transmission of at least one pathogen.

In embodiments, a disease is considered “invasive” when infection occurs in the blood, cerebrospinal fluid, pleural fluid, joint fluid, peritoneal fluid, or other normally sterile sites. Non-limiting examples of invasive diseases whose incidence rates are reduced by compositions and methods of the disclosure include pneumonia, bacteremia, septicemia, meningitis, septic arthritis, osteomyelitis, peritonitis, and endocarditis. In embodiments, a disease is considered “non-invasive” when infection occurs outside of the major organs and the blood. For example, non-invasive diseases whose incidence rates are reduced by compositions and methods of the disclosure include acute otitis media, sinusitis, and bronchitis. In embodiments, diseases whose incidence rates are reduced by compositions and methods of the disclosure include pneumococcal diseases including acute otitis media, sinusitis, bronchitis, pneumonia, bacteremia, septicemia, and meningitis. In embodiments, diseases whose incidence rates are reduced by compositions and methods of the disclosure include upper respiratory tract infections.

Acute otitis media (AOM) is a middle ear infection involving inflammation and a build-up of fluid behind the eardrum. In embodiments, infants between six and 15 months old are most commonly affected. Symptoms of AOM include: earache, fever, a lack of energy, hearing loss, coughing, runny nose, loss of balance, or a combination thereof. Most AOM clear up within 3 to 5 days without treatment. Paracetamol or ibuprofen is used to relieve pain and fever. Antibiotics may occasionally be prescribed if symptoms persist or are particularly severe.

Sinusitis is an inflammation of the lining of the sinuses, which are small, air-filled cavities behind the cheekbones and forehead. Channels in the sinuses allow mucus to drain into the nose, and these channels become blocked when the sinus lining is inflamed. Symptoms of sinusitis include: discharge from the nose; a blocked nose; pain and tenderness around the cheeks, eyes or forehead; a sinus headache; a fever; a toothache; a reduced sense of smell; bad breath; or a combination thereof. Sinusitis usually clears after a few weeks. Corticosteroid sprays or drops and/or antibiotics may be prescribed for persistent symptoms.

Bronchitis is an infection of the main airways of the lungs (bronchi), causing the bronchi to become irritated and inflamed. Symptoms can include: a cough, sometimes associated with phlegm; a sore throat; wheezing; or a combination thereof. Complications of bronchitis can lead to pneumonia. Pneumonia involves inflammation of the tissue in one or both lungs. Symptoms include: a cough with or without phlegm; difficulty breathing; rapid heartbeat; fever; sweating and shivering; loss of appetite; chest pain; coughing up blood; headaches; fatigue; nausea or vomiting; wheezing; joint and muscle pain; confusion and/or disorientation; or a combination thereof. Pneumonia can be treated by getting plenty of rest and fluids and/or by taking antibiotics. Complications of pneumonia include inflammation of the lining between the lungs and ribcage (pleurisy), a lung abscess, and/or an infection of the blood (bacteremia or septicemia).

Meningitis occurs when protective membranes surrounding the brain and spinal cord are infected. If not treated promptly, meningitis can lead to life-threatening septicemia and/or damage to the brain or nerves. Symptoms of meningitis include: fever); a headache; a blotchy rash; a stiff neck; sensitivity to bright lights; drowsiness; unresponsiveness; seizures; or a combination thereof. Meningitis can be treated with intravenous administration of antibiotics and/or fluids, or oxygen through a face mask.

The incidence rate of at least one disease caused by at least one pathogen within a population can be reduced by the presently disclosed methods and compositions. As used herein, “incidence rate” refers to the numbers or percentage of subjects within a population that have newly acquired a disease. Administering to at least one mammalian subject within a mammalian population a presently disclosed immunogenic composition can reduce the incidence rate within the population of a disease caused by at least one pathogen including S. pneumoniae by at least about 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100% as compared to a mammalian population in which none of the individual mammals were administered the presently disclosed immunogenic composition.

The presently disclosed immunogenic compositions can be administered in an effective amount in order to reduce the mammalian transmission of at least one pathogen or to reduce the incidence rate of a disease caused by at least one pathogen. The at least one pathogen can include S. pneumoniae, H. influenzae, and/or M. catarrhalis. In embodiments, an “effective amount” of an immunogenic composition can be an amount sufficient to achieve the desired result (i.e., induced immune response, reduced mammalian transmission of at least one pathogen, or reduced incidence rate of a disease caused by at least one pathogen). An immunogenic composition is administered to a subject prior to infection by at least one pathogen or prior to a disease caused by at least one pathogen or is administered to a subject that has been infected by at least one pathogen or that has a disease caused by at least one pathogen or is exhibiting symptoms of the same.

Generally, the presently disclosed immunogenic compositions are administered in order to reduce the transmission of at least one pathogen to other subjects within a population. In embodiments, administration of the presently disclosed immunogenic compositions prevents or reduces the level of colonization and/or the duration of colonization (e.g., of the nasopharynx or other body tissues) by at least one pathogen. In embodiments, an immunogenic composition of the disclosure functions prophylactically, and even therapeutically, in the subject to which it has been administered to prevent or reduce colonization and subsequent disease within the subject administered the immunogenic composition of the disclosure. In embodiments, an immunogenic composition of the disclosure confers protective immunity, allowing an individual administered an immunogenic composition of the disclosure to exhibit delayed onset of symptoms or reduced severity of symptoms, as the result of their exposure to the immunogenic composition. In embodiments, the reduction in severity of symptoms is at least 25%, 40%, 50%, 60%, 70%, 80%, or at least 90%. In embodiments, individuals administered an immunogenic composition of the disclosure may display no symptoms upon contact with at least one pathogen (e.g., S. pneumoniae, H. influenzae, and/or M. catarrhalis), do not become colonized by at least one pathogen, or both. In embodiments, administration of the presently disclosed immunogenic compositions prevents or reduces onset, duration, severity, or a combination thereof, of symptoms of at least one disease caused by at least one pathogen. The at least one pathogen can include S. pneumoniae, H. influenzae, and/or M. catarrhalis. The at least one disease is selected from the group consisting of acute otitis media, sinusitis, bronchitis, pneumonia, bacteremia, septicemia, and meningitis. In embodiments, the at least one disease is acute otitis media.

The specific effective dose level for any particular subject will depend upon a variety of factors including the specific immunogenic composition employed; the age, body weight, general health, sex and diet of the patient; the time of administration; the route of administration; the rate of excretion of the immunogenic composition employed; the duration of the treatment; drugs used in combination or coincidental with the specific compound employed; and like factors well known in the medical arts (see e.g., Koda-Kimble et al., (2004), Applied Therapeutics: The Clinical Use of Drugs, Lippincott Williams & Wilkins, ISBN 0781748453; Winter, (2003), Basic Clinical Pharmacokinetics, 4.sup.th ed., Lippincott Williams & Wilkins, ISBN 0781741475; Sharqel, (2004), Applied Biopharmaceutics & Pharmacokinetics, McGraw-Hill/Appleton & Lange, ISBN 0071375503). If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

In embodiments, an effective amount of an immunogenic composition includes a minimum of about 10³ recombinant live attenuated S. pneumoniae expressing on its surface at least one heterologous immunogenic protein, or an immunogenic fragment or variant thereof. In embodiments, an effective amount ranges from 10³ colony forming units/ml (CFU/ml) to 10¹⁵ CFU/ml, or 10³ CFU/ml to 10¹² CFU/ml, or 10³ CFU/ml to 10⁹ CFU/ml. The term “colony forming unit” or “CFU” is a unit of measure used to indicate the number of organisms (e.g., recombinant live attenuated S. pneumoniae expressing on its surface at least one heterologous immunogenic protein, or an immunogenic fragment or variant thereof) capable of replication in a given sample. This is based on the theory that a colony is derived from the replication of a pair/cluster or single cell of bacteria.

Suitable routes of administering the immunogenic compositions of the disclosure include: intranasal, topical, subcutaneous, transdermal, intradermal, intraperitoneal, transmucosal, transtympanic, intraorgan, intrathecal, intramuscular, intravenous, and intravascular administration.

In embodiments, an immunogenic composition described herein is administered to a subject by injection, inhalation (e.g., of an aerosol), by means of a catheter, or by means of an implant, the implant being of a porous, non-porous, or gelatinous material, including a membrane, such as a sialastic membrane, or a fiber.

Moreover, the administration may be by continuous infusion or by single or multiple boluses. In embodiments, an immunogenic composition can be infused over a period of less than 4 hours, less than 3 hours, less than 2 hours, or less than 1 hour. In embodiments, the infusion occurs slowly at first and then is increased overtime.

Administration of immunogenic compositions described herein can occur as a single event, a periodic event, or over a time course of treatment. For example, immunogenic compositions can be administered daily, weekly, bi-weekly, or monthly. As another example, immunogenic compositions can be administered in multiple treatment sessions, such as 2 weeks on, 2 weeks off, and then repeated twice; or every 3rd day for 3 weeks.

### Ix. Non-Limiting Embodiments Include

1. An immunogenic composition comprising:
a recombinant live attenuated Streptococcus pneumoniae (S. pneumoniae) expressing on its cell surface at least one heterologous immunogenic protein, or an immunogenic fragment or variant thereof.

2. The immunogenic composition of embodiment 1, wherein the at least one heterologous immunogenic protein, or an immunogenic fragment or variant thereof, comprises a Haemophilus influenzae (H. influenzae) polypeptide.

3. The immunogenic composition of embodiment 2, wherein the H. influenzae polypeptide comprises protein D.

4. The immunogenic composition of embodiment 3, wherein the protein D:
(i) comprises an amino acid sequence having at least 80% sequence identity to an amino acid sequence set forth as SEQ ID NO: 213, wherein said protein D retains immunogenicity;
(ii) comprises the amino acid sequence set forth as SEQ ID NO: 213;
(iii) is encoded by a nucleotide sequence having at least 80% sequence identity to a nucleotide sequence set forth as SEQ ID NO: 214, wherein the nucleotide sequence encodes a protein D that retains immunogenicity; and/or
(iv) is encoded by the nucleotide sequence set forth as SEQ ID NO: 214.

5. The immunogenic composition of any one of embodiments 1-4, wherein the at least one heterologous immunogenic protein, or an immunogenic fragment or variant thereof, comprises a Moraxella catarrhalis (M. catarrhalis) polypeptide.

6. The immunogenic composition of embodiment 5, wherein the M. catarrhalis polypeptide comprises a UspA polypeptide.

7. The immunogenic composition of embodiment 6, wherein the UspA polypeptide:
(i) comprises an amino acid sequence having at least 80% sequence identity to an amino acid sequence set forth as SEQ ID NO: 261 or 263, wherein the UspA polypeptide retains immunogenicity;
(ii) comprises the amino acid sequence set forth as SEQ ID NO: 261 or 263;
(iii) is encoded by a nucleotide sequence having at least 80% sequence identity to a nucleotide sequence set forth as SEQ ID NO: 262 or 264, wherein the nucleotide sequence encodes a UspA polypeptide that retains immunogenicity; and/or
(iv) is encoded by the nucleotide sequence set forth as SEQ ID NO: 262 or 264.

8. The immunogenic composition of any one of embodiments 1-7, wherein the at least one heterologous immunogenic protein, or an immunogenic fragment or variant thereof comprises an S. pneumoniae polypeptide.

9. The immunogenic composition of embodiment 8, wherein the S. pneumoniae polypeptide comprises:
(i) an amino acid sequence having at least 80% sequence identity to an amino acid sequence set forth as any one of SEQ ID NOs: 1-205, wherein the S. pneumoniae polypeptide retains immunogenicity; and/or
(ii) the amino acid sequence set forth as any one of SEQ ID NOs: 1-205.

10. The immunogenic composition of any one of embodiments 1-9, wherein the at least one heterologous immunogenic protein, or an immunogenic fragment or variant thereof, comprises a surface anchor moiety.

11. The immunogenic composition of embodiment 10, wherein the at least one heterologous immunogenic protein, or an immunogenic fragment or variant thereof, and the surface anchor moiety are linked as a fusion protein.

12. The immunogenic composition of embodiment 10 or 11, wherein the surface anchor moiety is C-terminal to the at least one heterologous immunogenic protein, or an immunogenic fragment or variant thereof.

13. The immunogenic composition of embodiment 10 or 11, wherein the surface anchor moiety is N-terminal to the at least one heterologous immunogenic protein, or an immunogenic fragment or variant thereof.

14. The immunogenic composition of any one of embodiments 11-13, wherein the fusion protein comprises:
(i) an amino acid sequence having at least 80% sequence identity to an amino acid sequence set forth as any of SEQ ID NOs: 219, 223, and 226, wherein said fusion protein retains immunogenicity and anchoring at the cell surface; and/or
(ii) the amino acid sequence set forth as any of SEQ ID NOs: 219, 223, and 226.

15. The immunogenic composition of any one of embodiments 11-14, wherein the fusion protein is encoded by:
(i) a nucleotide sequence having at least 80% sequence identity to a nucleotide sequence set forth asset forth as any of SEQ ID NOs: 220, 224, and 227, wherein the nucleotide sequence encodes a fusion protein that retains immunogenicity and anchoring at the cell surface; and/or
(ii) the nucleotide sequence set forth asset forth as any of SEQ ID NOs: 220, 224, and 227.

16. The immunogenic composition of any one of embodiments 10-15, wherein the surface anchor moiety comprises a choline-binding domain (CBD).

17. The immunogenic composition of embodiment 16, wherein the CBD comprises a choline binding repeat comprising:
(i) a consensus amino acid sequence having at least 80% sequence identity to an amino acid sequence set forth asset forth as SEQ ID NO: 215, wherein said CBD retains surface anchoring activity; and/or
(ii) the consensus amino acid sequence set forth asset forth as SEQ ID NO: 215.

18. The immunogenic composition of embodiment 16 or 17, wherein the CBD:
(i) comprises an amino acid sequence having at least 80% sequence identity to an amino acid sequence set forth asset forth as SEQ ID NO: 216, wherein said CBD retains surface anchoring activity;
(ii) comprises the amino acid sequence set forth as SEQ ID NO: 216;
(iii) is encoded by a nucleotide sequence having at least 80% sequence identity to a nucleotide sequence set forth as SEQ ID NO: 217, wherein the nucleotide sequence encodes a CBD that retains surface anchoring activity; and/or
(iv) is encoded by the nucleotide sequence set forth as SEQ ID NO: 217.

19. The immunogenic composition of any one of embodiments 10-15, wherein the surface anchor moiety comprises a sortase signal.

20. The immunogenic composition of embodiment 19, wherein the sortase signal comprises:
(i) a consensus amino acid sequence having at least 80% sequence identity to an amino acid sequence set forth as SEQ ID NO: 206, wherein said sortase signal retains surface anchoring activity; and/or
(ii) the consensus amino acid sequence set forth as SEQ ID NO: 206.

21. The immunogenic composition of embodiment 19 or 20, wherein the sortase signal:
(i) comprises an amino acid sequence having at least 80% sequence identity to an amino acid sequence set forth as SEQ ID NO: 221, wherein said sortase signal retains surface anchoring activity;
(ii) comprises the amino acid sequence set forth as SEQ ID NO: 221;
(iii) is encoded by a nucleotide sequence having at least 80% sequence identity to a nucleotide sequence set forth as SEQ ID NO: 222, wherein the nucleotide sequence encodes a sortase signal that retains surface anchoring activity; and/or
(iv) is encoded by the nucleotide sequence set forth as SEQ ID NO: 222.

22. The immunogenic composition of any one of embodiments 10-15, wherein the surface anchor moiety comprises a lipoprotein anchor.

23. The immunogenic composition of embodiment 22, wherein the lipoprotein anchor comprises a lipobox motif comprising:
(i) a consensus amino acid sequence having at least 80% sequence identity to an amino acid sequence set forth as X₁X₂X₃C, wherein said lipoprotein anchor retains surface anchoring activity; and/or
(ii) the consensus amino acid sequence set forth as X₁X₂X₃C, wherein X₁ is L, V, or I; X₂ is A, S, T, V, or I; and X₃ is G, A, or S.

24. The immunogenic composition of embodiment 22 or 23, wherein the lipoprotein anchor:
(i) comprises an amino acid sequence having at least 80% sequence identity to an amino acid sequence set forth as SEQ ID NO: 210, wherein said lipoprotein anchor retains surface anchoring activity;
(ii) comprises the amino acid sequence set forth as SEQ ID NO: 210;
(iii) is encoded by a nucleotide sequence having at least 80% sequence identity to a nucleotide sequence set forth as SEQ ID NO: 228, wherein the nucleotide sequence encodes a lipoprotein anchor that retains surface anchoring activity; and/or
(iv) is encoded by the nucleotide sequence set forth as SEQ ID NO: 228.

25. The immunogenic composition of any one of embodiments 1-24, wherein the at least one heterologous immunogenic protein, or an immunogenic fragment or variant thereof, is attached to the surface covalently or non-covalently.

26. The immunogenic composition of any one of embodiments 1-25, wherein the recombinant live attenuated S. pneumoniae expresses on its surface at least two heterologous immunogenic proteins, or immunogenic fragments or variants thereof.

27. The immunogenic composition of embodiment 26, wherein the at least two heterologous immunogenic proteins, or immunogenic fragments or variants thereof are from a single pathogen.

28. The immunogenic composition of embodiment 27, wherein the single pathogen is H. influenzae.

29. The immunogenic composition of embodiment 27, wherein the single pathogen is S. pneumoniae.

30. The immunogenic composition of embodiment 27, wherein the single pathogen is M. catarrhalis.

31. The immunogenic composition of embodiment 26, wherein the at least two heterologous immunogenic proteins, or immunogenic fragments or variants thereof, are from different pathogens.

32. The immunogenic composition of embodiment 31, wherein the different pathogens comprise a combination of pathogens selected from S. pneumoniae, H. influenzae, and M. catarrhalis.

33. The immunogenic composition of any one of embodiments 10-25, wherein the recombinant live attenuated S. pneumoniae expresses on its surface at least two heterologous immunogenic proteins, or immunogenic fragments or variants thereof, and wherein the at least two heterologous immunogenic proteins, or immunogenic fragments or variants thereof, and the surface anchor moiety are expressed as one fusion protein.

34. The immunogenic composition of any one of embodiments 1-33, wherein the recombinant live attenuated S. pneumoniae comprises a disruption of an ftsY gene in its genome.

35. The immunogenic composition of any one of embodiments 1-34, wherein the recombinant live attenuated S. pneumoniae comprises in its genome the nucleotide sequence set forth as any of SEQ ID NOs: 229, 230, 231, 232, 233, and 234.

36. The immunogenic composition of any one of embodiments 1-35, further comprising an immunological adjuvant.

37. The immunogenic composition of any one of embodiments 1-36, wherein the immunogenic composition is formulated for intranasal administration.

38. The immunogenic composition of any one of embodiments 1-37, wherein the immunogenic composition is a vaccine composition.

39. A method for inducing an immune response, the method comprising administering to a subject in need thereof a therapeutically effective amount of the immunogenic composition of any one of embodiments 1-38.

40. The method of embodiment 39, wherein the induced immune response is an antibody immune response and/or a cell-mediated immune response.

41. The method of embodiment 39 or 40, wherein the administering prevents or reduces onset, duration, severity, or a combination thereof, of symptoms of at least one disease caused by at least one pathogen.

42. The method of embodiment 41, wherein the at least one pathogen comprises S. pneumoniae.

43. The method of any one of embodiment 39-42, wherein the administering reduces the incidence rate of the at least one disease in a mammalian population.

44. The method of any one of embodiments 41-43, wherein the at least one pathogen further comprises H. influenzae.

45. The method of any one of embodiments 41-44, wherein the at least one pathogen further comprises M. catarrhalis.

46. The method of any one of embodiments 41-45, wherein the immunogenic composition is administered to the subject intranasally.

47. The method of any one of embodiments 41-46, wherein the method reduces the transmission of the at least one pathogen from a mother to its offspring.

48. The method of any one of embodiments 41-47, wherein the at least one disease is selected from the group consisting of acute otitis media, sinusitis, bronchitis, pneumonia, bacteremia, septicemia, and meningitis.

49. The method of embodiment 48, wherein the at least one disease is acute otitis media.

50. The method of any one of embodiments 43-49, wherein the reduction in the incidence rate is greater as compared to a control population.

51. The method of embodiment 50, wherein the control population has not been administered the immunogenic composition of any one of embodiments 1-38 or has been administered an immunogenic composition comprising the same recombinant live attenuated S. pneumoniae of the immunogenic composition of any one of embodiments 1-38 but not expressing on its cell surface the at least one heterologous immunogenic protein, or an immunogenic fragment or variant thereof.

52. A method for reducing mammalian transmission of at least one pathogen comprising S. pneumoniae comprising administering to a mammalian subject infected with or at risk of infection by the at least one pathogen comprising S. pneumoniae the immunogenic composition of any one of embodiments 1-38.

53. The method of embodiment 52, wherein the at least one pathogen further comprises Haemophilus influenzae (H. influenzae).

54. The method of embodiment 52 or 53, wherein the at least one pathogen further comprises Moraxella catarrhalis (M. catarrhalis).

55. The method of any one of embodiments 52-54, wherein the immunogenic composition is administered to the mammalian subject intranasally.

56. The method of any one of embodiments 52-55, wherein the method reduces the transmission of the at least one pathogen comprising S. pneumoniae from a mother to its offspring.

57. The method of any one of embodiments 52-56, wherein the reduction in mammalian transmission is greater as compared to a control population.

58. The method of embodiment 57, wherein the control population has not been administered the immunogenic composition of any one of embodiments 1-38 or has been administered an immunogenic composition comprising the same recombinant live attenuated S. pneumoniae of the immunogenic composition of any one of embodiments 1-38 but not expressing on its cell surface the at least one heterologous immunogenic protein, or an immunogenic fragment or variant thereof.

59. A method for preventing or reducing onset, duration, severity, or a combination thereof, of symptoms of acute otitis media comprising administering to a subject a therapeutically effective amount of the immunogenic composition of any one of embodiments 1-38.

60. The method of embodiment 59, wherein the symptoms comprise earache, fever, a lack of energy, hearing loss, coughing, runny nose, loss of balance, or a combination thereof.

61. The immunogenic composition of any one of embodiments 1-38 for use as a medicament.

62. The immunogenic composition for use of embodiment 61, wherein the medicament is used to prevent or reduce onset, duration, severity, or a combination thereof, of symptoms of acute otitis media.

63. The immunogenic composition of any one of embodiments 1-38 for use in preventing or reducing onset, duration, severity, or a combination thereof, of symptoms of acute otitis media.

64. A method for enhancing an immune response against S. pneumoniae, the method comprising administering to a subject in need thereof a therapeutically effective amount of the immunogenic composition of any one of embodiments 1-38, wherein the immune response is enhanced as compared to the subject administered a control immunogenic composition.

65. The method of embodiment 64, wherein the enhanced immune response is an antibody immune response and/or a cell-mediated immune response.

66. The method of embodiment 64 or 65, wherein the administering is intranasally.

67. The method of any one of embodiments 64-66, wherein the administering prevents or reduces onset, duration, severity, or a combination thereof, of symptoms of at least one disease caused by S. pneumoniae.

68. The method of embodiment 67, wherein the at least one disease is selected from the group consisting of acute otitis media, sinusitis, bronchitis, pneumonia, bacteremia, septicemia, and meningitis.

69. The method of embodiment 68, wherein the at least one disease is acute otitis media.

70. The method of any one of embodiments 64-69, wherein the control immunogenic composition does not contain at least one component of the immunogenic composition of any one of embodiments 1-38 or comprises the same recombinant live attenuated S. pneumoniae of the immunogenic composition of any one of embodiments 1-38 but not expressing on its cell surface the at least one heterologous immunogenic protein, or an immunogenic fragment or variant thereof.

71. A genetic construct comprising a polynucleotide encoding a first fusion protein comprising a protein D and a surface anchor moiety operably linked to a promoter.

72. The genetic construct of embodiment 71, wherein the protein D:
(i) comprises an amino acid sequence having at least 80% sequence identity to an amino acid sequence set forth as SEQ ID NO: 213, wherein said protein D retains immunogenicity;
(ii) comprises the amino acid sequence set forth as SEQ ID NO: 213;
(iii) is encoded by a nucleotide sequence having at least 80% sequence identity to a nucleotide sequence set forth as SEQ ID NO: 214, wherein the nucleotide sequence encodes a protein D that retains immunogenicity; and/or
(iv) is encoded by the nucleotide sequence set forth as SEQ ID NO: 214.

73. The genetic construct of embodiment 71 or 72, wherein the surface anchor moiety comprises a choline-binding domain (CBD).

74. The genetic construct of embodiment 73, wherein the CBD comprises a choline binding repeat comprising:
(i) a consensus amino acid sequence having at least 80% sequence identity to an amino acid sequence set forth as SEQ ID NO: 215, wherein said CBD retains surface anchoring activity; and/or
(ii) the consensus amino acid sequence set forth as SEQ ID NO: 215.

75. The genetic construct of embodiment 73 or 74, wherein the CBD:
(i) comprises an amino acid sequence having at least 80% sequence identity to an amino acid sequence set forth as SEQ ID NO: 216, wherein said CBD retains surface anchoring activity;
(ii) comprises the amino acid sequence set forth as SEQ ID NO: 216;
(iii) is encoded by a nucleotide sequence having at least 80% sequence identity to a nucleotide sequence set forth as SEQ ID NO: 217, wherein the nucleotide sequence encodes a CBD that retains surface anchoring activity; and/or
(iv) is encoded by the nucleotide sequence set forth as SEQ ID NO: 217.

76. The genetic construct of any one of embodiments 73-75, wherein the CBD is C-terminal to the protein D.

77. The genetic construct of embodiment 71 or 72, wherein the surface anchor moiety comprises a sortase signal.

78. The genetic construct of embodiment 77, wherein the sortase signal comprises:
(i) an amino acid sequence having at least 80% sequence identity to an amino acid sequence set forth as SEQ ID NO: 206, wherein said sortase signal retains surface anchoring activity; and/or
(ii) an amino acid sequence set forth as SEQ ID NO: 206.

79. The genetic construct of embodiment 77 or 78, wherein the sortase signal:
(i) comprises an amino acid sequence having at least 80% sequence identity to an amino acid sequence set forth as SEQ ID NO: 221, wherein said sortase signal retains surface anchoring activity;
(ii) comprises the amino acid sequence set forth as SEQ ID NO: 221;
(iii) is encoded by a nucleotide sequence having at least 80% sequence identity to a nucleotide sequence set forth as SEQ ID NO: 222, wherein the nucleotide sequence encodes a sortase signal that retains surface anchoring activity; and/or
(iv) is encoded by the nucleotide sequence set forth as SEQ ID NO: 222.

80. The genetic construct of any one of embodiments 77-79, wherein the sortase signal is C-terminal to the protein D.

81. The genetic construct of embodiment 71 or 72, wherein the surface anchor moiety comprises a lipoprotein anchor.

82. The genetic construct of embodiment 81, wherein the lipoprotein anchor comprises a lipobox motif comprising:
(i) a consensus amino acid sequence having at least 80% sequence identity to an amino acid sequence set forth as X₁X₂X₃C, wherein said lipoprotein anchor retains surface anchoring activity; and/or
(ii) the consensus amino acid sequence set forth as X₁X₂X₃C, wherein X₁ is L, V, or I; X₂ is A, S, T, V, or I; and X₃ is G, A, or S.

83. The genetic construct of embodiment 81 or 82, wherein the lipoprotein anchor:
(i) comprises an amino acid sequence having at least 80% sequence identity to an amino acid sequence set forth as SEQ ID NO: 210, wherein said lipoprotein anchor retains surface anchoring activity;
(ii) comprises an amino acid sequence set forth as SEQ ID NO: 210;
(iii) is encoded by a nucleotide sequence having at least 80% sequence identity to a nucleotide sequence set forth as SEQ ID NO: 228, wherein the nucleotide sequence encodes a lipoprotein anchor that retains surface anchoring activity; and/or
(iv) is encoded by a nucleotide sequence set forth as SEQ ID NO: 228.

84. The genetic construct of any one of embodiments 81-83, wherein the lipoprotein anchor is N-terminal to the protein D.

85. The genetic construct of any one of embodiments 71-84, wherein the promoter is a constitutive promoter or an inducible promoter.

86. The genetic construct of embodiment 85, wherein the constitutive promoter comprises a P3 promoter having
(i) a nucleotide sequence having at least 80% sequence identity to a nucleotide sequence set forth as SEQ ID NO: 207 or 236, wherein the nucleotide sequence retains P3 promoter function; and/or
(ii) the nucleotide sequence set forth as SEQ ID NO: 207 or 236.

87. The genetic construct of any one of embodiments 71-86, wherein the genetic construct comprises a terminator 3′ of the polynucleotide encoding the first fusion protein.

88. The genetic construct of embodiment 87, wherein the terminator comprises:
(i) a nucleotide sequence having at least 80% sequence identity to a nucleotide sequence set forth as SEQ ID NO: 235, wherein the nucleotide sequence retains transcription termination function; and/or
(ii) the nucleotide sequence set forth as SEQ ID NO: 235.

89. The genetic construct of any one of embodiments 71-88, wherein the genetic construct comprises at least one terminator 5′ of the promoter.

90. The genetic construct of embodiment 89, wherein the at least one terminator 5′ of the promoter comprises:
(i) a nucleotide sequence having at least 80% sequence identity to a nucleotide sequence set forth as SEQ ID NO: 237, wherein the nucleotide sequence retains transcription termination function; and/or
(ii) a nucleotide sequence set forth as SEQ ID NO: 237.

91. The genetic construct of any one of embodiments 71-90, wherein the polynucleotide encoding the first fusion protein comprises a signal sequence.

92. The genetic construct of embodiment 91, wherein the signal sequence encodes a signal peptide having
(i) an amino acid sequence having at least 80% sequence identity to an amino acid sequence set forth as any of SEQ ID NOs: 210, 211, and 212, wherein said signal peptide retains signal peptide function; and/or
(ii) the amino acid sequence set forth as any of SEQ ID NOs: 210, 211, and 212.

93. The genetic construct of embodiment 92, wherein the signal peptide is a lipoprotein anchor.

94. The genetic construct of any one of embodiments 71-93, further comprising a polynucleotide encoding a UspA polypeptide.

95. The genetic construct of embodiment 94, wherein the UspA polypeptide:
(i) comprises an amino acid sequence having at least 80% sequence identity to an amino acid sequence set forth as SEQ ID NO: 261 or 263, wherein said UspA polypeptide retains immunogenicity;
(ii) comprises the amino acid sequence set forth as SEQ ID NO: 261 or 263;
(iii) is encoded by a nucleotide sequence having at least 80% sequence identity to a nucleotide sequence set forth as SEQ ID NO: 262 or 264, wherein the nucleotide sequence encodes a UspA protein that retains immunogenicity; and/or
(iv) is encoded by the nucleotide sequence set forth as SEQ ID NO: 262 or 264.

96. The genetic construct of embodiment 94 or 95, wherein the first fusion protein further comprises the UspA polypeptide.

97. The genetic construct of any one of embodiments 71-96, wherein the first fusion protein comprises:
(i) an amino acid sequence having at least 80% sequence identity to an amino acid sequence set forth as any of SEQ ID NOs: 219, 223, and 226, wherein said fusion protein retains immunogenicity and anchoring at the cell surface; and/or
(ii) the amino acid sequence set forth as any of SEQ ID NOs: 219, 223, and 226.

98. The genetic construct of any one of embodiments 71-97, wherein the first fusion protein is encoded by:
(i) a nucleotide sequence having at least 80% sequence identity to a nucleotide sequence set forth as any of SEQ ID NOs: 220, 224, and 227, wherein the nucleotide sequence encodes a fusion protein that retains immunogenicity and anchoring at the cell surface; and/or
(ii) the nucleotide sequence set forth as any of SEQ ID NOs: 220, 224, and 227.

99. The genetic construct of embodiment 94 or 95, further comprising a polynucleotide encoding a second fusion protein comprising the UspA polypeptide and a surface anchor moiety operably linked to a promoter.

100. The genetic construct of any one of embodiments 71-99, wherein the genetic construct comprises a nucleotide sequence set forth as any of SEQ ID NOs: 229, 230, 231, 232, 233, and 234.

101. A cell comprising the genetic construct of any one of embodiments 71-100.

102. The cell of embodiment 101, wherein the cell comprises the nucleotide sequence set forth as any of SEQ ID NOs: 229, 230, 231, 232, 233, and 234.

103. The cell of embodiment 101 or 102, wherein the cell is an S. pneumoniae cell.

104. The cell of embodiment 103, wherein the S. pneumoniae cell is a live attenuated S. pneumoniae cell.

105. The cell of embodiment 104, wherein the live attenuated S. pneumoniae cell comprises a disruption in an ftsY gene in its genome.

106. A method of producing a recombinant S. pneumoniae bacterium, comprising introducing into a S. pneumoniae bacterium the genetic construct of any one of embodiments 71-100.

107. The method of embodiment 106, wherein the S. pneumoniae bacterium is a live attenuated S. pneumoniae comprising a disruption in an ftsY gene in its genome.

108. A method of producing an immunogenic composition, the method comprising:
introducing at least one fusion protein into a recombinant live attenuated Streptococcus pneumoniae (S. pneumoniae), wherein each fusion protein comprises:
(i) an immunogenic protein, or an immunogenic fragment or variant thereof; and
(ii) a surface anchor moiety, to produce a modified recombinant live attenuated S. pneumoniae, wherein each fusion protein is expressed at the cell surface of the modified recombinant live attenuated S. pneumoniae.

109. The method of embodiment 108, wherein introducing each fusion protein into the recombinant live attenuated S. pneumoniae comprises introducing at least one nucleic acid molecule comprising a nucleotide sequence encoding the at least one fusion protein.

110. The method of embodiment 108 or 109, wherein the recombinant live attenuated S. pneumoniae comprises a disruption of an ftsY gene in its genome.

111. The method of any one of embodiments 108-110, wherein the modified recombinant live attenuated S. pneumoniae comprises in its genome a nucleotide sequence set forth as any of SEQ ID NOs: 229, 230, 231, 232, 233, and 234.

112. The method of any one of embodiments 108-111, further comprising formulating the modified recombinant live attenuated S. pneumoniae with a pharmaceutically acceptable carrier.

113. The method of any one of embodiments 108-112, further comprising formulating the modified recombinant live attenuated S. pneumoniae with an immunological adjuvant.

114. The method of any one of embodiments 108-113, further formulating the modified recombinant live attenuated S. pneumoniae for intranasal administration.

### X. EXPERIMENTAL EXAMPLES

### Example 1. Generation of Protein D-Expressing Strains

Genetic constructs were designed to express heterologous bacterial proteins on the surface of Streptococcus pneumoniae. In particular, the genetic constructs were designed to express Haemophilus influenzae Protein D on the cell surface of a recombinant, live attenuated vaccine version of Streptococcus pneumoniae. Three different constructs were engineered, each with a unique method of anchoring (lipoprotein anchor, covalent peptidoglycan attachment via sortase, and inclusion of a choline binding domain for non-covalent attachment to the cell wall).

| | |
|---|---|
| Strains | BHN97 CEP-P3ΩProtein D-CBD; |
| | BHN97 CEP-P3ΩProteinD-sort; |
| | BHN97 CEP-P3ΩProteinD-lipo |
| Mutation | Insertion of Protein D with choline-binding domain, |
| | LPXTG type motif, or lipoprotein anchor domain |
| Confirmation | PCR and Overlapping Sanger sequencing |
| Antibiotic | None |
| Resistance | |

BHN97 CEP-P3ΩProteinD-CBD, BHN97 CEP-P3ΩProteinD-sort, and BHN97 CEP-P3ΩProteinD-lipo were generated through multiple PCR amplifications and a final transformation (FIGS. 1A-1G, Table 3). Splicing by overlapping extension (SOE) PCR was used to generate amplicons containing Protein D with a choline-binding domain (-CBD), LPXTG motif (-sort), or lipoprotein anchor domain (-lipo). Expression cassettes containing each of these three protein D genes was inserted in the CEP (chromosomal expression platform) locus in BHN97 CEPQPhunSweetErm, replacing the PhunSweetErm cassette. CEP is a transcriptionally silent site within a truncated IS 1167 element downstream of the ami operon (Guiral et al. Microbiology 2006, 152, 343-349; Sorg et al. ACS Synth. Biol. 2014, 4, 228-239). All three protein D genes were synthesized via Genscript (Piscataway, NJ) to codon optimize the sequence for S. pneumoniae and to include a strong promoter plus upstream terminators (P3), as well as downstream terminators (Term) described previously (Sorg et al. ACS Synth. Biol. 2015, 4, 228-239).

The following PCR amplifications were performed. PCR1 (FIG. 1A) generated an amplicon of flanking region upstream of the CEP insertion site from BHN97 genomic DNA (gDNA) using primers BW015 and BW021. PCR2 (FIG. 1A) generated an amplicon of flanking region downstream of the CEP insertion site from BHN97 gDNA using primers BW022 and BW018.

A number of PCR amplifications were performed (PCR 3-7) to generate amplicons of P3_Protein D_Term from Genscript plasmids. PCR3 (FIG. 1B) generated an amplicon of P3_Protein D-CBD_Term from GenScript ProteinD CBD_puc57 using primers BW023 and BW024. PCR4 (FIG. 1C) generated an amplicon of P3_ProteinD-sort_Term from GenScript ProteinD sort_puc57 using primers BW023 and BW024.

For P3_ProteinD-lipo_Term, GenScript was unable to synthesize the entire sequence and instead provided two fragments (Fragment A and Fragment B) in two separate plasmids. To generate the entire sequence, the two fragments were amplified separately and then spliced together via SOE PCR (PCR 5-7). PCR5 (FIG. 1D) generated an amplicon of P3_ProteinD-lipoFragA from GenScript ProteinD_lipo_FragA_pCC1 using primers BW025 and BW026. PCR6 (FIG. 1E) generated an amplicon of ProteinD-lipoFragB_Term from GenScript ProteinD_lipo_FragB_pUC57 using primers BW027 and BW028. PCR7 (FIG. 1F) generated an amplicon from SOE PCR to form the complete P3_ProteinD-lipo_Term using PCR5 and PCR6 as templates and using primers BW025 and BW028.

A number of SOE PCR amplifications were performed (PCR 8-10, FIG. 1G) to generate CEP-P3ΩProteinD-CBD, CEP-P3ΩProteinD-sort, and CEP-P3ΩProteinD-lipo amplicons using primers BW015 and BW018. PCR8 generated an amplicon from SOE PCR to form CEP-P3ΩProteinD-CBD using PCR1, PCR2, and PCR3. PCR9 generated an amplicon from SOE PCR to form CEP-P3ΩProteinD-sort using PCR1, PCR2, and PCR4. PCR10 generated an amplicon from SOE PCR to form CEP-P3ΩProteinD-lipo using PCR1, PCR2, and PCR7.

TABLE 3
Primers used to generate CEP-P3ΩProteinD-CBD,
CEP-P3ΩProteinD-sort, and CEP-P3ΩProteinD-
lipo. For BW021, BW022, BW025, BW027, and BW028,
nucleotides in bold are overlapping sequence of
5' of P3, 3' of terminator, 3' of upstream
flank of CEP site, 3' of ProteinD-lipoFragA,
and 5' of downstream flank of CEP site,
respectively.

| Name | Sequence |
|---|---|
| BW021 | CATAAGGTAAACTTTTGAGTGCTTTGAT |
| | CTGGTGTCTCAGTCTTTTATTTCTTGCG |
| | (SEQ ID NO: 238) |
| BW022 | CCCTGACAGGGCGCGGTTTTTTTTTTAATT |
| | CCCATAAAAATTGACATGGAAATTATAAA |
| | (SEQ ID NO: 239) |
| BW023 | AAAGCACTCAAAAGTTTACCTTATGGGTGC |
| | (SEQ ID NO: 240) |
| BW024 | AAAAAAAAACCGCGCCCTGTCAGGG |
| | (SEQ ID NO: 241) |
| BW025 | GAAATAAAAGACTGAGACACCAGATCAAAG |
| | CACTCAAAAGTTTACCTTATGGGTGC |
| | (SEQ ID NO: 242) |
| BW026 | CAAAGATTGAATTTCCTTCAAAGTAAAATC |
| | (SEQ ID NO: 243) |
| BW027 | TTTTACTTTGAAGGAAATTCAATCTTTGGA |
| | AATGACAGAAAATTTTGAAACTAAAGATGG |
| | (SEQ ID NO: 244) |
| BW028 | TAATTTCCATGTCAATTTTTATGGGAATTA |
| | AAAAAAAAACCGCGCCCTGTCAGGG |
| | (SEQ ID NO: 245) |

BHN97 CEPQPhunSweetErm was transformed with amplicons of PCR8, PCR9, or PCR10 to generate strains BHN97 CEP-P3ΩProteinD-CBD, BHN97 CEP-P3ΩProteinD-sort, and BHN97 CEP-P3ΩProteinD-lipo, respectively.

### Example 2. Confirmation of Protein D-Expressing Strains

Correct insertion of BHN97 CEP-P3ΩProteinD-CBD, BHN97 CEP-P3ΩProteinD-sort, and BHN97 CEP-P3ΩProteinD-lipo was confirmed through PCR (Table 4) and Sanger sequencing (Table 5). Double homologous recombination was confirmed by using primers BW019 and BW020 that hybridize to nucleic acid regions outside of the flanking regions in the PCR8, PCR9, and PCR10 amplicons.

TABLE 4
Primers used to confirm BHN97 CEP-P3ΩProteinD-
CBD, BHN97 CEP-P3ΩProteinD-sort, and BHN97
CEP-P3ΩProteinD-lipo insertion.

| Name | Sequence |
|---|---|
| BW019 | CTTTGGAAACACCCTCAATACCTG |
| | (SEQ ID NO: 246) |
| BW020 | GCTTTTGCCTTGCGTTCTGACTAC |
| | (SEQ ID NO: 247) |

A subset of clones with the correctly sized amplicon were Sanger sequenced to confirm insertion and correct sequence using primers in Table 3. Resulting sequences overlapped to output entire sequence of P3_ProteinD_Term insertion.

TABLE 5
Primers used to confirm
sequence via Sanger sequencing.

| Name | Sequence |
|---|---|
| BW027 | TTTTACTTTGAAGGAAATTCAATC |
| | TTTGGAAATGACAGAAAATTTTGA |
| | AACTAAAGATGG |
| | (SEQ ID NO: 248) |
| BW029 | GTGATCAACACGCTAGCCAGGCAT |
| | C (SEQ ID NO: 249) |
| BW030 | GGACGCCAATTTCAGTTTACCGAA |
| | AG (SEQ ID NO: 250) |
| BW031 | TAATTTTCCTCCTATTTAGATCTT |
| | GCATGTATAG |
| | (SEQ ID NO: 251) |
| BW032 | CTTTCAGCGGTACCAATCCCAGAT |
| | C (SEQ ID NO: 252) |

Example 3. Generation of an attenuated
vaccine protein D-expressing strain

| | |
|---|---|
| Strain | BHN97 CEP-P3ΩProteinD-lipoΔftsYin::PhunSweetErm |
| Mutation | ftsY (Sp1244): Deletion of internal 867 nucleotides |
| Confirmation | PCR |
| Antibiotic | Erythromycin |
| Resistance | |

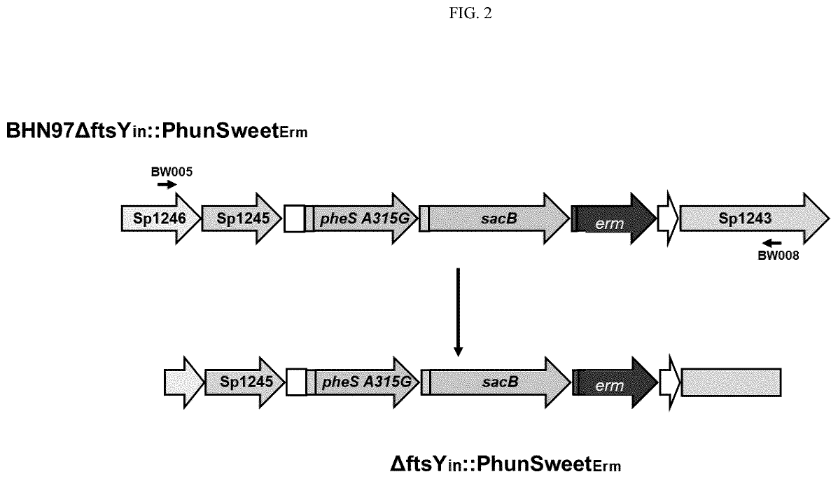

BHN97 CEP-P3ΩProteinD-lipoΔftsYin::PhunSweetErm strain, expressing H. influenzae protein D modified with a lipoprotein anchor and attenuated in virulence with a disruption of signal recognition pathway component ftsY, was generated through a single PCR amplification and transformation (FIG. 2, Table 6). A live, attenuated ΔftsY S. pneumoniae strain has been described in Rosch et al. EMBO Molecular Medicine 2014, 6(1), 141-154. PCR1 generated an amplicon of ΔftsYin::PhunSweetErm plus flanking regions from BHN97ΔftsYin::PhunSweetErm gDNA using primers BW005 and BW008.

The BHN97 CEP-P3ΩProteinD-lipo strain generated in Example 1 and confirmed in Example 2 was transformed with the PCR1 amplicon generated in this Example to create the BHN97 CEP-P3ΩProteinD-lipoΔftsYin::PhunSweetErm strain.

TABLE 6
Primers used to generate ΔftsYin::PhunSweetErm
from BHN97ΔftsYin::PhunSweetErm gDNA.

| Name | Sequence |
|---|---|
| BW005 | CTTGGTATCACAATGGATCAGGT |
| | CATG (SEQ ID NO: 253) |
| BW008 | CTTGGTAAGGAAATGATCCAAAG |
| | CATC (SEQ ID NO: 254) |

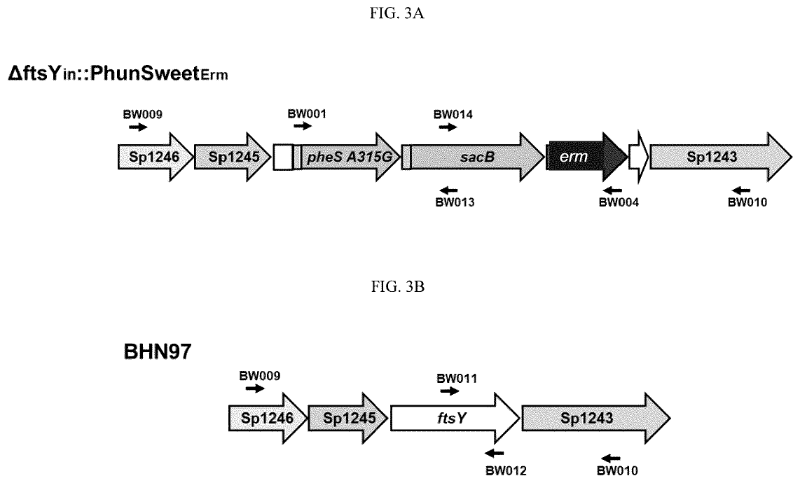

Disruption of ftsY in the BHN97 CEP-P3ΩProteinD-lipoΔftsYin::PhunSweetErm strain was confirmed through PCR using multiple primer sets (FIGS. 3A, 3B; Table 7).

TABLE 7
Primers used to confirm BHN97 CEP-P3ΩProteinD-
lipoΔftsYin::PhunSweetErm mutation.

| Name | Sequence |
|---|---|
| BW009 | GTAGCGAGTTTGGAAGATATCACAG |
| | (SEQ ID NO: 255) |
| BW010 | CACAATAAGCTCTTCTTCTTGTCTA |
| | TGG (SEQ ID NO: 256) |
| BW011 | GGAAGAACTGCTGATTATGAGTGAT |
| | GTTGG (SEQ ID NO: 257) |
| BW012 | CCTCGAGCAGTTCCATCAATCTTAG |
| | TC (SEQ ID NO: 258) |
| BW013 | CAGATGTAAATGTGGCTGAACCTGA |
| | C (SEQ ID NO: 259) |
| BW014 | GACGGAAAAATCCGTTTATTCTACA |
| | CTG (SEQ ID NO: 260) |

### Example 4. Confirmation of Expression of Protein D

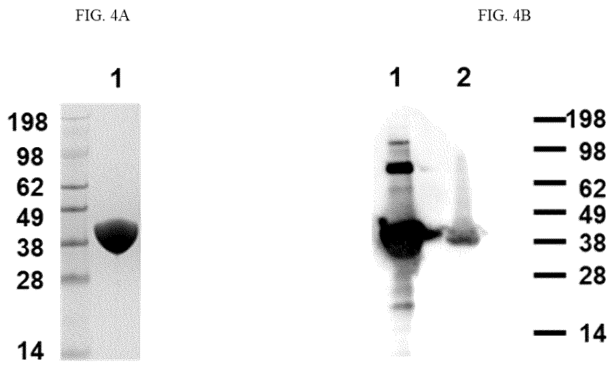

To confirm expression of Protein D, antibodies were first generated against the native H. influenzae Protein D expressed in E. coli. Purified H. influenzae Protein D expressed in E. coli was generated in the St. Jude Protein Production core (FIG. 4A) and sent to Rockland Immunochemicals (Limerick, PA) for generation of rabbit polyclonal antibody against H. influenzae Protein D. The anti-H. influenzae Protein D antibody was specific to H. influenzae Protein D and did not have any observed cross-reactivity to other H. influenzae proteins (FIG. 4B).

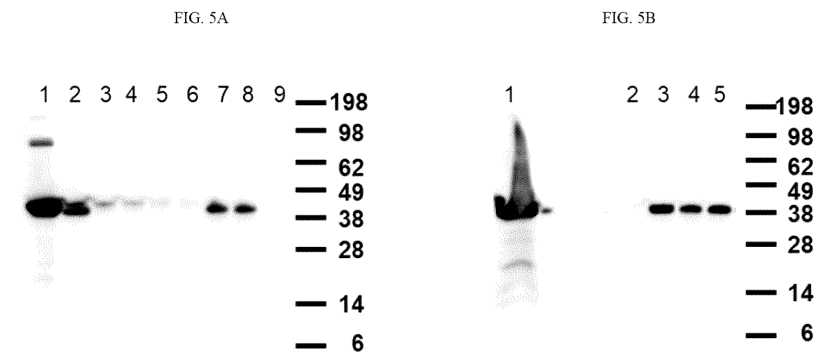

Production of H. influenzae Protein D and anchoring on the cell surface of recombinant H. influenzae Protein D-expressing S. pneumoniae strains were confirmed by Western Blot analysis (FIG. 5) and fluorescence microscopy (FIG. 6) using the polyclonal anti-H. influenzae Protein D antibody.

### Example 5. Vaccination of Mice with Live Attenuated Protein D-Expressing S. pneumoniae Strain

8 week old female Balb/C mice (Jackson) were vaccinated with the live attenuated Protein D-expressing S. pneumoniae strain.

### Vaccination/Infection Schedule

| Date | Procedure |
|---|---|
| Jan. 31, 2022 | Pre-bleed |
| Feb. 7, 2022 | 1st vaccination |
| Feb. 28, 2022 | 1st boost |
| Mar. 21, 2022 | 2nd boost |
| Apr. 4, 2022 | Post-bleed |
| Apr. 11, 2022 | Challenge 40 mice |
| Apr. 12, 2022 | Pictures & Harvest 40 mice |

8 week old female Balb/C mice anesthetized with 2.5% inhaled isoflurane were vaccinated with 10⁵ CFU of the BHN97 CEP-P3ΩProteinD-lipoΔftsY in::PhunSweetErm #1-15-1 strain in a volume of 25 μl phosphate-buffered saline (PBS) intranasally. Mock treated animals received PBS carrier alone. After 3 weeks mice were boosted twice at 3 week intervals. Serum was collected 2 weeks following the final boost. Mice received 3 total vaccinations.

Mice will be challenged 3 weeks after the final boost to determine vaccine efficacy (Table 8). To study acute otitis media (AOM), groups of mice will be anesthetized with 2.5% inhaled isoflurane (n=10 per group; Table 8) and challenged intranasally with 10⁶ CFUs of BHN97 or H. influenzae in 100 μl PBS as described previously (McCullers et al. PLoS Pathog, 2007, 3, e28). The BHN97 challenge strain (BHN97x) has been engineered to express luciferase as described to visually detect colonization in real time in the mice (Francis et al. Infect Immun 2001, 69, 3350-3358).

The results from the mice study is expected to show that bacterial colonization in the noses and/or ears of mice vaccinated with BHN97 CEP-P3ΩProteinD-lipoΔftsYin::PhunSweetErm #1-15-1 is reduced as compared to mice vaccinated with vehicle and/or the percentage of mice vaccinated with BHN97 CEP-P3ΩProteinD-lipoΔftsYin::PhunSweetErm #1-15-1 with detectable bacterial colonization in the noses and/or ears is reduced as compared to the percentage of mice vaccinated with vehicle with detectable bacterial colonization in the noses and/or ears.

TABLE 8
Experimental Groups for Vaccination with live attenuated
Protein D-expressing S. pneumoniae strain.

| | | Dose | | | Challenge | Challenge | Number |
|---|---|---|---|---|---|---|---|
| Group | Vaccination Description | CFU/vol | | Route | strain | CFU/vol | of mice |
| 4.1 | Vehicle (PBS) | 25 | μl | IN | BHN97x | 10⁶/100 μl | 10 |
| 4.2 | BHN97 CEP-P3ΩProteinD- | 10⁵/25 | μl | IN | BHN97x | 10⁶/100 μl | 10 |
| | lipoΔftsYin::PhunSweetErm #1-15-1 | | | | | | |
| 4.3 | Vehicle (PBS) | 25 | μl | IN | H. | 10⁶/100 μl | 10 |
| | | | | | influenzae | | |
| 4.4 | BHN97 CEP-P3ΩProteinD- | 10⁵/25 | μl | IN | H. | 10⁶/100 μl | 10 |
| | lipoΔftsYin::PhunSweetErm #1-15-1 | | | | influenzae | | |

IN = intranasally

### Example 6. Vaccination of Mice with Live Attenuated S. pneumoniae Strain Expressing Two Antigens: H. influenzae Protein D and Moraxella catarrhalis UspA

A live attenuated S. pneumoniae strain (BHN97 ΔftsYin::PhunSweetErm) was modified to express two heterologous antigens: an H. influenzae protein D and a Moraxella catarrhalis UspA polypeptide, both fused to a surface anchor moiety. The live attenuated S. pneumoniae strain expressing the two antigens (BHN97ΔftsY phun:sweet Protein-D-MoraC17) was generated similarly to the live attenuated S. pneumoniae strains expressing a surface anchored H. influenzae protein D as described in Examples 1-3.

Mice were vaccinated with the BHN97ΔftsY phun:sweet Protein-D-MoraC17 strain similarly to the protocol described in Example 5. The mice were also vaccinated with other strains for comparison: BHN97 ΔftsY phun:sweet (the live attenuated S. pneumoniae strain not expressing any surface-anchored heterologous antigen); production stock (the live attenuated S. pneumoniae strain not expressing any surface-anchored heterologous antigen produced in large scale by a manufacturer); BHN97 ΔftsY phun sweet Protein D (BHN97 CEP-P3ΩProteinD-lipoΔftsYin::PhunSweetErm described in Examples 1-5); and BHN97 ΔltyA ΔftsY phun sweet (the live attenuated S. pneumoniae strain with a further deletion of the ltyA gene to disrupt autolysis for potential ease of strain production). Mock treated animals were as described in Example 5.

Mice were challenged with BHN97x, H. influenzae, or M. catarrhalis similarly to the protocol described in Example 5.

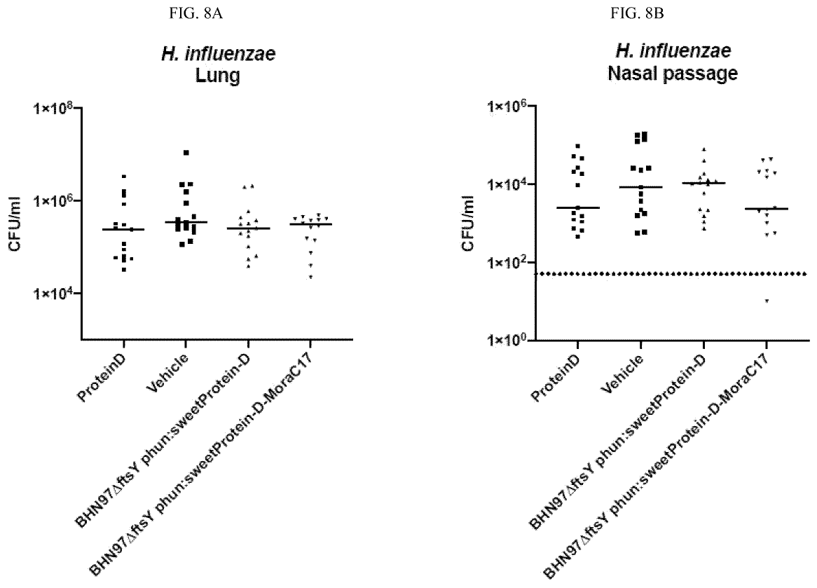

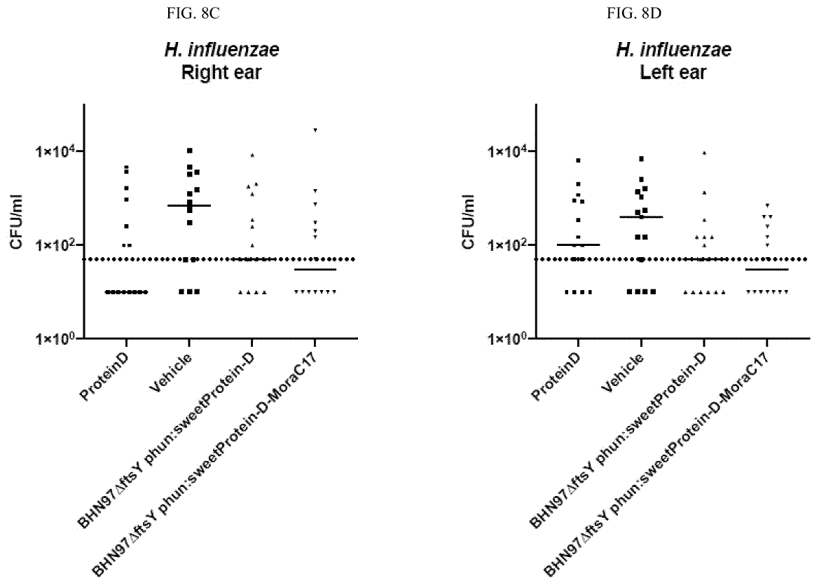

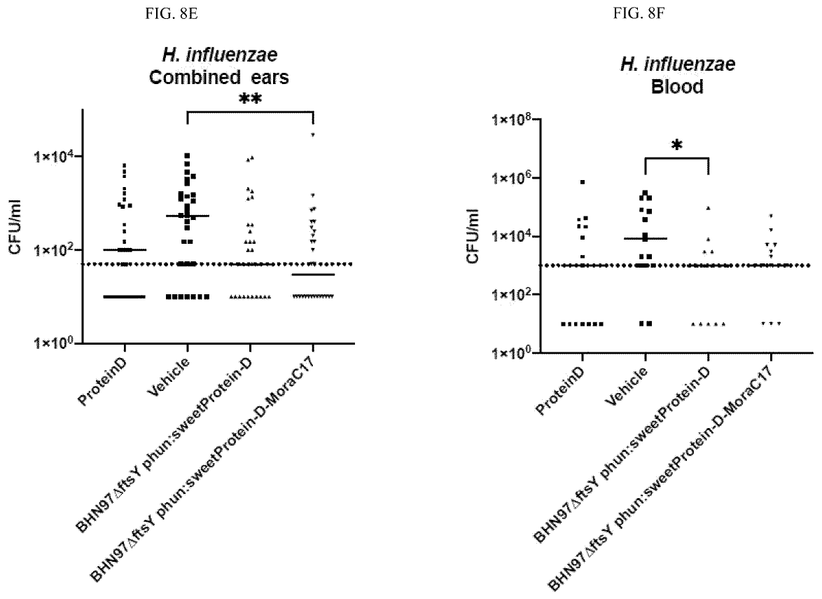

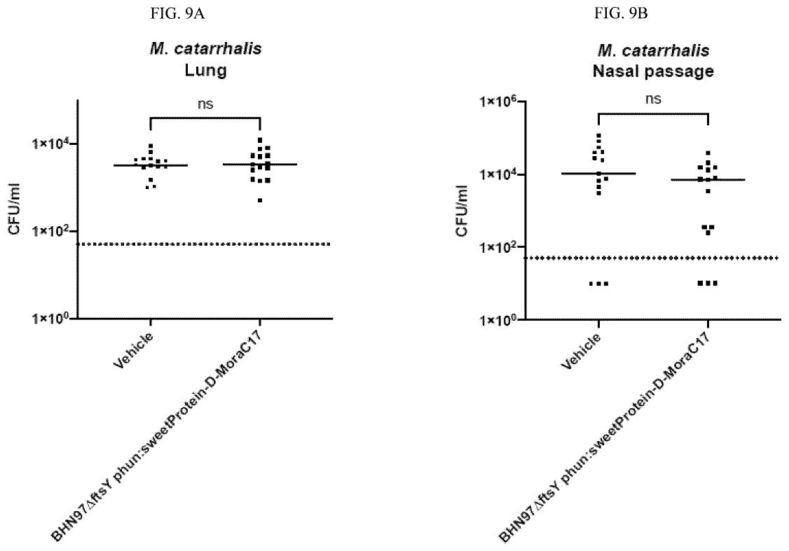

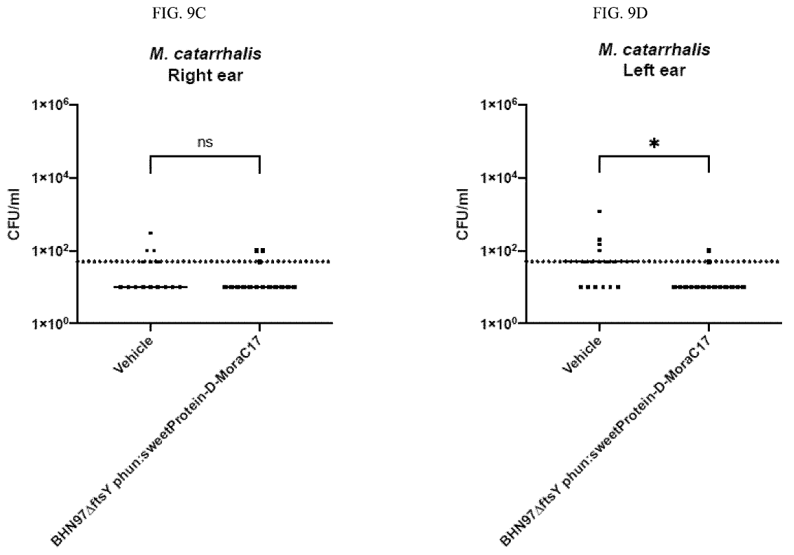

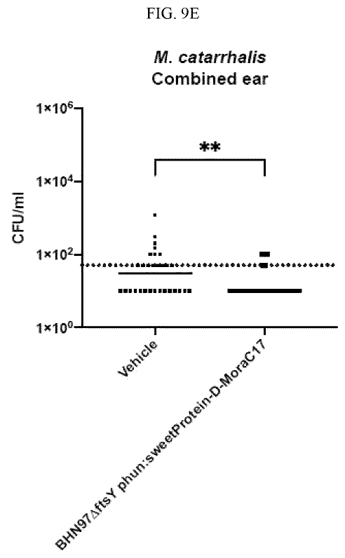

Results from these mice studies are shown in: FIGS. 7A-7F for vaccinated mice challenged with BHN97x, FIGS. 8A-8F for vaccinated mice challenged with H. influenzae, and FIGS. 9A-9E for vaccinated mice challenged with M. catarrhalis. Bacterial colonization in the lung, nasal passage, ears, and blood of the vaccinated mice were measured.

The combination vaccine comprising the live attenuated S. pneumoniae strain expressing a surface anchored H. influenzae protein D and a Moraxella catarrhalis UspA polypeptide (BHN97ΔftsY phun:sweet Protein-D-MoraC17) provided protection against all 3 pathogens (S. pneumoniae, H. influenzae, and M. catarrhalis; FIGS. 7A-7F, FIGS. 8A-8F, FIGS. 9A-9E). Strikingly, inclusion of the additional antigens of H. influenzae and M. catarrhalis on the surface of the live attenuated S. pneumoniae strain enhanced the protective capacity of the live attenuated vaccine against S. pneumoniae (compare BHN97 ΔftsY phun:sweet Protein-D-MoraC17 versus BHN97 ΔftsY phun:sweet in FIGS. 7A-7F).

### Example 7. Materials and Methods

### Bacterial Growth Conditions

Streptococcus pneumoniae strains were grown in CY media under static conditions at 37° C.+5% CO₂ for liquid culture and on TSA/blood agar at 37° C.+5% CO₂ for solid culture. TSA/blood agar plates were prepared from 40 mg/L tryptic soy agar (EMD Millipore, GranuCult, item number 105458) in distilled water, and then autoclaved for 45 minutes. After cooling to 55° C., 3% defibrinated sheep blood was added (Lampire biological, item number 7239001) and poured into 100×15 mm round petri dishes. Deletion mutants were selected on TSA/blood agar with 1 g/mL erythromycin. Chromosomal complementation mutants were selected on TSA/blood agar supplemented with 1 g/mL erythromycin and 150 g/mL spectinomycin.

### Cy Media was Prepared as Follows:

Supplement: The supplement “3 in 1” salts was prepared by adding 50 g MgCl₂ 6H₂O, 0.25 g CalCl₂ anhydrous and 0.1 mL (0.1 M) Manganese sulfate 4H₂O in 500 ml distilled water (dH₂O), mixing well, and autoclaving. 500 ml of each of 20% Glucose, 50% Sucrose, 2 mg/ml Adenosine, and 2 mg/ml Uridine were prepared and filter sterilized. All 5 components were combined at the following ratio: 60 ml “3 in 1” salts, 120 ml 20% Glucose, 6 ml 50% Sucrose, 120 ml 2 mg/ml Adenosine, 120 ml 2 mg/ml Uridine. The solution was mixed in a beaker and filter sterilized, labeled as Supplement, and stored at 4° C.
Adams Solutions: Adams I was prepared by combining the following chemicals: 30 mg Nicotinic Acid (Niacin stored at 4° C.), 35 mg Pyridoxine HCl (B6), 120 mg Ca-Pantothenate (stored at 4° C.), 32 mg Thiamine-HCl, 14 mg Riboflavin, and 0.06 ml Biotin (0.5 mg/ml stock). dH₂O was added to 200 ml, then 1-5 drops of 10N NaOH were add to dissolve chemicals. The solution was filter sterilized and store in foiled bottles at 4° C. Adams II was prepared by adding the following chemicals: 50 mg FeSO₄7H₂O, 50 mg CuSO₄, 50 mg ZnSO₄7H₂O, 20 mg MnCl₂ and 1 ml HCl, up to 100 ml dH₂O. The solution was filtered to sterilize and stored at 4° C. Adams III was prepared by adding the following 5 components: 800 mg Asparagine, 80 mg Choline Chloride, 64 ml Adams I, 16 ml Adams II and 0.64 ml CaCl2) (1% stock), to 400 ml dH2O. The solutions were filter sterilized and store in foiled bottle at 4° C.
Buffers: 1 M KH₂PO₄ and 1 M K₂HPO₄ (autoclaved) were prepared from stock solutions, where 26.5 ml 1 M KH₂PO₄ and 473 1 M K₂HPO₄ were mixed and stirred well, were not titrated, and were filtered to sterilize, and stored at 4° C.
PreC: PreC was prepared by mixing the following chemicals: 4.83 g Sodium Acetate (Anhydrous), 20 g Difco Casamino Acids/technical, 20 mg/L-Tryptophan, and 200 g/L Cysteine HCl, dissolve in 800 dH₂O. pH was adjusted to 7.4-7.6 by adding 10N NaOH. The solution was stirred well for 60 minutes, filled up to 4 liter dH₂O, mixed well, aliquoted into 400 ml portions in 500 ml flasks, autoclaved for 30 minutes, and stored at 4° C.
C+Y: 0.5 g glutamine was added to 500 ml dH₂O, filtered to sterilize, and stored at 4° C. 2 g pyruvic acid (stored at 4° C.) was added to 100 ml dH₂O, filtered to sterilize, and stored at 4° C. 5 g yeast was dissolved in 100 ml dH₂O (25 g in 500 ml), and autoclaved (filtered to sterilize if necessary). 6 of the following solutions was added to 400 ml PreC: 13 ml Supplement, 10 ml Glutamine, 10 ml Adams III, 5 ml Pyruvate, 15 ml K-Phosphate buffer, and 9 ml Yeast. The resulting solution was filter sterilized and stored at 4° C. CY sugar deplete media was made as above but with the omission of glucose, sucrose and yeast extract.

### Genomic DNA Extraction

Strains were grown in 10 mL C+Y to late logarithmic (OD₆₂₀˜0.8), and bacterial culture was pelleted at 6000×g, 10 min. For PCR (polymerase chain reaction) amplification, genomic DNA (gDNA) was obtained using aqueous/organic extraction. Briefly, the bacterial pellet was subjected to lysis in 1 mL PBS plus 50 μL 10% SDS, 50 μL 10% DOC, and 10 μL of 10 mg/mL Proteinase K (Sigma, St. Louis, MO, USA), followed by incubation at 37° C. until clear. Lysates were mixed with 500 μL of phenol: chloroform: isoamyl alcohol (Sigma) and transferred to phase-lock tubes (Quantabio, Beverly, MA, USA). Organic and aqueous phases were separated by centrifugation as per instructions. The aqueous phase was mixed with 500 μL chloroform: isoamyl alcohol in the phase-lock tube, separated again by centrifugation, and then transferred to 100% ethanol for DNA precipitation. Precipitated DNA was washed in 70% ethanol, dried at 65° C., and rehydrated in water. For confirmation of mutations, genomic DNA was extracted from pneumococcal strains using a modified version of the Wizard DNA extraction kit (Promega, Madison, WI, USA). The bacterial pellet was subjected to lysis in 500 μL PBS plus 50 μL 10% SDS and 50 μL 10% DOC, followed by incubation at 37° C. until clear. RNA was removed by addition of 3 μL of 4 mg/mL RNAseA (Promega) and incubation at 37° C. for 15 min. The remainder of the extraction followed the protocol provided with the kit, starting with step 3.

### PCR Amplification and Transformation

PCR products were created through splicing by overlap extension (SOE) PCR (Horton et al. Biotechniques 1990, 8, 528-535). All PCR products were amplified using exTaq polymerase (TAKARA, Mountain View, CA, USA) following the recommended guidelines. Primers used are listed in Tables 3 and 6.

To transform S. pneumoniae, PCR fragments were introduced to the relevant BHN97 strain grown to OD₆₂₀˜0.07 in C+Y, along with competence stimulating peptide 2 or 1, respectively (Pozzi et al. J. Bacteriol. 1996, 178, 6087-6090). Cells were incubated for 3 h at 37° C., and mutants were selected on TSA agar plates containing 3% sheep blood (and 1 mg/mL erythromycin for the cassette disrupting ftsY). Integration of the cassettes at the right location in the genome was confirmed by PCR and sequencing.

### Construction of BHN97 CEP-P3ΩProteinD-LipoΔftsYin::PhunSweetErm

Briefly, the coding region for ftsY was replaced with an erythromycin-resistance cassette by using homologous recombination. Transformants were selected on TSA plates supplemented with 3% sheep blood and erythromycin (1 mg/ml) after an overnight incubation at 37° C. in a 5% CO₂ humidified incubator. The ftsY disruption renders S. pneumoniae avirulent, with at least a 3-log difference in LD₅₀ compared to the parental strain. At the highest dosages of 10⁸ CFUs (colony-forming units), survival greater than 90% was observed for the live attenuated strain. Insertion of the erythromycin cassette does not significantly impact pneumococcal virulence, as previously observed (Mann et al. (2012) PLoS Pathog 8: e1002788).

### Western Blot

100 μL sample was combined with 30 μL 4× sample buffer (NuPAGE, Invitrogen) and boiled 10 minutes. 15 μL was loaded into 15 well 4-12% Bis-Tris precast gel, and run 1 hour 45 minutes at 80V in NuPAGE running buffer. Gels were transferred to nitrocellulose membranes at 30V for 90 minutes in NuPAGE transfer buffer supplemented with 20% methanol. Membranes were blocked 1 hour at room temperature in 4% non-fat dry milk in PBS supplemented with 0.1% Tween-20 (PBST). Membranes were incubated with polyclonal rabbit antibody in 4% non-fat dry milk in PBST overnight at 4° C. Blots were washed 3×10 minutes with PBST. Membranes were incubated with secondary antibody, goat anti-rabbit IgG-HRP (Invitrogen), in 4% non-fat dry milk in PBST 3 hours at room temperature. Blots were washed 3×10 minutes in PBST and 1×5 minutes in PBS. 2 mL each SuperSignal West Dura Extended Duration Substrate (Thermo Scientific) reagent was added to each blot and incubated for 5 minutes at room temperature. Blots were imaged on a ChemiDoc MP (BioRad) using ImageLab 5.0 software, automatic exposure settings for Chemiluminescence, high specificity, optimizing for bright bands. Purified protein blots were performed as above except 500 ng each protein was used as sample.

### Fluorescence Microscopy

Whole cells of Protein D-expressing S. pneumoniae strains were fixed in paraformaldehyde and then stained with a polyclonal rabbit antibody against Protein D and an Alexa-488 secondary antibody.

We claim:

1. An immunogenic composition comprising:
a recombinant live attenuated Streptococcus pneumoniae (S. pneumoniae) expressing on its cell surface at least one heterologous immunogenic protein, or an immunogenic fragment or variant thereof.

2. The immunogenic composition of claim 1, wherein the at least one heterologous immunogenic protein, or an immunogenic fragment or variant thereof, comprises a Haemophilus influenzae (H. influenzae) polypeptide.

3. The immunogenic composition of claim 2, wherein the H. influenzae polypeptide comprises protein D.

4. The immunogenic composition of claim 3, wherein the protein D:
(i) comprises an amino acid sequence having at least 80% sequence identity to an amino acid sequence set forth as SEQ ID NO: 213, wherein said protein D retains immunogenicity;
(ii) comprises the amino acid sequence set forth as SEQ ID NO: 213;
(iii) is encoded by a nucleotide sequence having at least 80% sequence identity to a nucleotide sequence set forth as SEQ ID NO: 214, wherein the nucleotide sequence encodes a protein D that retains immunogenicity; and/or
(iv) is encoded by the nucleotide sequence set forth as SEQ ID NO: 214.

5. The immunogenic composition of any one of claims 1-4, wherein the at least one heterologous immunogenic protein, or an immunogenic fragment or variant thereof, comprises a Moraxella catarrhalis (M. catarrhalis) polypeptide.

6. The immunogenic composition of claim 5, wherein the M. catarrhalis polypeptide comprises a UspA polypeptide.

7. The immunogenic composition of claim 6, wherein the UspA polypeptide:
(i) comprises an amino acid sequence having at least 80% sequence identity to an amino acid sequence set forth as SEQ ID NO: 261 or 263, wherein the UspA polypeptide retains immunogenicity;
(ii) comprises the amino acid sequence set forth as SEQ ID NO: 261 or 263;
(iii) is encoded by a nucleotide sequence having at least 80% sequence identity to a nucleotide sequence set forth as SEQ ID NO: 262 or 264, wherein the nucleotide sequence encodes a UspA polypeptide that retains immunogenicity; and/or
(iv) is encoded by the nucleotide sequence set forth as SEQ ID NO: 262 or 264.

8. The immunogenic composition of any one of claims 1-7, wherein the at least one heterologous immunogenic protein, or an immunogenic fragment or variant thereof comprises an S. pneumoniae polypeptide.

9. The immunogenic composition of claim 8, wherein the S. pneumoniae polypeptide comprises:
(i) an amino acid sequence having at least 80% sequence identity to an amino acid sequence set forth as any one of SEQ ID NOs: 1-205, wherein the S. pneumoniae polypeptide retains immunogenicity; and/or
(ii) the amino acid sequence set forth as any one of SEQ ID NOs: 1-205.

10. The immunogenic composition of any one of claims 1-9, wherein the at least one heterologous immunogenic protein, or an immunogenic fragment or variant thereof, comprises a surface anchor moiety.

11. The immunogenic composition of claim 10, wherein the at least one heterologous immunogenic protein, or an immunogenic fragment or variant thereof, and the surface anchor moiety are linked as a fusion protein.

12. The immunogenic composition of claim 10 or 11, wherein the surface anchor moiety is C-terminal to the at least one heterologous immunogenic protein, or an immunogenic fragment or variant thereof.

13. The immunogenic composition of claim 10 or 11, wherein the surface anchor moiety is N-terminal to the at least one heterologous immunogenic protein, or an immunogenic fragment or variant thereof.

14. The immunogenic composition of any one of claims 11-13, wherein the fusion protein comprises:
(i) an amino acid sequence having at least 80% sequence identity to an amino acid sequence set forth as any of SEQ ID NOs: 219, 223, and 226, wherein said fusion protein retains immunogenicity and anchoring at the cell surface; and/or
(ii) the amino acid sequence set forth as any of SEQ ID NOs: 219, 223, and 226.

15. The immunogenic composition of any one of claims 11-14, wherein the fusion protein is encoded by:
(i) a nucleotide sequence having at least 80% sequence identity to a nucleotide sequence set forth asset forth as any of SEQ ID NOs: 220, 224, and 227, wherein the nucleotide sequence encodes a fusion protein that retains immunogenicity and anchoring at the cell surface; and/or
(ii) the nucleotide sequence set forth asset forth as any of SEQ ID NOs: 220, 224, and 227.

16. The immunogenic composition of any one of claims 10-15, wherein the surface anchor moiety comprises a choline-binding domain (CBD).

17. The immunogenic composition of claim 16, wherein the CBD comprises a choline binding repeat comprising:
(i) a consensus amino acid sequence having at least 80% sequence identity to an amino acid sequence set forth asset forth as SEQ ID NO: 215, wherein said CBD retains surface anchoring activity; and/or
(ii) the consensus amino acid sequence set forth asset forth as SEQ ID NO: 215.

18. The immunogenic composition of claim 16 or 17, wherein the CBD:
(i) comprises an amino acid sequence having at least 80% sequence identity to an amino acid sequence set forth asset forth as SEQ ID NO: 216, wherein said CBD retains surface anchoring activity;
(ii) comprises the amino acid sequence set forth as SEQ ID NO: 216;
(iii) is encoded by a nucleotide sequence having at least 80% sequence identity to a nucleotide sequence set forth as SEQ ID NO: 217, wherein the nucleotide sequence encodes a CBD that retains surface anchoring activity; and/or
(iv) is encoded by the nucleotide sequence set forth as SEQ ID NO: 217.

19. The immunogenic composition of any one of claims 10-15, wherein the surface anchor moiety comprises a sortase signal.

20. The immunogenic composition of claim 19, wherein the sortase signal comprises:
(i) a consensus amino acid sequence having at least 80% sequence identity to an amino acid sequence set forth as SEQ ID NO: 206, wherein said sortase signal retains surface anchoring activity; and/or
(ii) the consensus amino acid sequence set forth as SEQ ID NO: 206.

21. The immunogenic composition of claim 19 or 20, wherein the sortase signal:
(i) comprises an amino acid sequence having at least 80% sequence identity to an amino acid sequence set forth as SEQ ID NO: 221, wherein said sortase signal retains surface anchoring activity;
(ii) comprises the amino acid sequence set forth as SEQ ID NO: 221;
(iii) is encoded by a nucleotide sequence having at least 80% sequence identity to a nucleotide sequence set forth as SEQ ID NO: 222, wherein the nucleotide sequence encodes a sortase signal that retains surface anchoring activity; and/or
(iv) is encoded by the nucleotide sequence set forth as SEQ ID NO: 222.

22. The immunogenic composition of any one of claims 10-15, wherein the surface anchor moiety comprises a lipoprotein anchor.

23. The immunogenic composition of claim 22, wherein the lipoprotein anchor comprises a lipobox motif comprising:
(i) a consensus amino acid sequence having at least 80% sequence identity to an amino acid sequence set forth as X₁X₂X₃C, wherein said lipoprotein anchor retains surface anchoring activity; and/or
(ii) the consensus amino acid sequence set forth as X₁X₂X₃C,
wherein X₁ is L, V, or I; X₂ is A, S, T, V, or I; and X₃ is G, A, or S.

24. The immunogenic composition of claim 22 or 23, wherein the lipoprotein anchor:
(i) comprises an amino acid sequence having at least 80% sequence identity to an amino acid sequence set forth as SEQ ID NO: 210, wherein said lipoprotein anchor retains surface anchoring activity;
(ii) comprises the amino acid sequence set forth as SEQ ID NO: 210;
(iii) is encoded by a nucleotide sequence having at least 80% sequence identity to a nucleotide sequence set forth as SEQ ID NO: 228, wherein the nucleotide sequence encodes a lipoprotein anchor that retains surface anchoring activity; and/or
(iv) is encoded by the nucleotide sequence set forth as SEQ ID NO: 228.

25. The immunogenic composition of any one of claims 1-24, wherein the at least one heterologous immunogenic protein, or an immunogenic fragment or variant thereof, is attached to the surface covalently or non-covalently.

26. The immunogenic composition of any one of claims 1-25, wherein the recombinant live attenuated S. pneumoniae expresses on its surface at least two heterologous immunogenic proteins, or immunogenic fragments or variants thereof.

27. The immunogenic composition of claim 26, wherein the at least two heterologous immunogenic proteins, or immunogenic fragments or variants thereof are from a single pathogen.

28. The immunogenic composition of claim 27, wherein the single pathogen is H. influenzae.

29. The immunogenic composition of claim 27, wherein the single pathogen is S. pneumoniae.

30. The immunogenic composition of claim 27, wherein the single pathogen is M. catarrhalis.

31. The immunogenic composition of claim 26, wherein the at least two heterologous immunogenic proteins, or immunogenic fragments or variants thereof, are from different pathogens.

32. The immunogenic composition of claim 31, wherein the different pathogens comprise a combination of pathogens selected from S. pneumoniae, H. influenzae, and M. catarrhalis.

33. The immunogenic composition of any one of claims 10-25, wherein the recombinant live attenuated S. pneumoniae expresses on its surface at least two heterologous immunogenic proteins, or immunogenic fragments or variants thereof, and wherein the at least two heterologous immunogenic proteins, or immunogenic fragments or variants thereof, and the surface anchor moiety are expressed as one fusion protein.

34. The immunogenic composition of any one of claims 1-33, wherein the recombinant live attenuated S. pneumoniae comprises a disruption of an ftsY gene in its genome.

35. The immunogenic composition of any one of claims 1-34, wherein the recombinant live attenuated S. pneumoniae comprises in its genome the nucleotide sequence set forth as any of SEQ ID NOs: 229, 230, 231, 232, 233, and 234.

36. The immunogenic composition of any one of claims 1-35, further comprising an immunological adjuvant.

37. The immunogenic composition of any one of claims 1-36, wherein the immunogenic composition is formulated for intranasal administration.

38. The immunogenic composition of any one of claims 1-37, wherein the immunogenic composition is a vaccine composition.

39. A method for inducing an immune response, the method comprising administering to a subject in need thereof a therapeutically effective amount of the immunogenic composition of any one of claims 1-38.

40. The method of claim 39, wherein the induced immune response is an antibody immune response and/or a cell-mediated immune response.

41. The method of claim 39 or 40, wherein the administering prevents or reduces onset, duration, severity, or a combination thereof, of symptoms of at least one disease caused by at least one pathogen.

42. The method of claim 41, wherein the at least one pathogen comprises S. pneumoniae.

43. The method of any one of claim 39-42, wherein the administering reduces the incidence rate of the at least one disease in a mammalian population.

44. The method of any one of claims 41-43, wherein the at least one pathogen further comprises H. influenzae.

45. The method of any one of claims 41-44, wherein the at least one pathogen further comprises M. catarrhalis.

46. The method of any one of claims 41-45, wherein the immunogenic composition is administered to the subject intranasally.

47. The method of any one of claims 41-46, wherein the method reduces the transmission of the at least one pathogen from a mother to its offspring.

48. The method of any one of claims 41-47, wherein the at least one disease is selected from the group consisting of acute otitis media, sinusitis, bronchitis, pneumonia, bacteremia, septicemia, and meningitis.

49. The method of claim 48, wherein the at least one disease is acute otitis media.

50. The method of any one of claims 43-49, wherein the reduction in the incidence rate is greater as compared to a control population.

51. The method of claim 50, wherein the control population has not been administered the immunogenic composition of any one of claims 1-38 or has been administered an immunogenic composition comprising the same recombinant live attenuated S. pneumoniae of the immunogenic composition of any one of claims 1-38 but not expressing on its cell surface the at least one heterologous immunogenic protein, or an immunogenic fragment or variant thereof.

52. A method for reducing mammalian transmission of at least one pathogen comprising S. pneumoniae comprising administering to a mammalian subject infected with or at risk of infection by the at least one pathogen comprising S. pneumoniae the immunogenic composition of any one of claims 1-38.

53. The method of claim 52, wherein the at least one pathogen further comprises Haemophilus influenzae (H. influenzae).

54. The method of claim 52 or 53, wherein the at least one pathogen further comprises Moraxella catarrhalis (M. catarrhalis).

55. The method of any one of claims 52-54, wherein the immunogenic composition is administered to the mammalian subject intranasally.

56. The method of any one of claims 52-55, wherein the method reduces the transmission of the at least one pathogen comprising S. pneumoniae from a mother to its offspring.

57. The method of any one of claims 52-56, wherein the reduction in mammalian transmission is greater as compared to a control population.

58. The method of claim 57, wherein the control population has not been administered the immunogenic composition of any one of claims 1-38 or has been administered an immunogenic composition comprising the same recombinant live attenuated S. pneumoniae of the immunogenic composition of any one of claims 1-38 but not expressing on its cell surface the at least one heterologous immunogenic protein, or an immunogenic fragment or variant thereof.

59. A method for preventing or reducing onset, duration, severity, or a combination thereof, of symptoms of acute otitis media comprising administering to a subject a therapeutically effective amount of the immunogenic composition of any one of claims 1-38.

60. The method of claim 59, wherein the symptoms comprise earache, fever, a lack of energy, hearing loss, coughing, runny nose, loss of balance, or a combination thereof.

61. The immunogenic composition of any one of claims 1-38 for use as a medicament.

62. The immunogenic composition for use of claim 61, wherein the medicament is used to prevent or reduce onset, duration, severity, or a combination thereof, of symptoms of acute otitis media.

63. The immunogenic composition of any one of claims 1-38 for use in preventing or reducing onset, duration, severity, or a combination thereof, of symptoms of acute otitis media.

64. A method for enhancing an immune response against S. pneumoniae, the method comprising administering to a subject in need thereof a therapeutically effective amount of the immunogenic composition of any one of claims 1-38, wherein the immune response is enhanced as compared to the subject administered a control immunogenic composition.

65. The method of claim 64, wherein the enhanced immune response is an antibody immune response and/or a cell-mediated immune response.

66. The method of claim 64 or 65, wherein the administering is intranasally.

67. The method of any one of claims 64-66, wherein the administering prevents or reduces onset, duration, severity, or a combination thereof, of symptoms of at least one disease caused by S. pneumoniae.

68. The method of claim 67, wherein the at least one disease is selected from the group consisting of acute otitis media, sinusitis, bronchitis, pneumonia, bacteremia, septicemia, and meningitis.

69. The method of claim 68, wherein the at least one disease is acute otitis media.

70. The method of any one of claims 64-69, wherein the control immunogenic composition does not contain at least one component of the immunogenic composition of any one of claims 1-38 or comprises the same recombinant live attenuated S. pneumoniae of the immunogenic composition of any one of claims 1-38 but not expressing on its cell surface the at least one heterologous immunogenic protein, or an immunogenic fragment or variant thereof.

71. A genetic construct comprising a polynucleotide encoding a first fusion protein comprising a protein D and a surface anchor moiety operably linked to a promoter.

72. The genetic construct of claim 71, wherein the protein D:
(i) comprises an amino acid sequence having at least 80% sequence identity to an amino acid sequence set forth as SEQ ID NO: 213, wherein said protein D retains immunogenicity;
(ii) comprises the amino acid sequence set forth as SEQ ID NO: 213;
(iii) is encoded by a nucleotide sequence having at least 80% sequence identity to a nucleotide sequence set forth as SEQ ID NO: 214, wherein the nucleotide sequence encodes a protein D that retains immunogenicity; and/or
(iv) is encoded by the nucleotide sequence set forth as SEQ ID NO: 214.

73. The genetic construct of claim 71 or 72, wherein the surface anchor moiety comprises a choline-binding domain (CBD).

74. The genetic construct of claim 73, wherein the CBD comprises a choline binding repeat comprising:
(i) a consensus amino acid sequence having at least 80% sequence identity to an amino acid sequence set forth as SEQ ID NO: 215, wherein said CBD retains surface anchoring activity; and/or
(ii) the consensus amino acid sequence set forth as SEQ ID NO: 215.

75. The genetic construct of claim 73 or 74, wherein the CBD:
(i) comprises an amino acid sequence having at least 80% sequence identity to an amino acid sequence set forth as SEQ ID NO: 216, wherein said CBD retains surface anchoring activity;
(ii) comprises the amino acid sequence set forth as SEQ ID NO: 216;
(iii) is encoded by a nucleotide sequence having at least 80% sequence identity to a nucleotide sequence set forth as SEQ ID NO: 217, wherein the nucleotide sequence encodes a CBD that retains surface anchoring activity; and/or
(iv) is encoded by the nucleotide sequence set forth as SEQ ID NO: 217.

76. The genetic construct of any one of claims 73-75, wherein the CBD is C-terminal to the protein D.

77. The genetic construct of claim 71 or 72, wherein the surface anchor moiety comprises a sortase signal.

78. The genetic construct of claim 77, wherein the sortase signal comprises:
(i) an amino acid sequence having at least 80% sequence identity to an amino acid sequence set forth as SEQ ID NO: 206, wherein said sortase signal retains surface anchoring activity; and/or
(ii) an amino acid sequence set forth as SEQ ID NO: 206.

79. The genetic construct of claim 77 or 78, wherein the sortase signal:
(i) comprises an amino acid sequence having at least 80% sequence identity to an amino acid sequence set forth as SEQ ID NO: 221, wherein said sortase signal retains surface anchoring activity;
(ii) comprises the amino acid sequence set forth as SEQ ID NO: 221;
(iii) is encoded by a nucleotide sequence having at least 80% sequence identity to a nucleotide sequence set forth as SEQ ID NO: 222, wherein the nucleotide sequence encodes a sortase signal that retains surface anchoring activity; and/or
(iv) is encoded by the nucleotide sequence set forth as SEQ ID NO: 222.

80. The genetic construct of any one of claims 77-79, wherein the sortase signal is C-terminal to the protein D.

81. The genetic construct of claim 71 or 72, wherein the surface anchor moiety comprises a lipoprotein anchor.

82. The genetic construct of claim 81, wherein the lipoprotein anchor comprises a lipobox motif comprising:
(i) a consensus amino acid sequence having at least 80% sequence identity to an amino acid sequence set forth as X₁X₂X₃C, wherein said lipoprotein anchor retains surface anchoring activity; and/or
(ii) the consensus amino acid sequence set forth as X₁X₂X₃C,
wherein X₁ is L, V, or I; X₂ is A, S, T, V, or I; and X₃ is G, A, or S.

83. The genetic construct of claim 81 or 82, wherein the lipoprotein anchor:
(i) comprises an amino acid sequence having at least 80% sequence identity to an amino acid sequence set forth as SEQ ID NO: 210, wherein said lipoprotein anchor retains surface anchoring activity;
(ii) comprises an amino acid sequence set forth as SEQ ID NO: 210;
(iii) is encoded by a nucleotide sequence having at least 80% sequence identity to a nucleotide sequence set forth as SEQ ID NO: 228, wherein the nucleotide sequence encodes a lipoprotein anchor that retains surface anchoring activity; and/or
(iv) is encoded by a nucleotide sequence set forth as SEQ ID NO: 228.

84. The genetic construct of any one of claims 81-83, wherein the lipoprotein anchor is N-terminal to the protein D.

85. The genetic construct of any one of claims 71-84, wherein the promoter is a constitutive promoter or an inducible promoter.

86. The genetic construct of claim 85, wherein the constitutive promoter comprises a P3 promoter having (i) a nucleotide sequence having at least 80% sequence identity to a nucleotide sequence set forth as SEQ ID NO: 207 or 236, wherein the nucleotide sequence retains P3 promoter function; and/or
(ii) the nucleotide sequence set forth as SEQ ID NO: 207 or 236.

87. The genetic construct of any one of claims 71-86, wherein the genetic construct comprises a terminator 3′ of the polynucleotide encoding the first fusion protein.

88. The genetic construct of claim 87, wherein the terminator comprises:
(i) a nucleotide sequence having at least 80% sequence identity to a nucleotide sequence set forth as SEQ ID NO: 235, wherein the nucleotide sequence retains transcription termination function; and/or
(ii) the nucleotide sequence set forth as SEQ ID NO: 235.

89. The genetic construct of any one of claims 71-88, wherein the genetic construct comprises at least one terminator 5′ of the promoter.

90. The genetic construct of claim 89, wherein the at least one terminator 5′ of the promoter comprises:
i) a nucleotide sequence having at least 80% sequence identity to a nucleotide sequence set forth as SEQ ID NO: 237, wherein the nucleotide sequence retains transcription termination function; and/or
(ii) a nucleotide sequence set forth as SEQ ID NO: 237.

91. The genetic construct of any one of claims 71-90, wherein the polynucleotide encoding the first fusion protein comprises a signal sequence.

92. The genetic construct of claim 91, wherein the signal sequence encodes a signal peptide having
(i) an amino acid sequence having at least 80% sequence identity to an amino acid sequence set forth as any of SEQ ID NOs: 210, 211, and 212, wherein said signal peptide retains signal peptide function;
and/or (ii) the amino acid sequence set forth as any of SEQ ID NOs: 210, 211, and 212.

93. The genetic construct of claim 92, wherein the signal peptide is a lipoprotein anchor.

94. The genetic construct of any one of claims 71-93, further comprising a polynucleotide encoding a UspA polypeptide.

95. The genetic construct of claim 94, wherein the UspA polypeptide:
(i) comprises an amino acid sequence having at least 80% sequence identity to an amino acid sequence set forth as SEQ ID NO: 261 or 263, wherein said UspA polypeptide retains immunogenicity;
(ii) comprises the amino acid sequence set forth as SEQ ID NO: 261 or 263;
(iii) is encoded by a nucleotide sequence having at least 80% sequence identity to a nucleotide sequence set forth as SEQ ID NO: 262 or 264, wherein the nucleotide sequence encodes a UspA protein that retains immunogenicity; and/or
(iv) is encoded by the nucleotide sequence set forth as SEQ ID NO: 262 or 264.

96. The genetic construct of claim 94 or 95, wherein the first fusion protein further comprises the UspA polypeptide.

97. The genetic construct of any one of claims 71-96, wherein the first fusion protein comprises:
(i) an amino acid sequence having at least 80% sequence identity to an amino acid sequence set forth as any of SEQ ID NOs: 219, 223, and 226, wherein said fusion protein retains immunogenicity and anchoring at the cell surface; and/or
(ii) the amino acid sequence set forth as any of SEQ ID NOs: 219, 223, and 226.

98. The genetic construct of any one of claims 71-97, wherein the first fusion protein is encoded by:
(i) a nucleotide sequence having at least 80% sequence identity to a nucleotide sequence set forth as any of SEQ ID NOs: 220, 224, and 227, wherein the nucleotide sequence encodes a fusion protein that retains immunogenicity and anchoring at the cell surface; and/or
(ii) the nucleotide sequence set forth as any of SEQ ID NOs: 220, 224, and 227.

99. The genetic construct of claim 94 or 95, further comprising a polynucleotide encoding a second fusion protein comprising the UspA polypeptide and a surface anchor moiety operably linked to a promoter.

100. The genetic construct of any one of claims 71-99, wherein the genetic construct comprises a nucleotide sequence set forth as any of SEQ ID NOs: 229, 230, 231, 232, 233, and 234.

101. A cell comprising the genetic construct of any one of claims 71-100.

102. The cell of claim 101, wherein the cell comprises the nucleotide sequence set forth as any of SEQ ID NOs: 229, 230, 231, 232, 233, and 234.

103. The cell of claim 101 or 102, wherein the cell is an S. pneumoniae cell.

104. The cell of claim 103, wherein the S. pneumoniae cell is a live attenuated S. pneumoniae cell.

105. The cell of claim 104, wherein the live attenuated S. pneumoniae cell comprises a disruption in an ftsY gene in its genome.

106. A method of producing a recombinant S. pneumoniae bacterium, comprising introducing into a S. pneumoniae bacterium the genetic construct of any one of claims 71-100.

107. The method of claim 106, wherein the S. pneumoniae bacterium is a live attenuated S. pneumoniae comprising a disruption in an ftsY gene in its genome.

108. A method of producing an immunogenic composition, the method comprising:
introducing at least one fusion protein into a recombinant live attenuated Streptococcus pneumoniae (S. pneumoniae), wherein each fusion protein comprises:
(i) an immunogenic protein, or an immunogenic fragment or variant thereof; and
(ii) a surface anchor moiety,
to produce a modified recombinant live attenuated S. pneumoniae, wherein each fusion protein is expressed at the cell surface of the modified recombinant live attenuated S. pneumoniae.

109. The method of claim 108, wherein introducing each fusion protein into the recombinant live attenuated S. pneumoniae comprises introducing at least one nucleic acid molecule comprising a nucleotide sequence encoding the at least one fusion protein.

110. The method of claim 108 or 109, wherein the recombinant live attenuated S. pneumoniae comprises a disruption of an ftsY gene in its genome.

111. The method of any one of claims 108-110, wherein the modified recombinant live attenuated S. pneumoniae comprises in its genome a nucleotide sequence set forth as any of SEQ ID NOs: 229, 230, 231, 232, 233, and 234.

112. The method of any one of claims 108-111, further comprising formulating the modified recombinant live attenuated S. pneumoniae with a pharmaceutically acceptable carrier.

113. The method of any one of claims 108-112, further comprising formulating the modified recombinant live attenuated S. pneumoniae with an immunological adjuvant.

114. The method of any one of claims 108-113, further formulating the modified recombinant live attenuated S. pneumoniae for intranasal administration.
